(19) United States
(12) Patent Application Publication
(10) Pub. No.: US 2010/0022515 A1
(43) Pub. Date: Jan. 28, 2010

(54) COMPOUNDS AND COMPOSITIONS AS MODULATORS OF GPR119 ACTIVITY

(75) Inventors: Phillip B. Alper, San Diego, CA (US); Mihai D. Azimioara, La Jolla, CA (US); Christopher Cow, San Diego, CA (US); Robert Epple, San Diego, CA (US); Songchun Jiang, San Diego, CA (US); Gerald Lelais, San Diego, CA (US); Pierre-Yves Michellys, San Marcos, CA (US); Truc Ngoc Nguyen, San Diego, CA (US); Lucas Westcott-Baker, Goleta, CA (US); Baogen Wu, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 12/525,289

(22) PCT Filed: Jan. 22, 2008
(86) PCT No.: PCT/US08/00864

§ 371 Date: Jul. 30, 2009

Related U.S. Application Data
(60) Provisional application No. 60/888,033, filed on Feb. 2, 2007.

(51) Int. Cl. A61K 31/55; C07D 401/14; A61K 31/4709; A61K 31/5377; A61K 31/497; C07D 413/14; C07D 401/12

(52) U.S. Cl. 514/213.01; 546/153; 514/312; 514/235.2; 514/253.07; 544/363; 544/128; 540/593

(57) ABSTRACT

The invention provides compounds, pharmaceutical compositions comprising compound of following formula (I), and methods of using such compounds to treat or prevent diseases or disorders associated with the activity of GPR119 (G protein-coupled receptor 119) such as obesity, diabetes and hyperlipidemia.

### CROSS-REFERENCE TO RELATED APPLICATIONS

This application claims the benefit of priority to U.S. Provisional Patent Application No. 60/888,033, filed 2 Feb. 2007. The full disclosure of this application is incorporated herein by reference in its entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activity of GPR119.

2. Background

GPR119 is a G-protein coupled receptor (GPCR) that is mainly expressed in the pancreas, small intestine, colon and adipose tissue. The expression profile of the human GPR119 receptor indicates its potential utility as a target for the treatment of obesity and diabetes. The novel compounds of this invention modulate the activity of GPR119 and are, therefore, expected to be useful in the treatment of GPR119-associated diseases or disorders such as, but not limited to, diabetes, obesity and associated metabolic disorders.

### SUMMARY OF THE INVENTION

In one aspect, the present invention provides a compound of Formula I:

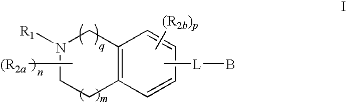

in which:

B is selected from C₆₋₁₀aryl, C₁₋₁₀heteroaryl, C₃₋₁₂cycloalkyl and C₃₋₈heterocycloalkyl; wherein said aryl, heteroaryl, cycloalkyl or heterocycloalkyl of B is substituted with one to three radicals selected from —R₃ and —OXₐR₃; wherein Xₐ is selected from a bond and C₁₋₃alkylene; and wherein any heterocycloalkyl of B can have a CH₂ group replaced with C(O);

n and p are independently selected from 0, 1, 2 and 3;

q is selected from 0, 1 and 2;

m is selected from 1, 2 and 3;

L is —X₁-A—X₂—B₁—X₃—; wherein A and B₁ are independently selected from a bond, —O—, —S(O)₀₋₂—, —C(O)—, —C(O)O—, —OC(O)—, —NR₄—, —C(O)NR₄—, —C(S)NR₄, —NR₄C(O)—, —CR₄(NR₄C(O)R₄)—, —C(═NOR₄)—, —CR₄(NR₄R₄)—, —CR₄(OR₄)—, —CR₄R₄C(O)OR₄—, —N(C(O)R₄)— and —NR₄C(S)—; wherein X₁, X₂ and X₃ are independently selected from a bond, C₁₋₆alkylene, C₂₋₆alkenylene, C₃₋₈cycloalkyl, C₆₋₁₀aryl, C₃₋₈heterocycloalkyl and C₁₋₆heteroarylene; wherein said cycloalkyl, aryl, heterocycloalkyl or heteroaryl of L can be optionally substituted with up to 3 radicals independently selected from hydroxyl, halo, C₁₋₆alkyl, C₁₋₆alkoxy, halo-substituted-C₁₋₆alkyl and halo-substituted-C₁₋₆alkoxy; each R₄ is independently selected from hydrogen, hydroxyl, halo, C₁₋₆alkyl, halo-substituted-C₁₋₆alkyl and halo-substituted-C₁₋₆alkoxy; with the proviso that when A and B are the same moiety, X₂ cannot be a bond; wherein any methylene of L can have the hydrogens replaced by a radical selected from halo, hydroxy, C₁₋₄alkyl, C₁₋₄alkoxy, hydroxy-substituted-C₁₋₄alkyl, —CR₄R₄C(O)OR₄, X₄OR₄ₐ, —X₄NR₄R₄ₐ, X₄NR₄ₐX₄OR₄ₐ, —X₄C(O)OR₄ₐ and —X₄C(O)R₄ₐ; wherein X₄ is selected from a bond and C₁₋₄alkylene; R₄ₐ is selected from hydrogen and C₄alkyl;

R₁ is selected from C₁₋₁₀alkyl, halo-substituted-C₁₋₁₀alkyl, C₆₋₁₀aryl, C₁₋₁₀heteroaryl, —X₅S(O)₀₋₂R₅ₐ, —X₅C(O)OR₅ₐ, —X₅C(O)R₅ₐ, and —X₅C(O)NR₅ₐR_{5b}; wherein X₅ is selected from a bond and C₁₋₃alkylene; R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy, —NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;

R₂ₐ and R_{2b} are independently selected from halo, cyano, hydroxy, C₁₋₄alkyl, amino, nitro, —C(O)OR₅ₑ, —C(O)R₅ₑ and —NR₅ₑR_{5f}; wherein R₅ₑ and R_{5f} are independently selected from hydrogen, C₁₋₆alkyl, C₃₋₁₂cycloalkyl, halo-substituted-C₁₋₆alkyl, halo-substituted-C₁₋₆cycloalkyl, C₆₋₁₀aryl and C₁₋₁₀heteroaryl; wherein said aryl or heteroaryl of R₅, or R_{5f} can be optionally substituted with 1 to 3 radicals independently selected from C₁₋₆alkyl, C₁₋₆alkoxy, halo-substituted-C₁₋₆alkyl and halo-substituted-C₁₋₆alkoxy;

R₃ is selected from hydrogen, C₁₋₁₀heteroaryl, C₆₋₁₀aryl, C₃₋₈heterocycloalkyl, —C(O)OR₆ₐ, —C(O)R₆ₐ, —S(O)₀₋₂R₆ₐ, —C(O)R₇, —C(O)X₅NR₆ₐC(O)OR_{6b}, —C(S)OR₆ₐ, —C(S)R₆ₐ, —C(S)R₇ and —C(S)X₅NR₆ₐC(O)OR_{6b}; wherein X₅ is selected from a bond and C₁₋₆alkylene; R₆ₐ and R_{6b} are independently selected from hydrogen, C₁₋₆alkyl, halo-substituted-C₁₋₆alkyl, C₃₋₁₂cycloalkyl optionally substituted with C₁₋₄alkyl, halo-substituted-C₁₋₆cycloalkyl; R₇ is selected from C₁₋₈alkyl, C₃₋₈cycloalkyl, C₆₋₁₀aryl, C₁₋₁₀heteroaryl, halo-substituted C₁₋₈alkyl, halo-substituted-C₃₋₈cycloalkyl, halo-substituted-C₆₋₁₀aryl and halo-substituted-C₆₋₁₀heteroaryl; wherein said aryl, heteroaryl or heterocycloalkyl of R₃ is optionally substituted with 1 to 3 radicals independently selected from halo, cyano, —X₅ₐNR₅ₐR_{5b}, —X₅ₐNR₅ₐR₉, —X₅ₐNR₅ₐC(O)OR_{5b}, —X₅ₐC(O)OR₈ₐ, —X₅ₐOR₈ₐ, —X₅ₐOX_{5b}OR₈ₐ, —X₅ₐC(O)R₈ₐ, —X₅ₐR₉, C₁₋₆alkyl, C₁₋₆alkoxy, halo-substituted-C₁₋₆alkyl and halo-substituted-C₁₋₆alkoxy; wherein R₈ₐ and R_{9b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl; wherein said aryl, heteroaryl, cycloalkyl or heterocycloalkyl of R₉ is optionally substituted with 1 to 3 radicals independently selected from halo, C₁₋₄alkyl and C₁₋₄alkoxy; or the pharmaceutically acceptable salts thereof.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of GPR119 activity can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which GPR119 activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be straight-chained, branched, cyclic or spiro. C₁₋₆alkoxy includes methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group. “Heteroaryl” is as defined for aryl where one or more of the ring members are a heteroatom. For example, C₁₋₁₀heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, 1H-pyridin-2-onyl, 6-oxo-1,6-dihydro-pyridin-3-yl, etc. “C₆₋₁₀arylC₀₋₄alkyl” means an aryl as described above connected via a alkylene grouping. For example, C₆₋₁₀arylC₀₋₄alkyl includes phenethyl, benzyl, etc. Heteroaryl also includes the N-oxide derivatives, for example, pyridine N-oxide derivatives with the following structure:

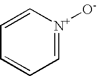

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, 2-oxo-pyrrolidin-1-yl, 2-oxo-piperidin-1-yl, etc.

GPR119 means G protein-coupled receptor 119 (GenBank® Accession No. AAP72125) is also referred to in the literature as RUP3 and GPR116. The term GPR119 as used herein includes the human sequences found in GeneBank accession number AY288416, naturally-occurring allelic variants, mammalian orthologs, and recombinant mutants thereof.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases in which modulation of GPR119 activity can prevent, inhibit or ameliorate the pathology and/or symptomology of the diseases, which method comprises administering to the animal a therapeutically effective amount of a compound of Formula I.

In one embodiment, with reference to compounds of Formula I, are compounds of Formula Ia:

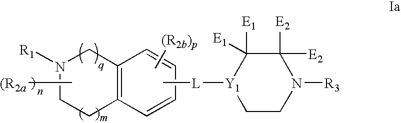

in which:

n and p are independently selected from 0, 1, 2 and 3;

q is selected from 0 and 1;

m is selected from 1, 2 and 3;

E₁ is hydrogen or both E₁ radicals, together with the carbon atom to which they are attached, can form C(═O);

E₂ is hydrogen or both E₂ radicals, together with the carbon atom to which they are attached, can form C(═O);

L is selected from C₁₋₁₀heteroarylene, —X₂OX₃—, —OX₂X₃—, —C(O)X₂—, —X₂X₃—, —OX₂O—, —OX₂C(O)X₃—, —OX₂C(O)OX₃—, —CR₄(NR₄R₄)X₂—, —CR₄(NR₄C(O)R₄)X₂—, —C(═NOR₄)X₂—, —NR₄C(O)X₂—, —C(O)NR₄X₂—, —NR₄X₂—, —N(C(O)R₄)X₂— and —OC(O)NR₄X₂—; wherein X₂ and X₃ are independently selected from a bond, C₁₋₆alkylene, C₂₋₆alkenylene, C₆₋₁₀aryl, C₃₋₈cycloalkyl and C₁₋₁₀heteroarylene; R₄ is selected from hydrogen and C₁₋₆alkyl; wherein any methylene of L can have the hydrogens replaced by a radical selected from halo, hydroxy, C₁₋₄alkyl, C₁₋₄alkoxy, hydroxy-substituted-C₁₋₄alkyl and —CR₄R₄C(O)OR₄;

R₁ is selected from C₁₋₁₀alkyl, halo-substituted-C₁₋₁₀alkyl, C₆₋₁₀aryl, C₁₋₁₀heteroaryl, —X₅S(O)₀₋₂R₅ₐ, —X₅C(O)OR₅ₐ, —X₅C(O)R₅ₐ, and —X₅C(O)NR₅ₐR_{5b}; wherein X₅ is selected from a bond and C₁₋₃alkylene; R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy, —NR_{5c}R_{5d}, —C(O)OR₅, and C₆₋₁₀aryl-C₀₋₄alkyl; wherein R_{5c} and R_{5d} are independently selected from hydrogen and C₁₋₆alkyl;

R₂ₐ and R_{2b} are independently selected from halo, methyl, cyano and nitro;

R₃ is selected from aryl, C₁₋₁₀heteroaryl and —C(O)OR₆ₐ; wherein R₆ₐ is selected from hydrogen, C₁₋₆alkyl and C₃₋₁₂cycloalkyl optionally substituted with C₁₋₄alkyl; wherein said heteroaryl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐR₉, C₁₋₆alkyl, C₁₋₆alkoxy and halo-substituted-C₁₋₆alkyl; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl-C₀₋₄alkyl; wherein said aryl, heteroaryl, cycloalkyl or heterocycloalkyl of R₉ is optionally substituted with 1 to 3 radicals independently selected from halo, C₁₋₄alkyl and C₁₋₄alkoxy; and

Y₁ is selected from CH and N.

In a further embodiment, L is selected from 3,5-1,2,4-oxadiazolylene, (1,2,4-oxadiazol-5-yl)methoxy, (1,2,4-oxadiazol-5-yl)methyl, (1,2,4-oxadiazol-5-yl)ethyl, (1,2,4-oxadiazol-5-yl)propyl, phenoxy, phenoxy-methyl, —C(O)NHCH₂—, —C(O)NH(CH₂)₂—, —CH₂OCH₂—, —C(O)NH(CH₂)₃—, —CH((CH₂)₂OH)(CH₂)₃—, —CH(CH₂C(O)OCH₃)(CH₂)₃—, —C(O)(CH₂)₃—, —CH(OH)(CH₂)₃—, —CH(Cl)(CH₂)₃—, —C(CH₃)(OH)(CH₂)₃—, —CH(N(CH₃)₂)(CH₂)₃—, —CH(NH₂)(CH₂)₃—, —CH(NHC(O)H)(CH₂)₃—, —CF₂(CH₂)₃—, —O(CH₂)₂—, —(CH₂)₂—, —(CH₂)₃—, —(CH₂)₄—, —O(CH₂)₄—, —O(CH₂)₃—, —NH(CH₂)₂—, —NH(CH₂)₃—, —C(═NOCH₃)(CH₂)₃—, —C(═NOH)(CH₂)₃—, —NHC(O)(CH₂)₃—, —NH(CH₂)₄—, —NCH₃(CH₂)₄—, —N(C(O)CH₃)(CH₂)₃—, —NC₂H₅(CH₂)₃—, —NC₃H₇(CH₂)₃—, —O(CH₂)₃O—, —O(CH₂)₂O—, —CH═CH(CH₂)₂—; —CH═CH—; —OCH₂CH(CH₂OH)O—; —C(O)CH(N(CH₂)₂—O—(CH₂)₂)—(CH₂)₂—; —NCH₃(CH₂)₃—; —N(CH(CH₃)₂)(CH₂)₃—; —NHC(O)(CH₂)₂—; —CH₂—O—(CH₂)₂—; —CH₂—O—(CH₂)₃—; —CH₂—O—(CH₂)₄—; —CH═CHCH₂—; —CH(CH₂COOH)(CH₂)₃—; —CH(OCH₃)(CH₂)₃—; —CH(F)(CH₂)₃—; —C(OH)(CH₂OH)(CH₂)₃—; —CH(CH₂OH)(CH₂)₃—;

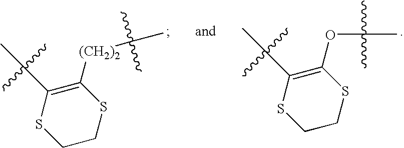

In a further embodiment, R₁ is selected from methyl-sulfonyl, butyl-sulfonyl, phenyl-sulfonyl, isopropyl-sulfonyl, ethyl-sulfonyl, ethenyl-sulfonyl, isopropoxy-carbonyl, benzyloxy-carbonyl, ethoxy-carbonyl, methyl-sulfonyl-ethyl, methoxy-carbonyl, t-butoxy-carbonyl and trifluoromethyl-sulfonyl.

In a further embodiment, R₃ is selected from t-butoxy-carbonyl, dimethylamino-carbonyl, methyl-sulfonyl, isopropoxy-carbonyl(ethyl)amino-methyl, isopropoxy-carbonyl-amino-methyl, benzyl(ethyl)amino-methyl, piperidinyl, quinazolinyl, isopropoxy-carbonyl, thieno[2,3-d]pyrimidin-4-yl, 4H-1,2,4-triazolyl, cyclopropoxy-carbonyl, (1,2,4-oxadiazol-5-yl), tetrazolyl, thiazolyl, triazolyl, pyrimidinyl, pyrazinyl, pyridinyl, phenyl, benzimidazolyl and pyridazinyl; wherein said cyclopropoxy, quinazolinyl, thieno[2,3-d]pyrimidinyl, thiazolyl, oxadiazolyl, tetrazolyl, pyrimidinyl, pyrazinyl, pyridinyl, phenyl, benzimidazolyl or pyridazinyl can be optionally substituted by 1 to 2 radicals independently selected from halo, cyano, methyl, methoxy-carbonyl, carboxyl, isopropyl, t-butyl, cyclopropyl, morpholino, methyl-piperazinyl-methyl, morpholino-methyl, ethoxy-methoxy-methyl, hydroxy-methyl, methoxy-ethoxy-methyl, methoxy-methoxy-methyl, ethoxy, trifluoromethyl, pentyl, phenyl, methoxy, dimethylamino, dimethylamino-methyl, dimethylamino-ethyl, aminoethyl, methoxy-carbonyl-methyl, methoxy-ethyl, hydroxyl-ethyl, pyrrolidinoethyl, t-butoxycarbonylamino-propoxy-methyl, morpholino-ethyl, aminopropoxy-methyl, dimethylamino-methyl, diethylamino-methyl, isopropyl-piperazino-ethyl, methoxy-ethoxy-ethoxy-methyl, methoxy-methyl, propyl and ethyl.

In a further embodiment, are compounds selected from: tert-butyl 4-(2-(2-(methylsulfonyl)-1,2,3,4-tetrahydroisoquinolin-5-yloxy)ethyl)piperidine-1-carboxylate; tert-butyl 4-(3-(2-(methylsulfonyl)-1,2,3,4-tetrahydroisoquinolin-5-yloxy)propyl)piperidine-1-carboxylate; tert-butyl 4-(2-(2-(methylsulfonyl)-1,2,3,4-tetrahydroisoquinolin-6-yloxy)ethyl)piperidine-1-carboxylate; tert-butyl 4-(3-(2-(methylsulfonyl)-1,2,3,4-tetrahydroisoquinolin-6-yloxy)propyl)piperidine-1-carboxylate; tert-butyl 4-(2-(2-(methylsulfonyl)-1,2,3,4-tetrahydroisoquinolin-7-yloxy)ethyl)piperidine-1-carboxylate; tert-butyl 4-(3-(2-(methylsulfonyl)-1,2,3,4-tetrahydroisoquinolin-7-yloxy)propyl)piperidine-1-carboxylate; tert-butyl 4-(3-(1-(methylsulfonyl)-1,2,3,4-tetrahydroquinolin-6-yloxy)propyl)piperidine-1-carboxylate; isopropyl 4-(2-(2-(methylsulfonyl)-1,2,3,4-tetrahydroisoquinolin-6-yloxy)ethyl)piperidine-1-carboxylate; isopropyl 4-(4-(2-(methylsulfonyl)-1,2,3,4-tetrahydroisoquinolin-6-yloxy)butyl)piperidine-1-carboxylate; isopropyl 4-(2-(2-(ethylsulfonyl)-1,2,3,4-tetrahydroisoquinolin-6-yloxy)ethyl)piperidine-1-carboxylate; isopropyl 4-(3-(2-(methylsulfonyl)-1,2,3,4-tetrahydroisoquinolin-6-yloxy)propyl)piperidine-1-carboxylate; isopropyl 4-(5-((2-(methylsulfonyl)-1,2,3,4-tetrahydroisoquinolin-6-yloxy)methyl)-1,2,4-oxadiazol-3-yl)piperidine-1-carboxylate; isopropyl 4-(2-(2-(isopropylsulfonyl)-1,2,3,4-tetrahydroisoquinolin-6-yloxy)ethyl)piperidine-1-carboxylate; isopropyl 4-(2-(2-(vinylsulfonyl)-1,2,3,4-tetrahydroisoquinolin-6-yloxy)ethyl)piperidine-1-carboxylate; isopropyl 6-(2-(1-(isopropoxycarbonyl)piperidin-4-yl)ethoxy)-3,4-dihydroisoquinoline-2(1H)-carboxylate; isopropyl 4-(2-(2-(butylsulfonyl)-1,2,3,4-tetrahydroisoquinolin-6-yloxy)ethyl)piperidine-1-carboxylate; isopropyl 4-(2-(2-(phenylsulfonyl)-1,2,3,4-tetrahydroisoquinolin-6-yloxy)ethyl)piperidine-1-carboxylate; ethyl 6-(2-(1-(isopropoxycarbonyl)piperidin-4-yl)ethoxy)-3,4-dihydroisoquinoline-2(1H)-carboxylate; benzyl 6-(2-(1-(isopropoxycarbonyl)piperidin-4-yl)ethoxy)-3,4-dihydroisoquinoline-2(1H)-carboxylate; isopropyl 4-(4-(2-(methylsulfonyl)-1,2,3,4-tetrahydroisoquinolin-6-yloxy)butyl)piperidine-1-carboxylate; methyl 6-(2-(1-(isopropoxycarbonyl)piperidin-4-yl)ethoxy)-3,4-dihydroisoquinoline-2(1H)-carboxylate; isopropyl 4-(2-(2-(trifluoromethylsulfonyl)-1,2,3,4-tetrahydroisoquinolin-6-yloxy)ethyl)piperidine-1-carboxylate; 6-(3-(1-(5-ethylpyrimidin-2-yl)piperidin-4-yl)propoxy)-2-(methylsulfonyl)-1,2,3,4-tetrahydroisoquinoline; 6-(2-(1-(5-ethylpyrimidin-2-yl)piperidin-4-yl)ethoxy)-2-(methylsulfonyl)-1,2,3,4-tetrahydroisoquinoline; Isopropyl 4-(2-(2-(methylsulfonyl)-1,2,3,4-tetrahydroisoquinolin-6-ylamino)ethyl)-piperidine-1-carboxylate; Isopropyl 4-(3-(2-(methylsulfonyl)-1,2,3,4-tetrahydroisoquinolin-6-ylamino)propyl)-piperidine-1-carboxylate; Isopropyl 4-(3-(2-(methylsulfonyl)-1,2,3,4-tetrahydroisoquinolin-6-ylamino)butyl)-piperidine-1-carboxylate; Tert-butyl 6-(3-(1-(isopropoxycarbonyl)piperidin-4-yl)propylamino)-3,4-dihydroisoquinoline-2(1H)-carboxylate; Tert-butyl 6-(4-(1-(isopropoxycarbonyl)piperidin-4-yl)butylamino)-3,4-dihydroisoquinoline-2(1H)-carboxylate; Isopropyl 4-(3-(methyl(2-(methylsulfonyl)-1,2,3,4-tetrahydroisoquinolin-6-yl)amino)butyl)piperidine-1-carboxylate; isopropyl 4-(3-(methyl(2-(methylsulfonyl)-1,2,3,4-tetrahydroisoquinolin-6-yl)amino)propyl)piperidine-1-carboxylate; isopropyl 4-(3-(ethyl(2-(methylsulfonyl)-1,2,3,4-tetrahydroisoquinolin-6-yl)amino)propyl)piperidine-1-carboxylate; isopropyl 4-(3-((2-(methylsulfonyl)-1,2,3,4-tetrahydroisoquinolin-6-yl)(propyl)amino)propyl)piperidine-1-carboxylate; isopropyl 4-(3-(isopropyl(2-(methylsulfonyl)-1,2,3,4-tetrahydroisoquinolin-6-yl)amino)propyl)piperidine-1-carboxylate; isopropyl 4-(3-(N-(2-(methylsulfonyl)-1,2,3,4-tetrahydroisoquinolin-6-yl)acetamido)propyl)piperidine-1-carboxylate; isopropyl 4-(4-(2-(methylsulfonyl)-1,2,3,4-tetrahydroisoquinolin-6-ylamino)-4-oxobutyl)piperidine-1-carboxylate; tert-butyl 4-(3-(2-(methylsulfonyl)-1,2,3,4-tetrahydroisoquinolin-6-ylamino)-3-oxopropyl)piperidine-1-carboxylate; tert-butyl 4-(4-(2-(methylsulfonyl)-1,2,3,4-tetrahydroisoquinolin-6-ylamino)-4-oxobutyl)piperidine-1-carboxylate; Tert-butyl 4-((2-(methylsulfonyl)-1,2,3,4-tetrahydroisoquinoline-6-carboxamido)methyl)piperidine-1-carboxylate; Isopropyl 4-(2-(2-(methylsulfonyl)-1,2,3,4-tetrahydroisoquinoline-6-carboxamido)ethyl)piperidine-1-carboxylate; Isopropyl 4-(2-(2-(methylsulfonyl)-1,2,3,4-tetrahydroisoquinoline-6-carboxamido)propyl)piperidine-1-carboxylate; Isopropyl 4-(((2-(methylsulfonyl)-1,2,3,4-tetrahydroisoquinolin-6-yl)methoxy)methyl)piperidine-1-carboxylate; isopropyl 4-(2-((2-(methylsulfonyl)-1,2,3,4-tetrahydroisoquinolin-6-yl)methoxy)ethyl)piperidine-1-carboxylate; isopropyl 4-(3-((2-(methylsulfonyl)-1,2,3,4-tetrahydroisoquinolin-6-yl)methoxy)propyl)piperidine-1-carboxylate; isopropyl 4-(4-((2-(methylsulfonyl)-1,2,3,4-tetrahydroisoquinolin-6-yl)methoxy)butyl)piperidine-1-carboxylate; isopropyl 4-(5-(2-(methylsulfonyl)-1,2,3,4-tetrahydroisoquinolin-6-yl)-1,2,4-oxadiazol-3-yl)piperidine-1-carboxylate; Isopropyl 4-((5-(2-(methylsulfonyl)-1,2,3,4-tetrahydroisoquinolin-6-yl)-1,2,4-oxadiazol-3-yl)methyl)piperidine-1-carboxylate; Isopropyl 4-(2-(5-(2-(methylsulfonyl)-1,2,3,4-tetrahydroisoquinolin-6-yl)-1,2,4-oxadiazol-3-yl)ethyl)piperidine-1-carboxylate; Tert-butyl 4-((5-(2-(methylsulfonyl)-1,2,3,4-tetrahydroisoquinolin-6-yl)-1,2,4-oxadiazol-3-yl)methyl)piperidine-1-carboxylate; 3-((1-(5-ethylpyrimidin-2-yl)piperidin-4-yl)methyl)-5-(2-(methylsulfonyl)-1,2,3,4-tetrahydroisoquinolin-6-yl)-1,2,4-oxadiazole; 5-(2-(methylsulfonyl)-1,2,3,4-tetrahydroisoquinolin-6-yl)-3-((1-(pyrimidin-2-yl)piperidin-4-yl)methyl)-1,2,4-oxadiazole; 5-(2-(methylsulfonyl)-1,2,3,4-tetrahydroisoquinolin-6-yl)-3-((1-(pyridin-2-yl)piperidin-4-yl)methyl)-1,2,4-oxadiazole; 3-((1-(6-ethylpyridazin-3-yl)piperidin-4-yl)methyl)-5-(2-(methylsulfonyl)-1,2,3,4-tetrahydroisoquinolin-6-yl)-1,2,4-oxadiazole; 3-((1-(3-chloro-5-(trifluoromethyl)pyridin-2-yl)piperidin-4-yl)methyl)-5-(2-(methylsulfonyl)-1,2,3,4-tetrahydroisoquinolin-6-yl)-1,2,4-oxadiazole; 3-((1-(6-bromopyridin-3-yl)piperidin-4-yl)methyl)-5-(2-(methylsulfonyl)-1,2,3,4-tetrahydroisoquinolin-6-yl)-1,2,4-oxadiazole; 3-((1-(5-fluoropyridin-2-yl)piperidin-4-yl)methyl)-5-(2-(methylsulfonyl)-1,2,3,4-tetrahydroisoquinolin-6-yl)-1,2,4-oxadiazole; 5-(2-(methylsulfonyl)-1,2,3,4-tetrahydroisoquinolin-6-yl)-3-((1-(5-(trifluoromethyl)pyridin-2-yl)piperidin-4-yl)methyl)-1,2,4-oxadiazole; 2-(methylsulfonyl)-6-(3-((1-(5-(trifluoromethyl)pyridin-2-yl)piperidin-4-yl)methyl)-1,2,4-oxadiazol-5-yl)-1,2,3,4-tetrahydroisoquinolin-1-ol; 1-methylcyclopropyl 4-((5-(2-(methylsulfonyl)-1,2,3,4-tetrahydroisoquinolin-6-yl)-1,2,4-oxadiazol-3-yl)methyl)piperidine-1-carboxylate; Tert-butyl 4-((3-(2-(methylsulfonyl)-1,2,3,4-tetrahydroisoquinolin-6-yl)-1,2,4-oxadiazol-5-yl)methyl)piperidine-1-carboxylate; tert-butyl 4-(2-(3-(2-(methylsulfonyl)-1,2,3,4-tetrahydroisoquinolin-6-yl)-1,2,4-oxadiazol-5-yl)ethyl)piperidine-1-carboxylate; tert-butyl 4-(3-(3-(2-(methylsulfonyl)-1,2,3,4-tetrahydroisoquinolin-6-yl)-1,2,4-oxadiazol-5-yl)propyl)piperidine-1-carboxylate; isopropyl 4-((3-(2-(methylsulfonyl)-1,2,3,4-tetrahydroisoquinolin-6-yl)-1,2,4-oxadiazol-5-yl)methyl)piperidine-1-carboxylate; Isopropyl 4-((3-(2-(methylsulfonyl)-1,2,3,4-tetrahydroisoquinolin-6-yl)-1,2,4-oxadiazol-5-yl)methyl)piperidine-1-carboxylate; 5-((1-(5-ethylpyrimidin-2-yl)piperidin-4-yl)methyl)-3-(2-(methylsulfonyl)-1,2,3,4-tetrahydroisoquinolin-6-yl)-1,2,4-oxadiazole; (E)-isopropyl 4-(4-(2-(methylsulfonyl)-1,2,3,4-tetrahydroisoquinolin-6-yl)but-3-enyl)piperidine-1-carboxylate; (E)-isopropyl 4-(3-(2-(methylsulfonyl)-1,2,3,4-tetrahydroisoquinolin-6-yl)allyl)piperidine-1-carboxylate; (E)-isopropyl 4-(2-(2-(methylsulfonyl)-1,2,3,4-tetrahydroisoquinolin-6-yl)vinyl)piperidine-1-carboxylate; Isopropyl 4-(4-(2-(methylsulfonyl)-1,2,3,4-tetrahydroisoquinolin-6-yl)butyl)piperidine-1-carboxylate; isopropyl 4-(3-(2-(methylsulfonyl)-1,2,3,4-tetrahydroisoquinolin-6-yl)propyl)piperidine-1-carboxylate; isopropyl 4-(2-(2-(methylsulfonyl)-1,2,3,4-tetrahydroisoquinolin-6-yl)ethyl)piperidine-1-carboxylate; Isopropyl 4-(3-(2-(methylsulfonyl)-1,2,3,4-tetrahydroisoquinolin-6-yl)phenoxy)piperidine-1-carboxylate; Isopropyl 4-((3-(2-(methylsulfonyl)-1,2,3,4-tetrahydroisoquinolin-6-yl)phenoxy)methyl)piperidine-1-carboxylate; Isopropyl 4-(4-(2-(methylsulfonyl)-1,2,3,4-tetrahydroisoquinolin-6-yl)-4-oxobutyl)piperidine-1-carboxylate; Isopropyl 4-(4,4-difluoro-4-(2-(methylsulfonyl)-1,2,3,4-tetrahydroisoquinolin-6-yl)butyl)piperidine-1-carboxylate; Isopropyl 4-(4-(1-(methylsulfonyl)-2,3,4,5-tetrahydro-1H-benzo[b]azepin-7-yloxy)butyl)piperidine-1-carboxylate; 2-(methylsulfonyl)-6-(3-(1-(5-pentylpyrimidin-2-yl)piperidin-4-yl)propoxy)-1,2,3,4-tetrahydroisoquinoline; 2-(methylsulfonyl)-6-(3-(1-(5-propylpyrimidin-2-yl)piperidin-4-yl)propoxy)-1,2,3,4-tetrahydroisoquinoline; 2-(methylsulfonyl)-6-(3-(1-(5-phenylpyrimidin-2-yl)piperidin-4-yl)propoxy)-1,2,3,4-tetrahydroisoquinoline; 6-(3-(1-(5-bromopyrimidin-2-yl)piperidin-4-yl)propoxy)-2-(methylsulfonyl)-1,2,3,4-tetrahydroisoquinoline; 6-(3-(1-(5-fluoropyrimidin-2-yl)piperidin-4-yl)propoxy)-2-(methylsulfonyl)-1,2,3,4-tetrahydroisoquinoline; 2-(methylsulfonyl)-6-(3-(1-(4-(trifluoromethyl)pyrimidin-2-yl)piperidin-4-yl)propoxy)-1,2,3,4-tetrahydroisoquinoline; 6-(3-(1-(4-methoxypyrimidin-2-yl)piperidin-4-yl)propoxy)-2-(methylsulfonyl)-1,2,3,4-tetrahydroisoquinoline; N,N-dimethyl-2-(4-(3-(2-(methylsulfonyl)-1,2,3,4-tetrahydroisoquinolin-6-yloxy)propyl)piperidin-1-yl)pyrimidin-4-amine; 2-(methylsulfonyl)-6-(3-(1-(4-phenylpyrimidin-2-yl)piperidin-4-yl)propoxy)-1,2,3,4-tetrahydroisoquinoline; 6-(3-(1-(4-methylpyrimidin-2-yl)piperidin-4-yl)propoxy)-2-(methylsulfonyl)-1,2,3,4-tetrahydroisoquinoline; 2-(methylsulfonyl)-6-(3-(1-(pyrimidin-2-yl)piperidin-4-yl)propoxy)-1,2,3,4-tetrahydroisoquinoline; 2-(methylsulfonothioyl)-6-(3-(1-(pyrazin-2-yl)piperidin-4-yl)propoxy)-1,2,3,4-tetrahydroisoquinoline; 2-(methylsulfonyl)-6-(3-(1-(pyrimidin-4-yl)piperidin-4-yl)propoxy)-1,2,3,4-tetrahydroisoquinoline; 6-(4-(3-(2-(methylsulfonyl)-1,2,3,4-tetrahydroisoquinolin-6-yloxy)propyl)piperidin-1-yl)nicotinonitrile; 6-(3-(1-(5-chloropyridin-2-yl)piperidin-4-yl)propoxy)-2-(methylsulfonyl)-1,2,3,4-tetrahydroisoquinoline; 2-(methylsulfonyl)-6-(3-(1-(5-(trifluoromethyl)pyridin-2-yl)piperidin-4-yl)propoxy)-1,2,3,4-tetrahydroisoquinoline; methyl 6-(4-(3-(2-(methylsulfonyl)-1,2,3,4-tetrahydroisoquinolin-6-yloxy)propyl)piperidin-1-yl)nicotinate; 6-(3-(1-(3-chloro-5-(trifluoromethyl)pyridin-2-yl)piperidin-4-yl)propoxy)-2-(methylsulfonyl)-1,2,3,4-tetrahydroisoquinoline; 6-(3-(1-(5-methoxypyridin-2-yl)piperidin-4-yl)propoxy)-2-(methylsulfonyl)-1,2,3,4-tetrahydroisoquinoline; 6-(3-(1-(5-bromopyridin-2-yl)piperidin-4-yl)propoxy)-2-(methylsulfonyl)-1,2,3,4-tetrahydroisoquinoline; 6-(3-(1-(6-chloropyridazin-3-yl)piperidin-4-yl)propoxy)-2-(methylsulfonyl)-1,2,3,4-tetrahydroisoquinoline; 6-(3-(1-(6-methylpyridazin-3-yl)piperidin-4-yl)propoxy)-2-(methylsulfonyl)-1,2,3,4-tetrahydroisoquinoline; 2-(methylsulfonyl)-6-(3-(1-(6-phenylpyridazin-3-yl)piperidin-4-yl)propoxy)-1,2,3,4-tetrahydroisoquinoline; 6-(4-(3-(2-(methylsulfonyl)-1,2,3,4-tetrahydroisoquinolin-6-yloxy)propyl)piperidin-1-yl)nicotinic acid; <<101>>6-(3-(1-(6-ethylpyridazin-3-yl)piperidin-4-yl)propoxy)-2-(methylsulfonyl)-1,2,3,4-tetrahydroisoquinoline; 2-(methylsulfonyl)-6-(3-(1-(6-propylpyridazin-3-yl)piperidin-4-yl)propoxy)-1,2,3,4-tetrahydroisoquinoline; 6-(3-(1-(6-isopropylpyridazin-3-yl)piperidin-4-yl)propoxy)-2-(methylsulfonyl)-1,2,3,4-tetrahydroisoquinoline; 6-(3-(1-(6-tert-butylpyridazin-3-yl)piperidin-4-yl)propoxy)-2-(methylsulfonyl)-1,2,3,4-tetrahydroisoquinoline; 6-(3-(1-(6-cyclopropylpyridazin-3-yl)piperidin-4-yl)propoxy)-2-(methylsulfonyl)-1,2,3,4-tetrahydroisoquinoline; 6-(3-(1-(6-methoxypyridazin-3-yl)piperidin-4-yl)propoxy)-2-(methylsulfonyl)-1,2,3,4-tetrahydroisoquinoline; 4-(2-(4-(3-(2-(methylsulfonyl)-1,2,3,4-tetrahydroisoquinolin-6-yloxy)propyl)piperidin-1-yl)pyrimidin-5-yl)morpholino; 2-(methylsulfonyl)-6-(3-(1-(pyrimidin-5-yl)piperidin-4-yl)propoxy)-1,2,3,4-tetrahydroisoquinoline; 4-(5-(4-(3-(2-(methylsulfonyl)-1,2,3,4-tetrahydroisoquinolin-6-yloxy)propyl)piperidin-1-yl)pyrimidin-2-yl)morpholino; 6-(3-(1-(2-methoxypyrimidin-5-yl)piperidin-4-yl)propoxy)-2-(methylsulfonyl)-1,2,3,4-tetrahydroisoquinoline; 2-(methylsulfonyl)-6-(3-(1-(pyridin-2-yl)piperidin-4-yl)propoxy)-1,2,3,4-tetrahydroisoquinoline; 6-(3-(1-(5-((4-methylpiperazin-1-yl)methyl)pyridin-2-yl)piperidin-4-yl)propoxy)-2-(methylsulfonyl)-1,2,3,4-tetrahydroisoquinoline; 4-((6-(4-(3-(2-(methylsulfonyl)-1,2,3,4-tetrahydroisoquinolin-6-yloxy)propyl)piperidin-1-yl)pyridin-3-yl)methyl)morpholino; 6-(3-(1-(5-methylpyridin-2-yl)piperidin-4-yl)propoxy)-2-(methylsulfonyl)-1,2,3,4-tetrahydroisoquinoline; 6-(3-(1-(5-fluoropyridin-2-yl)piperidin-4-yl)propoxy)-2-(methylsulfonyl)-1,2,3,4-tetrahydroisoquinoline; 2-(methylsulfonyl)-6-(3-(1-(pyridin-3-yl)piperidin-4-yl)propoxy)-1,2,3,4-tetrahydroisoquinoline; 6-(3-(1-(6-methylpyridin-3-yl)piperidin-4-yl)propoxy)-2-(methylsulfonyl)-1,2,3,4-tetrahydroisoquinoline; 6-(3-(1-(6-ethoxypyridin-3-yl)piperidin-4-yl)propoxy)-2-(methylsulfonyl)-1,2,3,4-tetrahydroisoquinoline; 6-(3-(1-(6-methoxypyridin-3-yl)piperidin-4-yl)propoxy)-2-(methylsulfonyl)-1,2,3,4-tetrahydroisoquinoline; 2-(methylsulfonyl)-6-(3-(1-(pyridin-4-yl)piperidin-4-yl)propoxy)-1,2,3,4-tetrahydroisoquinoline; 3-isopropyl-5-(4-(3-(2-(methylsulfonyl)-1,2,3,4-tetrahydroisoquinolin-6-yloxy)propyl)piperidin-1-yl)-1,2,4-oxadiazole; 3-isopropyl-5-(4-(2-(2-(methylsulfonyl)-1,2,3,4-tetrahydroisoquinolin-6-yloxy)ethyl)piperidin-1-yl)-1,2,4-oxadiazole; 6-(3-(1-(1H-tetrazol-5-yl)piperidin-4-yl)propoxy)-2-(methylsulfonyl)-1,2,3,4-tetrahydroisoquinoline; 6-(3-(1-(2-methyl-2H-tetrazol-5-yl)piperidin-4-yl)propoxy)-2-(methylsulfonyl)-1,2,3,4-tetrahydroisoquinoline; 6-(3-(1-(4-methyl-4H-1,2,4-triazol-3-yl)piperidin-4-yl)propoxy)-2-(methylsulfonyl)-1,2,3,4-tetrahydroisoquinoline; 6-(3-(1-(5-(H-tetrazol-5-yl)pyridin-2-yl)piperidin-4-yl)propoxy)-2-(methylsulfonyl)-1,2,3,4-tetrahydroisoquinoline; 6-(3-(1-(5-(2-methyl-2H-tetrazol-5-yl)pyridin-2-yl)piperidin-4-yl)propoxy)-2-(methylsulfonyl)-1,2,3,4-tetrahydroisoquinoline; 6-(3-(1-(5-(1-methyl-1H-tetrazol-5-yl)pyridin-2-yl)piperidin-4-yl)propoxy)-2-(methylsulfonyl)-1,2,3,4-tetrahydroisoquinoline; Isopropyl 4-(4-hydroxy-4-(2-(methylsulfonyl)-1,2,3,4-tetrahydroisoquinolin-6-yl)butyl)piperidine-1-carboxylate; Isopropyl 4-(4-hydroxy-4-(2-(methylsulfonyl)-1,2,3,4-tetrahydroisoquinolin-6-yl)pentyl)piperidine-1-carboxylate; Isopropyl 4-(4-(dimethylamino)-4-(2-(methylsulfonyl)-1,2,3,4-tetrahydroisoquinolin-6-yl)butyl)piperidine-1-carboxylate; Isopropyl 4-(4-formamido-4-(2-(methylsulfonyl)-1,2,3,4-tetrahydroisoquinolin-6-yl)butyl)piperidine-1-carboxylate; Isopropyl 4-(4-amino-4-(2-(methylsulfonyl)-1,2,3,4-tetrahydroisoquinolin-6-yl)butyl)piperidine-1-carboxylate; Isopropyl 4-(6-methoxy-4-(2-(methylsulfonyl)-1,2,3,4-tetrahydroisoquinolin-6-yl)-6-oxohexyl)piperidine-1-carboxylate; Isopropyl 4-(6-hydroxy-4-(2-(methylsulfonyl)-1,2,3,4-tetrahydroisoquinolin-6-yl)hexyl)piperidine-1-carboxylate; 6-(1-(isopropoxycarbonyl)piperidin-4-yl)-3-(2-(methylsulfonyl)-1,2,3,4-tetrahydroisoquinolin-6-yl)hexanoic acid; Isopropyl 4-(4-methoxy-4-(2-(methylsulfonyl)-1,2,3,4-tetrahydroisoquinolin-6-yl)butyl)piperidine-1-carboxylate; Isopropyl 4-(4-fluoro-4-(2-(methylsulfonyl)-1,2,3,4-tetrahydroisoquinolin-6-yl)butyl)piperidine-1-carboxylate; Tert-Butyl 4-(4-(2-(methylsulfonyl)-1,2,3,4-tetrahydroisoquinolin-6-yl)-4-oxobutyl)piperidine-1-carboxylate; 4-(1-(5-ethylpyrimidin-2-yl)piperidin-4-yl)-1-(2-(methylsulfonyl)-1,2,3,4-tetrahydroisoquinolin-6-yl)butan-1-one; 1-methylcyclopropyl 4-(4-(2-(methylsulfonyl)-1,2,3,4-tetrahydroisoquinolin-6-yl)-4-oxobutyl)piperidine-1-carboxylate; 4-(1-(5-fluoropyridin-2-yl)piperidin-4-yl)-1-(2-(methylsulfonyl)-1,2,3,4-tetrahydroisoquinolin-6-yl)butan-1-one; 6-(4-(1-(5-ethylpyrimidin-2-yl)piperidin-4-yl)-1,1-difluorobutyl)-2-(methylsulfonyl)-1,2,3,4-tetrahydroisoquinoline; 1-methylcyclopropyl 4-(4,4-difluoro-4-(2-(methylsulfonyl)-1,2,3,4-tetrahydroisoquinolin-6-yl)butyl)piperidine-1-carboxylate; Isopropyl 4-(3-(1,2,3,4-tetrahydro-2-methanesulfonyl-5-oxo-2,6-naphthyridin-6(5H)-yl)propyl)piperidine-1-carboxylate; 6-(3-(1-(5-ethylpyrimidin-2-yl)piperidin-4-yl)propoxy)-4-methyl-2-(methylsulfonyl)-1,2,3,4-tetrahydroisoquinoline; 6-(3-(1-(5-ethylpyrimidin-2-yl)piperidin-4-yl)propoxy)-5,7-difluoro-2-(methylsulfonyl)-1,2,3,4-tetrahydroisoquinoline; 6-(3-(1-(5-ethylpyrimidin-2-yl)piperidin-4-yl)propoxy)-4,4-dimethyl-2-(methylsulfonyl)-1,2,3,4-tetrahydroisoquinoline; 1-methylcyclopropyl 4-(3-(4,4-dimethyl-2-(methylsulfonyl)-1,2,3,4-tetrahydroisoquinolin-6-yloxy)propyl)piperidine-1-carboxylate; 6-(3-(1-(5-ethylpyrimidin-2-yl)piperidin-4-yl)propoxy)-7-fluoro-2-(methylsulfonyl)-1,2,3,4-tetrahydroisoquinoline; 6-(3-(1-(5-((ethoxymethoxy)methyl)pyrimidin-2-yl)piperidin-4-yl)propoxy)-2-(methylsulfonyl)-1,2,3,4-tetrahydroisoquinoline; isopropyl 4-(2-(5,7-difluoro-2-(methylsulfonyl)-1,2,3,4-tetrahydroisoquinolin-6-yloxy)ethyl)piperidine-1-carboxylate; 6-methyl-4-(4-(3-(2-(methylsulfonyl)-1,2,3,4-tetrahydroisoquinolin-6-yloxy)propyl)piperidin-1-yl)thieno[2,3-d]pyrimidine; 6-(3-(1-(4,6-dimethoxypyrimidin-2-yl)piperidin-4-yl)propoxy)-2-(methylsulfonyl)-1,2,3,4-tetrahydroisoquinoline; isopropyl 4-(3-(1-(methylsulfonyl)-1,2,3,4-tetrahydroquinolin-5-yloxy)propyl)piperidine-1-carboxylate; isopropyl 4-(4-(1-(methylsulfonyl)-1,2,3,4-tetrahydroquinolin-5-yloxy)butyl)piperidine-1-carboxylate; 5-(4-(1-(5-ethylpyrimidin-2-yl)piperidin-4-yl)butoxy)-1-(methylsulfonyl)-1,2,3,4-tetrahydroquinoline; isopropyl 4-(4-(2-(methylsulfonyl)-1,2,3,4-tetrahydroisoquinolin-5-yloxy)butyl)piperidine-1-carboxylate; 1-methylcyclopropyl 4-(3-(5,7-difluoro-2-(methylsulfonyl)-1,2,3,4-tetrahydroisoquinolin-6-yloxy)propyl)piperidine-1-carboxylate; 6-(4-(1-(5-ethylpyrimidin-2-yl)piperidin-4-yl)butoxy)-5,7-difluoro-2-(methylsulfonyl)-1,2,3,4-tetrahydroisoquinoline; 1-methylcyclopropyl 4-(3-(2-(methylsulfonyl)-1,2,3,4-tetrahydroisoquinolin-6-yloxy)propyl)piperidine-1-carboxylate; Tert-butyl 4-(4-(hydroxyimino)-4-(2-(methylsulfonyl)-1,2,3,4-tetrahydroisoquinolin-6-yl)butyl)piperidine-1-carboxylate; Tert-butyl 4-(4-(methoxyimino)-4-(2-(methylsulfonyl)-1,2,3,4-tetrahydroisoquinolin-6-yl)butyl)piperidine-1-carboxylate; 1-methylcyclopropyl 4-(4-hydroxy-4-(2-(methylsulfonyl)-1,2,3,4-tetrahydroisoquinolin-6-yl)butyl)piperidine-1-carboxylate; and 1-methylcyclopropyl 4-(4-chloro-4-(2-(methylsulfonyl)-1,2,3,4-tetrahydroisoquinolin-6-yl)butyl)piperidine-1-carboxylate; 1-methylcyclopropyl 4-(3-(2-(methylsulfonyl)-1,2,3,4-tetrahydroisoquinolin-6-yloxy)propyl)piperazine-1-carboxylate; 6-(3-(4-(5-ethylpyrimidin-2-yl)piperazin-1-yl)propoxy)-2-(methylsulfonyl)-1,2,3,4-tetrahydroisoquinoline; tert-butyl 4-(4,5-dihydroxy-4-(2-(methylsulfonyl)-1,2,3,4-tetrahydroisoquinolin-6-yl)pentyl)piperidine-1-carboxylate; N,N-dimethyl-2-(5-(4-(3-(2-(methylsulfonyl)-1,2,3,4-tetrahydroisoquinolin-6-yloxy)propyl)piperidin-1-yl)-2H-tetrazol-2-yl)ethanamine; 2-(5-(4-(3-(2-(methylsulfonyl)-1,2,3,4-tetrahydroisoquinolin-6-yloxy)propyl)piperidin-1-yl)-2H-tetrazol-2-yl)ethanamine; methyl 2-(5-(4-(3-(2-(methylsulfonyl)-1,2,3,4-tetrahydroisoquinolin-6-yloxy)propyl)piperidin-1-yl)-2H-tetrazol-2-yl)acetate; 6-(3-(1-(2-(2-methoxyethyl)-2H-tetrazol-5-yl)piperidin-4-yl)propoxy)-2-(methylsulfonyl)-1,2,3,4-tetrahydroisoquinoline; 2-(5-(4-(3-(2-(methylsulfonyl)-1,2,3,4-tetrahydroisoquinolin-6-yloxy)propyl)piperidin-1-yl)-2H-tetrazol-2-yl)ethanol; 6-(3-(1-(5-ethylpyrimidin-2-yl)piperidin-4-yl)propoxy)-2-(2-(methylsulfonyl)ethyl)-1,2,3,4-tetrahydroisoquinoline; 1-methylcyclopropyl 4-(3-(2-(2-(methylsulfonyl)ethyl)-1,2,3,4-tetrahydroisoquinolin-6-yloxy)propyl)piperidine-1-carboxylate; 2-(methylsulfonyl)-6-(3-(1-(2-(2-(pyrrolidin-1-yl)ethyl)-2H-tetrazol-5-yl)piperidin-4-yl)propoxy)-1,2,3,4-tetrahydroisoquinoline; tert-butyl 3-(4-(4-(3-(2-(methylsulfonyl)-1,2,3,4-tetrahydroisoquinolin-6-yloxy)propyl)piperidin-1-yl)benzyloxy)propylcarbamate; 4-(2-(5-(4-(3-(2-(methylsulfonyl)-1,2,3,4-tetrahydroisoquinolin-6-yloxy)propyl)piperidin-1-yl)-2H-tetrazol-2-yl)ethyl)morpholine; 3-(4-(4-(3-(2-(methylsulfonyl)-1,2,3,4-tetrahydroisoquinolin-6-yloxy)propyl)piperidin-1-yl)benzyloxy)propan-1-amine; N,N-dimethyl-3-(5-(4-(3-(2-(methylsulfonyl)-1,2,3,4-tetrahydroisoquinolin-6-yloxy)propyl)piperidin-1-yl)-2H-tetrazol-2-yl)propan-1-amine; N,N-diethyl-2-(5-(4-(3-(2-(methylsulfonyl)-1,2,3,4-tetrahydroisoquinolin-6-yloxy)propyl)piperidin-1-yl)-2H-tetrazol-2-yl)ethanamine; 2-(methylsulfonyl)-6-(3-(1-(2-(2-(piperidin-1-yl)ethyl)-2H-tetrazol-5-yl)piperidin-4-yl)propoxy)-1,2,3,4-tetrahydroisoquinoline; 6-(3-(1-(2-(2-(4-isopropylpiperazin-1-yl)ethyl)-2H-tetrazol-5-yl)piperidin-4-yl)propoxy)-2-(methylsulfonyl)-1,2,3,4-tetrahydroisoquinoline; 1-methylcyclopropyl 4-(2-(3-(2-(methylsulfonyl)-1,2,3,4-tetrahydroisoquinolin-6-yl)-5,6-dihydro-1,4-dithiin-2-yl)ethyl)piperidine-1-carboxylate; tert-butyl 4-(3-(2-(methylsulfonyl)-1,2,3,4-tetrahydroisoquinolin-6-yloxy)propyl)piperazine-1-carboxylate; 4-(5-ethylpyrimidin-2-yl)-1-(3-(2-(methylsulfonyl)-1,2,3,4-tetrahydroisoquinolin-6-yloxy)propyl)piperazin-2-one; tert-butyl 4-(5-hydroxy-4-(2-(methylsulfonyl)-1,2,3,4-tetrahydroisoquinolin-6-yl)pentyl)piperidine-1-carboxylate; 6-(4-(1-(5-ethylpyrimidin-2-yl)piperidin-4-yloxy)pyridin-2-yl)-2-(methylsulfonyl)-1,2,3,4-tetrahydroisoquinoline; 6-(3-(1-(1H-benzo[d]imidazol-2-yl)piperidin-4-yl)propoxy)-2-(methylsulfonyl)-1,2,3,4-tetrahydroisoquinoline; 6-(3-(1-(1-methyl-1H-benzo[d]imidazol-2-yl)piperidin-4-yl)propoxy)-2-(methylsulfonyl)-1,2,3,4-tetrahydroisoquinoline; 4-(3-(2-(methylsulfonyl)-1,2,3,4-tetrahydroisoquinolin-6-yloxy)propyl)-1-(pyridin-2-yl)piperazin-2-one; 2-(1-(5-ethylpyrimidin-2-yl)piperidin-4-yloxy)-3-(2-(methylsulfonyl)-1,2,3,4-tetrahydroisoquinolin-6-yloxy)propan-1-ol; 1-methylcyclopropyl 4-(4-(2-(methylsulfonyl)-1,2,3,4-tetrahydroisoquinolin-6-yl)-3-morpholino-4-oxobutyl)piperidine-1-carboxylate.

In another embodiment are compounds of Formula Ib:

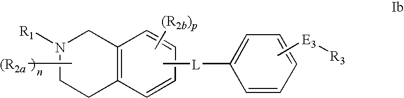

in which:

n and p are independently selected from 0, 1, 2 and 3;

E₃ is selected from a bond, 0 and OCH₂;

L is selected from C₁₋₁₀heteroarylene, —X₂OX₃—, —OX₂X₃—, —C(O)X₂—, —X₂X₃—, —OX₂O—, —OX₂C(O)X₃—, —OX₂C(O)OX₃—, —CR₄(NR₄R₄)X₂—, —CR₄(NR₄C(O)R₄)X₂—, —C(═NOR₄)X₂—, —NR₄C(O)X₂—, —C(O)NR₄X₂—, —NR₄X₂—, —N(C(O)R₄)X₂— and —OC(O)NR₄X₂—; wherein X₂ and X₃ are independently selected from a bond, C₁₋₆alkylene, C₂₋₆alkenylene, C₆₋₁₀aryl, C₃₋₈cycloalkyl and C₁₋₁₀heteroarylene; R₄ is selected from hydrogen and C₁₋₆alkyl; wherein any methylene of L can have the hydrogens replaced by a radical selected from halo, hydroxy, C₁₋₄alkyl, C₁₋₄alkoxy, hydroxy-substituted-C₁₋₄alkyl and —CR₄R₄C(O)OR₄;

R₁ is selected from C₁₋₁₀alkyl, halo-substituted-C₁₋₁₀alkyl, C₆₋₁₀aryl, C₁₋₁₀heteroaryl, —X₅S(O)₀₋₂R₅ₐ, —X₅C(O)OR₅ₐ, —X₅C(O)R₅ₐ, and —X₅C(O)NR₅ₐR_{5b}; wherein X₅ is selected from a bond and C₁₋₃alkylene; R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy-NR₅CR_{5d}, —C(O)OR_{5c} and C₆₋₁₀aryl-C₀₋₄alkyl; wherein R_{5c} and R_{5d} are independently selected from hydrogen and C₁₋₆alkyl;

R₂ₐ and R_{2b} are independently selected from halo, methyl, cyano and nitro; and

R₃ is selected from hydrogen, SO₂R₆ₐ, C₆₋₁₀aryl, C₁₋₁₀heteroaryl and —C(O)OR₆ₐ and —OC(O)NR₆ₐR_{6b}; wherein R₆ₐ and R_{6b} are independently selected from hydrogen, C₁₋₆alkyl and C₃₋₁₂cycloalkyl optionally substituted with C₁₋₄alkyl; wherein said heteroaryl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐR₉, C₁₋₆alkyl, C₁₋₆alkoxy and halo-substituted-C₁₋₆alkyl; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl-C₀₋₄alkyl; wherein said aryl, heteroaryl, cycloalkyl or heterocycloalkyl of R₉ is optionally substituted with 1 to 3 radicals independently selected from halo, C₁₋₄alkyl and C₁₋₄alkoxy.

In a further embodiment, L is selected from 3,5-1,2,4-oxadiazolylene, (1,2,4-oxadiazol-5-yl)methoxy, (1,2,4-oxadiazol-5-yl)methyl, (1,2,4-oxadiazol-5-yl)ethyl, (1,2,4-oxadiazol-5-yl)propyl, phenoxy, phenoxy-methyl, —C(O)NHCH₂—, —C(O)NH(CH₂)₂—, —CH₂OCH₂—, —C(O)NH(CH₂)₃—, —CH((CH₂)₂OH)(CH₂)₃—, —CH(CH₂C(O)OCH₃)(CH₂)₃—, —C(O)(CH₂)₃—, —CH(OH)(CH₂)₃—, —CH(Cl)(CH₂)₃—, —C(CH₃)(OH)(CH₂)₃—, —CH(N(CH₃)₂)(CH₂)₃—, —CH(NH₂)(CH₂)₃—, —CH(NHC(O)H)(CH₂)₃—, —CF₂(CH₂)₃—, —O(CH₂)₂—, —(CH₂)₂—, —(CH₂)₃—, —(CH₂)₄—, —O(CH₂)₄—, —O(CH₂)₃—, —NH(CH₂)₂—, —NH(CH₂)₃—, —C(═NOCH₃)(CH₂)₃—, —C(═NOH)(CH₂)₃—, —NHC(O)(CH₂)₃—, —NH(CH₂)₄—, —NCH₃(CH₂)₄—, —N(C(O)CH₃)(CH₂)₃—, —NC₂H₅(CH₂)₃—, —NC₃H₇(CH₂)₃—, —O(CH₂)₃O—, —O(CH₂)₂O—, —CH═CH(CH₂)₂—; —CH═CH—; —OCH₂CH(CH₂OH)O—; —C(O)CH(N(CH₂)₂—O—(CH₂)₂)—(CH₂)₂—; —NCH₃(CH₂)₃—; —N(CH(CH₃)₂)(CH₂)₃—; —NHC(O)(CH₂)₂—; —CH₂—O—(CH₂)₂—; —CH₂—O—(CH₂)₃—; —CH₂—O—(CH₂)₄—; —CH═CHCH₂—; —CH(CH₂COOH)(CH₂)₃—; —CH(OCH₃)(CH₂)₃—; —CH(F)(CH₂)₃—; —C(OH)(CH₂OH)(CH₂)₃—; —CH(CH₂OH)(CH₂)₃—;

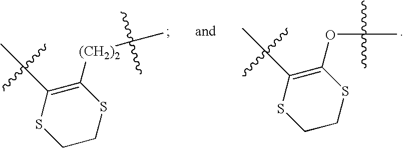

In a further embodiment, R₁ is selected from methyl-sulfonyl, butyl-sulfonyl, phenyl-sulfonyl, isopropyl-sulfonyl, ethyl-sulfonyl, ethenyl-sulfonyl, isopropoxy-carbonyl, benzyloxy-carbonyl, ethoxy-carbonyl, methyl-sulfonyl-ethyl, methoxy-carbonyl, t-butoxy-carbonyl and trifluoromethyl-sulfonyl.

In a further embodiment, R₃ is selected from t-butoxy-carbonyl, dimethylamino-carbonyl, methyl-sulfonyl, isopropoxy-carbonyl(ethyl)amino-methyl, isopropoxy-carbonyl-amino-methyl, benzyl(ethyl)amino-methyl, piperidinyl, quinazolinyl, isopropoxy-carbonyl, thieno[2,3-d]pyrimidin-4-yl, 4H-1,2,4-triazolyl, cyclopropoxy-carbonyl, (1,2,4-oxadiazol-5-yl), tetrazolyl, thiazolyl, triazolyl, pyrimidinyl, pyrazinyl, pyridinyl, phenyl, benzimidazolyl and pyridazinyl; wherein said cyclopropoxy, quinazolinyl, thieno[2,3-d]pyrimidinyl, thiazolyl, oxadiazolyl, tetrazolyl, pyrimidinyl, pyrazinyl, pyridinyl, phenyl, benzimidazolyl or pyridazinyl can be optionally substituted by 1 to 2 radicals independently selected from halo, cyano, methyl, methoxy-carbonyl, carboxyl, isopropyl, t-butyl, cyclopropyl, morpholino, methyl-piperazinyl-methyl, morpholino-methyl, ethoxy-methoxy-methyl, hydroxy-methyl, methoxy-ethoxy-methyl, methoxy-methoxy-methyl, ethoxy, trifluoromethyl, pentyl, phenyl, methoxy, dimethylamino, dimethylamino-methyl, dimethylamino-ethyl, aminoethyl, methoxy-carbonyl-methyl, methoxy-ethyl, hydroxyl-ethyl, pyrrolidinoethyl, t-butoxycarbonylamino-propoxy-methyl, morpholino-ethyl, aminopropoxy-methyl, dimethylamino-methyl, diethylamino-methyl, isopropyl-piperazino-ethyl, methoxy-ethoxy-ethoxy-methyl, methoxy-methyl, propyl and ethyl.

In a further embodiment are compounds selected from: 3-tert-butyl-5-(4-((2-(methylsulfonyl)-1,2,3,4-tetrahydroisoquinolin-6-yloxy)methyl)phenyl)-1,2,4-oxadiazole; 3-(2-(methylsulfonyl)-1,2,3,4-tetrahydroisoquinolin-6-yl)-5-(4-(pyrimidin-2-yl)benzyl)-1,2,4-oxadiazole; 5-(4-bromophenethyl)-3-(2-(methylsulfonyl)-1,2,3,4-tetrahydroisoquinolin-6-yl)-1,2,4-oxadiazole; 5-(4-(5-methylpyridin-2-yl)benzyl)-3-(2-(methylsulfonyl)-1,2,3,4-tetrahydroisoquinolin-6-yl)-1,2,4-oxadiazole; 5-(4-(5-methylpyridin-2-yl)phenethyl)-3-(2-(methylsulfonyl)-1,2,3,4-tetrahydroisoquinolin-6-yl)-1,2,4-oxadiazole; 5-(4-(5-bromopyrimidin-2-yl)phenethyl)-3-(2-(methylsulfonyl)-1,2,3,4-tetrahydroisoquinolin-6-yl)-1,2,4-oxadiazole; 2-(methylsulfonyl)-6-(3-(4-(pyrimidin-2-yl)phenyl)propoxy)-1,2,3,4-tetrahydroisoquinoline; 4-(5-(4-(3-(2-(methylsulfonyl)-1,2,3,4-tetrahydroisoquinolin-6-yloxy)propyl)phenyl)pyrimidin-2-yl)morpholino; 2-(methylsulfonyl)-6-(3-(4-(5-(trifluoromethyl)pyridin-2-yl)phenyl)propoxy)-1,2,3,4-tetrahydroisoquinoline; 2-(methylsulfonyl)-6-(3-(4-(pyrazin-2-yl)phenyl)propoxy)-1,2,3,4-tetrahydroisoquinoline; 6-(3-(4-(5-ethylpyrimidin-2-yl)phenyl)propoxy)-5,7-difluoro-2-(methylsulfonyl)-1,2,3,4-tetrahydroisoquinoline; 5-tert-butyl-3-(4-((2-(methylsulfonyl)-1,2,3,4-tetrahydroisoquinolin-6-yloxy)methyl)phenyl)-1,2,4-oxadiazole; 6-(4-(5-ethylpyrimidin-2-yl)phenethoxy)-2-(methylsulfonyl)-1,2,3,4-tetrahydroisoquinoline; N-benzyl-N-(4-((2-(methylsulfonyl)-1,2,3,4-tetrahydroisoquinolin-6-yloxy)methyl)benzyl)ethanamine; 6-(3-(4-(5-ethylpyrimidin-2-yl)phenyl)propoxy)-2-(methylsulfonyl)-1,2,3,4-tetrahydroisoquinoline; 6-(4-iodophenethoxy)-2-(methylsulfonyl)-1,2,3,4-tetrahydroisoquinoline; 5-tert-butyl-3-(4-(2-(2-(methylsulfonyl)-1,2,3,4-tetrahydroisoquinolin-6-yloxy)ethyl)phenyl)-1,2,4-oxadiazole; isopropyl ethyl(4-(3-(2-(methylsulfonyl)-1,2,3,4-tetrahydroisoquinolin-6-yloxy)propoxy)benzyl)carbamate; isopropyl ethyl(3-(3-(2-(methylsulfonyl)-1,2,3,4-tetrahydroisoquinolin-6-yloxy)propoxy)benzyl)carbamate; isopropyl ethyl(4-(2-(2-(methylsulfonyl)-1,2,3,4-tetrahydroisoquinolin-6-yloxy)ethoxy)benzyl)carbamate; isopropyl ethyl(3-(2-(2-(methylsulfonyl)-1,2,3,4-tetrahydroisoquinolin-6-yloxy)ethoxy)benzyl)carbamate; isopropyl 4-(2-(2-(methylsulfonyl)-1,2,3,4-tetrahydroisoquinolin-6-yloxy)ethoxy)benzylcarbamate; 6-(3-(4-(6-cyclopropylpyridazin-3-yl)phenyl)propoxy)-2-(methylsulfonyl)-1,2,3,4-tetrahydroisoquinoline; 3-(4-bromobenzyl)-5-(2-(methylsulfonyl)-1,2,3,4-tetrahydroisoquinolin-6-yl)-1,2,4-oxadiazole; 5-(2-(methylsulfonyl)-1,2,3,4-tetrahydroisoquinolin-6-yl)-3-(4-(pyrazin-2-yl)phenethyl)-1,2,4-oxadiazole; 3-(2-(4-(5-ethylpyrimidin-2-yl)cyclohexa-1,5-dienyl)ethyl)-5-(2-(methylsulfonyl)-1,2,3,4-tetrahydroisoquinolin-6-yl)-1,2,4-oxadiazole; 5-(2-(methylsulfonyl)-1,2,3,4-tetrahydroisoquinolin-6-yl)-3-(2-(4-(pyrimidin-2-yl)cyclohexa-1,5-dienyl)ethyl)-1,2,4-oxadiazole; 2-(4-(3-(2-(methylsulfonyl)-1,2,3,4-tetrahydroisoquinolin-6-yloxy)propyl)phenyl)thiazole; 6-(3-(4-(5-((methoxymethoxy)methyl)pyrimidin-2-yl)phenyl)propoxy)-2-(methylsulfonyl)-1,2,3,4-tetrahydroisoquinoline; 6-(3-(4-(5-((2-methoxyethoxy)methyl)pyrimidin-2-yl)phenyl)propoxy)-2-(methylsulfonyl)-1,2,3,4-tetrahydroisoquinoline; (2-(4-(3-(2-(methylsulfonyl)-1,2,3,4-tetrahydroisoquinolin-6-yloxy)propyl)phenyl)pyrimidin-5-yl)methanol; 4-(3-(2-(methylsulfonyl)-1,2,3,4-tetrahydroisoquinolin-6-yloxy)propyl)benzonitrile; 6-(3-(4-(1H-tetrazol-5-yl)phenyl)propoxy)-2-(methylsulfonyl)-1,2,3,4-tetrahydroisoquinoline; N,N-dimethyl-1-(2-(4-(3-(2-(methylsulfonyl)-1,2,3,4-tetrahydroisoquinolin-6-yloxy)propyl)phenyl)pyrimidin-5-yl)methanamine; 6-(3-(4-(5-ethylpyrimidin-2-yl)phenyl)propoxy)-7-fluoro-2-(methylsulfonyl)-1,2,3,4-tetrahydroisoquinoline; and 6-(3-(4-(5-ethylpyrimidin-2-yl)phenyl)propoxy)-4-methyl-2-(methylsulfonyl)-1,2,3,4-tetrahydroisoquinoline; 6-(3-(4-(5-ethylpyrimidin-2-yl)phenyl)propoxy)-1,2,3,4-tetrahydroisoquinoline; 6-(3-(4-(5-ethylpyrimidin-2-yl)phenyl)propoxy)-2-(vinylsulfonyl)-1,2,3,4-tetrahydroisoquinoline; 6-(3-(3-(5-ethylpyrimidin-2-yloxy)phenyl)propoxy)-2-(methylsulfonyl)-1,2,3,4-tetrahydroisoquinoline; 2-(methylsulfonyl)-6-(3-(4-(pyrimidin-2-yloxy)phenyl)propoxy)-1,2,3,4-tetrahydroisoquinoline; 6-(1-(4-(5-ethylpyrimidin-2-yl)phenyl)pyrrolidin-3-yloxy)-2-(methylsulfonyl)-1,2,3,4-tetrahydroisoquinoline; 6-(3-(4-(5-ethylpyrimidin-2-yl)-3-fluorophenyl)propoxy)-5,7-difluoro-2-(methylsulfonyl)-1,2,3,4-tetrahydroisoquinoline; 6-(3-(4-(5-ethylpyrimidin-2-yl)-3-fluorophenyl)propoxy)-4-methyl-2-(methylsulfonyl)-1,2,3,4-tetrahydroisoquinoline; 6-(3-(4-(5-ethylpyrimidin-2-yl)-3-fluorophenyl)propoxy)-7-fluoro-2-(methylsulfonyl)-1,2,3,4-tetrahydroisoquinoline; 6-(3-(4-(5-ethylpyrimidin-2-yl)-3-fluorophenyl)propoxy)-2-(methylsulfonyl)-1,2,3,4-tetrahydroisoquinoline; 6-(3-(4′-butylbiphenyl-4-yl)propoxy)-2-(methylsulfonyl)-1,2,3,4-tetrahydroisoquinoline; 6-(3-(4-(benzyloxy)phenyl)propoxy)-2-(methylsulfonyl)-1,2,3,4-tetrahydroisoquinoline; 4-(3-(2-(methylsulfonyl)-1,2,3,4-tetrahydroisoquinolin-6-yloxy)propyl)phenyl dimethylcarbamate; 6-(3-(4-(5-ethylpyrimidin-2-yloxy)phenyl)propoxy)-2-(methylsulfonyl)-1,2,3,4-tetrahydroisoquinoline; benzyl 6-(3-(4-(5-ethylpyrimidin-2-yl)phenyl)propoxy)-3,4-dihydroisoquinoline-2(1H)-carboxylate; 2-(methylsulfonyl)-6-(3-(4-(pyrazin-2-yloxy)phenyl)propoxy)-1,2,3,4-tetrahydroisoquinoline; 6-(3-(4-(5-ethylpyrimidin-2-yl)-3-methylphenyl)propoxy)-2-(methylsulfonyl)-1,2,3,4-tetrahydroisoquinoline; 6-(3-(4-(5-((2-(2-methoxyethoxy)ethoxy)methyl)pyrimidin-2-yl)phenyl)propoxy)-2-(methylsulfonyl)-1,2,3,4-tetrahydroisoquinoline; 6-(3-(4-(5-(methoxymethyl)pyrimidin-2-yl)phenyl)propoxy)-2-(methylsulfonyl)-1,2,3,4-tetrahydroisoquinoline; 2-(methylsulfonyl)-6-(3-(4-(pyridin-2-yloxy)phenyl)propoxy)-1,2,3,4-tetrahydroisoquinoline; 2-(methylsulfonyl)-6-(3-(4-(pyridin-3-yloxy)phenyl)propoxy)-1,2,3,4-tetrahydroisoquinoline; 2-(methylsulfonyl)-6-(3-(4-(pyridin-4-yloxy)phenyl)propoxy)-1,2,3,4-tetrahydroisoquinoline; 6-(3-(4-(4-methoxypyrimidin-2-yloxy)phenyl)propoxy)-2-(methylsulfonyl)-1,2,3,4-tetrahydroisoquinoline; 6-(3-(4-(4-methylpyrimidin-2-yloxy)phenyl)propoxy)-2-(methylsulfonyl)-1,2,3,4-tetrahydroisoquinoline; N,N-dimethyl-2-(4-(3-(2-(methylsulfonyl)-1,2,3,4-tetrahydroisoquinolin-6-yloxy)propyl)phenoxy)pyrimidin-4-amine; 3-(3-(2-(methylsulfonyl)-1,2,3,4-tetrahydroisoquinolin-6-yloxy)propyl)phenyl methanesulfonate; 3-(3-(2-(methylsulfonyl)-1,2,3,4-tetrahydroisoquinolin-6-yloxy)propyl)phenol; 2-(Methylsulfonyl)-6-(3-(3-(pyrimidin-2-yloxy)phenyl)propoxy)-1,2,3,4-tetrahydroisoquinoline; 2-(Methylsulfonyl)-6-(3-(4-(pyrimidin-2-yloxy)phenyl)propoxy)-1,2,3,4-tetrahydroisoquinoline; 6-(3-(4-(Benzyloxy)phenyl)propoxy)-2-(methylsulfonyl)-1,2,3,4-tetrahydroisoquinoline; 4-(3-(2-(Methylsulfonyl)-1,2,3,4-tetrahydroisoquinolin-6-yloxy)propyl)phenyl dimethylcarbamate; 2-(Methylsulfonyl)-6-(3-(4-(pyrazin-2-yloxy)phenyl)propoxy)-1,2,3,4-tetrahydroisoquinoline; 3-(3-(2-(Methylsulfonyl)-1,2,3,4-tetrahydroisoquinolin-6-yloxy)propyl)phenyl methanesulfonate; 4-(3-(2-(Methylsulfonyl)-1,2,3,4-tetrahydroisoquinolin-6-yloxy)propyl)phenol; 6-(3-(4-(5-ethylpyrimidin-2-yloxy)phenyl)propoxy)-2-(methylsulfonyl)-1,2,3,4-tetrahydroisoquinoline; 2-(methylsulfonyl)-6-(3-(4-(pyrimidin-5-yloxy)phenyl)propoxy)-1,2,3,4-tetrahydroisoquinoline; 2-(methylsulfonyl)-6-(3-(4-(pyridin-2-yloxy)phenyl)propoxy)-1,2,3,4-tetrahydroisoquinoline; 2-(methylsulfonyl)-6-(3-(4-(pyridin-3-yloxy)phenyl)propoxy)-1,2,3,4-tetrahydroisoquinoline; 2-(methylsulfonyl)-6-(3-(4-(pyridin-4-yloxy)phenyl)propoxy)-1,2,3,4-tetrahydroisoquinoline; 6-(3-(4-(4-methoxypyrimidin-2-yloxy)phenyl)propoxy)-2-(methylsulfonyl)-1,2,3,4-tetrahydroisoquinoline; 6-(3-(4-(4-methylpyrimidin-2-yloxy)phenyl)propoxy)-2-(methylsulfonyl)-1,2,3,4-tetrahydroisoquinoline; and N,N-dimethyl-2-(4-(3-(2-(methylsulfonyl)-1,2,3,4-tetrahydroisoquinolin-6-yloxy)propyl)phenoxy)pyrimidin-4-amine.

In another embodiment, are compounds of Formula Ic:

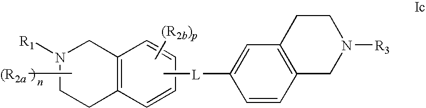

in which:

n and p are independently selected from 0, 1, 2 and 3;

L is selected from C₁₋₁₀heteroarylene, —X₂OX₃—, —X₂X₃—, —C(O)X₂—, —X₂X₃—, —OX₂O—, —OX₂C(O)X₃—, —OX₂C(O)OX₃—, —CR₄(NR₄R₄)X₂—, —CR₄(NR₄C(O)R₄)X₂—, —C(═NOR₄)X₂—, —NR₄C(O)X₂—, —C(O)NR₄X₂—, —NR₄X₂—, —N(C(O)R₄)X₂— and —OC(O)NR₄X₂—; wherein X₂ and X₃ are independently selected from a bond, C₁₋₆alkylene, C₂₋₆alkenylene, C₆₋₁₀aryl, C₃₋₈cycloalkyl and C₁₋₁₀heteroarylene; R₄ is selected from hydrogen and C₁₋₆alkyl; wherein any methylene of L can have the hydrogens replaced by a radical selected from halo, hydroxy, C₁₋₄alkyl, C₁₋₄alkoxy, hydroxy-substituted-C₁₋₄alkyl and —CR₄R₄C(O)OR₄;

R₁ is selected from C₁₋₁₀alkyl, halo-substituted-C₁₋₁₀alkyl, C₆₋₁₀aryl, C₁₋₁₀heteroaryl, —XS(O)₀₋₂R₅ₐ, —X₅C(O)OR₅ₐ, —X₅C(O)R₅ₐ, and —X₅C(O)NR₅ₐR_{5b}; wherein X₅ is selected from a bond and C₁₋₃alkylene; R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy —NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;

R₂ₐ and R_{2b} are independently selected from halo, methyl, cyano and nitro; and

R₃ is selected from aryl, C₁₋₁₀heteroaryl and —C(O)OR₆ₐ; wherein R₆ₐ is selected from hydrogen, C₁₋₆alkyl and C₃₋₁₂cycloalkyl optionally substituted with C₁₋₄alkyl; wherein said heteroaryl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐR₉, C₁₋₆alkyl, C₁₋₆alkoxy and halo-substituted-C₁₋₆alkyl; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl-C₀₋₄alkyl; wherein said aryl, heteroaryl, cycloalkyl or heterocycloalkyl of R₉ is optionally substituted with 1 to 3 radicals independently selected from halo, C₁₋₄alkyl and C₁₋₄alkoxy.

In a further embodiment, L is selected from 3,5-1,2,4-oxadiazolylene, (1,2,4-oxadiazol-5-yl)methoxy, (1,2,4-oxadiazol-5-yl)methyl, (1,2,4-oxadiazol-5-yl)ethyl, (1,2,4-oxadiazol-5-yl)propyl, phenoxy, phenoxy-methyl, —C(O)NHCH₂—, —C(O)NH(CH₂)₂—, —CH₂OCH₂—, —C(O)NH(CH₂)₃—, —CH((CH₂)₂OH)(CH₂)₃—, —CH(CH₂C(O)OCH₃)(CH₂)₃—, —C(O)(CH₂)₃—, —CH(OH)(CH₂)₃—, —CH(CI)(CH₂)₃—, —C(CH₃)(OH)(CH₂)₃—, —CH(N(CH₃)₂)(CH₂)₃—, —CH(NH₂)(CH₂)₃—, —CH(NHC(O)H)(CH₂)₃—, —CF₂(CH₂)₃—, —O(CH₂)₂—, —(CH₂)₂—, —(CH₂)₃—, —(CH₂)₄—, —O(CH₂)₄—, —O(CH₂)₃—, —NH(CH₂)₂—, —NH(CH₂)₃—, —C(═NOCH₃)(CH₂)₃—, —C(═NOH)(CH₂)₃—, —NHC(O)(CH₂)₃—, —NH(CH₂)₄—, —NCH₃(CH₂)₄—, —N(C(O)CH₃)(CH₂)₃—, —NC₂H₅(CH₂)₃—, —NC₃H₇(CH₂)₃—, —O(CH₂)₃O—, —O(CH₂)₂O—, —CH═CH(CH₂)₂—; —CH═CH—; —OCH₂CH(CH₂OH)O—; —C(O)CH(N(CH₂)₂—O—(CH₂)₂)—(CH₂)₂—; —NCH₃(CH₂)₃—; —N(CH(CH₃)₂)(CH₂)₃—; —NHC(O)(CH₂)₂—; —CH₂—O—(CH₂)₂—; —CH₂—O—(CH₂)₃—; —CH₂—O—(CH₂)₄—; —CH═CHCH₂—; —CH(CH₂COOH)(CH₂)₃—; —CH(OCH₃)(CH₂)₃—; —CH(F)(CH₂)₃—; —C(OH)(CH₂OH)(CH₂)₃—; —CH(CH₂OH)(CH₂)₃—;

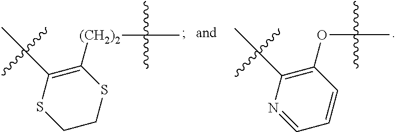

In a further embodiment, R₁ is selected from methyl-sulfonyl, butyl-sulfonyl, phenyl-sulfonyl, isopropyl-sulfonyl, ethyl-sulfonyl, ethenyl-sulfonyl, methyl-sulfonyl-ethyl, isopropoxy-carbonyl, benzyloxy-carbonyl, ethoxy-carbonyl, methoxy-carbonyl, t-butoxy-carbonyl and trifluoromethyl-sulfonyl.

In a further embodiment, R₃ is selected from t-butoxy-carbonyl, dimethylamino-carbonyl, methyl-sulfonyl, isopropoxy-carbonyl(ethyl)amino-methyl, isopropoxy-carbonyl-amino-methyl, benzyl(ethyl)amino-methyl, piperidinyl, quinazolinyl, isopropoxy-carbonyl, thieno[2,3-d]pyrimidin-4-yl, 4H-1,2,4-triazolyl, cyclopropoxy-carbonyl, (1,2,4-oxadiazol-5-yl), tetrazolyl, thiazolyl, triazolyl, pyrimidinyl, pyrazinyl, pyridinyl, phenyl, benzimidazolyl and pyridazinyl; wherein said cyclopropoxy, quinazolinyl, thieno[2,3-d]pyrimidinyl, thiazolyl, oxadiazolyl, tetrazolyl, pyrimidinyl, pyrazinyl, pyridinyl, phenyl, benzimidazolyl or pyridazinyl can be optionally substituted by 1 to 2 radicals independently selected from halo, cyano, methyl, methoxy-carbonyl, carboxyl, isopropyl, t-butyl, cyclopropyl, morpholino, methyl-piperazinyl-methyl, morpholino-methyl, ethoxy-methoxy-methyl, hydroxy-methyl, methoxy-ethoxy-methyl, methoxy-methoxy-methyl, ethoxy, trifluoromethyl, pentyl, phenyl, methoxy, dimethylamino, dimethylamino-methyl, dimethylamino-ethyl, aminoethyl, methoxy-carbonyl-methyl, methoxy-ethyl, hydroxyl-ethyl, pyrrolidinoethyl, t-butoxycarbonylamino-propoxy-methyl, morpholino-ethyl, aminopropoxy-methyl, dimethylamino-methyl, diethylamino-methyl, isopropyl-piperazino-ethyl, methoxy-ethoxy-ethoxy-methyl, methoxy-methyl, propyl and ethyl.

In a further embodiment are compounds selected from: 2-(5-bromopyrimidin-2-yl)-6-((2-(methylsulfonyl)-1,2,3,4-tetrahydroisoquinolin-6-yloxy)methyl)-1,2,3,4-tetrahydroisoquinoline; 2-(methylsulfonyl)-6-((2-(pyrazin-2-yl)-1,2,3,4-tetrahydroisoquinolin-6-yl)methoxy)-1,2,3,4-tetrahydroisoquinoline; 2-(6-((2-(methylsulfonyl)-1,2,3,4-tetrahydroisoquinolin-6-yloxy)methyl)-3,4-dihydroisoquinolin-2(1H)-yl)quinazoline; 2-(methylsulfonyl)-6-((2-(pyrimidin-2-yl)-1,2,3,4-tetrahydroisoquinolin-6-yl)methoxy)-1,2,3,4-tetrahydroisoquinoline; tert-butyl 6-((2-(methylsulfonyl)-1,2,3,4-tetrahydroisoquinolin-6-yloxy)methyl)-3,4-dihydroisoquinoline-2(1H)-carboxylate; isopropyl 6-((2-(methylsulfonyl)-1,2,3,4-tetrahydroisoquinolin-6-yloxy)methyl)-3,4-dihydroisoquinoline-2(1H)-carboxylate; 2-(5-ethylpyrimidin-2-yl)-6-((2-(methylsulfonyl)-1,2,3,4-tetrahydroisoquinolin-6-yloxy)methyl)-1,2,3,4-tetrahydroisoquinoline; isopropyl 6-(3-(2-(methylsulfonyl)-1,2,3,4-tetrahydroisoquinolin-6-yl)-1,2,4-oxadiazol-5-yl)-3,4-dihydroisoquinoline-2(1H)-carboxylate; and 5-(2-(methylsulfonyl)-1,2,3,4-tetrahydroisoquinolin-6-yl)-3-((2-(5-(trifluoromethyl)pyridin-2-yl)-1,2,3,4-tetrahydroisoquinolin-6-yl)methyl)-1,2,4-oxadiazole.

In another embodiment are compounds of Formula Id:

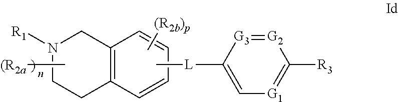

in which:

n and p are independently selected from 0, 1, 2 and 3;

L is selected from C₁₋₁₀heteroarylene, —X₂OX₃—, —OX₂X₃—, —C(O)X₂—, —X₂X₃—, —OX₂O—, —OX₂C(O)X₃—, —OX₂C(O)OX₃—, —CR₄(NR₄R₄)X₂—, —CR₄(NR₄C(O)R₄)X₂—, —C(═NOR₄)X₂—, —NR₄C(O)X₂—, —C(O)NR₄X₂—, —NR₄X₂—, —N(C(O)R₄)X₂— and —OC(O)NR₄X₂—; wherein X₂ and X₃ are independently selected from a bond, C₁₋₆alkylene, C₂₋₆alkenylene, C₆₋₁₀aryl, C₃₋₈cycloalkyl and C₁₋₁₀heteroarylene; R₄ is selected from hydrogen and C₁₋₆alkyl; wherein any methylene of L can have the hydrogens replaced by a radical selected from halo, hydroxy, C₁₋₄alkyl, C₁₋₄alkoxy, hydroxy-substituted-C₁₋₄alkyl and —CR₄R₄C(O)OR₄;

R₁ is selected from C₁₋₁₀alkyl, halo-substituted-C₁₋₁₀alkyl, C₆₋₁₀aryl, C₁₋₁₀heteroaryl, —X₅S(O)₀₋₂R₅ₐ, —X₅C(O)OR₅ₐ, —X₅C(O)R₅ₐ, and —X₅C(O)NR₅ₐR_{5b}; wherein X₅ is selected from a bond and C₁₋₃alkylene; R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy-NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;

R₂ₐ and R_{2b} are independently selected from halo, methyl, cyano and nitro;

G₁, G₂ and G₃ are independently selected from N and CH; with the proviso that at least one of G₁, G₂ or G₃ is N;

is selected from aryl, C₁₋₁₀heteroaryl and —C(O)OR₆ₐ; wherein R₆ₐ is selected from hydrogen, C₁₋₆alkyl and C₃₋₁₂cycloalkyl optionally substituted with C₁₋₄alkyl; wherein said heteroaryl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐR₉, C₁₋₆alkyl, C₁₋₆alkoxy and halo-substituted-C₁₋₆alkyl; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl-C₀₋₄alkyl; wherein said aryl, heteroaryl, cycloalkyl or heterocycloalkyl of R₉ is optionally substituted with 1 to 3 radicals independently selected from halo, C₁₋₄alkyl and C₁₋₄alkoxy.

In a further embodiment, L is selected from 3,5-1,2,4-oxadiazolylene, (1,2,4-oxadiazol-5-yl)methoxy, (1,2,4-oxadiazol-5-yl)methyl, (1,2,4-oxadiazol-5-yl)ethyl, (1,2,4-oxadiazol-5-yl)propyl, phenoxy, phenoxy-methyl, —C(O)NHCH₂—, —C(O)NH(CH₂)₂—, —CH₂OCH₂—, —C(O)NH(CH₂)₃—, —CH((CH₂)₂OH)(CH₂)₃—, —CH(CH₂C(O)OCH₃)(CH₂)₃—, —C(O)(CH₂)₃—, —CH(OH)(CH₂)₃—, —CH(Cl)(CH₂)₃—, —C(CH₃)(OH)(CH₂)₃—, —CH(N(CH₃)₂)(CH₂)₃—, —CH(NH₂)(CH₂)₃—, —CH(NHC(O)H)(CH₂)₃—, —CF₂(CH₂)₃—, —O(CH₂)₂—, —(CH₂)₂—, —CH₂)₃—, —CH₂)₄—, —O(CH₂)₄—, —O(CH₂)₃—, —NH(CH₂)₂—, —NH(CH₂)₃—, —C(═NOCH₃)(CH₂)₃—, —C(═NOH)(CH₂)₃—, —NHC(O)(CH₂)₃—, —NH(CH₂)₄—, —NCH₃(CH₂)₄—, —N(C(O)CH₃)(CH₂)₃—, —NC₂H₅(CH₂)₃—, —NC₃H₇(CH₂)₃—, —O(CH₂)₃O—, —O(CH₂)₂O—, —CH═CH(CH₂)₂—; —CH═CH—; —OCH₂CH(CH₂OH)O—; —C(O)CH(N(CH₂)₂—O—(CH₂)₂)—(CH₂)₂—; —NCH₃(CH₂)₃—; —N(CH(CH₃)₂)(CH₂)₃—; —NHC(O)(CH₂)₂—; —CH₂—O—(CH₂)₂—; —CH₂—O—(CH₂)₃—; —CH₂—O—(CH₂)₄—; —CH═CHCH₂—; —CH(CH₂COOH)(CH₂)₃—; —CH(OCH₃)(CH₂)₃—; —CH(F)(CH₂)₃—; —C(OH)(CH₂OH)(CH₂)₃—; —CH(CH₂OH)(CH₂)₃—;

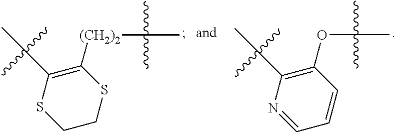

In a further embodiment, R₁ is selected from methyl-sulfonyl, butyl-sulfonyl, phenyl-sulfonyl, isopropyl-sulfonyl, ethyl-sulfonyl, ethenyl-sulfonyl, methyl-sulfonyl-ethyl, isopropoxy-carbonyl, benzyloxy-carbonyl, ethoxy-carbonyl, methoxy-carbonyl, t-butoxy-carbonyl and trifluoromethyl-sulfonyl.

In a further embodiment, In a further embodiment, R₃ is selected from t-butoxy-carbonyl, dimethylamino-carbonyl, methyl-sulfonyl, isopropoxy-carbonyl(ethyl)amino-methyl, isopropoxy-carbonyl-amino-methyl, benzyl(ethyl)amino-methyl, piperidinyl, quinazolinyl, isopropoxy-carbonyl, thieno[2,3-d]pyrimidin-4-yl, 4H-1,2,4-triazolyl, cyclopropoxy-carbonyl, (1,2,4-oxadiazol-5-yl), tetrazolyl, thiazolyl, triazolyl, pyrimidinyl, pyrazinyl, pyridinyl, phenyl, benzimidazolyl and pyridazinyl; wherein said cyclopropoxy, quinazolinyl, thieno[2,3-d]pyrimidinyl, thiazolyl, oxadiazolyl, tetrazolyl, pyrimidinyl, pyrazinyl, pyridinyl, phenyl, benzimidazolyl or pyridazinyl can be optionally substituted by 1 to 2 radicals independently selected from halo, cyano, methyl, methoxy-carbonyl, carboxyl, isopropyl, t-butyl, cyclopropyl, morpholino, methyl-piperazinyl-methyl, morpholino-methyl, ethoxy-methoxy-methyl, hydroxy-methyl, methoxy-ethoxy-methyl, methoxy-methoxy-methyl, ethoxy, trifluoromethyl, pentyl, phenyl, methoxy, dimethylamino, dimethylamino-methyl, dimethylamino-ethyl, aminoethyl, methoxy-carbonyl-methyl, methoxy-ethyl, hydroxyl-ethyl, pyrrolidinoethyl, t-butoxycarbonylamino-propoxy-methyl, morpholino-ethyl, aminopropoxy-methyl, dimethylamino-methyl, diethylamino-methyl, isopropyl-piperazino-ethyl, methoxy-ethoxy-ethoxy-methyl, methoxy-methyl, propyl and ethyl.

In a further embodiment is a compound selected from: 6-(3-(2-(4-ethylpiperidin-1-yl)pyrimidin-5-yl)propoxy)-2-(methylsulfonyl)-1,2,3,4-tetrahydroisoquinoline; 2-(methylsulfonyl)-6-(3-(6-phenylpyridin-3-yl)propoxy)-1,2,3,4-tetrahydroisoquinoline; 2-(methylsulfonyl)-6-(3-(5-phenylpyridin-2-yl)propoxy)-1,2,3,4-tetrahydroisoquinoline; 4-(5-(3-(2-(methylsulfonyl)-1,2,3,4-tetrahydroisoquinolin-6-yloxy)propyl)pyridin-2-yl)morpholino; 2-(Methylsulfonyl)-6-(3-(6-phenylpyridin-3-yl)propoxy)-1,2,3,4-tetrahydroisoquinoline; 2-(Methylsulfonyl)-6-(3-(5-phenylpyridin-2-yl)propoxy)-1,2,3,4-tetrahydroisoquinoline; and 4-(5-(3-(2-(Methylsulfonyl)-1,2,3,4-tetrahydroisoquinolin-6-yloxy)propyl)pyridin-2-yl)morpholine.

Further compounds of the invention are detailed in the Examples and Table I, infra.

### Pharmacology and Utility

Compounds of the invention modulate the activity of GPR119 and, as such, are useful for treating diseases or disorders in which the activity of GPR119 contributes to the pathology and/or symptomology of the disease. This invention further provides compounds of this invention for use in the preparation of medicaments for the treatment of diseases or disorders in which GPR119 activity contributes to the pathology and/or symptomology of the disease.

The resultant pathologies of Type II diabetes are impaired insulin signaling at its target tissues and failure of the insulin-producing cells of the pancreas to secrete an appropriate degree of insulin in response to a hyperglycemic signal. Current therapies to treat the latter include inhibitors of the β-cell ATP-sensitive potassium channel to trigger the release of endogenous insulin stores, or administration of exogenous insulin. Neither of these achieves accurate normalization of blood glucose levels and both carry the risk of inducing hypoglycemia. For these reasons, there has been intense interest in the development of pharmaceuticals that function in a glucose-dependent action, i.e. potentiators of glucose signaling. Physiological signaling systems which function in this manner are well-characterized and include the gut peptides GLP-1, GIP and PACAP. These hormones act via their cognate G-protein coupled receptor to stimulate the production of cAMP in pancreatic β-cells. The increased cAMP does not appear to result in stimulation of insulin release during the fasting or pre-prandial state. However, a series of biochemical targets of cAMP signaling, including the ATP-sensitive potassium channel, voltage-sensitive potassium channels and the exocytotic machinery, are modified in such a way that the insulin secretory response to a postprandial glucose stimulus is markedly enhanced. Accordingly, agonists of novel, similarly functioning, β-cell GPCRs, including GPR119, would also stimulate the release of endogenous insulin and consequently promote normoglycemia in Type II diabetes. It is also established that increased cAMP, for example as a result of GLP-1 stimulation, promotes β-cell proliferation, inhibits β-cell death and thus improves islet mass. This positive effect on β-cell mass is expected to be beneficial in both Type II diabetes, where insufficient insulin is produced, and Type I diabetes, where β-cells are destroyed by an inappropriate autoimmune response.

Some β-cell GPCRs, including GPR119, are also present in the hypothalamus where they modulate hunger, satiety, decrease food intake, controlling or decreasing weight and energy expenditure. Hence, given their function within the hypothalamic circuitry, agonists or inverse agonists of these receptors mitigate hunger, promote satiety and therefore modulate weight.

It is also well-established that metabolic diseases exert a negative influence on other physiological systems. Thus, there is often the codevelopment of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which clearly occur secondary to diabetes (e.g. kidney disease, peripheral neuropathy). Thus, it is expected that effective treatment of the diabetic condition will in turn be of benefit to such interconnected disease states.

In an embodiment of the invention is a method for treatment of a metabolic disease and/or a metabolic-related disorder in an individual comprising administering to the individual in need of such treatment a therapeutically effective amount of a compound of the invention or a pharmaceutical composition thereof. The metabolic diseases and metabolic-related disorders are selected from, but not limited to, hyperlipidemia, type 1 diabetes, type 2 diabetes mellitus, idiopathic type 1 diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.

In an embodiment of the invention are therapeutic benefits of GPR119 activity modulators derived from increasing levels of GIP and PPY. For example, neuroprotection, learning and memory, seizures and peripheral neuropathy.

GLP-1 and GLP-1 receptor agonists have been shown to be effective for treatment of neurodegenerative diseases and other neurological disorders. GLP-1 and exendin-4 have been shown to stimulate neurite outgrowth and enhance cell survival after growth factor withdrawal in PC12 cells. In a rodent model of neurodegeneration, GLP-1 and exendin-4 restore cholinergic marker activity in the basal forebrain. Central infusion of GLP-1 and exendin-4 also reduce the levels of amyloid-P peptide in mice and decrease amyloid precursor protein amount in cultured PC12 cells. GLP-1 receptor agonists have been shown to enhance learning in rats and the GLP-1 receptor knockout mice show deficiencies in learning behavior. The knockout mice also exhibit increased susceptibility to kainate-induced seizures which can be prevented by administration of GLP-1 receptor agonists. GLP-1 and exendin-4 has also been shown to be effective in treating pyridoxine-induced peripheral nerve degeneration, an experimental model of peripheral sensory neuropathy.

Glucose-dependent insulinotropic polypeptide (GIP) has also been shown to have effects on proliferation of hippocampal progenitor cells and in enhancing sensorimotor coordination and memory recognition.

In an embodiment of the invention are therapeutic benefits of GPR119 activity modulators. For example, GLP-2 and short bowel syndrome (SBS). Several studies in animals and from clinical trials have shown that GLP-2 is a trophic hormone that plays an important role in intestinal adaptation. Its role in regulation of cell proliferation, apoptosis, and nutrient absorption has been well documented. Short bowel syndrome is characterized by malabsorption of nutrients, water and vitamins as a result of disease or surgical removal of parts of the small intestine (eg. Crohn's disease). Therapies that improve intestinal adaptation are thought to be beneficial in treatment of this disease. In fact, phase II studies in SBS patients have shown that teduglutide, a GLP-2 analog, modestly increased fluid and nutrient absorption.

In an embodiment of the invention are therapeutic benefits of GPR119 activity modulators derived from increasing levels of GIP and PPY. For example, GLP-1, GIP and osteoporosis. GLP-1 has been shown to increase calcitonin and calcitonin related gene peptide (CGRP) secretion and expression in a murine C-cell line (CA-77). Calcitonin inhibits bone resorption by osteoclasts and promotes mineralization of skeletal bone. Osteoporosis is a disease that is characterized by reduced bone mineral density and thus GLP-1 induced increase in calcitonin might be therapeutically beneficial.

GIP has been reported to be involved in upregulation of markers of new bone formation in osteoblasts including collagen type I mRNA and in increasing bone mineral density. Like GLP-1, GIP has also been shown to inhibit bone resorption.

In an embodiment of the invention are therapeutic benefits of GPR119 activity modulators derived from increasing levels of GIP and PPY. For example, PPY and gastric emptying. GPR119 located on the pancreatic polypeptide (PP) cells of the islets has been implicated in the secretion of PPY. PPY has been reported to have profound effects on various physiological processes including modulation of gastric emptying and gastrointestinal motility. These effects slow down the digestive process and nutrient uptake and thereby prevent the postprandial elevation of blood glucose. PPY can suppress food intake by changing the expression of hypothalamic feeding-regulatory peptides. PP-overexpressing mice exhibited the thin phenotype with decreased food intake and gastric emptying rate.

In accordance with the foregoing, the present invention further provides a method for preventing or ameliorating the symptamology of any of the diseases or disorders described above in a subject in need thereof, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

Compounds of the invention can be administered in therapeutically effective amounts in combination with one or more therapeutic agents (pharmaceutical combinations).

For example, synergistic effects can occur with other anti-obesity agents, anorectic agents, appetite suppressant and related agents. Diet and/or exercise can also have synergistic effects. Anti-obesity agents include, but are not limited to, apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agents, β3 adrenergic receptor agonists, dopamine agonists (for example, bromocriptine), melanocyte-stimulating hormone receptor analogs, cannabinoid 1 receptor antagonists [for example, compounds described in WO2006/047516), melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion).

Where compounds of the invention are administered in conjunction with other therapies, dosages of the co-administered compounds will of course vary depending on the type of co-drug employed, on the specific drug employed, on the condition being treated and so forth.

A combined preparation or pharmaceutical composition can comprise a compound of the invention as defined above or a pharmaceutical acceptable salt thereof and at least one active ingredient selected from:

a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, N,N-57-05441 and N,N-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid described in the patent application WO 03/043985, as compound 19 of Example 4, a non-glitazone type PPAR gamma agonist e.g. GI-262570; Diacylglycerol acetyltransferase (DGAT) inhibitors such as those disclosed in WO 2005044250, WO 2005013907, WO 2004094618 and WO 2004047755;

b) hypolipidemic agents such as 3-hydroxy-3-methyl-glutaryl coenzyme A (HMG-CoA) reductase inhibitors, e.g., lovastatin and related compounds such as those disclosed in U.S. Pat. No. 4,231,938, pitavastatin, simvastatin and related compounds such as those disclosed in U.S. Pat. Nos. 4,448,784 and 4,450,171, pravastatin and related compounds such as those disclosed in U.S. Pat. No. 4,346,227, cerivastatin, mevastatin and related compounds such as those disclosed in U.S. Pat. No. 3,983,140, velostatin, fluvastatin, dalvastatin, atorvastatin, rosuvastatin and related statin compounds disclosed in U.S. Pat. No. 5,753,675, rivastatin, pyrazole analogs of mevalonolactone derivatives as disclosed in U.S. Pat. No. 4,613,610, indene analogs of mevalonolactone derivatives as disclosed in PCT application WO 86/03488, 6-[2-(substituted-pyrrol-1-yl)-alkyl)pyran-2-ones and derivatives thereof as disclosed in U.S. Pat. No. 4,647,576, Searle's SC-45355 (a 3-substituted pentanedioic acid derivative) dichloroacetate, imidazole analogs of mevalonolactone as disclosed in PCT application WO 86/07054, 3-carboxy-2-hydroxy-propane-phosphonic acid derivatives as disclosed in French Patent No. 2,596,393, 2,3-disubstituted pyrrole, furan and thiophene derivatives as disclosed in European Patent Application No. 0221025, naphthyl analogs of mevalonolactone as disclosed in U.S. Pat. No. 4,686,237, octahydronaphthalenes such as disclosed in U.S. Pat. No. 4,499,289, keto analogs of mevinolin (lovastatin) as disclosed in European Patent Application No. 0,142,146 A2, and quinoline and pyridine derivatives disclosed in U.S. Pat. Nos. 5,506,219 and 5,691,322. In addition, phosphinic acid compounds useful in inhibiting HMG CoA reductase suitable for use herein are disclosed in GB 2205837; squalene synthase inhibitors; FXR (framesoid X receptor) and LXR (liver X receptor) ligands; cholestyramine; fibrates; nicotinic acid and aspirin;

c) an anti-obesity agent or appetite regulating agent such as a CB1 activity modulator, melanocortin receptor (MC4R) agonists, melanin-concentrating hormone receptor (MCHR) antagonists, growth hormone secretagogue receptor (GHSR) antagonists, galanin receptor modulators, orexin antagonists, CCK agonists, GLP-1 agonists, and other Pre-proglucagon-derived peptides; NPY1 or NPY5 antagonist, NPY2 and NPY4 modulators, corticotropin releasing factor agonists, histamine receptor-3 (H3) modulators, aP2 inhibitors, PPAR gamma modulators, PPAR delta modulators, acetyl-CoA carboxylase (ACC) inhibitors, 1′-β-HSD-1 inhibitors, adinopectin receptor modulators; beta 3 adrenergic agonists, such as AJ9677 (Takeda/Dainippon), L750355 (Merck), or CP331648 (Pfizer) or other known beta 3 agonists as disclosed in U.S. Pat. Nos. 5,541,204, 5,770,615, 5, 491,134, 5,776,983 and 5,488,064, a thyroid receptor beta modulator, such as a thyroid receptor ligand as disclosed in WO 97/21993 (U. Cal SF), WO 99/00353 (KaroBio) and GB98/284425 (KaroBio), a SCD-1 inhibitor as disclosed in WO2005011655, a lipase inhibitor, such as orlistat or ATL-962 (Alizyme), serotonin receptor agonists, (e.g., BVT-933 (Biovitrum)), monoamine reuptake inhibitors or releasing agents, such as fenfluramine, dexfenfluramine, fluvoxamine, fluoxetine, paroxetine, sertraline, chlorphentermine, cloforex, clortermine, picilorex, sibutramine, dexamphetamine, phentermine, phenylpropanolamine or mazindol, anorectic agents such as topiramate (Johnson & Johnson), CNTF (ciliary neurotrophic factor)/Axokine® (Regeneron), BDNF (brain-derived neurotrophic factor), leptin and leptin receptor modulators, phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine;

d) anti-hypertensive agents such as loop diuretics such as ethacrynic acid, furosemide and torsemide; diuretics such as thiazide derivatives, chlorithiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; beta-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; aldosterone synthase inhibitors; and dual ET/AII antagonist such as those disclosed in WO 00/01389.

e) a HDL increasing compound;

f) Cholesterol absorption modulator such as Zetia® and KT6-971;

g) Apo-A1 analogues and mimetics;

h) thrombin inhibitors such as Ximelagatran;

i) aldosterone inhibitors such as anastrazole, fadrazole, eplernone;

j) Inhibitors of platelet aggregation such as aspirin, clopidogrel bisulfate;

k) estrogen, testosterone, a selective estrogen receptor modulator, a selective androgen receptor modulator;

l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib ({N-{5-[4-(4-methyl-piperazino-methyl)-benzoylamido]-2-methylphenyl}-4-(3-pyridyl)-2-pyrimidine-amine}) described in the European patent application EP-A-0 564 409 as example 21 or 4-Methyl-N-[3-(4-methyl-imidazol-1-yl)-5-trifluoromethyl-phenyl]-3-(4-pyridin-3-yl-pyrimidin-2-ylamino)-benzamide described in the patent application WO 04/005281 as example 92; and

m) an agent interacting with a 5-HT₃ receptor and/or an agent interacting with 5-HT₄ receptor such as tegaserod described in the U.S. Pat. No. 5,510,353 as example 13, tegaserod hydrogen maleate, cisapride, cilansetron;

n) an agent for treating tobacco abuse, e.g., nicotine receptor partial agonists, bupropion hypochloride (also known under the tradename Zyban®) and nicotine replacement therapies;

o) an agent for treating erectile dysfunction, e.g., dopaminergic agents, such as apomorphine), ADD/ADHD agents (e.g., Ritalin®, Strattera®, Concerta® and Adderall®);

p) an agent for treating alcoholism, such as opioid antagonists (e.g., naltrexone (also known under the tradename ReVia®) and nalmefene), disulfuram (also known under the tradename Antabuse®), and acamprosate (also known under the tradename Campral®)). In addition, agents for reducing alcohol withdrawal symptoms may also be co-administered, such as benzodiazepines, beta-blockers, clonidine, carbamazepine, pregabalin, and gabapentin (Neurontin®);

q) other agents that are useful including anti-inflammatory agents (e.g., COX-2 inhibitors); antidepressants (e.g., fluoxetine hydrochloride (Prozac®)); cognitive improvement agents (e.g., donepezil hydrochloride (Aircept®) and other acetylcholinesterase inhibitors); neuroprotective agents (e.g., memantine); antipsychotic medications (e.g., ziprasidone (Geodon®), risperidone (Risperdal®), and olanzapine (Zyprexa®));

or, in each case a pharmaceutically acceptable salt thereof; and optionally a pharmaceutically acceptable carrier.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

In the following schemes, several methods of preparing the compounds of the present invention are illustrative. One of skill in the art will appreciate that these methods are representative, and in no way inclusive of all methods for preparing the compounds of the present invention. The radicals in the schemes, R₁, R₂ₐ, R_{2b}, L and B, are described in the Summary of the Invention.

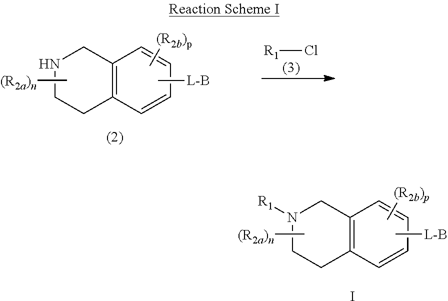

A compound of Formula I can be prepared by reacting a compound of formula 2 with a compound of formula 3 in the presence of a suitable solvent (for example, methylene chloride, and the like) and a suitable base (for example, pyridine, triethylamine, and the like). The reaction proceeds at a temperature of about 0° C. to about 50° C. and can take up to 24 hours to complete.

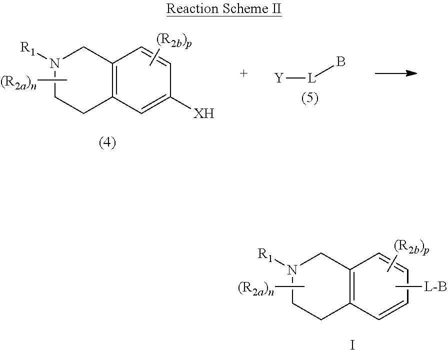

A compound of Formula I can be prepared by reacting a compound of formula 4 with a compound of formula 5, where Y is a leaving group (for example OMs, Br and the like) and X is O or N and the like, in the presence of a suitable solvent (for example, dimethylformamide, and the like) and a suitable base (for example, pyridine, triethylamine, K₂CO₃ and the like). The reaction proceeds at a temperature of about 0° C. to about 120° C. and can take up to 24 hours to complete.

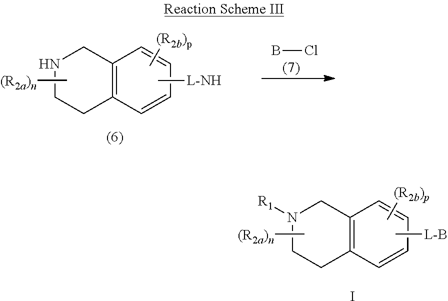

A compound of Formula I can be prepared by reacting a compound of formula 5 with a compound of formula 7 in the presence of a suitable solvent (for example, dimethylformamide, and the like) and a suitable base (for example, pyridine, triethylamine, K₂CO₃ and the like). The reaction proceeds at a temperature of about 0° C. to about 160° C. and can take up to 24 hours to complete.

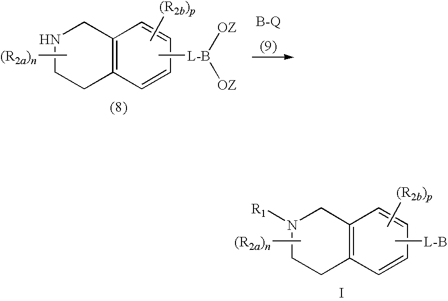

A compound of Formula I can be prepared by reacting a compound of formula 8 with a compound of formula 9 (where Q is a halogen, OMs, OTf and the like; Z is H, alkyl, and the like) in the presence of a suitable solvent (for example, dioxane, water and the like), a suitable base (for example, Na₂CO₃ and the like) and a catalyst ((Pd (PPh₃)₄ and the like). The reaction proceeds at a temperature of about 0° C. to about 160° C. and can take up to 24 hours to complete.

Detailed descriptions of the synthesis of compounds of the Invention are given in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme I; and
(b) optionally converting a compound of the invention into a pharmaceutically acceptable salt;
(c) optionally converting a salt form of a compound of the invention to a non-salt form;
(d) optionally converting an unoxidized form of a compound of the invention into a pharmaceutically acceptable N-oxide;
(e) optionally converting an N-oxide form of a compound of the invention to its unoxidized form;
(f) optionally resolving an individual isomer of a compound of the invention from a mixture of isomers;
(g) optionally converting a non-derivatized compound of the invention into a pharmaceutically acceptable prodrug derivative; and
(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following Examples that illustrate the preparation of compounds of the invention.

### Example 1

### Isopropyl 4-(2-(2-(methylsulfonyl)-1,2,3,4-tetrahydroisoquinolin-6-yloxy)ethyl)piperidine-1-carboxylate

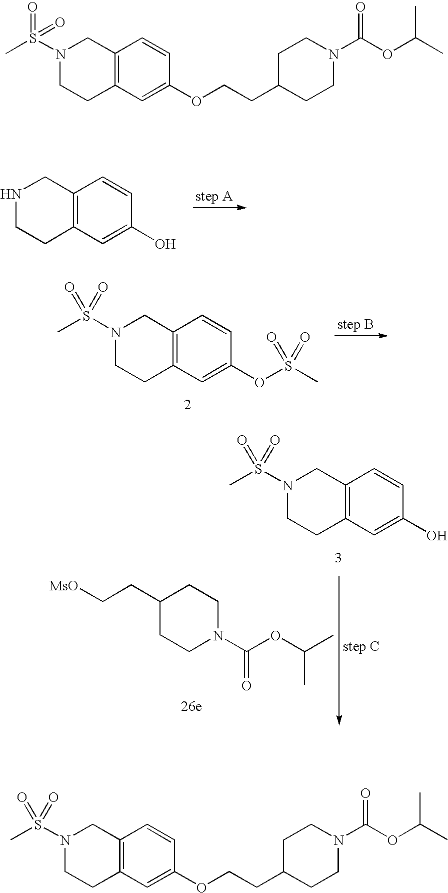

Step A 2-(Methylsulfonyl)-1,2,3,4-tetrahydroisoquinolin-6-yl methanesulfonate (2). To a mixture of 1,2,3,4-tetrahydroisoquinolin-6-ol HBr salt (1 g, 4.3 mmol) and triethylamine (1.8 mL, 12.9 mmol) in dichloromethane (30 mL) was slowly added methanesulfonyl chloride (0.5 mL, 6.4 mmol) at 0° C. The reaction mixture was stirred overnight at rt. Methylene chloride (20 mL) was added and the mixture was washed with saturated NH₄Cl. The organics were dried and solvents were removed under reduced pressure to give the desired product which was used directly for the next step without purification. MS calcd. for [M+H]⁺ C₁₁H₁₆NO₅S₂: 306.0; found: 306.0.

Step B 2-(Methylsulfonyl)-1,2,3,4-tetrahydroisoquinolin-6-ol (3). To a suspension of 2 in methanol (20 mL) was added aqueous 10% NaOH (20 mL), and the reaction mixture was stirred at 80° C. for 2 h. The mixture was cooled to rt, poured into ethyl acetate (30 mL) and the organics were separated, washed with saturated NH₄Cl, brine, dried and filtered. Solvents were removed under reduced pressure and the crude was purified on silica gel (EtOAc:Hexanes=1:1) to afford desired product as a white solid. ¹H-NMR (400 MHz, CDCl₃) δ 6.95 (1H, d, J=8.4 Hz), 6.70 (1H, dd, J=1.2 Hz, J=8 Hz), 6.63 (1H, d, J=1.2 Hz), 4.38 (3H, s), 3.53 (3H, m), 2.91 (3H, m), 2.82 (3H, s). MS calcd. for [M+H]⁺ C₁₀H₁₄NO₃S: 228.1; found: 228.1

Step C Isopropyl 4-(2-(2-(methylsulfonyl)-1,2,3,4-tetrahydroisoquinolin-6-yloxy)ethyl)piperidine-1-carboxylate A reaction vessel was charged with 2-(methylsulfonyl)-1,2,3,4-tetrahydroisoquinolin-6-ol (3) (30 mg, 0.13 mmol), isopropyl 4-(2-(methylsulfonyloxy)ethyl)piperidine-1-carboxylate (26 e) (46.4 mg, 0.16 mmol), cesium carbonate (85 mg, 0.26 mmol) and acetonitrile (3 mL). The mixture was stirred at 80° C. for 2 h. It was filtered through a celite pad. Solvents were removed under reduced pressure and the residue was purified by reverse phase HPLC to afford the title compound as a white solid. MS calcd. for [M+H]⁺ C₂₁H₃₃N₂O₅S: 425.2; found: 425.2.

Examples 8, 9, 11, 12, 21 were prepared by analogous method from example 1.

### Example 2

### tert-Butyl 4-(2-(2-(methylsulfonyl)-1,2,3,4-tetrahydroisoquinolin-7-yloxy)-ethyl)piperidine-1-carboxylate

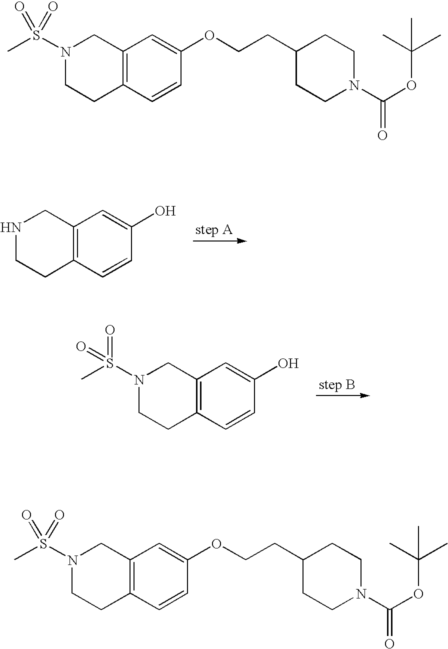

Step A 2-(Methylsulfonyl)-1,2,3,4-tetrahydroisoquinolin-7-ol. 2-(methylsulfonyl)-1,2,3,4-tetrahydroisoquinolin-7-ol was prepared following the method detailed for compound 3 in Example 1 using 1,2,3,4-tetrahydroisoquinolin-7-ol as starting material. MS calcd. for [M+H]⁺ C₁₀H₁₄NO₃S: 228.1; found: 228.1

Step B tert-Butyl 4-(2-(2-(methylsulfonyl)-1,2,3,4-tetrahydroisoquinolin-7-yloxy)ethyl)piperidine-1-carboxylate. The title compound was synthesized according to Example 1 from the corresponding 2-(methylsulfonyl)-1,2,3,4-tetrahydroisoquinolin-7-ol (7) and tert-butyl 4-(2-(methylsulfonyloxy)ethyl)piperidine-1-carboxylate. MS calcd. for [M+H]⁺ C₂₂H₃₅N₂O₅S: 439.2; found: 439.2.

### Example 3

### tert-Butyl 4-(2-(2-(methylsulfonyl)-1,2,3,4-tetrahydroisoquinolin-5-yloxy)-ethyl)piperidine-1-carboxylate

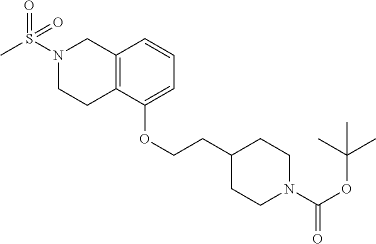

tert-Butyl 4-(2-(2-(methylsulfonyl)-1,2,3,4-tetrahydroisoquinolin-5-yloxy)ethyl)piperidine-1-carboxylate. The title compound was synthesized according to Example 1 from the corresponding 2-(methylsulfonyl)-1,2,3,4-tetrahydroisoquinolin-5-ol and tert-butyl 4-(2-(methylsulfonyloxy)ethyl)piperidine-1-carboxylate. MS calcd. for [M+H]⁺ C₂₂H₃₅N₂O₅S: 439.2; found: 439.2.

### Example 4

### tert-Butyl 4-(3-(1-(methylsulfonyl)-1,2,3,4-tetrahydroquinolin-6-yloxy)-propyl)piperidine-1-carboxylate

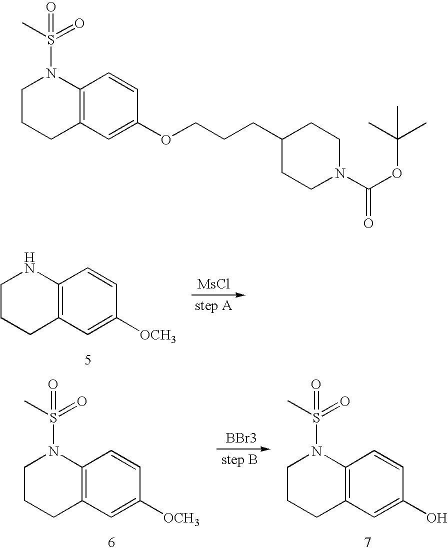

Step A 1-(Methylsulfonyl)-1,2,3,4-tetrahydroquinolin-6-yl methanesulfonate (6). To a solution of commercially available 6-methoxy-1,2,3,4-tetrahydroquinoline (5) (500 mg, 3.1 mmol) in dichloromethane (20 mL) was added triethylamine (864 μL, 6.2 mmol). Methanesulfonyl chloride (482 uL, 6.2 mmol) was added slowly at 0° C. and the mixture was stirred for 3 h. Water (1 mL) was added to quench the reaction and the mixture was washed with brine, dried over Na₂SO₄ and filtered. Solvents were removed in vacuo and the crude material was purified by silica gel chromatography (EtOAc:Hexanes=1:1) to afford the desired product as a white solid. MS Calcd for [M+H]⁺: C₁₁H₁₆NO₃S: 242.1; found: 242.0

Step B 1-(Methylsulfonyl)-1,2,3,4-tetrahydroquinolin-6-ol. A solution of 6 (200 mg, 0.88 mmol) in dichloromethane was cooled to −78° C. in a dry ice/acetone bath. BBr₃ in dichloromethane (2.4 mL, 1.0 M, 2.4 mmol) was added dropwise. The cooling bath was removed and the mixture was allowed to warm to rt. After stirring for 1 h at rt, saturated sodium bicarbonate was added and the mixture was extracted with dichloromethane. The organics were combined and washed with brine, dried, concentrated under reduced pressure. The residue was purified by silica gel chromatography (EtOAc:Hexanes=1:2) to give the desired product. MS Calcd for [M+H]⁺: C₁₀H₁₄NO₃S: 228.1; found: 228.1

Step C tert-Butyl 4-(3-(1-(methylsulfonyl)-1,2,3,4-tetrahydroquinolin-6-yloxy)propyl)piperidine-1-carboxylate. The title compound was synthesized according to Example 1 from the corresponding 1-(methylsulfonyl)-1,2,3,4-tetrahydroquinolin-6-ol and tert-butyl 4-(3-(methylsulfonyloxy)propyl)piperidine-1-carboxylate. MS calcd. for [M+H]⁺ C₂₃H₃₇N₂O₅S: 453.2; found: 453.2.

### Example 5

### Isopropyl 4-(2-(2-(ethylsulfonyl)-1,2,3,4-tetrahydroisoquinolin-6-yloxy)ethyl)-piperidine-1-carboxylate

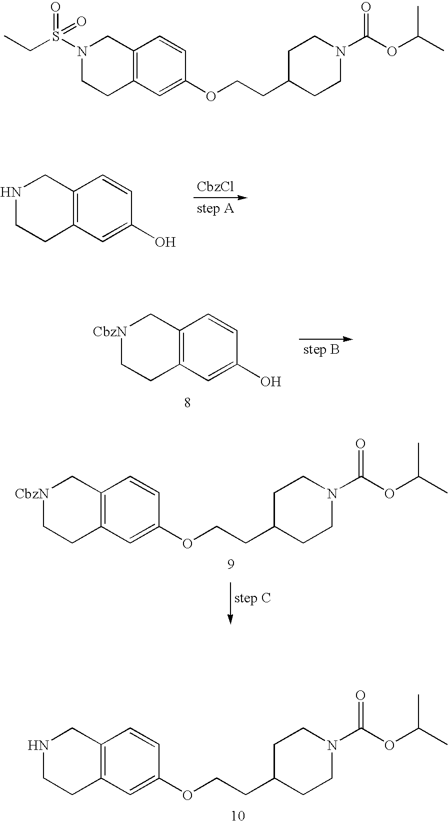

Step A Benzyl 6-hydroxy-3,4-dihydroisoquinoline-2(1H)-carboxylate (8). A solution of 1,2,3,4-tetrahydroisoquinolin-6-ol (HBr salt) (1 g, 4.3 mmol) in dioxane/water (1:1, 20 mL) was adjusted to pH 9 by adding 1N NaOH aqueous solution. The solution was cooled to 0° C. in an ice water bath, and then benzyl chloroformate was added over 5 minutes while maintaining the reaction temperature at 0° C. and the pH between 9 and 9.5. The completion of the reaction was monitored by LC-MS. The mixture was then poured into water (20 mL) and extracted with ethyl acetate. The organics were combined, dried and concentrated under reduced pressure. The crude was purified by silica gel chromatography (EtOAc:Hexane=1:1) to afford the desired product. MS calcd. for [M+H]⁺: C₁₇H₁₆NO₃: 284.1; found: 284.1.

Step B Benzyl 6-(2-(1-(isopropoxycarbonyl)piperidin-4-yl)ethoxy)-3,4-dihydroisoquinoline-2(1H)-carboxylate (9). Intermediate 9 was synthesized according to Example 1 from the corresponding benzyl 6-hydroxy-3,4-dihydroisoquinoline-2(1H)-carboxylate (8) (500 mg, 1.8 mmol) and isopropyl 4-(2-(methylsulfonyloxy)-ethyl)piperidine-1-carboxylate (4). MS calcd. for [M+H]⁺: C₂₈H₃₆N₂O₅: 481.3; found: 481.3.

Step C Isopropyl 4-(2-(1,2,3,4-tetrahydroisoquinolin-6-yloxy)ethyl)piperidine-1-carboxylate (10). Intermediate 9 (864 mg, 1.8 mmol) was dissolved in methanol (30 mL) and palladium on carbon (10%, 300 mg) was added. The mixture was stirred under a hydrogen atmosphere for 30 minutes and then filtered through Celite. Removal of solvent under reduced pressure afforded intermediate 10 as a yellow oil. MS calcd. for [M+H]⁺: C₂₀H₃₀N₂O₃: 347.2; found: 347.2.

Isopropyl 4-(2-(2-(ethylsulfonyl)-1,2,3,4-tetrahydroisoquinolin-6-yloxy)ethyl)piperidine-1-carboxylate. To a solution of amine 10 (10 mg, 0.029 mmol), and triethylamine (8.2 uL, 0.058 mmol) in dichloromethane was added ethanesulfonyl chloride (5.5 uL, 0.058 mmol) at 0° C. The mixture was stirred at rt for 1 h. Water was added and organic layer was separated, washed with brine, dried (Na₂SO₄) and filtered. Solvents were removed under reduced pressure and the crude product was purified via preparative HPLC to afford the title compound as a white solid. MS calcd. for [M+H]⁺ C₂₂H₃₅N₂O₅S: 439.2; found: 439.2.

Examples 14, 15, 16, 17, 18, 19, 20, 22, 23 were prepared by analogous method from example 25.

### Example 6

### Isopropyl 4-(5-((2-(methylsulfonyl)-1,2,3,4-tetrahydroisoquinolin-6-yloxy)methyl)-1,2,4-oxadiazol-3-yl)piperidine-1-carboxylate (6)

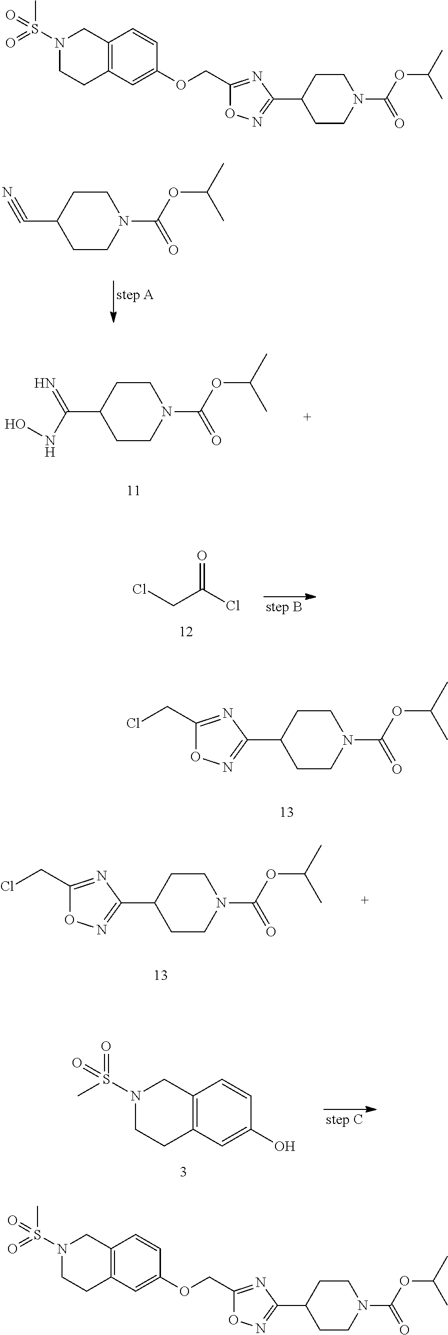

Step A Isopropyl 4-(N-hydroxycarbamimidoyl)piperidine-1-carboxylate (11) A mixture of isopropyl 4-cyanopiperidine-1-carboxylate (1.96 gram, 10 mmol) and hydroxylamine (5 mL) in propanol (50 mL) was heated under reflux for 5 hour. The mixture was filtered and solid was collected, washed with water (5 mL) and air dried to provided the desired product. MS calcd. for [M+H]⁺: C₁₀H₂₀N₃O₃: 230.1; found: 230.1.

Step B Isopropyl 4-(5-(chloromethyl)-1,2,4-oxadiazol-3-yl)piperidine-1-carboxylate (13). To a solution of 11 (30 mg, 0.1 mmol) in dichloromethane (3 mL) was added triethylamine (100 uL, 0.7 mmol) and the mixture was stirred at rt for 10 minutes. Chloroacetyl chloride (50 uL, 0.62 mmol) was added slowly and the resulting mixture was stirred at rt for 24 hours. Water was added and the mixture was extracted with dichloromethane (2×5 mL). The organics were combined, washed with brine and dried (MgSO₄). Solvents were removed under reduced pressure; the residue was dried under high vacuum overnight and used directly for the next step without purification.

Step C The above intermediate was dissolved in DMF (2 mL) followed by addition of intermediate 3 (12 mg, 0.04 mmol) and Cs₂CO₃ (50 mg, 0.16 mmol). The mixture was stirred overnight at rt. Water was added, and the mixture was extracted with ethyl acetate (3×5 mL). The organics were washed with water, brine, dried over Na₂SO₄, and filtered. Solvents were removed under reduced pressure and the crude was purified via silica gel flash chromatography (EtOAc:Hexanes=1:1) to give the title compound as a white solid. MS calcd. for [M+H]⁺ C₂₂H₃₁N₄O₆S: 479.2; found: 479.2.

### Example 24

### 6-(3-(1-(5-Ethylpyrimidin-2-yl)piperidin-4-yl)propoxy)-2-(methylsulfonyl)-1,2,3,4-tetrahydroisoquinoline

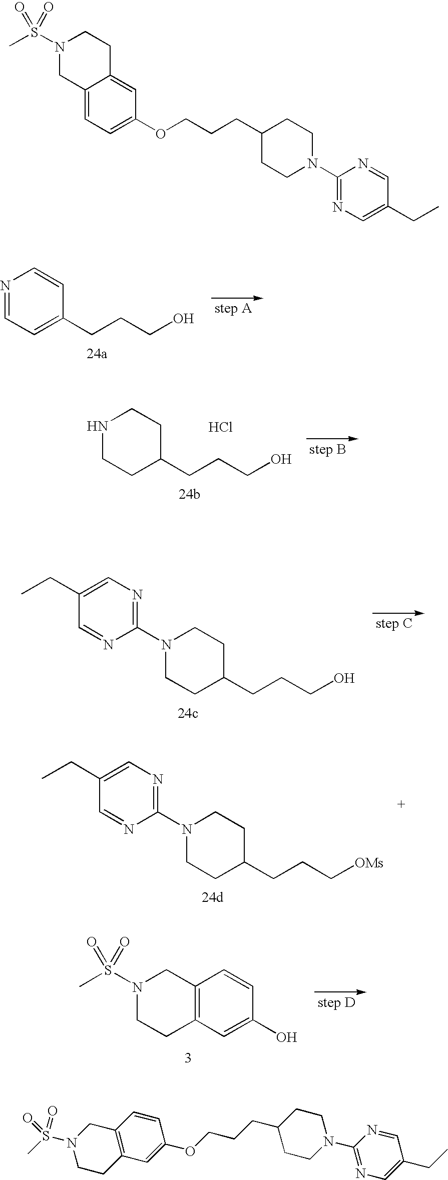

Step A 3-(Piperidin-4-yl)propan-1-ol hydrochloride (24b). To a 500 mL hydrogenation flask was added a solution of 3-(pyridin-4-yl)propan-1-ol (25 g, 182.5 mmol) in ethanol (200 mL). Concentrated HCl (25 mL) was added followed by addition of PtO₂ (200 mg). The mixture was subjected to H₂ (60 psi) in a Parr shaker for 20 h. Then solvents were removed under reduced pressure and the product was dried in vacuo overnight to afford intermediate 24b. MS calcd. for [M+H]⁺ C₈H₁₈NO: 144.1; found: 144.1.

Step B 3-(1-(5-Ethylpyrimidin-2-yl)piperidin-4-yl)propan-1-ol (24c). A round bottom flask was charged with 3-(piperidin-4-yl)propan-1-ol hydrochloride (2) (1.8 g, 10 mmol), 2-chloro-5-ethylpyrimidin (1.44 g, mmol), Cs₂CO₃ (7 g, 10.1 mmol) and DMF (25 mL). The mixture was heated at 120° C. for 20 h. Then it was cooled to rt and EtOAc (100 mL) was added followed by water (50 mL). The mixture was separated, and the organic layer was washed with water (3×30 mL) and brine (30 mL), dried over Na₂SO₄ and filtered. The solvents were removed under reduced pressure and the crude was purified via flash column chromatography (EtOAc:Hexanes=2:1) to give intermediate 24c as a solid. MS calcd. for [M+H]⁺ C₁₄H₂₄N₃O: 250.1; found: 250.1.

Step C 3-(1-(5-Ethylpyrimidin-2-yl)piperidin-4-yl)propyl methanesulfonate (24d). To a solution of 3-(1-(5-ethylpyrimidin-2-yl)piperidin-4-yl)propan-1-ol (1.25 g, 5 mmol) in CH₂Cl₂ (20 mL) was added Et₃N (1 mL, 7.2 mmol). The mixture was cooled to 0° C., and MsCl (0.41 mL) was added slowly. After the addition was completed, the reaction mixture was stirred for 3 h at rt, then quenched with water. CH₂Cl₂ (20 mL) was added and the mixture was washed with water (20 mL) and brine (2×20 mL). The organics were dried over Na₂SO₄. After the solvent was removed under reduced pressure, the crude was filtered through a short silica gel plug (10 g, washed with EtOAc:Hexanes=1:2). Removal of solvents under reduced pressure afforded the desired product 24d. MS calcd. for [M+H]⁺ C₁₅H₂₆N₃O₃S: 328.1; found: 328.1.

Step D 6-(3-(1-(5-Ethylpyrimidin-2-yl)piperidin-4-yl)propoxy)-2-(methylsulfonyl)-1,2,3,4-tetrahydroisoquinoline. A dry flask was charged with 3-(1-(5-ethylpyrimidin-2-yl)piperidin-4-yl)propyl methanesulfonate (0.52 g, 1.6 mmol), 2-(methylsulfonyl)-1,2,3,4-tetrahydroisoquinolin-6-ol, Cs₂CO₃ (0.7 g, 2.18 mmol) and DMF (8 mL). The mixture was stirred at rt for 12 h. EtOAc (50 mL) was added and the organics were washed with saturated NH₄Cl (50 mL), water (2×30 mL), brine (50 mL), dried over Na₂SO₄, and filtered. The solvents were removed under reduced pressure and the residue was dissolved in CH₂Cl₂, filtered through a short silica gel plug (EtOAc:Hexanes=1:1). Solvents were removed under reduced pressure to give crude product. Recrystallization of the crude from EtOH afforded the title compound as a white solid. MS calcd. for [M+H]⁺ C₂₄H₃₅N₄O₃S: 459.2; found: 459.2.

Example 25 was prepared by analogous method from example 24.

### Example 26

### Isopropyl 4-(2-(2-(methylsulfonyl)-1,2,3,4-tetrahydroisoquinolin-6-ylamino)ethyl)-piperidine-1-carboxylate

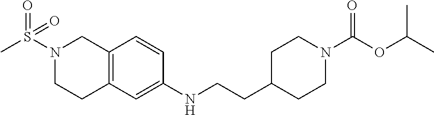

Intermediate 26c: 2-(Methylsulfonyl)-6-amino-1,2,3,4-tetrahydroisoquinoline:

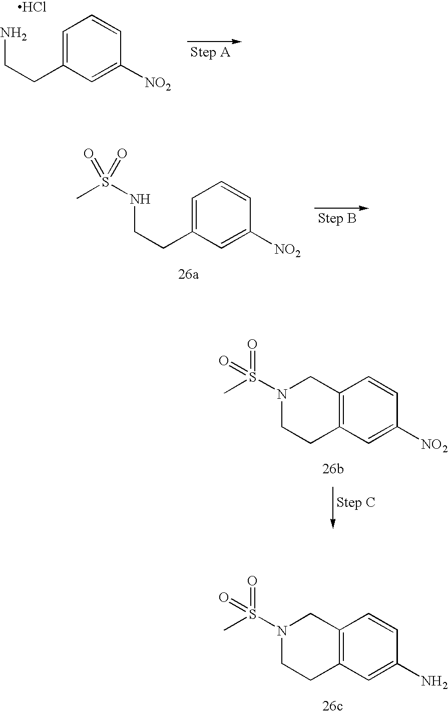

Step A Commercially available 3-nitrophenethylamine hydrochloride (4.52 g, 22.3 mmol) was dissolved/suspended in CH₂Cl₂ (150 ml) and treated with NEt₃ (6.84 ml, 49.0 mmol). The mixture was then cooled to 0° C. and methanesulfonyl chloride (1.9 ml, 24.4 mmol) was added dropwise. Upon completed addition, stirring was continued overnight at rt. The mixture was then diluted with CH₂Cl₂, washed with 50% sat. NH₄Cl and brine. The organic phase was dried over Na₂SO₄, filtered, and concentrated in vacuo to afford N-(3-nitrophenethyl)methanesulfonamide 26a as a white solid. The compound was used in the next step without further purification. MS calcd. for [M+H]⁺ C₉H₁₃N₂O₄S: 245.0; found: 245.0.

Step B Intermediate 26a (5.45 g, 22.3 mmol) was placed in a flask and cold H₂SO₄/AcOH solution (3:2 v/v, 50 ml) was added, followed by solid paraformaldehyde (1.36 g, 45.3 mmol). The mixture was then stirred at 45° C. for 3 h. The mixture was poured into ice and extracted with CH₂Cl₂. The organics were washed with sat. aqueous Na₂CO₃ and brine, dried over Na₂SO₄, filtered and concentrated in vacuo. Crystallization of the crude compound (EtOAc) yielded 2-(methylsulfonyl)-6-nitro-1,2,3,4-tetrahydroisoquinoline (26b) as a white solid. ¹H-NMR (400 MHz, CDCl₃) δ=8.10 (m, 2H), 7.30 (m, 1H), 4.57 (s, 2H), 3.63 (t, J=6.0 Hz, 2H), 3.13 (t, J=6.0 Hz, 2H), 2.92 (s, 3H); MS calcd. for [M+H]⁺ C₁₀H₁₃N₂O₄S: 257.0; found: 256.9.

Step C A round bottom flask was charged with intermediate 26b (3.93 g, 15.3 mmol) and EtOH/THF/CH₂Cl₂ (66:30:20 mL). AcOH (0.1 mL) was added, followed by Pd/C (10% wet, 400 mg). The flask was evacuated, flushed with hydrogen, and the mixture was stirred under H₂ (1 atm) for 48 h. The flask was then flashed with Ar, and the mixture was filtered through celite, washed with CH₂Cl₂ and MeOH. Concentration of the filtrate afforded 2-(methylsulfonyl)-6-amino-1,2,3,4-tetrahydroisoquinoline (26c) as a yellow solid. ¹H-NMR (400 MHz, CDCl₃) δ=6.88 (d, J=8.4 Hz, 1H), 6.55 (dd, J=8.4, 2.4 Hz, 1H), 6.47 (d, J=2.4 Hz, 1H), 4.35 (s, 2H), 3.62 (br. s, 2H), 3.52 (t, J=6.0 Hz, 2H), 2.87 (t, J=6.0 Hz, 2H), 2.81 (s, 3H); MS calcd. for [M+H]⁺ C₁₀H₁₅N₂O₂S: 227.1; found: 227.1.

Intermediate 26e: (1-(Isopropoxycarbonyl)piperidin-4-yl)ethyl methanesulfonate

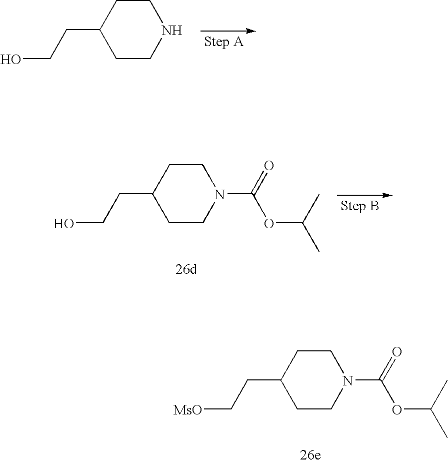

Step A Commercially available (piperidin-4-yl)ethanol (1.13 g, 8.7 mmol) was dissolved in dry dimethoxyethane (7.0 mL). NEt₃ (2.0 mL, 14.2 mmol) was added in one portion. To the resulting mixture, a solution of isopropyl chloroformate in toluene (1.0M, 9.5 mL) was added dropwise, with vigorous stirring, over 5 min. A white precipitate formed, and the suspension was stirred at rt overnight. The white precipitate was filtered off, washed with EtOAc, and discarded. The filtrate was concentrated in vacuo to yield isopropyl 4-(2-hydroxyethyl)piperidine-1-carboxylate (26d) as an oil. ¹H-NMR (400 MHz, DMSO-d₆) δ=4.74 (septet, J=6.3 Hz, 1H), 4.37 (t, J=6.2 Hz, 1H), 3.93 (d, J=11.1 Hz, 2H), 3.43 (td, J=6.6, 5.1 Hz, 2H), 2.70 (br. s, 2H), 1.62 (d, J=13.3 Hz, 2H), 1.54 (m, 1H), 1.35 (q, J=6.6 Hz, 2H), 1.17 (d, J=6.3 Hz, 6H), 0.96 (ddd, J=19.8, 12.8, 4.4 Hz, 2H).

Step B A sample of intermediate 26d (4.20 g, 19.5 mmol) was dissolved in dry CH₂Cl₂ (30 mL), then NEt₃ (4.0 mL, 28.5 mmol) was added. The resulting mixture was cooled to 0° C. Methanesulfonyl chloride (1.7 mL, 21.9 mmol) was added dropwise, with vigorous stirring, over 5 min. The ice-bath was removed and the resulting solution was stirred at rt for 30 min. The reaction mixture was added to water (40 mL) and extracted with CH₂Cl₂ (2×40 mL). The combined organic extracts were washed with sat. aqueous NH₄Cl, dried (MgSO₄), and concentrated in vacuo to yield isopropyl 4-(2-(methylsulfonyloxy)ethyl)piperidine-1-carboxylate (26e) as an oil. ¹H-NMR (400 MHz, DMSO-d₆) δ=4.75 (septet, J=6.3 Hz, 1H), 4.24 (t, J=6.2 Hz, 2H), 3.94 (d, J=15.0 Hz, 2H), 3.18 (s, 3H), 2.73 (br s, 2H), 1.61 (m, 5H), 1.17 (d, J=6.3 Hz, 6H), 1.03 (m, 2H); MS calcd. for C₁₂H₂₄NO₅S [M+H]⁺ 294.1; found: 294.1.

A sample of intermediate 26c (50 mg, 0.22 mmol), mesylate 26e (71 mg, 0.24 mmol), and Cs₂CO₃ (144 mg, 0.44 mmol) were dissolved/suspended in MeCN (1 mL) and stirred at 90° C. overnight. The mixture was then diluted with MeCN and filtered. The filtrate was purified by reverse-phase HPLC to yield the title compound 26. ¹H-NMR (400 MHz, CD₃CN) δ=7.20 (d, J=8.4 Hz, 1H), 7.09-7.05 (m, 2H), 5.26 (br s, 1H), 4.83 (septet, J=6.0 Hz, 1H), 4.40 (s, 2H), 4.05 (d, J=12.4 Hz, 2H), 3.50 (t, J=6.0 Hz, 2H), 3.31 (t, J=7.2 Hz, 2H), 2.96 (t, J=6.0 Hz, 2H), 2.86 (s, 3H), 2.80-2.67 (m, 2H), 1.73-1.52 (m, 5H), 1.22 (d, J=6.0 Hz, 6H), 1.15-1.00 (m, 2H); MS calcd. for [M+H]⁺ C₂₁H₃₄N₃O₄S: 424.2; found: 424.2.

### Example 27

### Isopropyl 4-(3-(2-(methylsulfonyl)-1,2,3,4-tetrahydroisoquinolin-6-ylamino)propyl)-piperidine-1-carboxylate

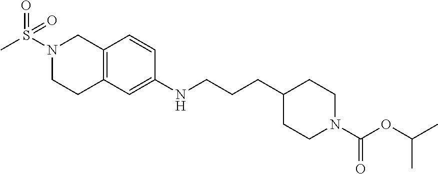

Intermediate 27c: Isopropyl 4-(3-(methylsulfonyloxy)propyl)piperidine-1-carboxylate

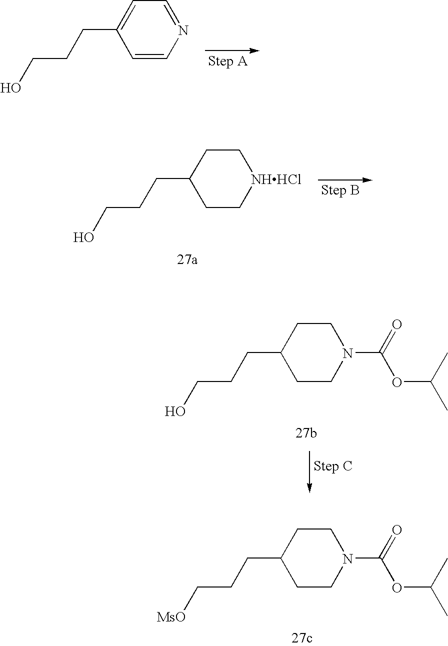

Step A Commercially available 4-pyridinepropanol (25 g, 182 mmol) was charged into a Parr-shaker flask and HCl in dioxane (4M, 100 mL) was added, followed by PtO₂ (4.72 g, 20.8 mmol). The mixture was shaked for 48 h under H₂ (60 psi). The mixture was then evacuated and placed under N₂, filtered through celite and washed with H₂O. Concentration of the filtrate afforded 3-(piperidin-4-yl)propan-1-ol hydrochloride (27a) as a yellow oil. The compound was used in the next step without further purification. ¹H-NMR (600 MHz, CD₃OD) δ=3.51 (t, J=6.6 Hz, 2H), 3.32 (br. d, J=12.6 Hz, 2H), 2.88 (t, J=12.6 Hz, 2H), 1.87 (d, J=13.8 Hz, 2H), 1.57-1.45 (m, 3H), 1.32-1.23 (m, 4H); MS calcd. for [M+H]⁺ C₈H₁₈NO: 144.1; found: 144.1.

Step B The crude compound from Step A (22.3 g, 124 mmol) was suspended in dry DMA (100 mL), then NEt₃ (43 mL, 308 mmol) was added. The resulting mixture was cooled to 0° C. A solution of isopropyl chloroformate in toluene (1.0M, 150 mL) was added dropwise. A white precipitate formed and the suspension was stirred at rt overnight. The white precipitate was filtered off, washed with EtOAc, and discarded. The filtrate was concentrated in vacuo to yield isopropyl 4-(3-hydroxypropyl)piperidine-1-carboxylate (27b) as an oil. ¹H-NMR (400 MHz, CDCl₃) δ=4.90 (septet, J=6.4 Hz, 1H), 4.18 (br. s, 2H), 3.64 (q, J=6.4 Hz, 2H), 2.70 (t, J=12.0 Hz, 2H), 1.67 (br. d, J=12.8 Hz, 2H), 1.60-1.57 (m, 2H), 1.46-1.35 (m, 1H), 1.33-1.27 (m 2H), 1.23 (d, J=6.4 Hz, 6H), 1.08 (ddd, J=12.4, 12.4, 4.0 Hz, 2H).

Step C A sample of intermediate 27b (13 g, 56.7 mmol) was dissolved in dry CH₂Cl₂ (107 mL), then EtN(i-Pr)₂ (15 mL, 87.6 mmol) was added. The resulting mixture was cooled to 0° C. Methanesulfonyl chloride (4.9 mL, 63.1 mmol) was added dropwise, with vigorous stirring, over 5 min. The ice-bath was removed and the resulting solution was stirred at rt overnight. The reaction mixture was poured into 1M HCl and extracted with CH₂Cl₂. The combined organic extracts were washed with brine, dried (Na₂SO₄), and concentrated in vacuo to yield isopropyl 4-(3-(methylsulfonyloxy)propyl)piperidine-1-carboxylate (27c) as an oil. ¹H-NMR (400 MHz, CDCl₃) δ=4.92 (septet, J=6.4 Hz, 1H), 4.24 (t, J=6.4 Hz, 2H), 4.15 (br. S, 2H), 3.03 (s, 3H), 2.72 (t, J=12.4 Hz, 2H), 1.83-1.76 (m, 2H), 1.70 (br. s, 2H), 1.48-1.34 (m, 3H), 1.25 (d, J=6.4 Hz, 6H), 1.12 (ddd, J=12.4, 12.4, 4.0 Hz, 2H).

A sample of intermediate 26c (50 mg, 0.22 mmol) and mesylate 27c (75 mg, 0.24 mmol) were dissolved in DMPU (1.5 mL). EtN(i-Pr)₂ (76 μL, 0.44 mmol) was added and the mixture was stirred at 130° C. overnight. The mixture was then diluted with MeCN and filtered. The filtrate was purified by reverse-phase HPLC to yield the title compound 27. ¹H-NMR (400 MHz, CD₃CN) δ=7.18 (d, J=8.4 Hz, 1H), 7.07 (d, J=8.4 Hz, 1H), 7.05 (s, 1H), 4.83 (septet, J=6.0 Hz, 1H), 4.40 (s, 2H), 4.05 (br. d, J=12.8 Hz, 2H), 3.50 (t, J=6.0 Hz, 2H), 3.25 (t, J=7.6 Hz, 2H), 2.96 (t, J=6.0 Hz, 2H), 2.86 (s, 3H), 2.77-2.68 (m, 2H), 1.73-1.64 (m, 3H), 1.55-1.40 (m, 2H), 1.36-1.28 (m, 2H), 1.21 (d, J=6.0 Hz, 6H), 1.02 (ddd, J=12.4, 12.4, 4.0 Hz, 2H); MS calcd. for [M+H]⁺ C₂₂H₃₆N₃O₄S: 438.2; found: 438.3.

### Example 28

### Isopropyl 4-(3-(2-(methylsulfonyl)-1,2,3,4-tetrahydroisoquinolin-6-ylamino)butyl)-piperidine-1-carboxylate

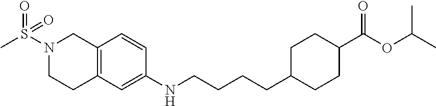

Intermediate 28c: Isopropyl 4-(3-(methylsulfonyloxy)butyl)piperidine-1-carboxylate

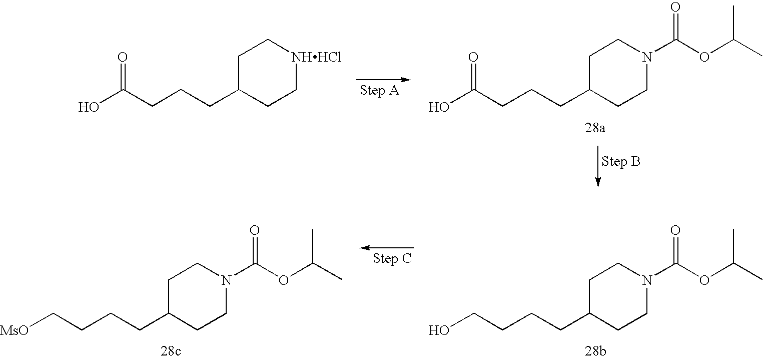

Step A Commercially available 4-piperidine butyric acid hydrochloride (20 g, 96 mmol) was converted to 4-(1-(isopropoxycarbonyl)piperidin-4-yl)butanoic acid (28a) following the same procedure described for the preparation of 27b. ¹H-NMR (400 MHz, CDCl₃) δ=4.92 (septet, J=6.4 Hz, 1H), 4.14 (br. s, 2H), 2.72 (t, J=12.4 Hz, 2H), 2.37 (t, J=7.2 Hz, 2H), 1.72-1.64 (m, 4H), 1.48-1.38 (m, 1H), 1.34-1.28 (m, 2H), 1.25 (d, J=6.4 Hz, 6H), 1.11 (ddd, J=12.4, 12.4, 4.0 Hz, 2H); MS calcd. for [M+H]⁺ C₁₃H₂₄NO₄: 258.2; found: 258.1.

Step B Acid 28a (3 g, 11.7 mmol) was dissolved in THF (30 mL), treated with a solution of BH₃ in THF (1M, 23 mL, 230 mmol), and stirred at rt for 4 h. The solvent was then evaporated, EtOAc was added and the mixture was washed with 1M HCl, and brine. The organic phase was dried over Na₂SO₄, and concentrated in vacuo to give isopropyl 4-(4-hydroxybutyl)piperidine-1-carboxylate (28b) as a colorless oil. ¹H-NMR (400 MHz, CDCl₃) δ=4.90 (septet, J=6.4 Hz, 1H), 4.11 (br. s, 2H), 3.64 (t, J=6.4, 2H), 2.69 (t, J=12.0 Hz, 2H), 1.70-1.62 (m, 2H), 1.59-1.52 (m, 2H), 1.43-1.33 (m, 4H), 1.29-1.25 (m, 2H), 1.23 (d, J=6.4 Hz, 6H), 1.07 (ddd, J=12.4, 12.4, 4.0 Hz, 2H); MS calcd. for [M+H]⁺ C₁₃H₂₆NO₃: 244.2; found: 244.2.

Step C The alcohol 28b (3.1 g, 12.7 mmol) was converted to isopropyl 4-(3-(methylsulfonyloxy)butyl)piperidine-1-carboxylate (28c) following the same procedure described for the preparation of 27c. ¹H-NMR (600 MHz, CDCl₃) δ=4.92 (septet, J=6.0 Hz, 1H), 4.24 (t, J=6.6 Hz, 2H), 4.13 (br. s, 2H), 3.02 (s, 3H), 2.71 (br. t, J=12.0 Hz, 2H), 1.78-1.74 (m, 2H), 1.70-1.64 (m, 2H), 1.48-1.38 (m, 3H), 1.32-1.26 (m, 2H), 1.25 (d, J=6.0 Hz, 6H), 1.14-1.06 (m, 2H); MS calcd. for [M+H]⁺ C₁₄H₂₈NO₅S: 322.2; found: 322.2.

Following the procedure for Example 27, intermediate 26c (50 mg, 0.22 mmol) and mesylate 28c (78 mg, 0.24 mmol) were converted to the title compound (Example 28). ¹H-NMR (400 MHz, CD₃CN) δ=7.15 (d, J=8.4 Hz, 1H), 7.00 (d, J=8.4 Hz, 1H), 6.97 (s, 1H), 4.83 (septet, J=6.4 Hz, 1H), 4.38 (s, 2H), 4.05 (br. d, J=13.2 Hz, 2H), 3.49 (t, J=6.0 Hz, 2H), 3.23 (t, J=7.6 Hz, 2H), 2.94 (t, J=6.0 Hz, 2H), 2.85 (s, 3H), 2.78-2.68 (m, 2H), 1.70-1.62 (m, 4H), 1.46-1.36 (m, 3H), 1.30-1.24 (m, 2H), 1.21 (d, J=6.4 Hz, 6H), 1.02 (ddd, J=12.4, 12.4, 4.0 Hz, 2H); MS calcd. for [M+H]⁺ C₂₄H₃₉N₂O₄S: 451.3; found: 451.2.

### Example 29

### tert-Butyl 6-(3-(1-(isopropoxycarbonyl)piperidin-4-yl)propylamino)-3,4-dihydroisoquinoline-2(1H)-carboxylate

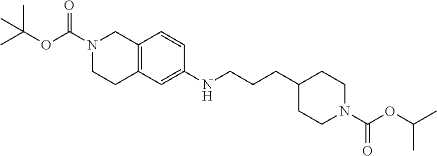

tert-Butyl 6-amino-3,4-dihydroisoquinoline-2(1H)-carboxylate (99.3 mg, 0.4 mmol) and mesylate 27c (123 mg, 0.4 mmol) were dissolved in MeCN (1 mL). Cs₂CO₃ (261 mg, 0.8 mmol) was added and the mixture was stirred at 90° C. overnight. The mixture was then diluted with MeCN and filtered. The filtrate was purified by reverse-phase HPLC to yield the title compound (Example 29). ¹H-NMR (400 MHz, CD₃CN) δ=7.04 (d, J=8.4 Hz, 1H), 6.81 (dd, J=8.4, 2.4 Hz, 1H), 6.76 (s, 1H), 4.83 (septet, J=6.4 Hz, 1H), 4.47 (s, 2H), 4.04 (br. d, J=13.2 Hz, 2H), 3.58 (t, J=6.0 Hz, 2H), 3.15 (t, J=7.6 Hz, 2H), 2.76 (t, J=6.0 Hz, 2H), 2.76-2.68 (m, 2H), 1.69-1.61 (m, 4H), 1.47 (s, 9H), 1.47-1.40 (m, 1H), 1.34-1.28 (m, 2H), 1.21 (d, J=6.4 Hz, 6H), 1.02 (ddd, J=12.8, 12.4, 4.0 Hz, 2H); MS calcd. for [M+2H−Boc]⁺ C₂₁H₃₄N₃O₂: 360.2; found: 360.1.

### Example 30

### tert-Butyl 6-(4-(1-(isopropoxycarbonyl)piperidin-4-yl)butylamino)-3,4-dihydroisoquinoline-2(1H)-carboxylate

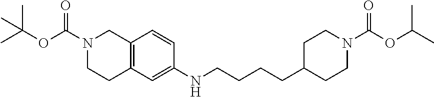

Following the procedure for Example 29, tert-butyl 6-amino-3,4-dihydroisoquinoline-2(1H)-carboxylate (99.3 mg, 0.4 mmol) and mesylate 28c (129 mg, 0.4 mmol) were converted to the title compound (Example 30). ¹H-NMR (400 MHz, CDCl₃) δ=6.93 (d, J=8.0 Hz, 1H), 6.48 (dd, J=8.0, 2.4 Hz, 1H), 6.39 (d, J=2.4 Hz, 1H), 4.93 (septet, J=6.0 Hz, 1H), 4.48 (s, 2H), 4.20-4.10 (m, 2H), 3.62 (br. s, 2H), 3.11 (t, J=7.2 Hz, 2H), 2.82-2.68 (m, 4H), 1.69-1.60 (m, 4H), 1.50 (s, 9H), 1.45-1.39 (m, 3H), 1.32-1.27 (m, 2H), 1.25 (d, J=6.0 Hz, 6H), 1.15-1.05 (m, 2H); MS calcd. for [M+2H−Boc]⁺ C₂₂H₃₆N₃O₂: 374.3; found: 374.1.

### Example 31

### Isopropyl 4-(3-(methyl(2-(methylsulfonyl)-1,2,3,4-tetrahydroisoquinolin-6-yl)amino)butyl)piperidine-1-carboxylate

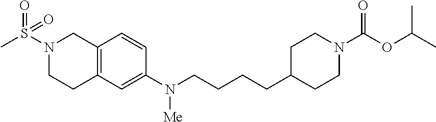

A sample of isopropyl 4-(3-(2-(methylsulfonyl)-1,2,3,4-tetrahydroisoquinolin-6-ylamino)butyl)-piperidine-1-carboxylate (example 28) (13.8 mg, 0.02 mmol) was dissolved in 1,3-dimethyl-3,4,5,6-tetrahydro-2(1H)— pyrimidinone (DMPU, 0.5 mL). Iodomethane (15 μL, 0.24 mmol) was added followed by EtN(i-Pr)₂ (11 μL, 0.06 mmol). The mixture was stirred at 130° C. for 2 h. The mixture was then diluted with MeCN and filtered. The filtrate was purified by reverse-phase HPLC to yield the title compound (Example 31). ¹H-NMR (400 MHz, CD₃CN) δ=7.00 (d, J=8.4 Hz, 1H), 6.82 (d, J=8.4 Hz, 1H), 6.77 (s, 1H), 4.71 (septet, J=6.4 Hz, 1H), 4.25 (s, 2H), 3.92 (br. d, J=13.6 Hz, 2H), 3.38 (t, J=6.0 Hz, 2H), 3.25 (t, J=7.6 Hz, 2H), 2.87 (s, 3H), 2.84 (t, J=6.0 Hz, 2H), 2.74 (s, 3H), 2.66-2.50 (m, 2H), 1.83-1.78 (m, 2H), 1.54 (br. d, J=12.0 Hz, 2H), 1.44-1.36 (m, 2H), 1.33-1.18 (m, 3H), 1.21 (d, J=6.0 Hz, 6H), 0.89 (ddd, J=12.8, 12.8, 4.0 Hz, 2H); MS calcd. for [M+H]⁺ C₂₄H₄₀N₃O₄S: 466.3; found: 466.2.

Examples 32-35 (see table below) were synthesized by analogous methods from derivative 27 and appropriate alkylhalides.

### Example 36

### Isopropyl 4-(3-(N-(2-(methylsulfonyl)-1,2,3,4-tetrahydroisoquinolin-6-yl)acetamido)propyl)piperidine-1-carboxylate

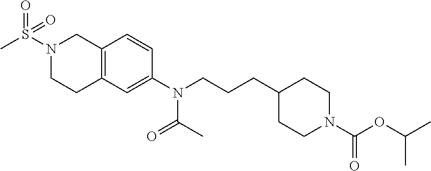

Isopropyl 4-(3-(2-(methylsulfonyl)-1,2,3,4-tetrahydroisoquinolin-6-ylamino)propyl)-piperidine-1-carboxylate (example 27, TFA-salt, 10 mg, 0.02 mmol) was dissolved in CH₂Cl₂, NEt₃ (16 μL, 0.11 mmol) was added followed by acetylchloride (7 μL, 0.10 mmol). The mixture was stirred overnight at rt, diluted with MeCN and filtered. The filtrate was purified by reverse-phase HPLC to yield the title compound (Example 36). ¹H-NMR (400 MHz, CD₃CN) δ=7.15 (d, J=8.8 Hz, 1H), 7.02-7.00 (m, 2H), 4.71 (septet, J=6.4 Hz, 1H), 4.31 (s, 2H), 3.92 (br. d, J=13.2 Hz, 2H), 3.56 (t, J=7.6 Hz, 2H), 3.41 (t, J=6.0 Hz, 2H), 2.88 (t, J=6.0 Hz, 2H), 2.77 (s, 3H), 2.70-2.55 (m, 4H), 1.74 (s, 3H), 1.52 (br. d, J=10.8 Hz, 2H), 1.43-1.35 (m, 3H), 1.10 (d, J=6.4 Hz, 6H), 0.89 (ddd, J=12.4, 12.4, 4.0 Hz, 2H); MS calcd. for [M+H]⁺ C₂₄H₃₈N₃O₅S: 480.3; found: 480.2.

### Example 37

### Isopropyl 4-(4-(2-(methylsulfonyl)-1,2,3,4-tetrahydroisoquinolin-6-ylamino)-4-oxobutyl)piperidine-1-carboxylate

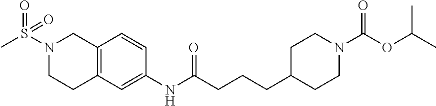

A sample of intermediate 26c (50 mg, 0.22 mmol) and intermediate 28a (62 mg, 0.024 mmol) were dissolved in NMP (1 mL). EtN(i-Pr)₂ (76 μL, 0.44 mmol) was then added followed by HATU (100 mg, 0.26 mmol). The mixture was stirred at 70° C. for 48 h. The mixture was then diluted with MeCN and filtered. The filtrate was purified by reverse-phase HPLC to yield the title compound (Example 37). ¹H-NMR (400 MHz, CDCl₃) δ=7.52 (br. s, 1H), 7.23 (d, J=8.4 Hz, 1H), 7.07-7.05 (m, 2H), 4.92 (septet, J=6.4 Hz, 1H), 4.44 (s, 2H), 4.14 (br. s, 2H), 3.57 (t, J=6.0 Hz, 2H), 2.99 (t, J=6.0 Hz, 2H), 2.86 (s, 3H), 2.72 (t, J=12.8 Hz, 2H), 2.37 (t, J=7.2 Hz, 2H), 1.81-1.66 (m, 4H), 1.48-1.41 (m, 1H), 1.37-1.31 (m, 2H), 1.25 (d, J=6.4 Hz, 6H), 1.18-1.08 (m, 2H); MS calcd. for [M+H]⁺ C₂₃H₃₆N₃O₅S: 466.2; found: 466.2.

Examples 38 and 39 (see table below) were synthesized by analogous methods from derivative 26c and the appropriate acids.

### Example 40

### tert-Butyl 4-((2-(methylsulfonyl)-1,2,3,4-tetrahydroisoquinoline-6-carboxamido)methyl)piperidine-1-carboxylate

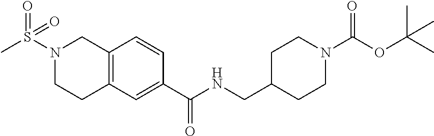

Intermediate 40b: 2-(Methylsulfonyl)-1,2,3,4-tetrahydroisoquinoline-6-carboxylic acid

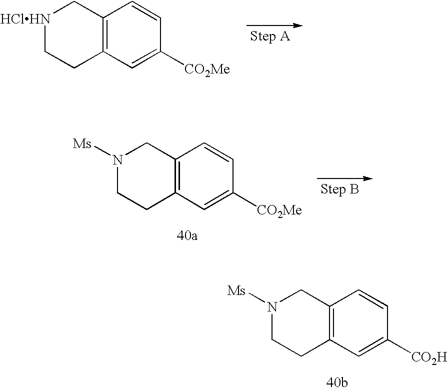

Step A Commercially available 6-(methoxycarbonyl)-1,2,3,4-tetrahydro-isoquinoline hydrochloride (17.4 g, 76.4 mmol) was converted to methyl 2-(methylsulfonyl)-1,2,3,4-tetrahydroisoquinoline-6-carboxylate (40a) following the same procedure described for the preparation of intermediate 27c. ¹H-NMR (400 MHz, CDCl₃) δ=7.90-7.87 (m, 2H), 7.19 (d, J=8.4 Hz, 1H), 4.53 (s, 2H), 3.94 (s, 3H), 3.61 (t, J=6.0 Hz, 2H), 3.06 (t, J=6.0 Hz, 2H), 2.88 (s, 3H); MS calcd. for [M+H]⁺ C₁₂H₁₆NO₄S: 270.1; found: 270.1.

Step B Ester 40a (6.16 g, 22.9 mmol) was suspended in MeOH (60 mL) and a solution of NaOH (10%, 60 mL) was added. The mixture was stirred for 4 h. 1M HCl was then added until a clear solution was obtained. The mixture was extracted with EtOAc. The aqueous phase was acidified to pH 1 with 1M HCl and the resulting precipitate was filtered, washed with EtOAc, and dried to afford 2-(methylsulfonyl)-1,2,3,4-tetrahydroisoquinoline-6-carboxylic acid (40b). ¹H-NMR (400 MHz, CD₃OD) δ=7.86 (s, 1H), 7.85 (d, J=8.4 Hz, 1H), 7.26 (d, J=8.4 Hz, 1H), 4.50 (s, 2H), 3.56 (t, J=6.0 Hz, 2H), 3.04 (t, J=6.0 Hz, 2H), 2.92 (s, 3H); MS calcd. for [M+H]⁺ C₁₁H₁₄NO₄S: 256.1, found: 256.1. The aqueous phase was then extracted with EtOAc. The combined organics were dried (Na₂SO₄) and concentrated in vacuo to afford additional acid 40b.

Following the procedure for Example 37, acid 40b (38.3 mg, 0.15 mmol) was coupled with commercially available 1-Boc-4-(aminomethyl)-piperidine (35.4 mg, 0.17 mmol) to afford the title compound (Example 40). ¹H-NMR (400 MHz, CDCl₃) δ=7.53 (s, 1H), 7.51 (d, J=8.0 Hz, 1H), 7.14 (d, J=8.0, 1H), 6.96 (br. t, 1H), 4.36 (s, 2H), 3.95 (br. d, J=13.2, 2H), 3.43 (t, J=6.0 Hz, 2H), 3.14 (t, J=6.4 Hz, 2H), 2.92 (t, J=6.0 Hz, 2H), 2.76 (s, 3H), 2.61 (br. s, 2H), 1.67-1.59 (m, 3H), 1.33 (s, 9H), 1.00 (ddd, J=12.4, 12.4, 4.4 Hz, 2H); MS calcd. for [M+2H−Boc]⁺ C₁₇H₂₆N₃O₃S: 352.1; found: 352.1.

### Example 41

### Isopropyl 4-(2-(2-(methylsulfonyl)-1,2,3,4-tetrahydroisoquinoline-6-carboxamido)ethyl)piperidine-1-carboxylate

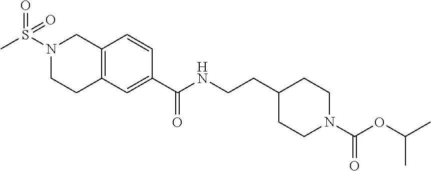

Intermediate 41b: Isopropyl 4-(2-aminoethyl)piperidine-1-carboxylate

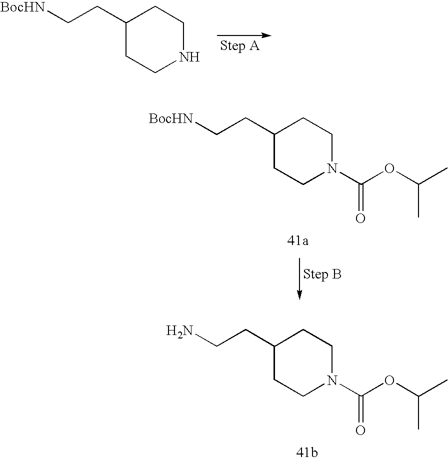

Step A Commercially available tert-butyl 2-(piperidin-4-yl)ethylcarbamate (1.91 g, 8.37 mmol) and NEt₃ (1.5 mL, 10.7 mmol) were dissolved in 1,2-dimethoxyethane (20 mL) and DMF (20 mL). A solution of isopropyl chloroformate in toluene (1M, 9.5 mL, 9.5 mmol) was added dropwise with stirring. The resulting mixture was stirred at rt for 16 h. EtOAc was added, and the organics were washed with water, sat. NH₄Cl, and brine, dried over MgSO₄, and filtered. Concentration of the filtrate yielded isopropyl 4-(2-(tert-butoxycarbonylamino)ethyl)piperidine-1-carboxylate (41a) as a thick oil. ¹H-NMR (400 MHz, DMSO-d₆) δ=6.79 (t, J=5.2 Hz, 1H), 4.74 (septet, J=6.3 Hz, 1H), 3.93 (d, J=10.4 Hz, 2H), 2.94 (dd, J=13.1, 6.8 Hz, 2H), 2.68 (br. s, 2H), 1.62 (d, J=12.5 Hz, 2H), 1.37 (s, 9H), 1.30 (m, 3H), 1.17 (d, J=6.3 Hz, 6H), 0.94 (ddd, J=12.5, 12.5, 4.2 Hz, 2H).

Step B Intermediate 41a (2.40 g, 7.63 mmol) was dissolved in CH₂Cl₂ (5 mL). Trifluoroacetic acid (4 mL) was added and the mixture was stirred at rt for 2 h. The solvent was evaporated, EtOAc was added to the residue and the resulting solution was neutralized with sat. aqueous NaHCO₃. The mixture was extracted with EtOAc. The combined organics were washed with brine, dried (MgSO₄) and concentrated in vacuo to yield isopropyl 4-(2-aminoethyl)piperidine-1-carboxylate (41b) as an oil. ¹H-NMR (400 MHz, DMSO-d₆) δ=4.76 (septet, J=6.3 Hz, 1H), 3.96 (d, J=11.0 Hz, 2H), 2.82 (m, 2H), 2.65 (br. s, 2H), 1.64 (d, J=12.8 Hz, 2H), 1.47 (m, 3H), 1.18 (d, J=6.3 Hz, 6H), 0.99 (ddd, J=12.5, 12.2, 4.2 Hz, 2H); MS calcd. for [M+H]⁺ C₁₁H₂₃N₂O₂: 215.2; found: 215.1.

Following the procedure for Example 37, acid 40b (38.3 mg, 0.15 mmol) was coupled with amine 41b (35.4 mg, 0.17 mmol) to afford the title compound (Example 41). ¹H-NMR (400 MHz, CD₃CN) δ=7.53 (s, 1H), 7.50 (d, J=8.0 Hz, 1H), 7.13 (d, J=8.0, 1H), 6.88 (br. t, 1H), 4.72 (septet, J=6.4 Hz, 1H), 4.36 (s, 2H), 3.95 (br. d, J=13.2, 2H), 3.42 (t, J=6.0 Hz, 2H), 3.29 (q, J=6.0 Hz, 2H), 2.92 (t, J=6.0 Hz, 2H), 2.76 (s, 3H), 2.63 (br. t, 2H), 1.64 (br. d, J=12.8, 2H), 1.50-1.39 (m, 3H), 1.11 (d, J=6.4 Hz, 6H), 0.98 (ddd, J=12.8, 12.4, 4.4 Hz, 2H); MS calcd. for [M+H]⁺ C₂₂H₃₄N₃O₅S: 452.2; found: 452.2.

### Example 42

### Isopropyl 4-(2-(2-(methylsulfonyl)-1,2,3,4-tetrahydroisoquinoline-6-carboxamido)propyl)piperidine-1-carboxylate

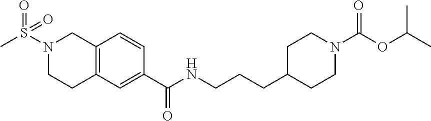

Intermediate 42b: Isopropyl 4-(3-aminopropyl)piperidine-1-carboxylate

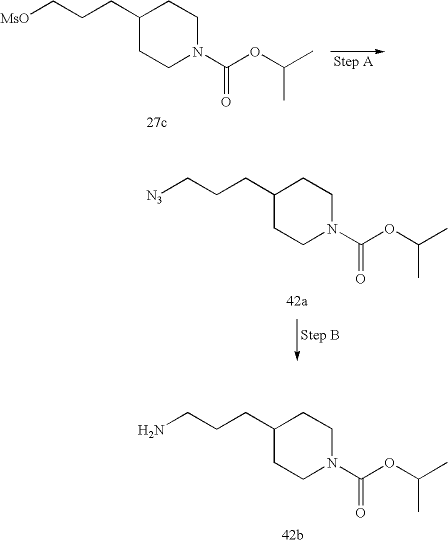

Step A Mesylate 27c (3.83 g, 12.5 mmol) was dissolved in DMF (24 mL). Cs₂CO₃ (8.12 g, 24.9 mmol) was added, followed by NaN₃ (1.3 g, 20 mmol). The mixture was heated at 90° C. for 2 h, cooled to rt, diluted with Et₂O and washed with 5% aqueous Na₂CO₃. The aqueous phase was extracted with Et₂O. The organics were combined, washed with brine, dried (Na₂SO₄) and concentrated in vacuo to afford isopropyl 4-(3-azidopropyl)piperidine-1-carboxylate (42a) as an oil. It was used in the next step without further purification. ¹H-NMR (400 MHz, CDCl₃) δ=4.92 (septet, J=6.4 Hz, 1H), 4.15 (br. s, 2H), 3.29 (d, J=7.2 Hz, 2H), 2.72 (t, J=12.4, 2H), 1.68-1.60 (m, 4H), 1.48-1.38 (m, 1H), 1.36-1.31 (m, 2H), 1.25 (d, J=6.4 Hz, 6H), 1.12 (ddd, J=12.8, 12.4, 4.4 Hz, 2H); MS calcd. for [M+H]⁺ C₁₂H₂₃N₄O₂: 255.2; found: 255.1.

Step B Azide 42a (2.08 g, 8.18 mmol) was dissolved in MeOH (86 mL). Pd/C (10%, 208 mg) was added, followed by AcOH (0.1 mL). The flask was evacuated and flushed with hydrogen, and the mixture was stirred overnight under H₂ (1 atm). The mixture was then evacuated and placed under Ar, filtered through celite and washed with MeOH. Concentration of the filtrate afforded isopropyl 4-(3-aminopropyl)piperidine-1-carboxylate (42b) as an oil. It was used in the next step without further purification. ¹H-NMR (400 MHz, CDCl₃) δ=4.92 (septet, J=6.4 Hz, 1H), 4.13 (br. s, 2H), 3.16 (br. s, 2H), 2.72 (t, J=7.2, 2H), 2.74-2.66 (m, 2H), 1.68 (br. d, J=12.8 Hz, 2H), 1.53-1.48 (m, 2H), 1.44-1.35 (m, 1H), 1.31-1.27 (m, 3H), 1.24 (d, J=6.4 Hz, 6H), 1.15-1.05 (m, 2H); MS calcd. for [M+H]⁺ C₁₂H₂₅N₂O₂: 229.2; found: 229.1.

Following the procedure for Example 37, acid 40b (38.3 mg, 0.15 mmol) was coupled with amine 42b (37.7 mg, 0.17 mmol) to afford the title compound (Example 42). ¹H-NMR (400 MHz, CD₃CN) δ=7.63 (s, 1H), 7.61 (d, J=8.0 Hz, 1H), 7.24 (d, J=8.0, 1H), 7.01 (br. t, 1H), 4.82 (septet, J=6.4 Hz, 1H), 4.46 (s, 2H), 4.05 (br. d, J=13.2, 2H), 3.53 (t, J=6.0 Hz, 2H), 3.33 (q, J=6.8 Hz, 2H), 3.02 (t, J=6.0 Hz, 2H), 2.86 (s, 3H), 2.73 (br. t, 2H), 1.70 (br. d, J=12.8, 2H), 1.65-1.57 (m, 2H), 1.53-1.42 (m, 1H), 1.34-1.28 (m, 2H), 1.21 (d, J=6.4 Hz, 6H), 1.04 (ddd, J=12.4, 12.4, 4.4 Hz, 2H); MS calcd. for [M+H]⁺ C₂₃H₃₆N₃O₅S: 466.2; found: 466.2.

### Example 43

### Isopropyl 4-(((2-(methylsulfonyl)-1,2,3,4-tetrahydroisoquinolin-6-yl)methoxy)methyl)piperidine-1-carboxylate

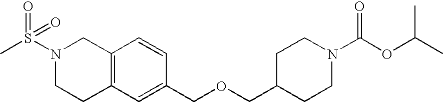

Intermediate 43a: (2-(Methylsulfonyl)-1,2,3,4-tetrahydroisoquinolin-6-yl)methanol

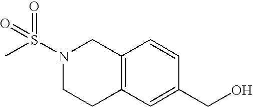

The ester 40a (523 mg, 1.94 mmol) was dissolved in THF (4 mL). A solution of LiAlH₄ in THF (1M, 1.94 mL) was added at rt and the resulting mixture was stirred for 1 h. Saturated aqueous Na₂SO₄ were then added until the gas evolution ceased. The mixture was filtered through a plug of celite and washed with EtOAc. Concentration of the filtrate in vacuo afforded (2-(methylsulfonyl)-1,2,3,4-tetrahydroisoquinolin-6-yl)methanol (43a) as a white solid. It was used in the next step without further purification. ¹H-NMR (400 MHz, CDCl₃) δ=7.22 (d, J=8.4 Hz, 1H), 7.21 (s, 1H), 7.11 (d, J=8.4 Hz, 1H), 4.69 (d, J=5.2 Hz, 2H), 4.48 (s, 2H), 3.59 (t, J=6.0 Hz, 2H), 3.01 (t, J=6.0 Hz, 2H), 2.86 (s, 3H), 1.68 (t, J=5.6 Hz, 1H); MS calcd. for [M+H]⁺ C₁₁H₁₆NO₃S: 242.1; found: 242.0.

Intermediate 43c: Isopropyl 4-((methylsulfonyloxy)methyl)piperidine-1-carboxylate

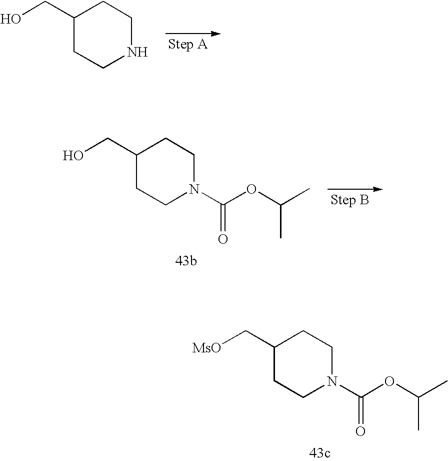

Step A Commercially available piperidin-4-yl-methanol (5.26 g, 45.7 mmol) was converted to 4-(hydroxymethyl)piperidine-1-carboxylate (43b) following the same procedure described for the preparation of 26d. ¹H-NMR (400 MHz, DMSO-d₆) δ=4.75 (quintet, J=6.2 Hz, 1H), 4.49 (t, J=5.3 Hz, 1H), 3.95 (dd, J=5.6, 5.6 Hz, 2H), 3.24 (br. s, 2H), 1.63 (dd, J=12.9, 2.0 Hz, 2H), 1.51 (m, 1H), 1.17 (d, J=6.2 Hz, 6H), 0.98 (m, 2H).

Step B Alcohol 43b (4.25 g, 21.1 mmol) was converted to isopropyl 4-((methylsulfonyloxy)methyl)piperidine-1-carboxylate (43c) following the same procedure described for the preparation of 27c. ¹H-NMR (400 MHz, CDCl₃) δ=4.74 (septet, J=6.2 Hz, 1H), 4.07 (d, J=6.4 Hz, 2H), 3.99 (d, J=11.0 Hz, 2H), 3.17 (s, 3H), 2.51 (br. s, 2H), 1.88 (dd, J=14.6, 1.6 Hz, 2H), 1.68 (m, 1H), 1.18 (d, J=6.2 Hz, 6H), 1.17 (m, 2H); MS calcd. for [M+H]⁺ C₁₁H₂₂NO₅S: 280.1; found: 280.2.

A sample of alcohol 43a (40 mg, 0.17 mmol) was dissolved in THF (0.5 mL). NaH (60% in oil, 6.7 mg, 0.17 mmol) was added at rt and the mixture was stirred for 15 minutes. A solution of mesylate 43c (51 mg, 0.18 mmol) in THF (0.5 mL) was then added and the mixture was stirred at 80° C. overnight. To the mixture was added additional NaH (60% in oil, 7 mg) and it was stirred at 110° C. for 10 h. The mixture was cooled to rt, then diluted with MeCN and filtered. The filtrate was purified by reverse-phase HPLC to yield the title compound (Example 43). ¹H-NMR (400 MHz, CD₃CN) δ=7.08-7.03 (m, 3H), 4.72 (septet, J=6.4 Hz, 1H), 4.34 (s, 2H), 4.30 (s, 2H), 3.97 (d, J=12.8 Hz, 2H), 3.40 (t, J=6.0 Hz, 2H), 3.23 (d, J=6.4 Hz, 2H), 2.86 (t, J=6.0 Hz, 2H), 2.74 (s, 3H), 2.65 (br. t, 2H), 1.74-1.66 (m, 1H), 1.62 (br. d, J=13.2 Hz, 2H), 1.11 (d, J=6.4 Hz, 6H), 1.01 (ddd, J=12.4, 12.4, 4.4 Hz, 2H); MS calcd. for [M+H]⁺ C₂₁H₃₃N₂O₅S: 425.2; found: 425.2.

Examples 44-46 (see table below) were synthesized by analogous methods from derivative 43a and the appropriate mesylates.

### Example 47

### Isopropyl 4-(5-(2-(methylsulfonyl)-1,2,3,4-tetrahydroisoquinolin-6-yl)-1,2,4-oxadiazol-3-yl)piperidine-1-carboxylate

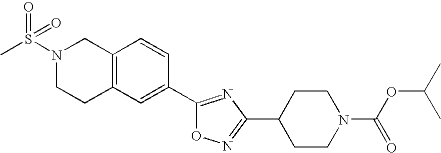

Intermediate 47b: Isopropyl 4-(N′-hydroxycarbamimidoyl)piperidine-1-carboxylate

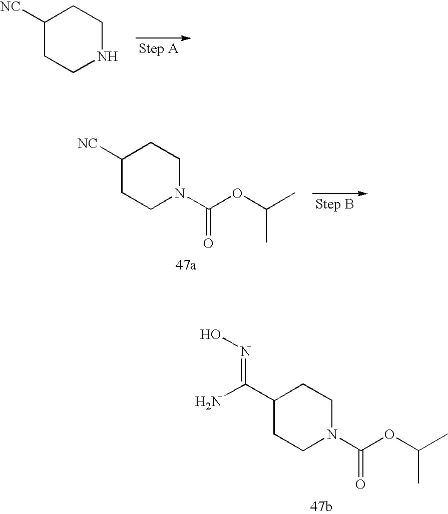

Step A Isopropyl 4-cyanopiperidine-1-carboxylate (47a) was prepared from 4-cyanopyperidine (1.36 g, 12.3 mmol) according to the same procedure described for the preparation of 26c, using EtOAc as solvent. ¹H-NMR (400 MHz, CDCl₃) δ=4.94 (septet, J=6.4 Hz, 1H), 3.74-3.68 (m, 2H), 3.44-3.38 (m, 2H), 2.84 (m, 1H), 1.95-1.87 (m, 2H), 1.87-1.78 (m, 2H), 1.26 (d, J=6.0 Hz, 6H); MS calcd. for [M+H]⁺ C₁₀H₁₇N₂O₂: 197.1; found: 197.1.

Step B Hydroxylamine (50% in water, 0.38 mL, 6.2 mmol) was added to a mixture of 47a (617 mg, 3.1 mmol) in EtOH (2 mL). The mixture was heated at 60° C. for 1.5 h and the solvent was removed under reduced pressure. Water was added and the mixture was extracted with CH₂Cl₂. The combined organic layers were dried (Na₂SO₄), filtered and concentrated in vacuo to afford isopropyl 4-(N′-hydroxycarbamimidoyl)piperidine-1-carboxylate (47b) as a white solid that was used in the next step without further purification. ¹H-NMR (400 MHz, CDCl₃) δ=6.87 (br s, 1H), 4.93 (septet, J=6.4 Hz, 1H), 4.51 (s, 2H), 4.23 (br s, 2H), 2.79 (t, J=12.4 Hz, 2H), 2.29 (tt, J=12.0, 3.6 Hz, 1H), 1.85 (d, J=12.4 Hz, 2H), 1.62-1.53 (m, 2H), 1.26 (d, J=6.0 Hz, 6H); MS calcd. for [M+H]⁺ C₁₀H₂₀N₃O₃: 230.1; found: 230.1.

Carbonyldiimidazole (24.3 mg, 0.15 mmol) was added to a solution of 40b (38.3 mg, 0.15 mmol) in DMF. After stirring at rt for 30 minutes, 47b (37.8 mg, 0.16 mmol) was added and the resulting mixture was stirred at rt overnight. Another equivalent of carbolylimidazole (24.3 mg, 0.15 mmol) was then added and the resulting mixture was heated at 115° C. for 8 h. After cooling, the mixture was diluted with MeCN and filtered. The filtrate was purified by reverse-phase HPLC to yield the title compound (Example 47). ¹H-NMR (400 MHz, CDCl₃) δ=7.99-7.96 (m, 2H), 7.28 (d, J=8.4 Hz, 1H), 4.97 (septet, J=6.4 Hz, 1H), 4.56 (s, 2H), 4.29-4.18 (m, 2H), 3.63 (t, J=6.0 Hz, 2H), 3.12-3.00 (m, 5H), 2.91 (s, 3H), 2.11-2.08 (m, 2H), 1.93-1.83 (m, 2H), 1.29 (d, J=6.0 Hz, 6H); MS calcd. for [M+H]⁺ C₂₁H₂₉N₄O₅S: 449.2; found: 449.2.

### Example 48

### Isopropyl 4-((5-(2-(methylsulfonyl)-1,2,3,4-tetrahydroisoquinolin-6-yl)-1,2,4-oxadiazol-3-yl)methyl)piperidine-1-carboxylate

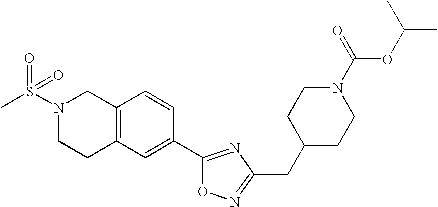

Intermediate 48b: Isopropyl 4-(2-amino-2-(hydroxyimino)ethyl)-piperidine-1-carboxylate

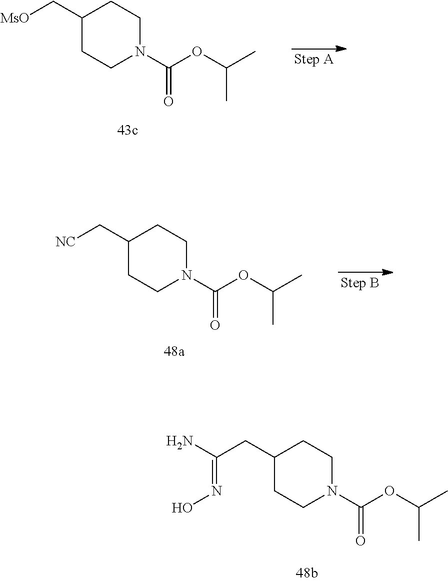

Step A To a solution of 43c (0.42 g, 1.5 mmol) in DMF (3 mL), KCN (0.15 g, 2.3 mmol) and Cs₂CO₃ (0.68 g, 2.1 mmol) were added, and the resulting mixture was heated to 60° C. for 18 h. After cooling to rt, water (20 mL) was added and the mixture was extracted with EtOAc. The combined extracts were washed with water, saturated aqueous NH₄Cl, brine, dried over MgSO₄ and concentrated to yield isopropyl 4-(cyanomethyl)piperidine-1-carboxylate (48a) as an oil. ¹H-NMR (400 MHz, DMSO-d₆) δ=4.75 (septet, J=6.2 Hz, 1H), 3.97 (d, J=11.8 Hz, 2H), 2.75 (br. s, 2H), 1.79 (m, 1H), 1.69 (m, 2H), 1.17 (d, J=6.2 Hz, 6H), 1.07 (m, 4H); MS calcd. for [M+H]⁺ C₁₁H₁₉N₂O₂: 211.1; found: 211.1.

Step B Isopropyl 4-(2-amino-2-(hydroxyimino)ethyl)-piperidine-1-carboxylate (48b) was prepared from 48a (560 mg, 2.66 mmol) according to the procedure described for the synthesis of 47b. ¹H-NMR (400 MHz, CDCl₃) δ=4.92 (septet, J=6.4 Hz, 1H), 4.54 (s, 1H), 4.16 (br s, 2H), 2.75 (t, J=12.0 Hz, 2H), 2.08 (d, J=6.8 Hz, 2H), 1.86-1.80 (m, 1H), 1.79-1.72 (m, 2H), 1.25 (d, J=6.4 Hz, 6H), 1.22-1.11 (m, 2H); MS calcd. for [M+H]⁺ C₁₁H₂₂N₃O₃: 244.1; found: 244.1.

Following the procedure for Example 47, acid 40b (38.3 mg, 0.15 mmol) was condensed with 48b (40.1 mg, 0.16 mmol) to afford the title compound (Example 48). ¹H-NMR (400 MHz, CD₃CN) δ=7.96 (s, 1H), 7.94 (d, J=8.0 Hz, 1H), 7.39 (d, J=8.0 Hz, 1H), 4.83 (septet, J=6.4 Hz, 1H), 4.51 (s, 2H), 4.08 (br. d, J=13.6 Hz, 2H), 3.56 (t, J=6.0 Hz, 2H), 3.09 (t, J=6.0 Hz, 2H), 2.88 (s, 3H), 2.82-2.73 (m, 2H), 2.73 (d, J=6.8 Hz, 2H), 2.08-1.95 (m, 1H), 1.75 (br. d, J=13.2 Hz, 2H), 1.30-1.16 (m, 2H), 1.22 (d, J=6.4 Hz, 6H); MS calcd. for [M+H]⁺ C₂₂H₃₁N₄O₅S: 463.2; found: 463.2.

### Example 49

### Isopropyl 4-(2-(5-(2-(methylsulfonyl)-1,2,3,4-tetrahydroisoquinolin-6-yl)-1,2,4-oxadiazol-3-yl)ethyl)piperidine-1-carboxylate

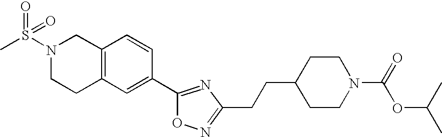

Intermediate 49a: Isopropyl 4-(3-amino-3-(hydroxyimino)propyl)-piperidine-1-carboxylate

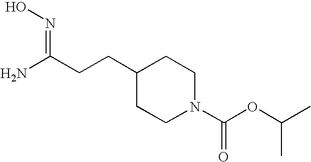

Isopropyl 4-(3-amino-3-(hydroxyimino)propyl)-piperidine-1-carboxylate (49a) was synthesized from mesylate 26e by analogous methods described for the synthesis of 48b. ¹H-NMR (400 MHz, CDCl₃) δ=4.92 (septet, J=6.4 Hz, 1H), 4.53 (s, 1H), 4.22-4.10 (m, 2H), 2.78-2.69 (m, 2H), 2.21-2.17 (m, 2H), 1.71 (br. d, J=12.8 Hz, 2H), 1.57-1.51 (m, 2H), 1.50-1.42 (m, 1H), 1.25 (d, J=6.4 Hz, 6H), 1.17-1.07 (m, 2H); MS calcd. for [M+H]⁺ C₁₂H₂₄N₃O₃: 258.2; found: 258.1.

Following the procedure for Example 47, acid 40b (38.3 mg, 0.15 mmol) was condensed with 49a (42.5 mg, 0.16 mmol) to afford the title compound (Example 49): MS calcd. for [M+H]⁺ C₂₃H₃₃N₄O₅S: 477.2; found: 477.2.

### Example 50

### tert-Butyl 4-((5-(2-(methylsulfonyl)-1,2,3,4-tetrahydroisoquinolin-6-yl)-1,2,4-oxadiazol-3-yl)methyl)piperidine-1-carboxylate

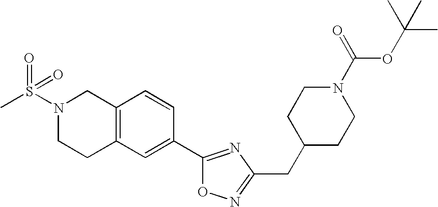

Intermediate 50a: tert-Butyl 4-(2-amino-2-(hydroxyimino)ethyl)-piperidine-1-carboxylate

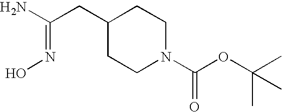

tert-Butyl 4-(2-amino-2-(hydroxyimino)ethyl)-piperidine-1-carboxylate (50a) was synthesized from the corresponding mesylate by analogous methods described for the synthesis of 48b. ¹H-NMR (400 MHz, CDCl₃) δ=4.46 (s, 2H), 4.02 (br s, 2H), 2.59 (t, J=12.0 Hz, 2H), 1.99 (d, J=6.0 Hz, 2H), 1.68-1.63 (m, 3H), 1.38 (s, 9H), 1.12-1.02 (m, 2H); MS calcd. for [M+2H−Boc]⁺ C₇H₁₆N₃O: 158.1; found: 158.1.

NaH (60% in oil, 178 mg, 4.94 mmol) was added to a mixture of 50a (1.27 g, 4.94 mmol) in THF (35 mL). The mixture was heated at 60° C. for 1.5 h, cooled to rt and treated with activated powdered 4 Å-molecular sieves. A solution of ester 40a (1 g, 3.7 mmol) in THF/dioxane (2/1, 12 mL) was then added, and the mixture was heated again at 60° C. overnight. After cooling to rt, the mixture was filtered through a celite plug and washed with EtOAc. The solvents were evaporated and the crude was purified by flash chromatography (EtOAc/hexane) to afford the title compound (Example 50) as a white solid. ¹H-NMR (400 MHz, CDCl₃) δ=7.91-7.87 (m, 2H), 7.19 (d, J=8.4 Hz, 1H), 4.47 (s, 2H), 4.04 (br. s, 2H), 3.54 (t, J=6.0 Hz, 2H), 3.01 (t, J=6.0 Hz, 2H), 2.81 (s, 3H), 2.71-2.62 (m, 2H), 2.67 (d, J=7.2 Hz, 2H), 2.01-1.90 (m, 1H), 1.67 (br. d, J=12.4 Hz, 2H), 1.38 (s, 9H), 1.25-1.14 (m, 2H); MS calcd. for [M+2H−Boc]⁺ C₁₈H₂₅N₄O₃S: 377.1; found: 377.1.

### Example 51

### 3-((1-(5-Ethylpyrimidin-2-yl)piperidin-4-yl)methyl)-5-(2-(methylsulfonyl)-1,2,3,4-tetrahydroisoquinolin-6-yl)-1,2,4-oxadiazole

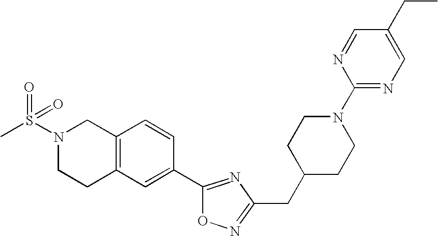

Intermediate 51a: 5-(2-(Methylsulfonyl)-1,2,3,4-tetrahydroisoquinolin-6-yl)-3-(piperidin-4-ylmethyl)-1,2,4-oxadiazole dihydrochloride

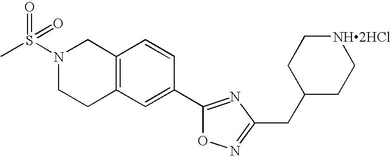

A solution of HCl in dioxane (4 M, 12 mL) was added at rt to a solution of 50 (1.25 g, 2.62 mmol) in dioxane (8 mL). After complexion of the reaction, the solvents were evaporated and the compound dried was under high vacuum to afford 5-(2-(methylsulfonyl)-1,2,3,4-tetrahydroisoquinolin-6-yl)-3-(piperidin-4-ylmethyl)-1,2,4-oxadiazole dihydrochloride (51a) as a white solid that was used in the next step without further purification. ¹H-NMR (400 MHz, CDCl₃) δ=9.64 (br. s, 1H), 9.36 (br. s, 1H), 7.89-7.86 (m, 2H), 7.20 (d, J=8.0 Hz, 1H), 4.47 (s, 2H), 3.54 (t, J=6.0 Hz, 2H), 3.45 (br. d, J=12.4 Hz, 2H), 3.02 (t, J=6.0, 2H), 2.86-2.79 (m, 2H), 2.82 (s, 3H), 2.74 (d, J=7.2 Hz, 2H), 2.13-2.02 (m, 1H), 1.94 (br. d, J=13.2 Hz, 2H), 1.81-1.71 (m, 2H); MS calcd. for [M+2H−Boc]⁺ C₁₈H₂₅N₄O₃S: 377.1; found: 377.1.

Method A: To a solution of 51a (50 mg, 0.11 mmol) and 2-chloro-5-ethyl pyrimidine (74 μL, 0.61 mmol) in DMA (0.5 mL) was added EtN(i-Pr)₂ (0.2 mL). The vial was sealed and heated at 150° C. for 48 h. After cooling to rt, the mixture was diluted with MeCN and filtered. The filtrate was purified by reverse-phase HPLC to yield the title compound (Example 51): ¹H-NMR (400 MHz, CD₃CN) δ=8.20 (s, 2H), 7.97 (s, 1H), 7.95 (d, J=8.0 Hz, 1H), 7.39 (d, J=8.0 Hz, 1H), 4.70 (br. d, J=13.6 Hz, 2H), 4.52 (s, 2H), 3.56 (t, J=6.0 Hz, 2H), 3.09 (t, J=6.0 Hz, 2H), 2.91-2.83 (m, 2H), 2.88 (s, 3H), 2.75 (d, J=6.8 Hz, 2H), 2.46 (q, J=7.6 Hz, 2H), 2.16-2.12 (m, 1H), 1.84-1.78 (m, 2H), 1.27 (ddd, J=12.4, 12.4, 4.4 Hz, 2H), 1.18 (t, J=7.6 Hz, 3H); MS calcd. for [M+H]⁺ C₂₄H₃₁N₆O₃S: 483.1; found: 482.9.

Method B: The above mentioned starting material was heated at 150° C. in microwave for 30 min in 1,3-dimethyl-3,4,5,6-tetrahydro-2(1H)-pyrimidinone (DMPU) in the presence of EtN(i-Pr)₂ to yield the desired product.

Examples 52-57 (see table below) were synthesized by analogous methods from derivative 51a and the appropriate heteroaromatics.

### Example 58

### 5-(2-(Methylsulfonyl)-1,2,3,4-tetrahydroisoquinolin-6-yl)-3-((1-(5-(trifluoromethyl)pyridin-2-yl)piperidin-4-yl)methyl)-1,2,4-oxadiazole

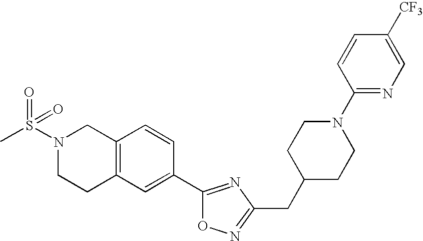

In a microwave vial, 51a (250 mg, 0.56 mmol), 2-chloro-5-trifluoromethyl pyridine (220 mg, 1.2 mmol), and K₂CO₃ (418 mg, 3.0 mmol) were dissolved/suspended in DMF (5 mL). The vial was sealed and heated in the microwave at 150° C. for 10 minutes. The mixture was subsequently heated in the microwave at 170° C. for 15 minutes. Methanesulfonyl chloride (47 μL, 0.6 mmol) was then added and stirring was continued at rt for 1 h. The mixture was then diluted with Et₂O and saturated aqueous NH₄Cl, and extracted with Et₂O. The combined organic phases were washed with brine, dried (Na₂SO₄) and concentrated in vacuo. The crude was purified by flash chromatography (EtOAc/hexane) to yield the title compound (Example 58) as well as the oxidized compound (Example 59). Compound 58: ¹H-NMR (400 MHz, CDCl₃) δ=8.39 (m, 1H), 7.98-7.97 (m, 2H), 7.62 (dd, J=8.8, 2.4 Hz, 1H), 7.28 (d, J=8.0 Hz, 1H), 6.66 (d, J=8.0 Hz, 1H), 4.56 (s, 2H), 4.44 (br. d, J=13.2 Hz, 2H), 3.63 (t, J=6.0 Hz, 2H), 3.11 (d, J=6.0 Hz, 2H), 2.99-2.92 (m, 2H), 2.91 (s, 3H), 2.79 (d, J=7.2 Hz, 2H), 2.26-2.14 (m, 1H), 1.92-1.88 (m, 2H), 1.40 (ddd, J=12.4, 12.4, 4.0 Hz, 2H); MS calcd. for [M+H]⁺ C₂₄H₂₇F₃N₅O₃S: 522.2; found: 522.2.

### Example 59

### 2-(Methylsulfonyl)-6-(3-((1-(5-(trifluoromethyl)pyridin-2-yl)piperidin-4-yl)methyl)-1,2,4-oxadiazol-5-yl)-1,2,3,4-tetrahydroisoquinolin-1-ol

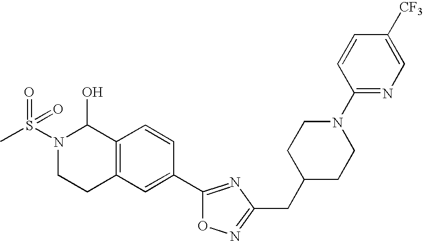

Compound 59 was obtained as a side product from example 58. ¹H-NMR (400 MHz, CD₃CN) δ=8.28 (m, 1H), 7.90-7.88 (m, 2H), 7.75 (dd, J=9.6, 2.4 Hz, 1H), 7.48 (d, J=8.0 Hz, 1H), 6.94 (d, J=8.0 Hz, 1H), 6.09 (s, 1H), 4.25 (br. d, J=16.8 Hz, 2H), 3.77-3.72 (m, 1H), 3.40-3.33 (m, 1H), 3.05-2.94 (m, 4H), 2.92 (s, 3H), 2.68 (d, J=6.8 Hz, 2H), 2.18-2.07 (m, 1H), 1.85-1.80 (m, 2H), 1.30 (ddd, J=12.8, 12.4, 4.0 Hz, 2H); MS calcd. for [M+H]⁺ C₂₄H₂₇F₃N₅O₄S: 538.2; found: 538.2.

### Example 60

### 1-Methylcyclopropyl 4-((5-(2-(methylsulfonyl)-1,2,3,4-tetrahydroisoquinolin-6-yl)-1,2,4-oxadiazol-3-yl)methyl)piperidine-1-carboxylate

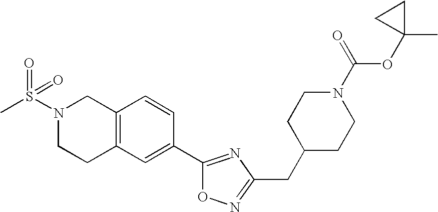

To a solution of 51a (200 mg, 0.44 mmol) and 1-methylcyclopropyl 4-nitrophenyl carbonate (115 mg, 0.48 mmol) in CH₂Cl₂ (2.5 mL) was added NEt₃ (0.5 mL). The resulting mixture was stirred at rt for 48 h. After dilution with CH₂Cl₂, the solution was washed with 1N NaOH followed by 1M HCl and brine. The organic layer was separated, dried (Na₂SO₄) and concentrated in vacuo. The crude was purified by flash chromatography (EtOAc/hexane) to yield the title compound (Example 60). ¹H-NMR (400 MHz, CDCl₃) δ=7.88-7.86 (m, 2H), 7.19 (d, J=8.0 Hz, 1H), 4.47 (s, 2H), 4.10-3.92 (m, 2H), 3.54 (t, J=6.0 Hz, 2H), 3.01 (t, J=6.0 Hz, 2H), 2.81 (s, 3H), 2.69-2.62 (m, 2H), 2.67 (d, J=6.8 Hz, 2H), 2.01-1.90 (m, 1H), 1.69-1.65 (m, 2H), 1.47 (s, 3H), 1.25-1.13 (m, 2H), 0.80-0.77 (m, 2H), 0.56-0.53 (m, 2H); MS calcd. for [M+Na]⁺ C₂₃H₃₀NaN₄O₅S: 497.1; found: 497.1.

### Example 61

### tert-Butyl 4-((3-(2-(methylsulfonyl)-1,2,3,4-tetrahydroisoquinolin-6-yl)-1,2,4-oxadiazol-5-yl)methyl)piperidine-1-carboxylate

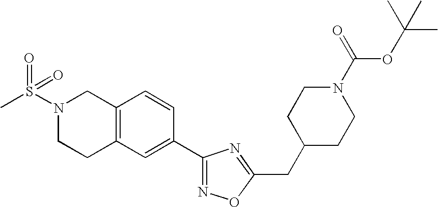

Intermediate 61c: NA-Hydroxy-2-(methylsulfonyl)-1,2,3,4-tetrahydroisoquinoline-6-carboximidamide

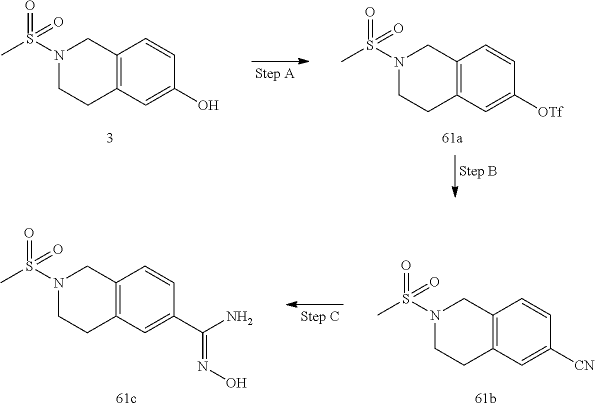

Step A A solution of 2-(methylsulfonyl)-1,2,3,4-tetrahydroisoquinolin-6-ol 3 (972 mg, 4.28 mmol) in CH₂Cl₂ (40 mL) was cooled to −78° C., treated with NEt₃ (1.2 mL, 8.6 mmol) and trifluoromethanesulfonic anhydride (0.79 mL, 4.7 mmol). The mixture was stirred at −78° C. for additional 30 minutes and then overnight at rt. Et₂O was added and the mixture was washed with 1M HCl. The aqueous phase was re-extracted with Et₂O. The combined organics were washed with brine, dried (Na₂SO₄) and concentrated in vacuo to give 2-(methylsulfonyl)-1,2,3,4-tetrahydroisoquinolin-6-yl trifluoromethanesulfonate (61a) that was used in the next step without further purification. ¹H-NMR (400 MHz, CDCl₃) δ=7.22-7.11 (m, 3H), 4.50 (s, 2H), 3.60 (t, J=6.0 Hz, 2H), 3.05 (t, J=6.0 Hz, 2H), 2.90 (s, 3H); MS calcd. for [M+H]⁺ C₁₁H₁₃F₃NO₅S: 360.0; found: 359.9.

Step B 61a (1.2 g, 3.34 mmol), Zn(CN)₂ (431 mg, 3.67 mmol), and Pd(PPh₃)₄ (386 mg, 0.33 mmol) were dissolved/suspended in DMF (3.5 mL) and heated at 110° C. overnight. After cooling to rt, the mixture was diluted with EtOAc, washed with brine, dried (Na₂SO₄), concentrated in vacuo and the crude was purified by flash chromatography (EtOAc/hexane) to yield 2-(methylsulfonyl)-1,2,3,4-tetrahydroisoquinoline-6-carbonitrile (61b). ¹H-NMR (400 MHz, CDCl₃) δ=7.53-7.50 (m, 2H), 7.23 (d, J=8.0 Hz, 1H), 4.53 (s, 2H), 3.60 (t, J=6.0 Hz, 2H), 3.05 (t, J=6.0 Hz, 2H), 2.91 (s, 3H); MS calcd. for [M+H]⁺ C₁₁H₁₃N₂O₂S: 237.1; found: 237.1.

Step C N′-Hydroxy-2-(methylsulfonyl)-1,2,3,4-tetrahydroisoquinoline-6-carboximidamide (61c) was synthesized from 61b (261 mg, 1.1 mmol) following the same procedure described for the preparation of 47b, using EtOAc as extracting solvent. ¹H-NMR (400 MHz, CDCl₃) δ=7.40-7.38 (m, 2H), 7.07 (d, J=8.0 Hz, 1H), 6.16 (br. s, 1H), 4.76 (br. s, 2H), 4.41 (s, 2H), 3.51 (t, J=6.0 Hz, 2H), 2.94 (t, J=6.0 Hz, 2H), 2.78 (s, 3H); MS calcd. for [M+H]⁺ C₁₁H₁₆N₃O₃S: 270.1; found: 270.0.

Following the procedure for Example 47, compound 61c (44.4 mg, 0.16 mmol) was condensed with 1-BOC-piperidin-4-yl-acetic acid (36.5 mg, 0.15 mmol) to afford the title compound (Example 61). ¹H-NMR (400 MHz, CD₃CN) δ=7.79-7.77 (m, 2H), 7.23 (d, J=8.4 Hz, 1H), 4.39 (s, 2H), 3.95 (br. d, J=12.8 Hz, 2H), 3.45 (t, J=6.0 Hz, 2H), 2.97 (t, J=6.0 Hz, 2H), 2.82 (t, J=7.2 Hz, 2H), 2.77 (s, 3H), 2.70-2.58 (m, 2H), 2.03-1.95 (m, 1H), 1.68-1.62 (m, 2H), 1.33 (s, 9H), 1.12 (ddd, J=12.0, 12.0, 4.0 Hz, 2H); MS calcd. for [M+2H−Boc]⁺ C₁₈H₂₅N₄O₃S: 377.1; found: 377.1.

Examples 62 and 63 (see table below) were synthesized by analogous methods from derivative 61c and the appropriate acids.

### Example 64

### Isopropyl 4-((3-(2-(methylsulfonyl)-1,2,3,4-tetrahydroisoquinolin-6-yl)-1,2,4-oxadiazol-5-yl)methyl)piperidine-1-carboxylate

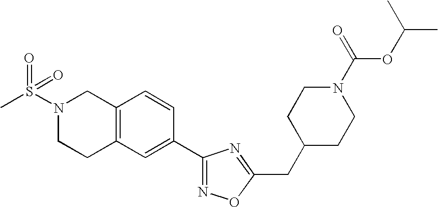

Intermediate 64a: 3-(2-(Methylsulfonyl)-1,2,3,4-tetrahydroisoquinolin-6-yl)-5-(piperidin-4-ylmethyl)-1,2,4-oxadiazole dihydrochloride

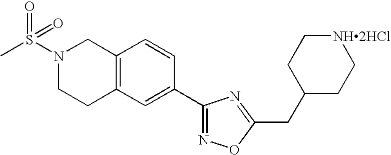

3-(2-(Methylsulfonyl)-1,2,3,4-tetrahydroisoquinolin-6-yl)-5-(piperidin-4-ylmethyl)-1,2,4-oxadiazole dihydrochloride (64a) was synthesized from 61 (1.83 g, 3.84 mmol) following the procedure described for the preparation of 51a. MS calcd. for [M+2H−Boc]⁺ C₁₈H₂₅N₄O₃S: 377.1; found: 377.1.

Following the procedure for the preparation of 27b, compound 64a (5.7 mg, 0.01 mmol) was converted to the title compound 64. The mixture was then diluted with MeCN and filtered. The filtrate was purified by reverse-phase HPLC to yield the title compound (Example 64). ¹H-NMR (400 MHz, CDCl₃) δ=7.82-7.80 (m, 2H), 7.14 (d, J=8.0 Hz, 1H), 4.42 (s, 2H), 4.14 (septet, J=6.0 Hz, 1H), 3.53 (t, J=6.0 Hz, 2H), 3.47 (br. d, J=12.0 Hz, 2H), 2.99 (t, J=6.0 Hz, 2H), 2.90 (t, J=7.2 Hz, 2H), 2.85-2.82 (m, 2H), 2.80 (s, 3H), 2.21-2.12 (m, 1H), 1.99-1.96 (m, 2H), 1.85-1.75 (m, 2H), 1.25 (d, J=6.0 Hz, 2H); MS calcd. for [M+2H−Boc]⁺ C₁₈H₂₅N₄O₃S: 377.1; found: 377.1.

### Example 65

### 5-((1-(5-Ethylpyrimidin-2-yl)piperidin-4-yl)methyl)-3-(2-(methylsulfonyl)-1,2,3,4-tetrahydroisoquinolin-6-yl)-1,2,4-oxadiazole

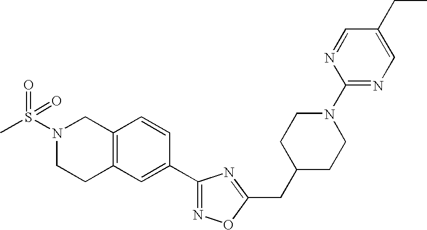

Following the procedure for Example 51 (method A), compound 64a (6.6 mg, 0.01 mmol) was converted to the title compound (Example 65). ¹H-NMR (400 MHz, DMSO-d₆) δ=8.23 (s, 2H), 7.84-7.81 (m, 2H), 7.39 (d, J=8.0 Hz, 1H), 4.62 (br. d, J=13.2 Hz, 2H), 4.45 (s, 2H), 3.47 (t, J=6.0 Hz, 2H), 3.04-2.98 (m, 4H), 2.98 (s, 3H), 2.90-2.83 (m, 2H), 2.42 (q, J=7.6 Hz, 2H), 2.21-2.11 (m, 1H), 1.77-1.73 (m, 2H), 1.23 (ddd, J=12.4, 12.4, 4.4 Hz, 2H), 1.12 (t, J=7.6 Hz, 3H); MS calcd. for [M+H]⁺ C₂₄H₃₁N₆O₃S: 483.1; found: 483.2.

### Example 66

### (E)-Isopropyl 4-(4-(2-(methylsulfonyl)-1,2,3,4-tetrahydroisoquinolin-6-yl)but-3-enyl)piperidine-1-carboxylate

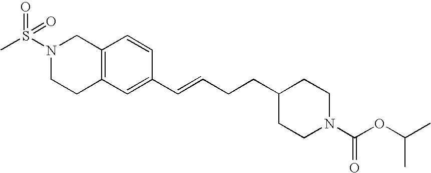

Intermediate 66a: 6-Bromo-2-(methylsulfonyl)-1,2,3,4-tetrahydroisoquinoline

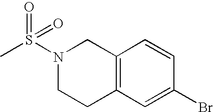

6-Bromo-2-(methylsulfonyl)-1,2,3,4-tetrahydroisoquinoline (66a) was prepared from 3-bromophenethylamine according to the same procedure described for the preparation of 26b. ¹H-NMR (400 MHz, CDCl₃) δ=7.36-7.34 (m, 2H), 6.99 (d, J=8.8 Hz, 1H), 4.42 (s, 2H), 3.57 (t, J=6.0 Hz, 2H), 2.98 (t, J=6.0 Hz, 2H), 2.87 (s, 3H); MS calcd. for [M+H]⁺ C₁₀H₁₃BrNO₂S: 289.9; found: 289.7.

Intermediate 66b: Isopropyl 4-(but-3-enyl)piperidine-1-carboxylate

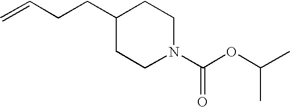

A mixture of 28c (535 mg, 1.66 mmol) in acetone (4 mL) was treated LiBr (434 mg, 5.0 mmol) and heated to 40° C. for 72 h. After removal of the solvent, the residue was partitioned between water and EtOAc. The organic phase was washed with water, dried (Na₂SO₄), and concentrated in vacuo. The residue was evaporated once from toluene, dissolved in THF (4 mL) and treated with ^{t}BuOK (934 mg, 8.32 mmol). After stirring overnight, the mixture was treated with sat. aqueous NH₄Cl and extracted with EtOAc. The combined organics were dried (Na₂SO₄), concentrated and the crude material was purified by flash chromatography (EtOAc/hexane) to yield isopropyl 4-(but-3-enyl)piperidine-1-carboxylate (66b) as a colorless oil. MS calcd. for [M+H]⁺ C₁₃H₂₄NO₂: 226.2; found: 226.1.

Dicyclohexylmethylamine (0.15 mL, 0.71 mmol) was added to a mixture of 66a (100 mg, 0.34 mmol), 66b (93 mg, 0.41 mmol), Pd₂(dba)₃ (4.73 mg, 0.005 mmol), and (^{t}Bu₃P)HBF₄ (3 mg, 0.01 mmol) in dioxane (1 mL). The vial was flushed with Ar, sealed and heated to 120° C. for 7 h. The mixture was partitioned between sat. aqueous NH₄C₁ and CH₂Cl₂, then extracted with CH₂Cl₂. The combined organics were dried (Na₂SO₄), concentrated and the crude material was purified by flash chromatography (EtOAc/hexane) to afford the title compound (Example 66). ¹H-NMR (400 MHz, CD₃CN) δ=7.14-7.09 (m, 2H), 6.99 (d, J=8.0 Hz, 1H), 6.29 (d, J=16.0 Hz, 1H), 6.20 (dt, J=16.0, 6.8 Hz, 1H), 4.72 (septet, J=6.0 Hz, 1H), 4.28 (s, 2H), 3.98-3.93 (m, 2H), 3.39 (t, J=6.0 Hz, 2H), 2.84 (t, J=6.0 Hz, 2H), 2.74 (s, 3H) 2.65 (br. s, 2H), 2.17-1.10 (m, 2H), 1.62 (br. d, J=13.2 Hz, 2H), 1.43-1.35 (m, 1H), 1.34-1.28 (m, 2H), 1.11 (d, J=6.0 Hz, 6H), 1.04-0.91 (m, 2H); MS calcd. for [M+H]⁺ C₂₃H₃₅N₂O₄S: 435.2; found: 435.2.

Examples 67 and 68 (see table below) were synthesized by analogous methods from derivative 66a and the appropriate alkene.

### Example 69

### Isopropyl 4-(4-(2-(methylsulfonyl)-1,2,3,4-tetrahydroisoquinolin-6-yl)butyl)piperidine-1-carboxylate

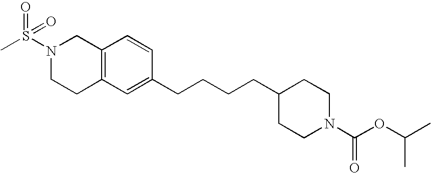

Example 66 (25 mg, 0.06 mmol) was dissolved in EtOAc/EtOH (1:1, 3 mL) and subjected to hydrogenolysis (H-cube, full-hydrogen mode, Thales nanotechnologies) at 60° C. Upon the completion of reaction, the solvent was evaporated and the crude product was purified by reverse-phase HPLC to yield the title compound (Example 69). ¹H-NMR (400 MHz, CD₃CN) δ=6.99-6.93 (m, 3H), 4.72 (septet, J=6.0 Hz, 1H), 4.27 (s, 2H), 3.93 (br. d, J=12.4 Hz, 2H), 3.38 (t, J=6.0 Hz, 2H), 2.83 (t, J=6.0 Hz, 2H), 2.74 (s, 3H) 2.61 (br. t, 2H), 2.48 (t, J=7.6 Hz, 2H), 1.56-1.44 (m, 4H), 1.36-1.15 (m, 5H), 1.10 (d, J=6.0 Hz, 6H), 0.99-0.85 (m, 2H); MS calcd. for [M+H]⁺ C₂₃H₃₇N₂O₄S: 437.2; found: 437.2.

Examples 70 and 71 (see table below) were synthesized by analogous methods from Examples 67 and 68.

### Example 72

### Isopropyl 4-(3-(2-(methylsulfonyl)-1,2,3,4-tetrahydroisoquinolin-6-yl)phenoxy)piperidine-1-carboxylate

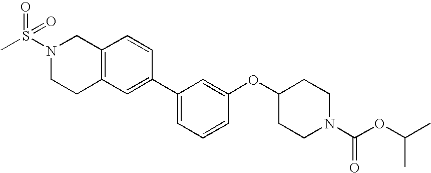

Intermediate 72a: 3-(2-(Methylsulfonyl)-1,2,3,4-tetrahydroisoquinolin-6-yl)phenol

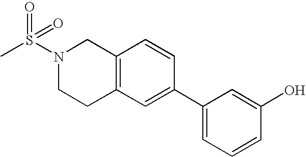

Intermediate 66a (100 mg, 0.34 mmol), 3-hydroxyphenylboronic acid (95 mg, 0.69 mmol), and Pd(PPh₃)₄ (12 mg, 0.01 mmol) were charged into a microwave vial. EtOH (1.3 mL) was added followed by a solution of Cs₂CO₃ (225 mg, 0.69 mmol) in water (0.7 mL). The vial was then sealed and heated in the microwave to 110° C. for 10 minutes. After removal of the solvent, the crude was purified by flash chromatography (EtOAc/hexane) to yield 3-(2-(Methylsulfonyl)-1,2,3,4-tetrahydroisoquinolin-6-yl)phenol (72a) as a white solid. ¹H-NMR (400 MHz, CDCl₃) δ=7.35 (dd, J=7.6, 1.6 Hz, 1H), 7.30 (m, 1H), 7.24 (t, J=7.6 Hz, 1H), 7.10-7.06 (m, 2H), 6.97 (dd, J=2.4, 1.6 Hz, 1H), 6.75 (ddd, J=8.0, 2.4, 0.8 Hz, 1H), 4.71 (septet, J=6.4 Hz, 1H), 4.44 (s, 2H), 3.54 (t, J=6.0 Hz, 2H), 2.98 (t, J=6.0 Hz, 2H), 2.80 (s, 3H); MS calcd. for [M+H]⁺ C₁₆H₁₈NO₃S: 304.1; found: 304.1.

Intermediate 72c: Isopropyl 4-(methylsulfonyloxy)piperidine-1-carboxylate

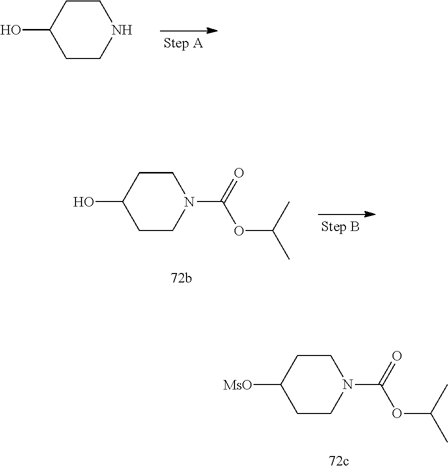

Step A NEt₃ (10.4 mL, 74.6 mmol) was added to a solution of 4-hydroxypiperidine (5.82 g, 57.5 mmol) in EtOAc (50 mL) at rt. The resulting suspension was cooled to 0° C., treated with a solution of isopropyl chloroformate in toluene (1.0M, 69 mL) and stirred at rt overnight. The mixture was quenched with water and stirred for 15 minutes, until a clear solution formed. The organic phase was separated and the aqueous layer was extracted with EtOAc. The combined organics were washed with brine, dried (Na₂SO₄), concentrated, and the crude material was distilled under high vacuum to afford isopropyl 4-hydroxypiperidine-1-carboxylate (72b) as a clear oil. ¹H-NMR (400 MHz, CDCl₃) δ=4.90 (septet, J=6.0 Hz, 1H), 3.95-3.82 (m, 2H), 3.10-3.03 (m, 2H), 1.90-1.83 (m, 2H), 1.70-1.61 (m, 1H), 1.51-1.42 (m, 2H), 1.24 (d, J=6.0 Hz, 6H); MS calcd. for [M+H]⁺ C₉H₁₈NO₃: 188.1; found: 188.1.

Step B Isopropyl 4-(methylsulfonyloxy)piperidine-1-carboxylate (72c) was prepared from 72b (1 g, 5.3 mmol) according to the procedure described for the preparation of 27c. ¹H-NMR (400 MHz, CDCl₃) δ=4.96-4.86 (m, 2H), 3.76-3.70 (m, 2H), 3.38-3.32 (m, 2H), 3.04 (s, 3H), 2.00-1.94 (m, 2H), 1.86-1.78 (m, 2H), 1.24 (d, J=6.4 Hz, 6H); MS calcd. for [M+H]⁺ C₁₀H₂₀NO₅S: 266.1; found: 266.1.

In a microwave vial DMA (0.5 mL) was added to a mixture of 72a (20 mg, 0.066 mmol), Cs₂CO₃ (43 mg, 0.13 mmol) and 72c (19 mg, 0.072 mmol). The vial was sealed and the mixture was heated at 150° C. for 20 minutes. After cooling to rt, the mixture was diluted with MeCN and filtered. The filtrate was purified by reverse-phase HPLC to yield the title compound (Example 72). ¹H-NMR (400 MHz, CD₃CN) δ=7.40-7.38 (m, 2H), 7.27 (t, J=8.0 Hz, 1H), 7.16-7.10 (m, 3H), 6.87-6.85 (m 1H), 4.75 (septet, J=6.4 Hz, 1H), 4.58-4.52 (m, 1H), 4.36 (s, 2H), 3.71-3.65 (m, 2H), 3.44 (t, J=6.0 Hz, 2H), 3.23-3.17 (m, 2H), 2.94 (t, J=6.0 Hz, 2H), 2.77 (s, 3H), 1.91-1.88 (m, 2H), 1.60-1.52 (m, 2H), 1.13 (d, J=6.4 Hz, 6H); MS calcd. for [M+H]⁺ C₂₅H₃₃N₂O₅S: 473.2; found: 473.2.

### Example 73

### Isopropyl 4-((3-(2-(methylsulfonyl)-1,2,3,4-tetrahydroisoquinolin-6-yl)phenoxy)methyl)piperidine-1-carboxylate

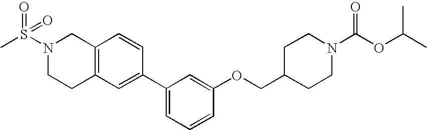

Following the procedure for Example 72, phenol 72a (20 mg, 0.066 mmol) was alkylated with 43c (20 mg, 0.071 mmol) to afford the title compound (Example 73). ¹H-NMR (400 MHz, CD₃CN) δ=7.40-7.37 (m, 2H), 7.28-7.25 (m, 1H), 7.15-7.08 (m, 3H), 6.82 (dd, J=8.4, 2.4 Hz, 1H), 4.75 (septet, J=6.4 Hz, 1H), 4.36 (s, 2H), 4.03 (br. d, J=12.8 Hz, 2H), 3.83 (d, J=6.4 Hz, 2H), 3.44 (t, J=6.0 Hz, 2H), 2.94 (t, J=6.0 Hz, 2H), 2.76 (s, 3H), 2.71 (br. t, 2H), 1.96-1.88 (m, 1H), 1.73 (br. d, J=12.8 Hz, 2H), 1.20-1.10 (m, 2H), 1.13 (d, J=6.4 Hz, 6H); MS calcd. for [M+H]⁺ C₂₆H₃₅N₂O₅S: 487.2; found: 487.2.

### Example 74

### Isopropyl 4-(4-(2-(methylsulfonyl)-1,2,3,4-tetrahydroisoquinolin-6-yl)-4-oxobutyl)piperidine-1-carboxylate

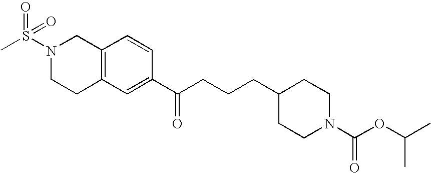

Intermediate 74a: N-Methoxy-N-methyl-2-(methylsulfonyl)-1,2,3,4-tetrahydroisoquinoline-6-carboxamide

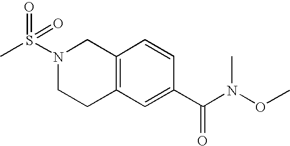

To a solution/suspension of 40b (5.89 g, 23.1 mmol) and N,O-dimethylhydroxylamine hydrochloride (2.58 g, 25.4 mmol) in CH₂Cl₂ (90 mL) was added EtN(i-Pr)₂ (8.9 mL, 52.0 mmol) followed by HATU (10.52 g, 27.7 mmol). The resulting mixture was stirred overnight at rt, diluted with CH₂Cl₂, and washed with 1M HCl, 1N NaOH and brine. The organic layer was dried (Na₂SO₄), concentrated and the crude was purified by flash chromatography (EtOAc/hexane) to yield N-methoxy-N-methyl-2-(methylsulfonyl)-1,2,3,4-tetrahydroisoquinoline-6-carboxamide (74a) as a white solid. ¹H-NMR (400 MHz, CDCl₃) δ=7.54 (d, J=8.0 Hz, 1H), 7.52 (s, 1H), 7.15 (d, J=8.0 Hz, 1H), 4.51 (s, 2H), 3.60 (t, J=6.0 Hz, 2H), 3.58 (s, 3H), 3.38 (s, 3H), 3.04 (t, J=6.0 Hz, 2H), 2.88 (s, 3H); MS calcd. for [M+H]⁺ C₁₃H₁₉N₂O₄S: 299.1; found: 298.9.

Intermediate 74b: Isopropyl 4-(3-bromopropyl)piperidine-1-carboxylate

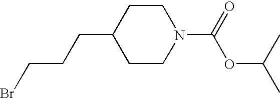

PPh₃ (4.80 g, 18.3 mmol) was added portionwise to a solution of 27b (2 g, 8.7 mmol) and CBr₄ (5.78 g, 17.4 mmol) in CH₂Cl₂ (17 mL) at 0° C. The resulting mixture was stirred at rt for 3 h, then filtered through a celite plug. The plug was washed with CH₂Cl₂, and the organics were concentrated. The crude material was purified by flash chromatography (EtOAc/hexane) to yield isopropyl 4-(3-bromopropyl)piperidine-1-carboxylate (74b) as a colorless oil. ¹H-NMR (400 MHz, CDCl₃) δ=4.91 (septet, J=6.4 Hz, 1H), 4.14 (br. s, 2H), 3.41 (t, J=6.8 Hz, 2H), 2.72 (br. t, J=12.4 Hz, 2H), 1.93-1.86 (m, 2H), 1.67 (br. d, J=12.8 Hz, 2H), 1.45-1.37 (m, 3H), 1.25 (d, J=6.4 Hz, 6H), 1.17-1.07 (m 2H); MS calcd. for [M+H]⁺ C₁₂H₂₃BrNO₂: 292.1; found: 292.0.

To a dry 2-neck flask charged with magnesium turnings (110 mg, 4.5 mmol) and dry THF (1 mL) was added a solution of 74b (1 g, 3.4 mmol) in dry THF (4 mL) at 50° C. Upon completed addition the mixture was stirred at 55° C. for 2.5 h and cooled to rt. This freshly prepared Grignard reagent solution was then cannulated into a solution of 74a (500 mg, 1.68 mmol) in THF (5 mL). After complexion of the reaction (3 h), the mixture was diluted with sat. aqueous NH₄Cl and extracted with EtOAc. The combined organics were washed with brine, dried (Na₂SO₄), concentrated and the crude material purified by flash chromatography (EtOAc/hexane) to yield the title compound (Example 74) as a white solid. ¹H-NMR (400 MHz, CDCl₃) δ=7.81-7.78 (m 2H), 7.21 (d, J=8.0 Hz, 1H), 4.92 (septet, J=6.4 Hz, 1H), 4.52 (s, 2H), 4.14 (br. s, 2H), 3.61 (t, J=6.0 Hz, 2H), 3.07 (t, J=6.0 Hz, 2H), 2.89 (s, 3H), 2.72 (br. t, J=12.4 Hz, 2H), 1.81-1.69 (m, 4H), 1.49-1.42 (m, 1H), 1.37-1.31 (m, 2H), 1.25 (d, J=6.4 Hz, 6H), 1.16-1.07 (m 2H); MS calcd. for [M+H]⁺ C₂₃H₃₅N₂O₅S: 451.2; found: 451.2.

### Example 75

### Isopropyl 4-(4,4-difluoro-4-(2-(methylsulfonyl)-1,2,3,4-tetrahydroisoquinolin-6-yl)butyl)piperidine-1-carboxylate

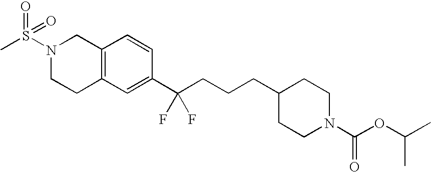

Intermediate 75a: Isopropyl 4-(3-(2-(2-(methylsulfonyl)-1,2,3,4-tetrahydroisoquinolin-6-yl)-1,3-dithiolan-2-yl)propyl)piperidine-1-carboxylate

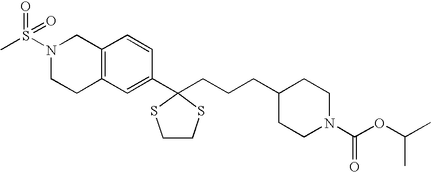

Ethanedithiol (37 μL, 0.44 mmol) and BF₃.2AcOH (62 μL, 0.44 mmol) were added to 74 (100 mg, 0.22 mmol) under N₂ atmosphere. The mixture was stirred for 10 minutes at rt, diluted with EtOAc, and washed with sat. NaHCO₃, 1N NaOH and brine. The organic layer was dried (Na₂SO₄), concentrated, and the crude purified by flash chromatography (EtOAc/hexane) to yield isopropyl 4-(3-(2-(2-(methylsulfonyl)-1,2,3,4-tetrahydroisoquinolin-6-yl)-1,3-dithiolan-2-yl)propyl)piperidine-1-carboxylate (75a) as a colorless oil. ¹H-NMR (400 MHz, CDCl₃) δ=7.52-7.50 (m 2H), 7.05 (d, J=8.4 Hz, 1H), 4.90 (septet, J=6.4 Hz, 1H), 4.45 (s, 2H), 4.09 (br. s, 2H), 3.57 (t, J=6.0 Hz, 2H), 3.43-3.35 (m, 2H), 3.30-3.22 (m, 2H), 3.00 (t, J=6.0 Hz, 2H), 2.87 (s, 3H), 2.66 (br. t, J=12.0 Hz, 2H), 2.33-2.29 (m, 2H), 1.59 (br. d, J=11.6 Hz, 2H), 1.38-1.20 (m, 5H), 1.23 (d, J=6.4 Hz, 6H), 1.07-0.97 (m 2H); MS calcd. for [M+H]⁺ C₂₅H₃₉N₂O₅S: 527.2; found: 527.2.

HF-pyridine (0.1 mL) was added dropwise to a suspension of 1,3-dimethyl-5,5-dimethylhydantoin (34 mg, 0.12 mmol) in CH₂Cl₂ (0.2 mL) at −78° C. The resulting colorless solution was then treated with a solution of 75a (38 mg, 0.07 mmol) in CH₂Cl₂ (0.2 mL) and stirred at −78° C. for 30 minutes. The mixture was then filtered through a plug of basic aluminium oxide (Brockmann I, Aldrich) and washed with CH₂Cl₂. The solvent was evaporated and the crude material was purified on reverse-phase HPLC to yield the title compound (Example 75). ¹H-NMR (400 MHz, CD₃CN) δ=7.36-7.34 (m 2H), 7.26 (d, J=8.4 Hz, 1H), 4.82 (septet, J=6.0 Hz, 1H), 4.46 (s, 2H), 4.03 (br. d, J=12.8 Hz, 2H), 3.53 (t, J=6.0 Hz, 2H), 3.01 (t, J=6.0 Hz, 2H), 2.87 (s, 3H), 2.70 (br. t, 2H), 2.23-2.11 (m, 2H), 1.62 (br. d, J=12.4 Hz, 2H), 1.46-1.35 (m, 3H), 1.29-1.25 (m, 2H), 1.21 (d, J=6.0 Hz, 6H), 0.99 (ddd, J=13.2, 12.8, 4.4, 2H); ¹⁹F-NMR (376 MHz, CD₃CN) δ=−94.585; MS calcd. for [M+H]⁺ C₂₃H₃₅F₂N₂O₄S: 473.2; found: 473.2.

### Example 76

### Isopropyl 4-(4-(1-(methylsulfonyl)-2,3,4,5-tetrahydro-1H-benzo[b]azepin-7-yloxy)butyl)piperidine-1-carboxylate

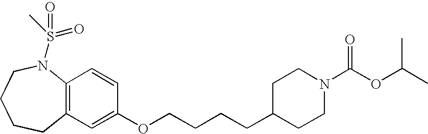

Intermediate 76e: 1-(Methylsulfonyl)-2,3,4,5-tetrahydro-1H-benzo[b]azepin-7-ol

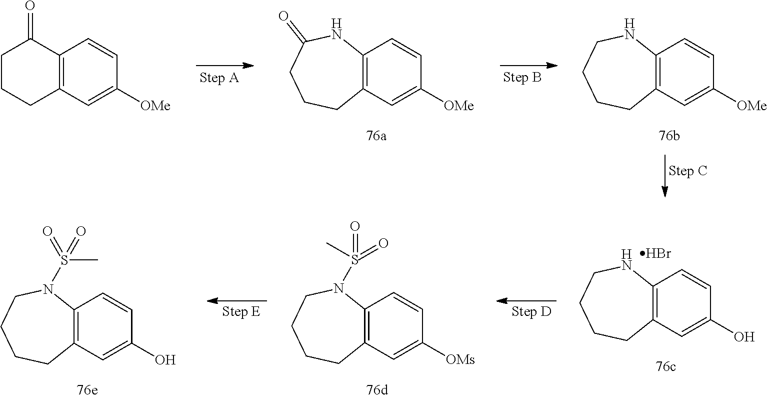

Step A NaN₃ (5.70 g, 87.7 mmol) was added in small portions to a solution of 6-methoxy-1-tetralone (15 g, 85.1 mmol) in concentrated HCl at 0° C. The resulting mixture was stirred at rt for 4 h, then carefully poured into a cold biphasic solution of CH₂Cl₂ (150 mL) and aqueous K₂CO₃ (150 g in 300 mL). The organic layer was separated and the aqueous layer was extracted with CH₂Cl₂. The combined organics were washed with brine, dried (Na₂SO₄), concentrated and the crude was purified by flash chromatography (EtOAc/hexane) to afford 7-methoxy-4,5-dihydro-1H-benzo[b]azepin-2(3H)-one (76a) as a white solid. ¹H-NMR (400 MHz, CDCl₃) δ=7.23 (br. s, 1H), 6.92 (d, J=8.0 Hz, 1H), 6.79-6.67 (m, 2H), 3.83 (s, 3H), 2.80 (t, J=7.2 Hz, 2H), 2.35 (t, J=7.2 Hz, 2H), 2.27-2.20 (m, 2H); MS calcd. for [M+H]⁺ C₁₁H₁₄NO₂: 192.1; found: 192.1.

Step B A solution of 76a (2.14 g, 11.2 mmol) in dioxane (15 mL) was added dropwise at 0° C. to a solution of LiAlH₄ in THF (1M, 39 mL, 39 mmol) under Ar atmosphere. Upon completed addition, the mixture was heated to reflux overnight. After cooling to rt, sat. aqueous Na₂SO₄ was added until the gas evolution ceased. The residue was filtered over celite, washed with EtOAc and discarded. The filtrate was concentrated to yield crude 7-methoxy-2,3,4,5-tetrahydro-1H-benzo[b]azepine (76b) that was used in the next step without further purification. ¹H-NMR (400 MHz, CDCl₃) δ=6.73-6.70 (m, 2H), 6.62 (dd, J=8.4, 2.8 Hz, 1H), 3.78 (s, 3H), 3.02-2.99 (m, 2H), 2.77-2.74 (m, 2H), 1.84-1.78 (m, 2H), 1.67-1.62 (m, 2H); MS calcd. for [M+H]⁺ C₁₁H₁₆NO: 178.1; found: 178.1.

Step C A solution of 76b (1.98 g, 11.2 mmol) in HBr (48%, 20 mL) was heated to reflux for 4 h. After removal of the solvents, the residue was dissolved in EtOH and filtered to remove any insoluble material. The filtrate was concentrated to afford 2,3,4,5-tetrahydro-1H-benzo[b]azepin-7-ol hydrobromide (76c) that was used in the next step without further purification. ¹H-NMR (400 MHz, CD₃OD) δ=7.20-7.17 (m, 1H), 6.78 (d, J=2.8 Hz, 1H), 6.71 (dd, J=8.8, 2.8 Hz, 1H), 3.37-3.34 (m, 2H), 2.91-2.89 (m, 2H), 2.17-2.11 (m, 2H), 1.85-1.78 (m, 2H); MS calcd. for [M+H]⁺ C₁₀H₁₄NO: 164.1; found: 164.1.

Step D Intermediate 76c (1.5 g, 6.14 mmol) was dissolved in dry CH₂Cl₂ (50 mL), then NEt₃ (2.57 mL, 18.4 mmol) was added. The resulting mixture was cooled to 0° C. Methanesulfonyl chloride (1 mL, 12.9 mmol) was added dropwise, with vigorous stirring, over 5 min. The ice-bath was removed and the resulting solution was stirred at rt overnight. The reaction mixture was added to water (40 mL) and extracted with CH₂Cl₂. The combined organic extracts were washed with sat. aqueous NH₄Cl, dried (Na₂SO₄), and concentrated in vacuo to yield 1-(methylsulfonyl)-2,3,4,5-tetrahydro-1H-benzo[b]azepin-7-yl methanesulfonate (76d) that was used in the next step without further purification. MS calcd. for [M+H]⁺ C₁₂H₁₈NO₅S₂: 320.1; found: 320.0.

Step E A suspension of 76d (1.96 g, 6.14 mmol) in MeOH (40 mL) and NaOH solution (10%, 40 mL) was heated to 80° C. for 1.5 h. After cooling to rt, the mixture was diluted with EtOAc, washed with sat. aqueous NH₄Cl and brine. The organic layer was dried (Na₂SO₄), concentrated and the crude was purified by flash chromatography (EtOAc/hexane) to afford 1-(methylsulfonyl)-2,3,4,5-tetrahydro-1H-benzo[b]azepin-7-ol (76e) as a white solid. ¹H-NMR (400 MHz, CDCl₃) δ=7.24 (d, J=8.4 Hz, 1H), 6.70 (d, J=3.2 Hz, 1H), 6.65 (dd, J=8.4, 3.2 Hz, 1H), 4.91 (s, 1H), 3.62 (br. s, 2H), 3.05 (s, 3H), 2.84-2.82 (m, 2H), 1.96-1.91 (m, 2H), 1.71 (br. s, 2H).

Following the procedure for Example 26, compound 76e (36.2 mg, 0.15 mmol) was alkylated with 28c (53 mg, 0.16 mmol) to afford the title compound (Example 76). ¹H-NMR (400 MHz, CD₃CN) δ=7.13 (d, J=8.4 Hz, 1H), 6.70 (d, J=2.8 Hz, 1H), 6.64 (dd, J=8.8, 3.2 Hz, 1H), 4.72 (septet, J=6.0 Hz, 1H), 3.95 (br. d, J=13.2 Hz, 2H), 3.88 (t, J=6.4 Hz, 2H), 3.42 (br. s, 2H), 2.94 (s, 3H), 2.73-2.70 (m, 2H), 2.63 (br. t, 2H), 1.80-1.75 (m, 2H), 1.70-1.57 (m, 6H), 1.42-1.30 (m, 3H), 1.24-1.18 (m, 2H), 1.11 (d, J=6.0 Hz, 6H), 0.93 (ddd, J=12.8, 12.4, 4.0 Hz, 2H); MS calcd. for [M+H]⁺ C₂₄H₃₉N₂O₅S: 467.2; found: 467.2.

### Example 77

### 2-(Methylsulfonyl)-6-(3-(1-(5-Dentylpyrimidin-2-yl)piperidin-4-yl)propoxy)-1,2,3,4-tetrahydroisoquinoline

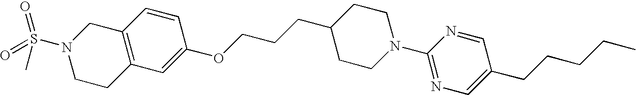

Intermediate 77c: 2-(Methylsulfonyl)-6-(3-(piperidin-4-yl)propoxy)-1,2,3,4-tetrahydroisoquinoline

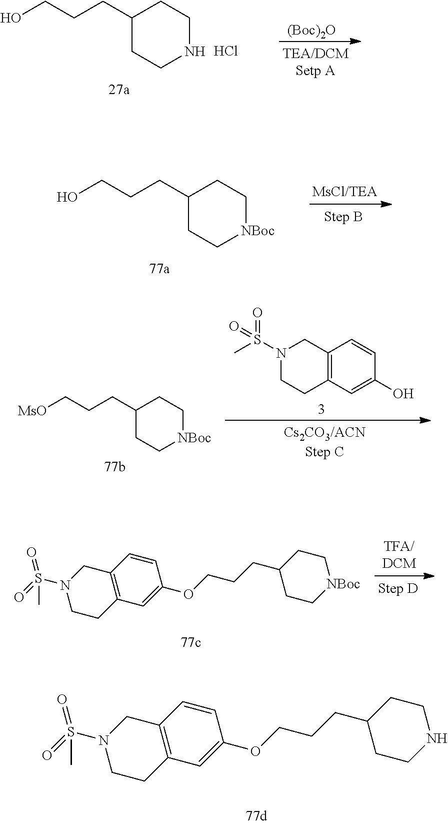

Step A To a suspension of 3-(piperidin-4-yl)propan-1-ol hydrochloride (27a) (30.00 g, 0.167 mol) and TEA (51.2 mL, 0.367 mol) in CH₂Cl₂ (150 mL) was slowly added (Boc)₂O (36.4 g, 0.167 mol) in CH₂Cl₂ at low temperature (the internal temperature was maintained below −5° C. during the addition). After completion of the addition, the cold bath was removed and the reaction was stirred at rt overnight. The resulting white precipitate was filtered and washed with ether. The filtrate was washed with brine (20 mL), dried over MgSO₄, and evaporated to afford tert-butyl 4-(3-hydroxypropyl)piperidine-1-carboxylate (77a) as a thick oil. ¹H NMR (400 MHz, CD₃CN) δ 4.00 (m, 2H), 3.46 (dd, J=4.8, 8.4 Hz, 2H), 2.67 (m, 2H), 2.50 (t, J=3.6 Hz, 1H), 1.65 (m, 2H), 1.49 (m, 2H), 1.45 (s, 9H), 1.38 (m, 1H), 1.25 (m, 2H), 0.99 (ddd, J=3.3, 9.6, 18.6 Hz, 2H).

Step B MsCl (14.3 mL, 0.184 mol) was slowly added to a stirred solution of 77a (43.6 g) in CH₂Cl₂ (150 mL) and pyridine (27 mL, 0.184 mol) at 0° C. over 30 min. The reaction was stirred at 0° C. for another hour, then at rt overnight. The mixture was quenched with water (50 mL) and extracted with EtOAc (3×100 mL). The organic layers were combined, washed with brine (25 mL), dried over MgSO₄, and evaporated to give a crude amber colored oil which was purified by flash chromatography (EtOAc/hexanes=0-100%) to give tert-butyl 4-(3-(methylsulfonyloxy)propyl)piperidine-1-carboxylate (77b) as a light yellow oil. ¹H NMR (400 MHz, CD₃CN) δ 4.18 (t, J=4.8 Hz, 2H), 4.00 (m, 2H), 2.99 (s, 3H), 2.67 (m, 2H), 1.72 (m, 2H), 1.65 (m, 2H), 1.43 (m, 1H), 1.41 (s, 9H), 1.30 (m, 2H), 1.01 (ddd, J=3.3, 9.6, 18.6 Hz, 2H).

Step C A suspension of 2-(methylsulfonyl)-1,2,3,4-tetrahydroisoquinolin-6-ol (3) (9.15 g, 40.3 mmol), tert-butyl 4-(3-(methylsulfonyloxy)-propyl)piperidine-1-carboxylate (77b) (12.9 g, 40.3 mmol) and Cs₂CO₃ (16.34 g, 50.3 mmol) in ACN (150 mL) was heated at 80° C. (oil bath) for 24 h under Argon. After cooling at rt, the mixture was filtered and the filter cake was washed with EtOAc (200 mL). The filtrate was evaporated to afford tert-butyl 4-(3-(2-(methylsulfonyl)-1,2,3,4-tetrahydroisoquinolin-6-yloxy)propyl)-piperidine-1-carboxylate (77c) a light pinkish solid.

Step D To a solution of compound 77c (22.42 g, 50 mmol) in CH₂Cl₂ (150 mL) was slowly added TFA (30 mL) at 0° C. After 30 min, the cold bath was removed and the mixture was stirred at rt for 3 h. After removal of the solvent, the residue was taken up by 50 mL of saturated NaHCO₃, and basified to pH-10 by 20% NaOH. The gummy precipitate was collected and purified by flash chromatography (MeOH/CH₂Cl₂=0-10%) to afford 2-(methylsulfonyl)-6-(3-(piperidin-4-yl)propoxy)-1,2,3,4-tetrahydroisoquinoline (77d) as an off white solid. ¹H NMR (400 MHz, CDCl₃) δ 6.99 (d, J=6.3 Hz, 1H), 6.74 (dd, J=1.8, 6.3 Hz, 1H), 6.65 (d, J=1.8 Hz, 1H), 4.39 (s, 2H), 3.92 (t, J=4.5 Hz, 2H), 3.54 (t, J=4.2 Hz, 2H), 3.37 (d, J=9.6 Hz, 2H), 2.96 (t, J=4.5 Hz, 2H), 2.87-2.79 (m, 2H), 2.81 (s, 3H), 1.89 (d, J=9.0 Hz, 2H), 1.79 (m, 2H), 1.60-1.46 (m, 5H); MS calcd. for [M+H]⁺ C₁₈H₂₉N₂O₃S: 353.2; found: 353.1.

A mixture of 2-(methylsulfonyl)-6-(3-(piperidin-4-yl)propoxy)-1,2,3,4-tetrahydroisoquinoline (77d) (100 mg, 0.283 mmol), 2-chloro-5-pentylpyrimidine (76 mg, 0.411 mmol) and Cs₂CO₃ (185 mg, 0.567 mmol) in dioxane (2 mL) was stirred in a sealed vial at 150° C. for 70 min. LC-MS indicated the reaction was complete. The reaction mixture was filtered through a syringe filter and purified by flash chromatography (EtOAc/hexanes=0-40%) to afford the title compound (Example 77) as an off white solid. ¹H NMR (400 MHz, CD₃CN) δ 8.16 (s, 2H), 7.04 (d, J=6.2 Hz, 1H), 6.75 (dd, J=1.8, 6.3 Hz, 1H), 6.72 (d, J=2.1 Hz, 1H), 4.65 (m, 2H), 4.31 (s, 2H), 3.94 (t, J=5.1 Hz, 2H), 3.45 (t, J=4.5 Hz, 2H), 2.91 (t, J=4.2 Hz, 2H), 2.82 (dt, J=1.8, 9.9 Hz, 2H), 2.81 (s, 3H), 2.40 (t, J=5.7 Hz, 2H), 1.80 (m, 4H), 1.53 (m, 3H), 1.41-1.25 (m, 6H), 1.09 (ddd, J=3.3, 9.6, 18.6 Hz, 2H), 0.89 (t, J=5.1 Hz, 3H); MS calcd. for [M+H]⁺ C₂₇H₄₁N₄O₃S: 501.3; found: 501.2.

Examples 78-99 (see table below) were synthesized by analogous method from Example 77.

### Example 100

### 2-(Methylsulfonyl)-6-(3-(1-(5-carboxypyridin-2-yl)piperidin-4-yl)propoxy)-1,2,3,4-tetrahydroisoquinoline

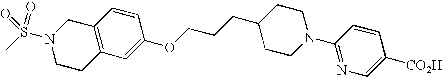

A solution of methyl ester (Example 95, 30 mg, 0.062 mmol) and LiOH (4 mg, 0.167 mmol) in a mixture of THF/MeOH/H₂O (3 mL/1 mL/1 mL) was stirred at 60° C. for 3 h. An additional 4 mg of LiOH was then added and the reaction continued at 60° C. for another 2 h. The reaction mixture was acidified (1N HCl) to pH-3, and concentrated to give a white precipitate which was collected by filtration (24 mg). Trituration of the precipitate into EtOAc (2 mL) for 1 h followed by filtration afforded the corresponding acid (2-(methylsulfonyl)-6-(3-(1-(5-carboxypyridin-2-yl)piperidin-4-yl)propoxy)-1,2,3,4-tetrahydroisoquinoline (Example 100). ¹H NMR (400 MHz, DMSO-d₆) δ 8.60 (d, J=1.8 Hz, 1H), 7.88 (dd, J=1.8, 6.9 Hz, 1H), 7.07 (d, J=6.3 Hz, 1H), 6.84 (d J=6.9 Hz, 1H), 6.76 (m, 2H), 4.46 (d, J=9.9 Hz, 2H), 4.27 (s, 2H), 3.93 (t, J=4.8 Hz, 2H), 3.38 (t, J=4.5 Hz, 2H), 2.92 (s, 3H), 2.86 (m, 4H), 1.73 (m, 4H), 1.59 (m, 1H), 1.35 (m, 2H), 1.07 (ddd, J=3.0, 9.6, 18.6 Hz, 2H); MS calcd. for [M+H]⁺ C₂₄H₃₂N₃O₅S: 474.2; found: 474.2.

### Example 101

### 6-(3-(1-(6-Ethylpyridazin-3-yl)piperidin-4-yl)propoxy)-2-(methylsulfonyl)-1,2,3,4-tetrahydroisoquinoline

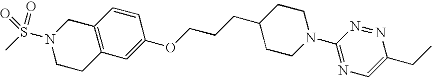

Intermediate 101a: 3-chloro-6-ethylpyridazine

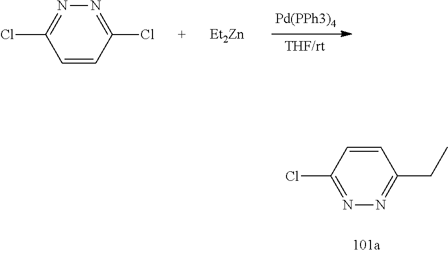

To a degassed solution of Pd(PPh₃)₄ (0.39 g, 0.34 mmol) and 3,6-dichloropyridazine (1.00 g, 6.71 mmol) in THF (20 mL) was slowly added a solution of Et₂Zn (0.5M in THF) at −78° C. The reaction mixture was allowed to warm-up to rt slowly, quenched with saturated NaHCO₃ (10 mL), and then filtered through celite plug which was subsequently washed with CH₂Cl₂ (100 mL). The organic layers were then dried over MgSO₄, filtrated and concentrated to afford a brown solid which was purified by flash chromatography (EtOAc/hexanes=0-30%) to afford 3-chloro-6-ethylpyridazine (101a) as a light yellow solid. ¹H NMR (400 MHz, CDCl₃) δ 7.43 (d, J=6.6 Hz, 1H), 7.32 (d, J=6.6 Hz, 1H), 3.01 (q, J=6.0, 10.8 Hz, 2H), 1.36 (t, J=6.0 Hz, 3H); MS calcd. for [M+H]⁺ C₆H₈ClN₂: 143.0; found: 143.0.

To a reaction vessel was charged with a mixture of 2-(methylsulfonyl)-6-(3-(piperidin-4-yl)propoxy)-1,2,3,4-tetrahydroisoquinoline (77d) (70 mg, 0.20 mmol), 3-chloro-6-ethylpyridazine (101a) (42 mg, 0.30 mmol), Pd₂ dba₃ (9 mg, 0.01 mmol), 2-dicyclohexylphosphino-2′,6′-dimethoxy-1,1′-biphenyl (17 mg, 0.041 mmol), NaO^{t}Bu (29 mg, 0.30 mmol) and toluene (1.5 mL). The mixture was degassed. The reaction vessel was then sealed and heated to 100° C. for 90 min. After cooling at rt, the mixture was filtrated and purified by HPLC to give the title compound 101 as an off white powder (TFA salt). ¹H NMR (400 MHz, CDCl₃) δ 7.47 (dd, J=7.2, 15.6 Hz, 2H), 6.96 (d, J=6.3 Hz, 1H), 6.67 (dd, J=2.1, 6.3 Hz, 1H), 6.64 (d, J=1.8 Hz, 1H), 4.22 (s, 2H), 4.19 (m, 2H), 3.86 (t, J=4.8 Hz, 2H), 3.36 (t, J=4.5 Hz, 2H), 2.98 (dt, J=1.8, 9.9 Hz, 2H), 2.80 (m, 4H), 2.72 (2, 3H), 1.78 (m, 2H), 1.69 (m, 2H), 1.58 (m, 1H), 1.33 (m, 2H), 1.19-1.13 (m, 5H); MS calcd. for [M+H]⁺ C₂₄H₃₅N₄O₃S: 459.2; found: 459.2.

Examples 102-120 (see table below) were synthesized by analogous method from Example 101.

3-Chloro-6-propylpyridazine was prepared according to the procedure described for the synthesis of 3-chloro-6-ethylpyridazine (110a). ¹H NMR (400 MHz, CDCl₃) δ 7.44 (d, J=6.6 Hz, 1H), 7.30 (d, J=6.6 Hz, 1H), 2.94 (q, J=5.7, 2H), 1.79 (sextet, J=5.7 Hz, 2H), 0.99 (t, J=5.7 Hz, 3H); MS calcd. for [M+H]⁺ C₇H₁₀ClN₂: 157.1; found: 157.0.

3-Chloro-6-isopropylpyridazine was prepared according to the procedure described for the synthesis of 3-chloro-6-ethylpyridazine (110a). ¹H NMR (400 MHz, CD₃CN) δ 7.57 (dd, J=6.6, 19.2 Hz, 2H), 3.29 (quintet, J=5.1 Hz, 1H), 1.35 (d, J=3.6 Hz, 6H); MS calcd. for [M+H]⁺ C₇H₁₀ClN₂: 157.1; found: 157.0.

Intermediate 104d: 3-Chloro-6-t-butylpyridazine

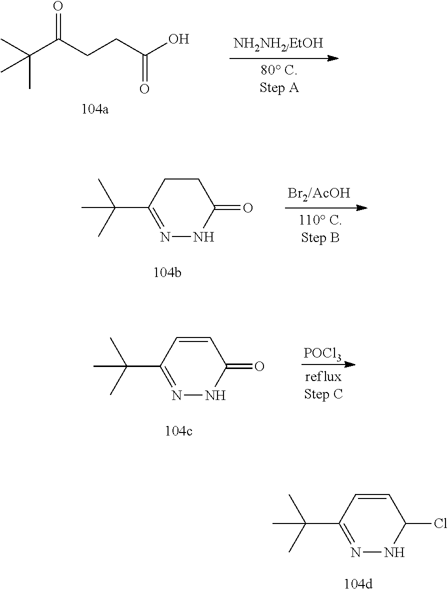

Step A A solution of 5,5-dimethyl-4-oxohexanoic acid (104a) (1.00 g, 6.32 mmo) and anhydrous hydrazine (0.24 g, 7.56 mmol) in anhydrous EtOH (10 mL) was heated to 80° C. in a sealed vial. After 4 h, the mixture was cooled to rt and the solvents were evaporated to give 6-tert-butyl-4,5-dihydropyridazin-3(2H)-one (104b) as a white solid. ¹H NMR (400 MHz, CDCl₃) δ 8.47 (s, 1H), 2.51-2.47 (m, 2H), 2.39-2.34 (m, 2H); MS calcd. for [M+H]⁺ C₈H₁₅N₂O: 155.1; found: 155.0.

Step B A solution of 104b in HOAc (10 mL) was heated to 100° C. Bromine (1.01 g, 6.3 mmol) in HOAc (1 mL) was then added dropwise in 10 min. Additional HOAc (4 mL) was then added. After the mixture was stirred at 110° C. for 1 h, the solvents were evaporated to afford 6-tert-butylpyridazin-3(2H)-one (104c) as an orange solid. MS calcd. for [M+H]⁺ C₈H₁₃N₂O: 153.1; found: 153.0.

Step C A mixture of 6-tert-butylpyridazin-3(2H)-one 104c was refluxed in POCl₃ (5 mL) for 1 h. The solvent was removed under reduced pressure and the dark residue was taken up by saturated NaHCO₃ (10 mL) and neutralized with 20% NaOH solution to afford a brown solid which was collected by filtration. The filtrate was washed with water, and then dried in vacuo to afford the product 104d as a mixture (LC-MS) of chloro and bromo compounds. MS calcd. for [M+H, Cl product]⁺ C₈H₁₂ClN₂: 171.1, found: 170.9; MS calcd. for [M+H, Br product]⁺ C₈H₁₂BrN₂: 215.0; found: 214.8.

3-Chloro-6-cyclopropylpyridazine. This compound was prepared according to the procedure described for the synthesis of 3-chloro-6-t-butylpyridazine (104d) as a mixture of chloro and bromo compounds. MS calcd. for [M+H, Cl product]+C₇H₈ClN₂: 154.0, found: 154.9; MS calcd. for [M+H, Br product]⁺ C₇H₈BrN₂: 198.0; found: 198.8.

### Example 121

### 3-Isopropyl-5-(4-(3-(2-(methylsulfonyl)-1,2,3,4-tetrahydroisoquinolin-6-yloxy)propyl)piperidin-1-yl)-1,2,4-oxadiazole

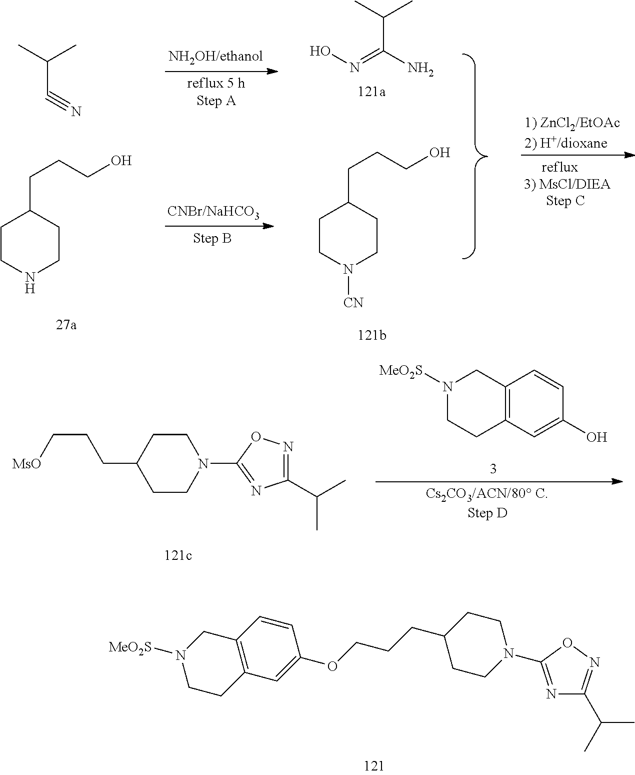

Step A A solution of isobutyronitrile (13.82 g, 0.20 mol) and hydroxylamine (50% in water, 49 mL, 0.80 mol) in 95% ethanol was refluxed overnight. The solvent was evaporated and the residual water was removed azeotropically with toluene to give N′-hydroxyisobutyrimidamide (121a) as a light yellow solid. ¹H NMR (400 MHz, CDCl₃) δ 7.00 (br s, 1H), 4.52 (s, 2H), 2.45 (quint. J=5.4 Hz, 1H), 1.16 (d, J=5.4 Hz, 6H).

Step B To a stirred a suspension of sodium bicarbonate (2.80 g, 33.3 mmol) and 4-piperidinepropanol hydrochloride salt (2.00 g, 11.1 μmol) in water (1.5 mL), CH₂Cl₂ (2 mL) was slowly added a solution of cyanogen bromide (1.42 g, 13.4 mmol) in CH₂Cl₂ (3 mL) at 0° C. (ice bath) over 1 h. The cold bath was removed and the reaction mixture was stirred overnight at rt. The mixture was diluted with CH₂Cl₂ (20 mL), basified with sodium carbonate (0.33 g), and dried over MgSO₄. The mixture was filtered and the filtrate was concentrated under reduced pressure to give 4-(3-Hydroxypropyl)piperidine-1-carbonitrile (121b) as an amber colored thick oil. ¹H NMR (400 MHz, CDCl₃) δ 3.64 (t, J=4.8 Hz, 2H), 3.42 (m, 2H), 2.99 (t, J=9.0 Hz, 2H), 1.73 (m, 2H), 1.55 (m, 2H), 1.49 (br s, 1H), 1.36-1.25 (m, 5H); MS calcd. for [M+H]⁺ C₉H₁₇N₂O: 169.1; found: 169.0.

Step C ZnCl₂ (16.7 mL, IN in ether) was slowly added to a solution of 4-(3-hydroxypropyl)piperidine-1-carbonitrile (121b) (1.87 g, 11.1 mmol) and Ar-hydroxyisobutyrimidamide (121a) (1.70 g, 16.7 mmol) in EtOAc (40 mL). A precipitate was formed during the addition. After addition, the reaction was stirred at rt for 15 min. The solvent was decanted and the solid was triturated with ether (40 mL) until a yellow suspension was obtained. The solid was collected by filtration, washed with ether (30 mL) and dried to afford the desired product as a yellow solid. MS calcd. for [M+H]⁺ C₁₄H₂₆N₃O₄S: 332.2; found: 332.0.

A suspension of above solid (422 mg) in dioxane (10 μL) and HCl in dioxane (4 M, 0.45 mL) was heated to 100° C. for 18 min. The reaction mixture was neutralized with 1N NaOH (4 mL) and concentrated. The white residue thus was obtained was dried in vacuo and used directly in the next step. MS calcd. for [M+H]⁺ C₁₃H₂₄N₃O₂: 254.2; found: 254.1.

DIEA (0.21 mL, 2.7 mmol) and MsCl (0.595 mL, 3.6 mmol) were added sequentially to the above crude (dissolved in 20 mL of CH₂Cl₂) at 0° C. and the resulting reaction mixture was stirred at rt overnight. The insoluble materials were filtered and washed with CH₂Cl₂. The organic layers were collected and concentrated to afford a yellow oil which was purified by flash chromatography (EtOAc/hexanes=20-80%) to give 3-(1-(3-isopropyl-1,2,4-oxadiazol-5-yl)piperidin-4-yl)propyl methanesulfonate (121c) as a light tan colored solid. ¹H NMR (400 MHz, CDCl₃) δ 4.23 (t, J=4.8 Hz, 2H), 4.13 (m, 2H), 3.02 (m, 2H), 3.01 (s, 3H), 2.88 (septet, J=5.1 Hz, 1H), 1.78 (m, 4H), 1.50 (m, 1H), 1.39 (m, 2H), 1.28 (d, J=5.1 Hz, 6H), 1.26 (m, 2H); MS calcd. for [M+H]⁺ C₁₄H₂₆N₃O₄S: 332.2; found: 332.1.

A suspension of 3-(1-(3-isopropyl-1,2,4-oxadiazol-5-yl)piperidin-4-yl)propyl methanesulfonate (121c) (12 mg, 0.053 mmol), 2-(methylsulfonyl)-1,2,3,4-tetrahydroisoquinolin-6-ol (3) (16 mg, 0.048 mmol) and Cs₂CO₃ (33 mg, 0.10 mmol) in anhydrous ACN (1 mL) was heated in a sealed vessel at 80° C. overnight. After cooling to rt, the reaction mixture was filtered, washed with EtOAc and concentrated. The residue was purified by flash chromatography (EtOAc/hexanes=10-50%) to afford 3-isopropyl-5-(4-(3-(2-(methylsulfonyl)-1,2,3,4-tetrahydroisoquinolin-6-yloxy)propyl)-piperidin-1-yl)-1,2,4-oxadiazole (121) as a white powder. ¹H NMR (400 MHz, CD₃CN) δ 7.04 (d, J=6.3 Hz, 1H), 6.77 (dd, J=1.8, 6.3 Hz, 1H), 6.72 (d, J=2.1 Hz, 1H), 4.31 (s, 2H), 4.03 (m, 2H), 3.94 (t, J=4.8 Hz, 2H), 3.45 (t, J=4.5 Hz, 2H), 3.05 (dt, J=2.1, 9.6 Hz, 2H), 2.91 (t, J=4.5 Hz, 2H), 2.82 (m, 1H), 2.81 (s, 3H), 1.76 (m, 4H), 1.53 (m, 1H), 1.41 (m, 2H), 1.21 (d, J=5.1 Hz, 6H), 1.25-1.15 (m, 2H); MS calcd. for [M+H]⁺ C₂₃H₃₅N₄O₄S: 463.2; found: 463.2.

### Example 123

### 6-(3-(1-(1H-Tetrazol-5-yl)piperidin-4-yl)propoxy)-2-(methylsulfonyl)-1,2,3,4-tetrahydroisoquinoline

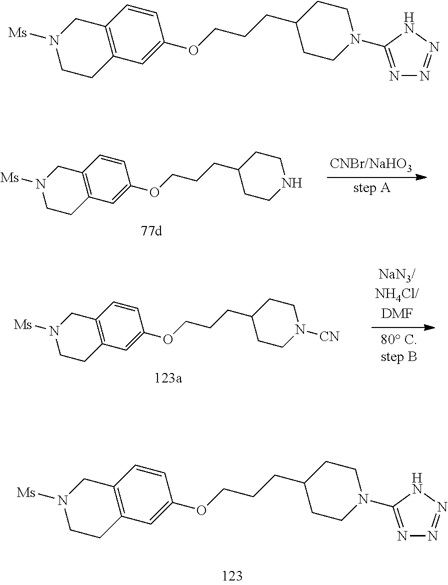

Step A Cyanogen bromide (36 mg, 0.34 mmol) was added in one portion to a stirring suspension of sodium bicarbonate (0.15 g) and 2-(methylsulfonyl)-6-(3-(piperidin-4-yl)propoxy)-1,2,3,4-tetrahydroisoquinoline (77d) (200 mg, 0.283 mmol) in water (0.1 mL) and CH₂Cl₂ (1 mL) at 0° C. The cold bath was then removed and the reaction mixture was stirred overnight at rt. The mixture was then diluted with CH₂Cl₂ (25 mL), washed with brine, dried over MgSO₄ and filtrated. Removal of solvents to afford 4-(3-(2-(methylsulfonyl)-1,2,3,4-tetrahydroisoquinolin-6-yloxy)propyl)-piperidine-1-carbonitrile (123a) as an off white solid. ¹H NMR (400 MHz, CD₃CN) δ 7.00 (d, J=6.3 Hz, 1H), 6.74 (dd, J=1.8. 6.3 Hz, 1H), 6.66 (d, J=2.1 Hz, 1H), 4.39 (s, 2H), 3.92 (t, J=4.8 Hz, 2H), 3.54 (t, J=4.5 Hz, 2H), 3.43 (m, 2H), 3.03-2.93 (m, 4H), 2.83 (s, 3H), 1.80-1.73 (m, 4H), 1.45-1.33 (m, 5H); MS calcd. for [M+H]⁺ C₁₉H₂₈N₃O₃S: 378.2; found: 378.1.

Step B A mixture of 4-(3-(2-(methylsulfonyl)-1,2,3,4-tetrahydroisoquinolin-6-yloxy)propyl)piperidine-1-carbonitrile (123a) (188 mg, 0.50 mmol), ammonium chloride (37 mg, 0.70 mmol) and NaN₃ (37 mg, 0.566 mmol) in DMF (1 mL) was heated in a sealed vial at 80° C. overnight. After cooling to rt, the reaction was quenched with water (10 mL) and the precipitate was collected by filtration. Recrystallization of the crude solid from hot MeOH and water afforded 6-(3-(1-(1H-tetrazol-5-yl)piperidin-4-yl)propoxy)-2-(methylsulfonyl)-1,2,3,4-tetrahydroisoquinoline (123) as an off white solid. ¹NMR (400 MHz, CD₃CN) δ 7.04 (d, J=6.3 Hz, 1H), 6.77-6.72 (m, 2H), 4.31 (s, 2H), 3.94 (t J=5.1 Hz, 2H), 3.86 (m, 2H), 3.45 (t, J=4.5 Hz, 2H), 2.97 (dt, J=2.1, 9.3 Hz, 2H), 2.91 (t, J=4.8 Hz, 2H), 2.81 (s, 3H), 1.81-1.74 (m, 2H), 1.50 (m, 1H), 1.42-1.35 (m, 2H), 1.25 (ddd, J=3.3, 9.3, 18.6 Hz, 2H); MS calcd. for [M+H]⁺ C₁₉H₂₉N₆O₃S: 421.2; found: 420.9.

### Examples 124

### 6-(3-(1-(2-Methyl-2H-tetrazol-5-yl)piperidin-4-yl)propoxy)-2-(methylsulfonyl)-1,2,3,4-tetrahydroisoquinoline (124)

### And

### Example 125

### 6-(3-(1-(1-Methyl-1H-tetrazol-5-yl)piperidin-4-yl)propoxy)-2-(methylsulfonyl)-1,2,3,4-tetrahydroisoquinoline (125)

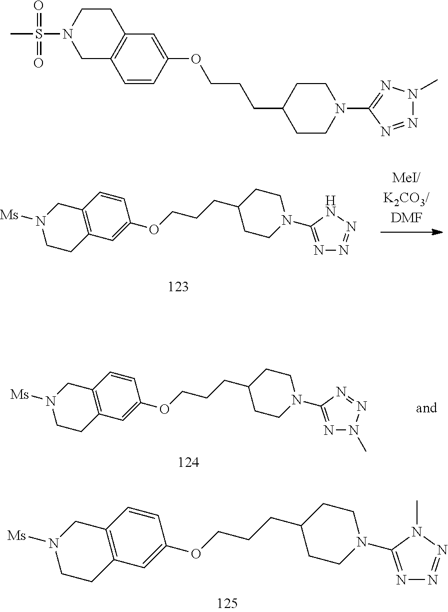

A mixture of 6-(3-(1-(1H-Tetrazol-5-yl)piperidin-4-yl)propoxy)-2-(methylsulfonyl)-1,2,3,4-tetrahydro-isoquinoline (123) (87 mg, 0.21 mmol), MeI (28 mg, 0.25 mmol) and K₂CO₃ (28 mg, 0.25 mmol) in DMF (1 mL) was stirred in sealed vial at rt overnight. The reaction mixture purified by HPLC to give 6-(3-(1-(2-methyl-2H-tetrazol-5-yl)piperidin-4-yl)propoxy)-2-(methylsulfonyl)-1,2,3,4-tetrahydroisoquinoline (124) as a major product and 6-(3-(1-(1-methyl-1H-tetrazol-5-yl)piperidin-4-yl)propoxy)-2-(methylsulfonyl)-1,2,3,4-tetrahydroisoquinoline (125) as a minor product.

124: ¹NMR (400 MHz, CD₃CN) δ 7.04 (d, J=6.3 Hz, 1H), 6.76-6.72 (m, 2H), 4.31 (s, 2H), 4.09 (s, 3H), 3.99-3.93 (m, 4H), 3.45 (t, J=4.5 Hz, 2H), 2.92-2.82 (m, 4H), 2.81 (s, 3H), 1.81-1.74 (m, 4H), 1.50 (m, 1H), 1.42-1.37 (m, 2H), 1.23 (ddd, J=3.3, 9.3, 18.3 Hz, 2H); MS calcd. for [M+H]⁺ C₂₀H₃₁N₆O₃S: 435.2; found: 434.9.
125: ¹NMR (400 MHz, CD₃CN) δ 7.04 (d, J=6.3 Hz, 1H), 6.77-6.72 (m, 2H), 4.31 (s, 2H), 3.95 (t, J=4.8 Hz, 2H), 3.81 (s, 3H), 3.58 (m, 2H), 3.45 (t, J=4.5, 2H), 2.96 (dd, J=1.8, 9.3 Hz, 2H), 2.91 (t, J=4.2 Hz, 2H), 2.81 (s, 3H); MS calcd. for [M+H]⁺ C₂₀H₃₁N₆O₃S: 435.2; found: 434.9.

### Example 126

### 6-(3-(1-(5-(1H-Tetrazol-5-yl)pyridin-2-yl)piperidin-4-yl)propoxy)-2-(methylsulfonyl)-1,2,3,4-tetrahydroisoquinoline

The title compound was prepared in a manner similar to Example 123 from Example 90.

### Example 127

### 6-(3-(1-(5-(2-Methyl-2H-tetrazol-5-yl)pyridin-2-yl)piperidin-4-yl)propoxy)-2-(methylsulfonyl)-1,2,3,4-tetrahydroisoquinoline

The title compound was prepared in a manner similar to Example 124 from Example 126.

### Example 128

### 6-(3-(1-(5-(1-Methyl-1H-tetrazol-5-yl)pyridin-2-yl)piperidin-4-yl)propoxy)-2-(methylsulfonyl)-1,2,3,4-tetrahydroisoquinoline

The title compound was prepared in a manner similar to Example 124 from Example 126.

### Example 129

### Isopropyl 4-(4-hydroxy-4-(2-(methylsulfonyl)-1,2,3,4-tetrahydroisoquinolin-6-yl)butyl)piperidine-1-carboxylate

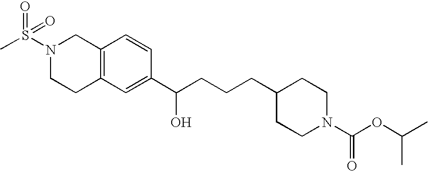

NaBH₄ (10 mg, 0.26 mmol) was added portionwise to a solution of 74 (34.1 mg, 0.07 mmol) in MeOH (0.5 mL). The solution was stirred at rt for 1 h, the solvent was evaporated and the residue was diluted with CH₂Cl₂ and H₂O. The aqueous phase was extracted with CH₂Cl₂, and the organic phase was combined, washed with brine, dried (Na₂SO₄) and concentrated. The crude product was purified on a reverse-phase HPLC to yield 129. ¹H-NMR (400 MHz, CD₃CN) δ=7.19-7.16 (m, 2H), 7.12 (d, J=8.0 Hz, 1H), 4.82 (septet, J=6.4 Hz, 1H), 4.58-4.54 (m, 1H), 4.40 (m, 2H), 4.03 (br. d, 2H), 3.50 (t, J=6.0 Hz, 2H), 3.18 (d, J=4.4 Hz, 1H), 2.96 (t, J=6.0 Hz, 2H), 2.85 (s, 3H), 2.74-2.66 (m, 2H), 1.68-1.57 (m, 4H), 1.46-1.34 (m, 2H), 1.30-1.23 (m, 3H), 1.21 (d, J=6.4 Hz, 6H), 1.05-0.94 (m 2H); MS calcd. for [M+H]⁺ C₂₃H₃₇N₂O₅S: 453.2; found: 453.2.

### Example 130

### Isopropyl 4-(4-hydroxy-4-(2-(methylsulfonyl)-1,2,3,4-tetrahydroisoquinolin-6-yl)pentyl)piperidine-1-carboxylate

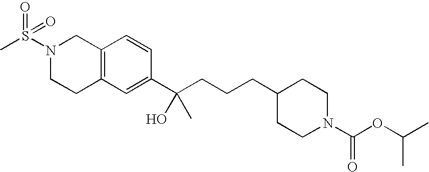

A solution of MeMgI (3 M, 0.1 mL, 0.26 mmol) in ether was added dropwise to a solution of 74 (30.9 mg, 0.07 mmol) in THF (0.5 mL) under N₂ atmosphere. The mixture was stirred at rt overnight, then diluted with MeCN and filtered. The filtrate was purified by reverse-phase HPLC to yield the title compound (Example 130). ¹H-NMR (400 MHz, CD₃CN) δ=7.26-7.23 (m 2H), 7.10 (d, J=8.4 Hz, 1H), 4.81 (septet, J=6.4 Hz, 1H), 4.38 (s, 2H), 4.04-3.96 (br. t, 2H), 3.48 (t, J=6.0 Hz, 2H), 2.96 (t, J=6.0 Hz, 2H), 2.85 (s, 3H), 2.73-2.60 (m, 2H), 1.77-1.63 (m, 2H), 1.58-1.55 (br.d, J=12.8 Hz, 2H), 1.44 (s, 3H), 1.38-1.25 (m, 2H), 1.19 (d, J=6.4 Hz, 6H), 1.17-1.12 (m 2H), 1.08-1.00 (m, 1H), 0.98-0.87 (m, 2H); MS calcd. for [M+H]⁺ C₂₄H₃₉N₂O₅S: 467.2; found: 467.2.

### Example 131

### Isopropyl 4-(4-(dimethylamino)-4-(2-(methylsulfonyl)-1,2,3,4-tetrahydroisoquinolin-6-yl)butyl)piperidine-1-carboxylate

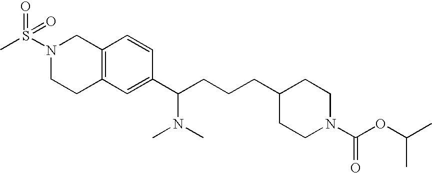

Dimethylamine hydrochloride (20 mg), followed by NaBH₃CN (10 mg) were added to a solution of 74 (21 mg, 0.05 mmol) in MeOH (0.5 mL). The resulting mixture was stirred at 80° C. overnight, diluted with MeCN and filtered. The filtrate was purified by reverse-phase HPLC to yield the title compound (Example 131). ¹H-NMR (400 MHz, CD₃CN) δ=7.40 (s, 1H) 7.38 (d, J=8.0 Hz, 1H), 7.24 (d, J=8.0 Hz, 1H), 4.70 (septet, J=6.4 Hz, 1H), 4.39 (s, 2H), 4.26-4.22 (m, 1H), 3.93-3.90 (br. d, 2H), 3.46 (t, J=6.0 Hz, 2H), 2.94 (t, J=6.0 Hz, 2H), 2.83 (s, 3H), 2.76 (br. s, 3H), 2.61 (br. s, 3H), 2.57-2.47 (m, 2H), 2.23-2.11 (m, 2H), 1.48-1.42 (m, 2H), 1.30-1.22 (m, 2H), 1.19-1.15 (m, 2H), 1.08 (d, J=6.4 Hz, 6H), 1.04-0.97 (m 1H), 0.89-0.76 (m, 2H); MS calcd. for [M+H]⁺ C₂₅H₄₂N₃O₄S: 480.3; found: 480.2.

### Example 132

### Isopropyl 4-(4-formamido-4-(2-(methylsulfonyl)-1,2,3,4-tetrahydroisoquinolin-6-yl)butyl)piperidine-1-carboxylate

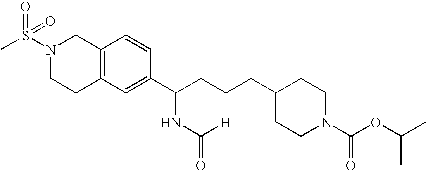

A solution of 74 (21 mg, 0.05 mmol) and ammonium formate (88 mg) in DMA (0.2 mL) was heated at 140° C. for 72 h. After cooling to rt, the mixture was diluted with MeCN and filtered. The filtrate was purified by reverse-phase HPLC to yield the title compound (Example 132). ¹H-NMR (400 MHz, CD₃CN) δ=7.98 (s, 1H) 7.05-6.99 (m, 3H), 6.73 (d, J=8.0 Hz, 0.8H), 6.65 (d, J=8.0 Hz, 0.2H), 4.73-4.61 (m, 2H), 4.28 (s, 2H), 3.95-3.87 (m, 2H), 3.38 (t, J=6.0 Hz, 2H), 2.84 (t, J=6.0 Hz, 2H), 2.73 (s, 3H), 2.64-2.55 (m, 2H), 1.63-1.48 (m, 4H), 1.30-1.24 (m, 2H), 1.18-1.13 (m, 3H), 1.09 (d, J=6.4 Hz, 6H), 0.93-0.83 (m, 2H); MS calcd. for [M+H]⁺ C₂₄H₃₈N₃O₅S: 480.2; found: 480.2.

### Example 133

### Isopropyl 4-(4-amino-4-(2-(methylsulfonyl)-1,2,3,4-tetrahydroisoquinolin-6-yl)butyl)piperidine-1-carboxylate

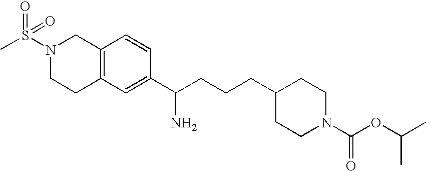

Concentrated HCl (0.15 mL) was added to a solution of 132 (7 mg, 0.01 mmol) in EtOH (0.2 mL) and the mixture was heated to 80° C. for 3 h. After cooling to rt, the mixture was diluted with MeCN and filtered. The filtrate was purified by reverse-phase HPLC to yield the title compound (Example 133). ¹H-NMR (400 MHz, CD₃CN) δ=7.68 (br. s, 3H), 7.18 (m, 2H), 7.11 (d, J=8.0 Hz, 1H), 4.70 (septet, J=6.4 Hz, 1H), 4.32 (m, 2H), 4.15-4.11 (m, 1H), 3.90 (br. d, J=12.4 Hz, 2H), 3.40 (t, J=6.0 Hz, 2H), 2.86 (t, J=6.0 Hz, 2H), 2.75 (s, 3H), 2.60-2.50 (m, 2H), 1.49-1.45 (m, 2H), 1.28-1.02 (m, 7H), 1.09 (d, J=6.4 Hz, 6H), 0.91-0.78 (m 2H); MS calcd. for [M+H]⁺ C₂₃H₃₈N₃O₄S: 451.2; found: 451.2.

### Example 134

### Isopropyl 4-(6-methoxy-4-(2-(methylsulfonyl)-1,2,3,4-tetrahydroisoquinolin-6-yl)-6-oxohexyl)piperidine-1-carboxylate

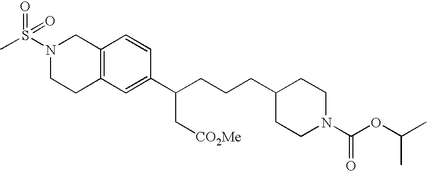

Trimethylphosphonoacetate (0.1 mL, 0.69 mmol) was added dropwise to a suspension of NaH (23 mg, 0.57 mmol) in dry THF (0.5 mL) at 0° C. under N₂ atmosphere. After stirring at rt for 30 minutes, a solution of compound 74 (50 mg, 0.11 mmol) in dry THF (0.3 mL) was added dropwise to the reaction mixture and the resulting solution was stirred overnight at rt. The mixture was then diluted with MeCN and filtered. The filtrate was purified by reverse-phase HPLC. The resulting compound was dissolved in a 1:1 mixture of EtOH/EtOAc (5 mL total), and hydrogenated under full hydrogen mode at 55° C. (H-cube, Thales nanotechnologies). Concentration followed by flash chromatography (EtOAc/hexane) yielded the title compound (Example 134). ¹H-NMR (400 MHz, CD₃CN) δ=7.10-7.07 (m, 2H), 7.05 (s, 1H), 4.81 (septet, J=6.4 Hz, 1H), 4.38 (s, 2H), 4.00 (br. d, J=12.8 Hz, 2H), 3.54 (s, 3H), 3.49 (t, J=6.0 Hz, 2H), 3.06-2.98 (m, 1H), 2.94 (t, J=6.0 Hz, 2H), 2.85 (s, 3H), 2.74-2.61 (m, 2H), 2.65 (dd, J=15.2, 6.4 Hz, 1H), 2.54 (dd, J=15.2, 8.8 Hz, 1H), 1.64-1.54 (m, 4H), 1.37-1.30 (m, 2H), 1.20 (d, J=6.4 Hz, 6H), 1.18-1.12 (m 2H), 1.01-0.88 (m, 3H); MS calcd. for [M+H]⁺ C₂₆H₄₁N₂O₆S: 509.2; found: 509.2.

### Example 135

### Isopropyl 4-(6-hydroxy-4-(2-(methylsulfonyl)-1,2,3,4-tetrahydroisoquinolin-6-yl)hexyl)piperidine-1-carboxylate

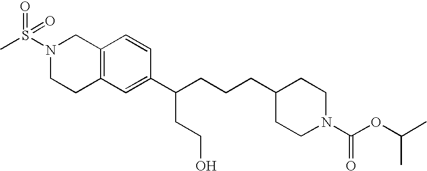

A solution of LiAlH₄ (1 M, 0.2 mL) in THF was added dropwise to a solution of 134 (4 mg, 0.01 mmol) in dry THF (0.2 mL). After addition, the mixture was stirred at rt for 2.5 h, then quenched with cold H₂O. The mixture was then diluted with MeCN and filtered. The filtrate was purified by reverse-phase HPLC to yield the title compound (Example 135). ¹H-NMR (400 MHz, CD₃CN) δ=7.00 (d, J=8.0 Hz, 1H), 6.96-6.94 (m, 2H), 4.70 (septet, J=6.4 Hz, 1H), 4.29 (s, 2H), 3.94-3.90 (m, 2H), 3.41 (t, J=6.0 Hz, 2H), 3.34-3.21 (m, 3H), 3.16-3.11 (m, 1H), 2.85 (t, J=6.0 Hz, 2H), 2.79 (s, 3H), 2.67-2.59 (m, 2H), 1.80-1.72 (m, 1H), 1.67-1.58 (m, 1H), 1.54-1.43 (m, 4H), 1.25-1.15 (m, 3H), 1.12-1.10 (m, 2H), 1.08 (d, J=6.4 Hz, 6H), 0.91-0.76 (m, 2H); MS calcd. for [M+H]⁺ C₂₅H₄₁N₂O₅S: 481.2; found: 481.2.

### Example 136

### 6-(1-(Isopropoxycarbonyl)piperidin-4-yl)-3-(2-(methylsulfonyl)-1,2,3,4-tetrahydroisoquinolin-6-yl)hexanoic acid

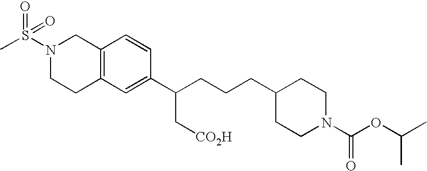

A mixture of 134 (4 mg, 0.01 mmol), 1N NaOH (0.5 mL) and MeOH (0.2 mL) was heated to 80° C. for 30 minutes. After cooling at rt and removal of the solvents, the mixture was acidified with 1M HCl, and then extracted with Et₂O. The organic layer was combined, dried (Na₂SO₄) and concentrated to yield the title compound (Example 136). ¹H-NMR (400 MHz, CD₃CN) δ=7.00-6.96 (m, 3H), 4.68 (septet, J=6.4 Hz, 1H), 4.26 (s, 2H), 3.91-3.88 (br. d, 2H), 3.38 (t, J=6.0 Hz, 2H), 2.96-2.88 (m, 1H), 2.82 (t, J=6.0 Hz, 2H), 2.76 (s, 3H), 2.62-2.48 (m, 2H), 2.52 (dd, J=15.6, 7.2 Hz, 1H), 2.42 (dd, J=15.6, 8.0 Hz, 1H), 1.61-1.44 (m, 4H), 1.26-1.13 (m, 3H), 1.12-1.07 (m, 2H), 1.05 (d, J=6.4 Hz, 6H), 0.88-0.74 (m, 2H); MS calcd. for [M+H]⁺ C₂₅H₃₉N₂O₆S: 495.2; found: 495.2.

### Example 137

### Isopropyl 4-(4-methoxy-4-(2-(methylsulfonyl)-1,2,3,4-tetrahydroisoquinolin-6-yl)butyl)piperidine-1-carboxylate

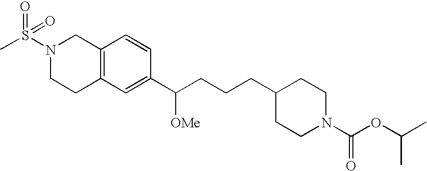

A solution of Example 129 (8 mg, 0.02 mmol) in dioxane (0.5 mL) was added dropwise into a suspension of NaH (15 mg, 0.37 mmol) in dioxane (0.2 mL) at 0° C. under N₂. The resulting mixture was stirred for additional 10 minutes at 0° C. and MeI (0.05 mL) was added. The mixture was then allowed to warm to rt and stirred overnight. The mixture was diluted with H₂O and MeCN and filtered. The filtrate was purified by reverse-phase HPLC to yield the title compound (Example 137). ¹H-NMR (400 MHz, CD₃CN) δ=7.06-7.03 (m, 2H), 7.01 (s, 1H), 4.70 (septet, J=6.4 Hz, 1H), 4.30 (s, 2H), 3.97 (dd, J=7.2, 6.0 Hz, 1H), 3.91 (br. s, 2H), 3.43-3.34 (m, 2H), 3.02 (s, 3H), 2.86 (t, J=6.0 Hz, 2H), 2.75 (s, 3H), 2.65-2.50 (m, 2H), 1.65-1.57 (m, 1H), 1.55-1.48 (m, 2H), 1.48-1.40 (m, 1H), 1.32-1.21 (m, 2H), 1.15-1.10 (m, 3H), 1.09 (d, J=6.4 Hz, 6H), 0.91-0.80 (m 2H); MS calcd. for [M+H]⁺ C₂₄H₃₉N₂O₅S: 467.2; found: 467.2.

### Example 138

### Isopropyl 4-(4-fluoro-4-(2-(methylsulfonyl)-1,2,3,4-tetrahydroisoquinolin-6-yl)butyl)piperidine-1-carboxylate

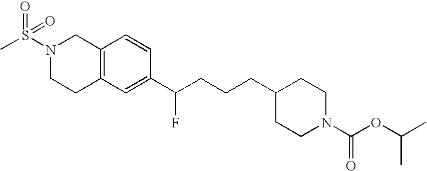

DAST (0.2 mL) was added to Example 129 (8 mg, 0.02 mmol) and the mixture was stirred at rt for 1 h. CH₂Cl₂ and sat. aqueous Na₂CO₃ were then added. The resulting mixture was extracted with CH₂Cl₂. The organic layers were combined, dried (Na₂SO₄), concentrated and the resulting residue was purified on a reverse-phase HPLC to yield the title compound (Example 138). ¹H-NMR (400 MHz, CD₃CN) δ=7.11-7.06 (m, 3H), 5.33 (ddd, J=48.0, 8.0, 5.2 Hz, 1H), 4.71 (septet, J=6.4 Hz, 1H), 4.31 (s, 2H), 3.92 (br. d, J=12.8 Hz, 2H), 3.40 (t, J=6.0 Hz, 2H), 2.87 (t, J=6.0 Hz, 2H), 2.74 (s, 3H), 2.65-2.54 (m, 2H), 1.72-1.61 (m, 1H), 1.57-1.50 (m, 2H), 1.41-1.22 (m, 3H), 1.20-1.13 (m, 3H), 1.10 (d, J=6.4 Hz, 6H), 0.94-0.84 (m 2H); ¹⁹F-NMR (376 MHz, CD₃CN) δ=−172.817; MS calcd. for [M+H]⁺ C₂₃H₃₆FN₂O₄S: 455.2; found: 455.2.

### Example 139

### tert-Butyl 4-(4-(2-(methylsulfonyl)-1,2,3,4-tetrahydroisoquinolin-6-yl)-4-oxobutyl)piperidine-1-carboxylate

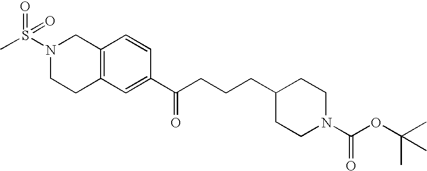

Following the procedure for Example 74, compound 74a was reacted with tert-butyl 4-(3-bromopropyl)piperidine-1-carboxylate to give the title compound (Example 139). ¹H-NMR (400 MHz, CDCl₃) δ=7.81-7.78 (m 2H), 7.21 (d, J=8.0 Hz, 1H), 4.53 (s, 2H), 4.10 (br. s, 2H), 3.61 (t, J=6.0 Hz, 2H), 3.07 (t, J=6.0 Hz, 2H), 2.96 (t, J=7.2 Hz, 2H), 2.89 (s, 3H), 2.74-2.63 (m, 2H), 1.81-1.74 (m, 2H), 1.72-1.67 (m, 2H), 1.47 (s, 9H), 1.44-1.39 (m, 1H), 1.36-1.31 (m, 2H), 1.17-1.06 (m 2H); MS calcd. for [M+H]⁺ C₂₄H₃₇N₂O₅S: 465.2; found: 465.2.

### Example 140

### 4-(1-(5-Ethylpyrimidin-2-yl)piperidin-4-yl)-1-(2-(methylsulfonyl)-1,2,3,4-tetrahydroisoquinolin-6-yl)butan-1-one

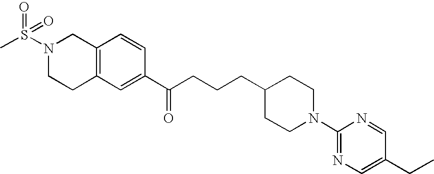

Intermediate 140a: 1-(2-(Methylsulfonyl)-1,2,3,4-tetrahydroisoquinolin-6-yl)-4-(piperidin-4-yl)butan-11-one hydrochloride

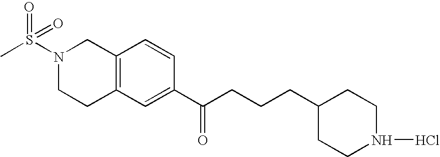

140a was synthesized from 139 (600 mg, 1.29 mmol) according to the procedure described for the synthesis of 51a; MS calcd. for [M+H]⁺ C₁₉H₂₉N₂O₃S: 365.2; found: 365.2.

140 was synthesized from 140a (165 mg, 0.45 mmol) following the same procedure described for the preparation of 27a. The mixture was then filtered through a syringe filter using MeCN as solvent and purified by flash column chromatography on silica gel (EtOAc/Hexane=0-80%) to yield 140. ¹H-NMR (400 MHz, CDCl₃) δ=8.17 (s, 2H), 7.79 (d, J=8.0 Hz, 1H), 7.78 (s, 1H), 7.20 (d, J=8.0 Hz, 1H), 4.69 (d, J=13.2 Hz, 2H), 4.52 (s, 2H), 3.61 (t, J=6.0 Hz, 2H), 3.07 (t, J=4.8 Hz, 2H), 2.97 (t, J=7.2 Hz, 2H), 2.88 (s, 3H), 2.85 (td, J=2.4, 12.4 Hz, 2H), 2.46 (q, J=7.6 Hz, 2H), 1.80 (m, 4H), 1.57 (m, 1H), 1.36 (m, 2H), 1.23 (m, 4H); MS calcd. for [M+H]⁺ C₂₅H₃₅N₄O₃S: 471.2; found: 471.2.

### Example 141

### 1-Methylcyclopropyl 4-(4-(2-(methylsulfonyl)-1,2,3,4-tetrahydroisoquinolin-6-yl)-4-oxobutyl)piperidine-1-carboxylate

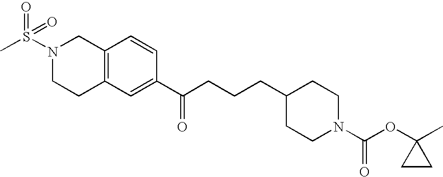

141 was synthesized from 140a (165 mg, 0.45 mmol) according to the procedure described for the preparation of Example 60. The mixture was purified by flash column chromatography on silica gel (EtOAc/Hexane=0-60%) to yield 141. ¹H-NMR (400 MHz, CDCl₃) δ=7.78 (m, 2H), 7.20 (d, J=8.0 Hz, 1H), 4.52 (s, 2H), 3.60 (t, J=6.0 Hz, 2H), 3.07 (t, J=6.0 Hz, 2H), 2.95 (t, J=7.2 Hz, 2H), 2.88 (s, 3H), 2.71 (m, 2H), 1.75 (m, 3H), 1.67 (s, 2H), 1.55 (s, 3H), 1.43 (m, 1H), 1.32 (m, 2H), 1.25 (d, J=6.4 Hz, 1H), 1.10 (m, 2H), 0.87 (t, J=6.4 Hz, 2H), 0.63 (t, J=6.4 Hz, 2H); MS calcd. for [M+H]⁺ C₂₄H₃₅N₂O₅S: 463.2; found: 463.2.

### Example 146

### 6-(3-(1-(5-Ethylpyrimidin-2-yl)piperidin-4-yl)propoxy)-5,7-difluoro-2-(methylsulfonyl)-1,2,3,4-tetrahydroisoquinoline

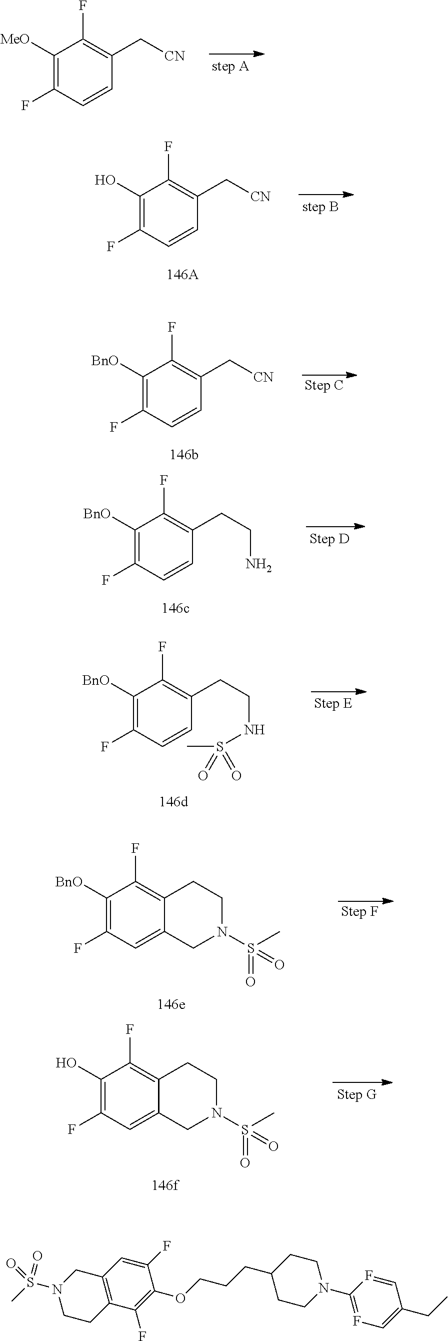

Step A 2-(2,4-Difluoro-3-hydroxyphenyl)acetonitrile (146a) A solution of BBr₃ in CH₂Cl₂ (1.0 M, 49 mL, 49 mmol) was added dropwise to a solution of 2,4-difluoro-3-methoxyphenylacetonitrile (3 g, 16.4 mmol) in CH₂Cl₂ (16 mL) at −78° C. The mixture was allowed to warm up to rt and stirred overnight. The solvent was removed and the crude was added to ice cold water, neutralized with saturated aqueous Na₂CO₃ and extracted with EtOAc. The organics were combined, washed with brine, dried (MgSO₄) and filtered. Removal of solvent under reduced pressure provided desired product 146a. MS calcd. for [M+H]⁺ C₈H₅F₂NO: 170.1; found: 170.1

Step B 2-(3-(Benzyloxy)-2,4-difluorophenyl)acetonitrile (146b) In a round bottom flask was added 2-(2,4-difluoro-3-hydroxyphenyl)acetonitrile (2.8 g, 16.4 mmol), benzyl bromide (3.9 mL, 32.8 mmol), potassium carbonate (4.5 g, 32.8 mmol), potassium iodide (3 g, 18.04 mmol) and acetone (20 mL). The mixture was refluxed overnight, cooled to rt, filtered and concentrated under reduced pressure. The residue was taken up with EtOAc and the organics was washed with water (3×20 mL), brine, dried (MgSO₄) and filtered. Removal of solvent under reduced pressure gave crude product as brown oil. Purification of the crude on silica gel (ethyl acetate:hexanes=1:1) afforded the desired product 146b as a yellow oil. MS calcd. for [M+H]⁺ C₁₅H, IF₂NO: 260.1; found: 260.0.

Step C 2-(3-(Benzyloxy)-2,4-difluorophenyl)ethanamine (146c). To a solution of the 146b (1 g, 3.9 mmol) in anhydrous THF (10 mL) was added a solution of BH₃ in THF (1 M, 16 mL, 16 mmol) dropwise at 0° C. (ice bath). The mixture was warmed up to rt and stirred for 1 hour, then it was refluxed for 1.5 hour, cooled back to 0° C. MeOH (5 mL) was slowly added. The solvent was removed and the resultant oily residue was dissolved in CH₂Cl₂, washed with saturated aqueous NaHCO₃, and dried (MgSO₄). Removal of solvent under reduced pressure afforded crude 138c which was used directly for the next step. MS calcd. for [M+H]⁺ C₁₅H, IF₂NO: 264.1; found: 264.1.

Step D N-(3-(Benzyloxy)-2,4-difluorophenethyl)methanesulfonamide (146d). To a solution of 146c (640 mg, 2.4 mmol) in CH₂Cl₂ (10 mL) was added Et₃N (1 mL, 7.2 mmol) followed by addition of methanesulfonyl chloride (283 uL, 3.6 mmol) at 0° C. After complexion of the reaction, water was added. The mixture was extracted with CH₂Cl₂ and washed with 1N HCl. Removal of solvent under reduced pressure provided the crude product. Purification of the crude on silica gel (EtOAc:Hexanes=1:2) yielded 146d as a colorless oil. MS calcd. for [M+H]⁺ C₁₆H₁₇F₂NO₃S: 342.1; found: 342.1.

Step E 6-(Benzyloxy)-5,7-difluoro-2-(methylsulfonyl)-1,2,3,4-tetrahydroisoquinoline (146e). To a solution of 146d (270 mg, 0.8 mmol) in dry DME (1.6 mL) was added boron trifluoroetherate (300 uL, 2.4 mmol) at rt. After stirring at rt overnight, the precipitate was collected, rinsed with ethyl acetate/hexane (1:9) and dried to afford 146e as a white solid. MS calcd. for [M+H]⁺ C₁₇H₁₇F₂NO₃S: 354.1; found: 354.1.

Step F 5,7-Difluoro-2-(methylsulfonyl)-1,2,3,4-tetrahydroisoquinolin-6-ol (146f). 6-(Benzyloxy)-5,7-difluoro-2-(methylsulfonyl)-1,2,3,4-tetrahydroisoquinoline (146e) (450 mg, 1.27 mmol) was dissolved in ethanol (20 mL) and ethyl acetate (20 mL). Pd/C (10 wt %, wet) was added and the mixture was stirred under H₂ atmosphere for 1 hour. It was filtrated through a short celite plug (rinsed with EtOAc). Removal of the solvents under reduced pressure afforded 146f. MS calcd. for [M+H]⁺ C₁₀H₁₁F₂NO₃S: 264.0; found: 263.8.

Step G 6-(3-(1-(5-Ethylpyrimidin-2-yl)piperidin-4-yl)propoxy)-5,7-difluoro-2-(methylsulfonyl)-1,2,3,4-tetrahydroisoquinoline (146). A mixture of 146f (30 mg, 0.11 mmol), 3-(1-(5-ethylpyrimidin-2-yl)piperidin-4-yl)propyl methanesulfonate (40 mg, 0.12 mmol), and Cs₂CO₃ (54 mg, 0.17 mmol) in DMF (2 mL) was heated at 80° C. overnight. The mixture was cooled to rt and filtered. The filtrate was concentrated under reduced pressure to afford the crude product. Purification of the crude on silica gel (EtOAc:Hexanes=1:1) afforded 138 as a white solid. MS calcd. for [M+H]⁺ C₂₄H₃₂F₂NO₃S: 495.2; found: 495.2.

Examples 151, 158 and 159 were prepared by analogous method from example 146.

### Example 147

### 6-(3-(1-(5-Ethylpyrimidin-2-yl)piperidin-4-yl)propoxy)-4,4-dimethyl-2-(methylsulfonyl)-1,2,3,4-tetrahydroisoquinoline

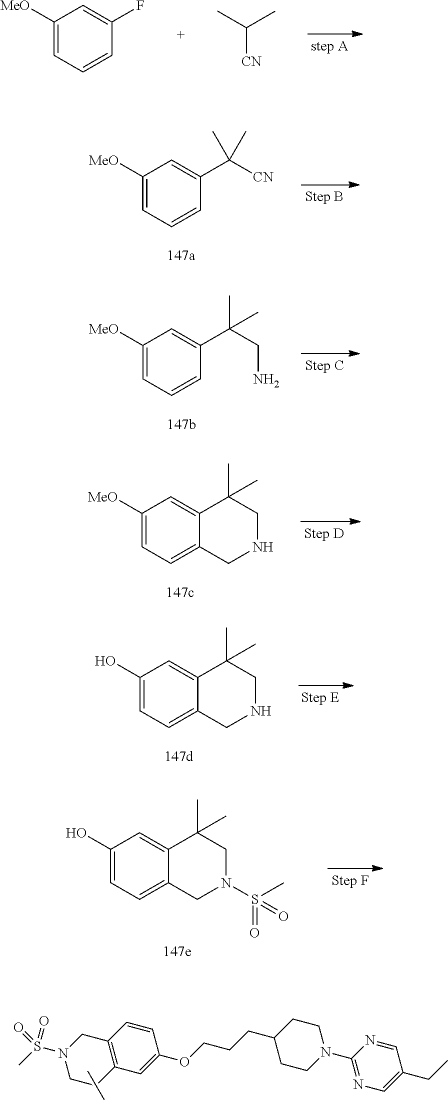

Step A 2-(3-Methoxyphenyl)-2-methylpropanenitrile (146a). A solution of KHMDS (0.5 M in THF, 120 mL) was added dropwise to a solution of 3-fluoroanisole (5 g, 40 mmol) and isobutyronitrile (14.2 mmol, 160 mmol) in toluene (50 mL) at rt. The mixture was then stirred overnight at 60° C., and then cooled to rt, carefully poured into 1 N HCl and extracted with EtOAc. The organic layers were combined, washed with water, brine, dried (MgSO₄), filtrated and solvents were removed under reduced pressure. The crude product was purified on silica gel (eluent: EtOAc/hexane) to afford 147a as an oil. MS calcd. for [M+H]⁺ C₁₀H₁₁F₂NO₃S: 176.1; found: 176.1

Step B 2-(3-Methoxyphenyl)-2-methylpropan-1-amine (147b). A solution of borane in THF (80 mL, 1 M) was added dropwise to a solution of 2-(3-methoxyphenyl)-2-methylpropanenitrile (147a) (2.8 g, 16 mmol) in anhydrous THF (10 mL) at 0° C. (ice bath). The mixture was allowed to warm up to rt, stirred for 1 hour, and cooled back to 0° C. MeOH was slowly added until gas evolution ceased. The solution was concentrated and the resulting oily residue was added to 1N HCl (60 mL). It was extracted with ethyl acetate (2×10 mL), and the aqueous layer was basified to pH 11 with 3N aqueous NaOH. The aqueous was extracted with 10% MeOH/CHCl₃ (3×20 mL). The MeOH/CHCl₃ extracts were combined, dried (MgSO₄), and filtrated. The solvents were removed to afford 147b as a pale yellow oil. MS calcd. for [M+H]⁺ C₁₁H₁₇NO: 180.1; found: 180.1.

Step C 6-Methoxy-4,4-dimethyl-1,2,3,4-tetrahydroisoquinoline (147c). Formic acid (1.4 mL) was slowly added to neat 147b (500 mg, 2.8 mmol) at 0° C. The solution was stirred at 0° C. for 5 minutes, paraformaldehyde (84 mg) was added and the resulting mixture was heated at 50° C. for 8 hours. The mixture was cooled to rt, diluted with water and poured into CH₂Cl₂ (20 mL). The organic layers were separated; the aqueous layer was basified with 50% NaOH solution and extracted with 10% MeOH/CHCl₃. The MeOH/CHCl₃ extracts were combined, dried (MgSO₄) and filtrated. Solvents were removed to afford 147c as colorless oil. MS calcd. for [M+H]⁺ C₁₂H₁₇NO: 192.1; found: 192.1.

Step D 4,4-Dimethyl-1,2,3,4-tetrahydroisoquinolin-6-ol (147d). A solution of 48% aq HBr (11.2 mL) was added to 6-methoxy-4,4-dimethyl-1,2,3,4-tetrahydroisoquinoline (147c) (560 mg, 2.8 mmol) at rt. The reaction vessel was sealed and the mixture was heated at 120° C. for 2.5 hours. The mixture was cooled to rt, diluted with water and the aq HBr was removed under reduced pressure. The crude material was triturated with EtOH and Et₂O. It was filtered and solid was collected and air dried to afford 147d. MS calcd. for [M+H]⁺ C₁₁H₁₅NO: 178.1; found: 178.1.

Step E 4,4-Dimethyl-2-(methylsulfonyl)-1,2,3,4-tetrahydroisoquinolin-6-ol (147e). Et₃N (889 uL, 6.4 mmol) was added dropwise at 0° C. to a solution of 147d (300 mg, 1.16 mmol) in CH₂Cl₂ followed by the addition of methanesulfonyl chloride (200 uL, 2.6 mmol). After complexion of the reaction, water was added and the mixture was extracted with CH₂Cl₂. The organics were combined, washed with 1N HCl, aqueous saturated NaHCO₃, dried (MgSO₄), and filtrated. Removal of solvents afforded di-mesylated product. The di-mesylate was dissolved in a solution of MeOH/10% aq NaOH (2:1) and heated at 80° C. until the mixture becomes homogeneous. The mixture was cooled to rt, acidified, concentrated to 10-20 mL, then extracted with CH₂Cl₂/MeOH (95:5). The organic layers were combined, dried (MgSO₄) and filtered. Solvents were removed to afford 147e as a white solid. MS calcd. for [M+H]⁺ C₁₂H₁₇NO₃S: 256.1; found: 256.1.

Step F 6-(3-(1-(5-Ethylpyrimidin-2-yl)piperidin-4-yl)propoxy)-4,4-dimethyl-2-(methylsulfonyl)-1,2,3,4-tetrahydroisoquinoline (147). Example 147 was synthesized according to the procedure described for example 146 (Step G) from the corresponding phenol 147e and 4,4-dimethyl-2-(methylsulfonyl)-1,2,3,4-tetrahydroisoquinolin-6-ol and 3-(1-(5-ethylpyrimidin-2-yl)piperidin-4-yl)propyl methanesulfonate. MS calcd. for [M+H]⁺ C₂₆H₃₈N₄O₃S: 487.3; found: 487.3.

Example 145 was prepared by analogous method from example 147.

### Example 149

### 6-(3-(1-(5-Ethylpyrimidin-2-yl)piperidin-4-yl)propoxy)-7-fluoro-2-(methylsulfonyl)-1,2,3,4-tetrahydroisoquinoline

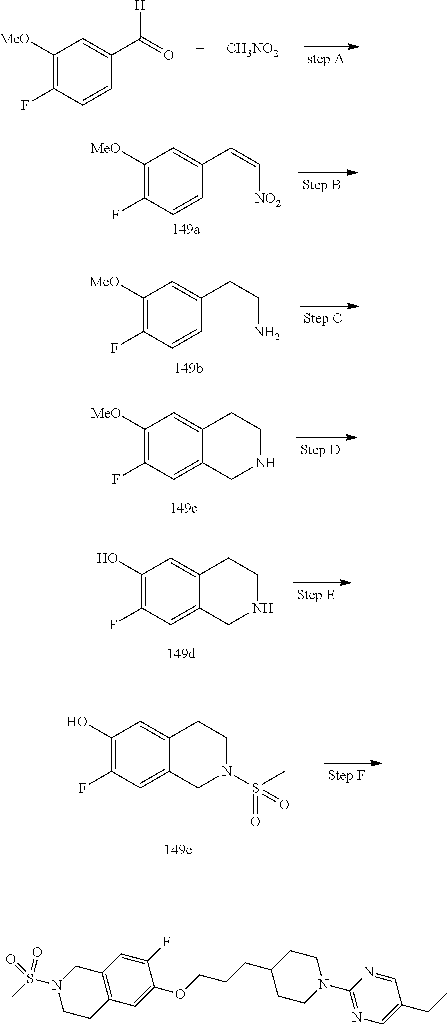

Step A 1-Fluoro-2-methoxy-4-(2-nitrovinyl)benzene (149a). A solution of aqueous NaOH (1.15 g in 4 mL of water) was added dropwise to a mixture of 4-fluoro-3-methoxybenzaldehyde (3.85 g, 25 mmol) and nitromethane (1.35 mL, 25 mmol) in MeOH (25 mL) at −10° C. After complexion of the addition, the mixture was stored in a fridge at 0° C. overnight. The resulting mixture was then carefully poured into aqueous HCl (10%) and yellow precipitates were obtained. The heterogeneous mixture was then cooled for 30 min. in an ice water bath, and filtered. Solids were collected, washed with water, and dried in a vacuum oven to afford 149a. MS calcd. for [M+H]⁺ C₉H₈FNO₃: 198.1; found: 198.1.

Step B 2-(4-Fluoro-3-methoxyphenyl)ethanamine (149b). To a suspension of AlLiH₄ (1.15 g, 30.4 mmol) in THF (30 mL) was added dropwise a solution of 149a (1.5 g, 7.6 mmol) in THF (100 mL) at 0° C. The mixture was stirred at 0° C. for 15 minutes, warmed to rt for 15 min and then refluxed for 2 hours. It was then cooled to 0° C. (ice bath), and Na₂SO₄.10H₂O (3.0 g) was slowly added. The resulting slurry was vigorously stirred at rt overnight, and filtrated through a pad of Celite, which was washed with additional THF. The filtrate was combined and solvents were removed. Aqueous HCl (1 N, 25 mL) was added to the residue. It was extracted with CH₂Cl₂, and the aqueous was basified to pH 11, and then extracted CHCl₃ (3×20 mL). The CHCl₃ extracts were combined, dried (MgSO₄), and filtrated. Solvents were removed to afford 149b as yellow oil. MS calcd. for [M+H]⁺ C₉H₁₂FNO: 170.1; found: 170.0.

Step C 7-Fluoro-6-methoxy-1,2,3,4-tetrahydroisoquinoline. Formic acid (2.1 mL) was slowly added to amine (149b) (721 mg, 4.3 mmol) at 0° C. After 5 minutes, paraformaldehyde (128 mg) was added and the resulting mixture was heated at 50° C. for 8 hours. The mixture was cooled to 0° C., diluted with water and extracted with CH₂Cl₂ (20 mL). The organic layers were separated and the aqueous was basified with 50% aqueous NaOH solution and then extracted with 10% MeOH/CHCl₃ (4×40 mL). The MeOH/CHCl₃ extracts were combined, dried (MgSO₄), and filtrated. Solvents were removed to afford 149c as light orange oil. MS calcd. for [M+H]⁺ C₁₀H₁₂FNO: 182.1; found: 182.1.

Step D 7-Fluoro-1,2,3,4-tetrahydroisoquinolin-6-ol (149d). A solution of HBr (48% aqueous, 16 mL, 4 mL/mmol) was added to 6-methoxy-4,4-dimethyl-1,2,3,4-tetrahydroisoquinoline (149c) (700 mg, 3.7 mmol). The reaction vessel was sealed and the mixture was heated at 120° C. for 2.5 hours. The mixture was cooled to 0° C., diluted with water and HBr removed under reduced pressure. The crude material was triturated with EtOAc; solids were collected and dried to afford 149d (HBr salt). MS calcd. for [M+H]⁺ C₉H₁₀FNO: 168.1; found: 168.1.

Step E Synthesis of 7-fluoro-2-(methylsulfonyl)-1,2,3,4-tetrahydroisoquinolin-6-ol (149e). To a mixture of 149d (750 mg, 3.0) and Et₃N (2.3 mL, 16.5 mmol) in CH₂Cl₂ was added methanesulfonyl chloride (513 uL, 6.6 mmol) dropwise at 0° C. After complexion of the reaction, the solvents were removed and the residue was triturated with EtOAc. The brown solids (dimesylate intermediate) were collected, the filtrate was concentrated and then purified on silica gel (EtOAc:hexane=1:1) to obtain additional desired intermediate (dimesylate). The dimesylate intermediate (150 mg, 0.46 mmol) was suspended in methanol (7 mL) and 10% aq sodium hydroxide (3 mL) and stirred at 80° C. for 2 hours. The mixture was neutralized with 1 N HCl; the solid was collected and air dried to afford the intermediate 149e. MS calcd. for [M+H]⁺ C₁₀H₁₂FNO₃S: 246.1; found: 246.1.

Step F 6-(3-(1-(5-Ethylpyrimidin-2-yl)piperidin-4-yl)propoxy)-7-fluoro-2-(methylsulfonyl)-1,2,3,4-tetrahydroisoquinoline (Example 149). Example 149 was synthesized from 149e and 3-(1-(5-ethylpyrimidin-2-yl)piperidin-4-yl)propyl methanesulfonate according to the procedure described for the synthesis of 146. MS calcd. for [M+H]⁺ C₂₄H₃₃FN₄O₃S: 477.2; found: 477.8.

### Example 150

### 6-(3-(1-(5-((2-Methoxyethoxy)methyl)pyrimidin-2-yl)piperidin-4-yl)propoxy)-2-(methylsulfonyl)-1,2,3,4-tetrahydroisoquinoline

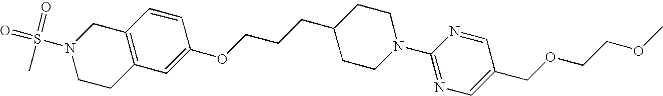

Step A Methyl 2-(4-(3-(2-(methylsulfonyl)-1,2,3,4-tetrahydroisoquinolin-6-yloxy)propyl)piperidin-1-yl)pyrimidine-5-carboxylate. To a microwave reaction vessel was added methyl 2-chloropyrimidine-5-carboxylate (138 mg), 2-(methylsulfonyl)-6-(3-(piperidin-4-yl)propoxy)-1,2,3,4-tetrahydroisoquinoline (175 mg), DMF (3 μL) and Cs₂CO₃ (350 mg). The mixture was irradiated in microwave reactor at 160° C. for 20 min. It was cooled and EtOAc (20 mL) was added. The mixture was washed with brine (10 mL), dried over Na₂SO₄ and filtered. Removal of solvents under reduced pressure and purification of the crude on silica gel (EtOAc:Hexanes=1:1) gave desired product. MS calcd. for [M+H]⁺ C₂₆H₃₅N₂O₅S: 487.2; found: 487.2.

Step B (2-(4-(3-(2-(Methylsulfonyl)-1,2,3,4-tetrahydroisoquinolin-6-yloxy)propyl)piperidin-1-yl)pyrimidin-5-yl)methanol. A solution of LiBH4 in THF (2 M, 0.2 mL) was added slowly to a solution of methyl 2-(4-(3-(2-(methylsulfonyl)-1,2,3,4-tetrahydroisoquinolin-6-yloxy)propyl)piperidin-1-yl)pyrimidine-5-carboxylate (50 mg) in dry THF (10 mL) at 0° C. After complexion of the reaction, the mixture was quenched with water and extracted with CHCl₃. The organic layers were combined, dried (MgSO₄), filtrated and concentrated to afford the crude. The crude was purified by silica gel column chromatography (EtOAc:Hexanes=3:1) to provide the desired product. MS calcd. for [M+H]⁺ C₂₃H₃₃N₄O₄S: 461.2; found: 461.2.

Step C 6-(3-(1-(5-((2-Methoxyethoxy)methyl)pyrimidin-2-yl)piperidin-4-yl)propoxy)-2-(methylsulfonyl)-1,2,3,4-tetrahydroisoquinoline. NaH (40 mg) was added to a solution of (2-(4-(3-(2-(methylsulfonyl)-1,2,3,4-tetrahydroisoquinolin-6-yloxy)propyl)piperidin-1-yl)pyrimidin-5-yl)methanol (30 mg) in dry DMF (5 mL) at 0° C. The resulting mixture was stirred at 0° C. for 1 h, then bromoethyl methyl ether (0.1 mL) was slowly added and the solution was allowed to warm up to rt and stirred overnight. The reaction was quenched with ice cold water at 0° C., and then extracted with EtOAc. The organic layers were combined, washed with water, brine, dried over Na₂SO₄ and filtered. Solvents were removed under reduced pressure to give crude product. Purification of the crude on silica gel column (EtOAc:Hexanes=3:1) gave the desired product 150. MS calcd. for [M+H]⁺ C₂₆H₃₉N₄O₅S: 519.2; found: 519.2.

### Example 163

### 5-(4-Bromophenethyl)-3-(2-(methylsulfonyl)-1,2,3,4-tetrahydroisoquinolin-6-yl)-1,2,4-oxadiazole

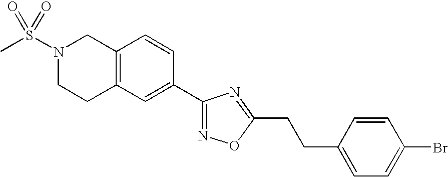

To a round bottom flask containing N-hydroxy-2-(methylsulfonyl)-1,2,3,4-tetrahydroisoquinoline-6-carboximidamide (2.7 g) in THF (30 mL) was added sequentially 3-(4-bromophenyl)propanoic acid (2.5 g) and HATU (7.1 g). After it became a clear solution, the mixture was heated at 60° C. overnight, cooled to rt, then diluted with 100 mL of EtOAc, washed with brine, dried over Na₂SO₄ and filtered. Solvents were removed under reduced pressure to give crude product. Purification of the crude (EtOAc:Hexanes=1:3) on silica gel afforded desired product 163. MS calcd. for [M+H]⁺ C₂₀H₂₁BrN₃O₅S: 462.0; found: 462.0.

### Example 165

### 5-(4-(5-Methylpyridin-2-yl)phenethyl)-3-(2-(methylsulfonyl)-1,2,3,4-tetrahydroisoquinolin-6-yl)-1,2,4-oxadiazole

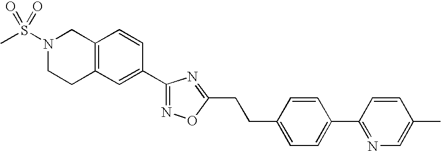

Step A 3-(2-(Methylsulfonyl)-1,2,3,4-tetrahydroisoquinolin-6-yl)-5-(4-(4,4,5,5-tetramethyl-1,3,2-dioxaborolan-2-yl)phenethyl)-1,2,4-oxadiazole. To a round bottom flask was added 5-(4-bromophenethyl)-3-(2-(methylsulfonyl)-1,2,3,4-tetrahydroisoquinolin-6-yl)-1,2,4-oxadiazole (340 mg), 4,4,4′,4′,5,5,5′,5′-octamethyl-2,2′-bi(1,3,2-dioxaborolane) (300 mg), (dppf)₂PdCl₂ (35 mg), KOAc (360 mg) and DMSO (5 mL). The mixture was degassed with nitrogen and heated at 80° C. for 5 hrs. After cooling to rt, EtOAc (50 μL) was added and the mixture was washed with water (3×25 mL), brine (2×20 mL), dried over Na₂SO₄ and concentrated. Flash chromatography of the residue on silica gave 3-(2-(methylsulfonyl)-1,2,3,4-tetrahydroisoquinolin-6-yl)-5-(4-(4,4,5,5-tetramethyl-1,3,2-dioxaborolan-2-yl)phenethyl)-1,2,4-oxadiazole. MS Calcd for [M+H]⁺: C₂₆H₃₃BN₃O₅S: 510.2; found: 510.2.

Step B 5-(4-(5-Methylpyridin-2-yl)phenethyl)-3-(2-(methylsulfonyl)-1,2,3,4-tetrahydroisoquinolin-6-yl)-1,2,4-oxadiazole. In a microwave vessel was added 3-(2-(methylsulfonyl)-1,2,3,4-tetrahydroisoquinolin-6-yl)-5-(4-(4,4,5,5-tetramethyl-1,3,2-dioxaborolan-2-yl)phenethyl)-1,2,4-oxadiazole (25 mg), 2-bromo-5-methylpyridine (20 mg), (PPh₃)₄Pd (3 mg), dioxane (2 mL), and aqueous Na₂CO₃ (1M, 1 mL). The vessel was sealed and irradiated in a microwave unit at 160° C. for 10 min. The mixture was then diluted with EtOAc (10 mL), washed with brine (2×5 mL) and dried (Na₂SO₄). Solvents were removed under reduced pressure to give crude product. Purification of the crude on silica afforded desired product. MS Calcd for [M+H]⁺ C₂₆H₂₇N₄O₃S: 475.2; found 475.2.

Examples 164, 166, 168, 169, 190, 193, 194, 195 and 196 were prepared by analogous method from example 165.

### Example 167

### 2-(Methylsulfonyl)-6-(3-(4-(pyrimidin-2-yl)phenyl)propoxy)-1,2,3,4-tetrahydroisoquinoline

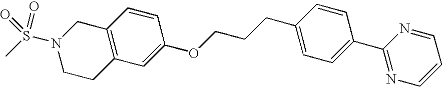

Step A 6-(3-(4-Bromophenyl)propoxy)-2-(methylsulfonyl)-1,2,3,4-tetrahydroisoquinoline. To a solution of 2-(methylsulfonyl)-1,2,3,4-tetrahydroisoquinolin-6-ol (2 g), 3-(4-bromophenyl)propyl methanesulfonate (2.5 g) in DMF (20 mL) was added Cs₂CO₃ (3.2 g). The mixture was stirred at rt overnight, diluted with EtOAc (150 mL). The organics was washed with water (3×50 mL) and brine (100 mL), dried (Na₂SO₄) and filtered. Solvents were removed under reduced pressure to give crude product. Purification of the crude on silica gel (EtOAc:Hexane=1:3) afforded 6-(3-(4-bromophenyl)propoxy)-2-(methylsulfonyl)-1,2,3,4-tetrahydroisoquinoline. MS calcd. for [M+H]⁺ C₁₉H₂₃BrNO₃S: 424.0; found: 424.0.

Step B 2-(Methylsulfonyl)-6-(3-(4-(4,4,5,5-tetramethyl-1,3,2-dioxaborolan-2-yl)phenyl)propoxy)-1,2,3,4-tetrahydroisoquinoline. 6-(3-(4-bromophenyl)propoxy)-2-(methylsulfonyl)-1,2,3,4-tetrahydroisoquinoline (1.24 g), 4,4,4′,4′,5,5,5′,5′-octamethyl-2,2′-bi(1,3,2-dioxaborolane) (0.88 g), KOAc (1.5 g), (dppf)₂PdCl₂ (120 mg) and DMSO (18 mL) were placed in a 100 mL reaction flask. The mixture was degassed with Ar, sealed, heated to 80° C. for 5 hrs and cooled to rt. EtOAc (100 mL) was added and the mixture was washed with water (3×20 mL), brine (50 mL), dried (Na₂SO₄) and concentrated. Flash chromatography of the residue (eluent:EtOAc:Hexane=1:3) gave 2-(methylsulfonyl)-6-(3-(4-(4,4,5,5-tetramethyl-1,3,2-dioxaborolan-2-yl)phenyl)propoxy)-1,2,3,4-tetrahydroisoquinoline. MS Calcd for [M+H]⁺ C₂₃H₃₁BNO₅S: 444.2; found: 444.2.

Step C 2-(Methylsulfonyl)-6-(3-(4-(pyrimidin-2-yl)phenyl)propoxy)-1,2,3,4-tetrahydroisoquinoline. 2-(methylsulfonyl)-6-(3-(4-(4,4,5,5-tetramethyl-1,3,2-dioxaborolan-2-yl)phenyl)propoxy)-1,2,3,4-tetrahydroisoquinoline (25 mg), 2-chloropyrimidine (25 mg), (Ph₃P)₄Ph (5 mg), dioxane (2.5 mL), Na₂CO₃ (1 M, 1 mL) were placed in a microwave reaction vessel and irradiated in microwave at 160° C. for 10 min. The mixture was cooled to rt, diluted with EtOAc (10 mL), washed with brine (5 mL), dried (Na₂SO₄) and filtered. Solvents were removed under reduced pressure to give crude product. Purification of the crude on silica (EtOAc:Hexane=1:2) afforded desired product. MS Calcd for [M+H]⁺ C₂₃H₂₆N₃O₃S: 424.2; found: 424.2.

Examples 168-170, 178, 180, 191 and 197 were prepared by analogous method from example 167.

### Example 171

### 6-(3-(4-(5-Ethylpyrimidin-2-yl)phenyl)propoxy)-5,7-difluoro-2-(methylsulfonyl)-1,2,3,4-tetrahydroisoquinoline

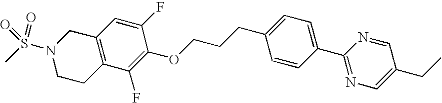

Synthesis of 6-(3-(4-(5-ethylpyrimidin-2-yl)phenyl)propoxy)-5,7-difluoro-2-(methylsulfonyl)-1,2,3,4-tetrahydroisoquinoline (171). To a rubber septum capped tube was combined 146f (30 mg, 0.11 mmol), 3-(4-(5-ethylpyrimidin-2-yl)phenyl)propyl methanesulfonate (38 mg, 0.12 mmol), and Cs₂CO₃ (54 mg, 0.17 mmol) in CH₃CN. The mixture was stirred at 80° C. overnight, filtered, and the filtrate was concentrated. The residue was purified on silica gel to afford the title compound 171 as white solids. MS Calcd for [M+H]⁺ C₂₅H₂₈F₂N₃O₃S: 489.2; found: 489.2.

### Example 176

### 3-tert-Butyl-5-(4-((2-(methylsulfonyl)-1,2,3,4-tetrahydroisoquinolin-6-yloxy)methyl)phenyl)-1,2,4-oxadiazole

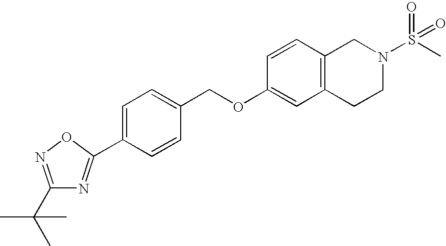

Step A 3-tert-Butyl-5-(4-(chloromethyl)phenyl)-1,2,4-oxadiazole To a dry round bottom flask was added N-hydroxypivalimidamide (0.45 g) and THF (25 mL). After complete dissolution, 4-(chloromethyl)benzoyl chloride (0.62 g) was added followed by addition of Et₃N (1 mL). The resulting mixture was heated at 60° C. overnight. It was cooled to rt, EtOAc (50 mL) was added and the mixture was washed with water, brine, dried over Na₂SO₄ and filtered. Solvents were removed under reduced pressure to give crude product. Purification of the crude on silica gel afforded 3-tert-butyl-5-(4-(chloromethyl)phenyl)-1,2,4-oxadiazole. MS calcd. for [M+H]⁺ C₁₃H₁₆ClN₂O: 251.1; found: 251.1.

Step B 3-tert-Butyl-5-(4-((2-(methylsulfonyl)-1,2,3,4-tetrahydroisoquinolin-6-yloxy)methyl)phenyl)-1,2,4-oxadiazole (176) To a reaction flask was added 3-tert-butyl-5-(4-(chloromethyl)phenyl)-1,2,4-oxadiazole (25 mg), 2-(methylsulfonyl)-1,2,3,4-tetrahydroisoquinolin-6-ol (20 mg), Cs₂CO₃ (60 mg) and DMF (2 mL). After complexion of the reaction, EtOAc (20 mL) was added and the resulting mixture was washed with water (3×10 mL), brine (10 mL), dried (MgSO₄) and filtered. Solvents were removed under reduced pressure to give crude product. Purification of the crude on silica gel (EtOAc:Hexanes=1:3) afforded desired product 176. MS Calcd for [M+H]⁺ C₂₃H₂₈N₃O₄S: 442.2; found: 442.2

### Example 177

### Isopropyl 6-((2-(methylsulfonyl)-1,2,3,4-tetrahydroisoquinolin-6-yloxy)methyl)-3,4-dihydroisoquinoline-2(1H)-carboxylate

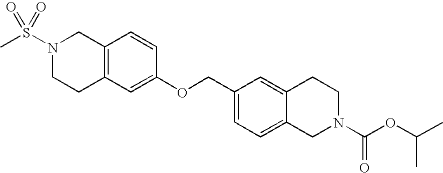

Step A 2-Isopropyl 6-methyl 3,4-dihydroisoquinoline-2,6(1H)-dicarboxylate To a solution of methyl 1,2,3,4-tetrahydroisoquinoline-6-carboxylate (500 mg) in DMF (10 mL) was added Et₃N (1 mL) at 0° C. Isopropyl carbonochloridate (400 mg) was added and the mixture was stirred at 0° C. to rt for 3 hrs. The reaction was quenched by addition of a solution of aqueous NH₄Cl and the mixture was extracted with Et₂O (3×25 mL). The organic layers were combined, washed with brine, dried (MgSO₄) and filtered. Solvents were removed under reduced pressure to give crude product. Purification of the crude on silica gel afforded 2-isopropyl 6-methyl 3,4-dihydroisoquinoline-2,6(1H)-dicarboxylate. MS calcd. for [M+H]⁺ C₁₅H₂₀NO₄: 278.1; found: 278.1.

Step B Isopropyl 6-(hydroxymethyl)-3,4-dihydroisoquinoline-2(1H)-carboxylate To a solution of 2-isopropyl 6-methyl 3,4-dihydroisoquinoline-2,6(1H)-dicarboxylate (560 mg) in THF (20 mL) was added LiBH4 (1 M, 5 mL). After stirring for 12 hrs, the reaction mixture was then heated at 60° C. for 3 hrs to bring the reaction to completion. The mixture was then cooled to 0° C. and water was added followed by the addition of aqueous NH₄Cl. It was extracted with EtOAc (3×25 mL). The organics were combined, washed with water, brine, dried over Na₂SO₄ and filtered. Solvents were removed under reduced pressure to give crude product. Purification of the crude on silica gel afforded isopropyl 6-(hydroxymethyl)-3,4-dihydroisoquinoline-2(1H)-carboxylate as solid. MS Calcd for [M+H]⁺ C₁₄H₂₀NO₃: 250.1; found: 250.1.

Step C Isopropyl 6-((methylsulfonyloxy)methyl)-3,4-dihydroisoquinoline-2(1H)-carboxylate To a dry flask was added isopropyl 6-(hydroxymethyl)-3,4-dihydroisoquinoline-2(1H)-carboxylate (100 mg) and CH₂Cl₂ (10 mL). After cooling to 0° C., methanesulfonic anhydride (100 mg) was added followed by 2,4,6-collidine (0.1 mL). The mixture was stirred at 0° C. for 4 hrs and quenched with water (1 mL), washed with brine, dried over Na₂SO₄ and filtered. Solvents were removed under reduced pressure to give crude product which was used directly for next step.

Step D Isopropyl 6-((2-(methylsulfonyl)-1,2,3,4-tetrahydroisoquinolin-6-yloxy)methyl)-3,4-dihydroisoquinoline-2(1H)-carboxylate 2-(methylsulfonyl)-1,2,3,4-tetrahydroisoquinolin-6-ol (45 mg), isopropyl 6-((methylsulfonyloxy)methyl)-3,4-dihydroisoquinoline-2(1H)-carboxylate (70 mg, crude from previous step), Cs₂CO₃ (120 mg) and DMF (5 mL) was placed in a reaction flask. The mixture was stirred at rt for 5 hrs. EtOAc (50 mL) was added and the resulting mixture was washed with water, brine, dried over Na₂SO₄ and filtered. Solvents were removed under reduced pressure to give crude product. Purification of the crude on silica gel (EtOAc:Hexanes=1:2) afforded the desired product. MS Calcd for [M+H]⁺ C₂₄H₃₁N₂O₅S: 458.2; found: 459.2.

Examples 172-175, 182 were prepared by analogous method from example 177.

### Example 179

### N-Benzyl-N-(4-((2-(methylsulfonyl)-1,2,3,4-tetrahydroisoquinolin-6-yloxy)methyl)benzyl)ethanamine

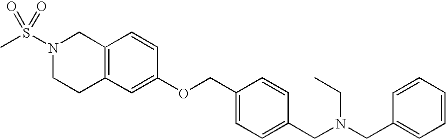

Intermediate 179b: N-Benzyl-N-(4-(chloromethyl)benzyl)-ethanamine

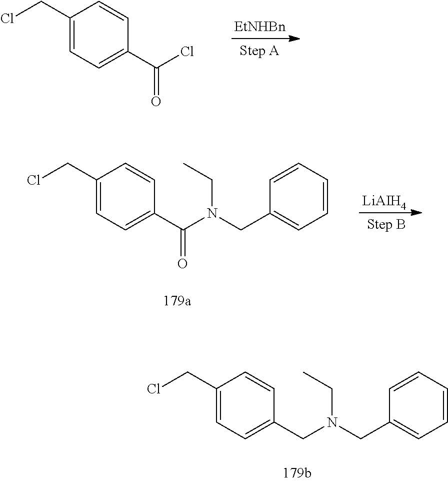

Step A 4-Chloromethylbenzoyl chloride (1 g, 5.29 mmol) was dissolved in dioxane (10 mL) and ethylbenzylamine (2.4 mL, 16.1 mmol) was added dropwise at rt. A white precipitate formed instantaneously. The mixture was stirred at rt for 2 h, diluted with CH₂Cl₂ and washed with sat. NH₄Cl and brine. The organic phase was dried (Na₂SO₄), concentrated in vacuo, and the crude was purified by flash chromatography to afford N-benzyl-4-(chloromethyl)-N-ethylbenzamide (179a) as a colorless oil. MS calcd. for [M+H]⁺ C₁₇H₁₉ClNO: 288.1; found: 288.1.

Step B A sample of 179a (666 mg, 2.31 mmol) was dissolved in THF (5 mL). The mixture was cooled to 0° C. and a solution of LiAlH₄ in THF (1 M, 2.31 mL, 2.31 mmol) was added dropwise. The mixture was then stirred at rt overnight then carefully quenched with saturated aqueous Na₂SO₄ until no more gas evolution was observed. The mixture was then filtered through celite and washed with EtOAc. Concentration of the organic phase yielded N-benzyl-N-(4-(chloromethyl)benzyl)-ethanamine (179b), which was used in the next step without further purification. ¹H-NMR (400 MHz, CDCl₃) δ=7.32-7.28 (m, 2H), 7.26-7.21 (m, 2H), 7.18-7.09 (m, 4H), 6.98-6.95 (m, 2H), 3.49 (s, 2H), 3.46 (s, 2H), 2.42 (q, J=7.2 Hz, 2H), 2.27 (s, 2H), 0.99 (t, J=7.2 Hz, 3H); MS calcd. for [M+H]⁺ C₁₇H₂₁ClN: 274.1; found: 274.1.

A sample of 3 (61.6 mg, 0.27 mmol), 179b (82 mg, 0.30 mmol), and Cs₂CO₃ (177 mg, 0.54 mmol) were dissolved/suspended in MeCN (1.5 mL) and stirred at 90° C. overnight. The mixture was then diluted with MeCN and filtered. The filtrate was purified by reverse-phase HPLC to yield the title compound (Example 179). ¹H-NMR (400 MHz, CDCl₃) δ=7.59-7.40 (m, 9H), 7.02 (d, J=8.4 Hz, 2H), 6.84 (dd, J=8.4, 2.5 Hz, 2H), 6.77 (d, J=2.5 Hz, 2H), 5.10 (s, 2H), 4.41 (s, 2H), 4.44-4.36 (m, 2H), 4.22-4.12 (m, 2H), 3.55 (t, J=6.0 Hz, 2H), 3.04 (q, J=7.2 Hz, 2H), 2.96 (t, J=6.0 Hz, 2H), 2.85 (s, 3H), 1.40 (t, J=7.2 Hz, 3H); MS calcd. for [M+H]⁺ C₂₇H₃₃N₂O₃S: 465.2; found: 465.2.

### Example 199

### 6-(3-(4-(5-((2-Methoxyethoxy)methyl)pyrimidin-2-yl)phenyl)propoxy)-2-(methylsulfonyl)-1,2,3,4-tetrahydroisoquinoline

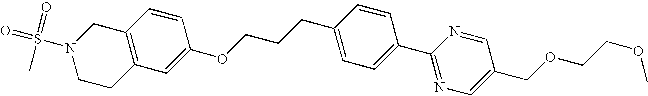

Step A (2-Chloropyrimidin-5-yl)methanol To a dry flask was added methyl 2-chloropyrimidine-5-carboxylate (17 mg) and THF (5 mL). The mixture was cooled to −78° C. and a solution of DIBAL-H in hexane (1 M, 1.2 mL) was added slowly. The resulting mixture was stirred at −78° C. to rt overnight, then quenched with saturated aqueous Na₂SO₄. The solution was filtered. Solvents were removed under reduced pressure to give (2-chloropyrimidin-5-yl)methanol. MS calcd. for [M+H]⁺ C₅H₆ClN₂O: 145.1; found: 145.1.

Step B (2-(4-(3-(2-(Methylsulfonyl)-1,2,3,4-tetrahydroisoquinolin-6-yloxy)propyl)phenyl)pyrimidin-5-yl)methanol To a reaction vessel was added 2-(methylsulfonyl)-6-(3-(4-(4,4,5,5-tetramethyl-1,3,2-dioxaborolan-2-yl)phenyl)propoxy)-1,2,3,4-tetrahydroisoquinoline (20 mg), (2-chloropyrimidin-5-yl)methanol (10 mg), Pd(PPh₃)₄ (2 mg), dioxane (2 mL) and Na₂CO₃ (1 M, 1 mL). The mixture was irradiated in a microwave at 160° C. for 10 min. The mixture was cooled to rt, extracted with CHCl₃. The extracts were combined, washed with water, dried (MgSO₄) and filtered. Solvents were removed under reduced pressure to give crude product. Purification of the crude on silica gel column (EtOAc:Hexanes=2:1) afforded the desired product. MS calcd. for [M+H]⁺ C₂₅H₂₉N₂O₄S: 453.2; found: 453.2.

Step C 6-(3-(4-(5-((2-Methoxyethoxy)methyl)pyrimidin-2-yl)phenyl)propoxy)-2-(methylsulfonyl)-1,2,3,4-tetrahydroisoquinoline To a dry flask was added (2-(4-(3-(2-(methylsulfonyl)-1,2,3,4-tetrahydroisoquinolin-6-yloxy)propyl)phenyl)pyrimidin-5-yl)methanol (90 mg) and DMF (3 mL). The solution was cooled to 0° C. and NaH (40 mg) was added portionwise. The resulting mixture was stirred for 10 min and 2-bromoethyl methyl ether (0.1 mL) was added. After complexion of the reaction, water was added and the mixture was extracted with CHCl₃. The organic layers were combined and washed with water, brine, dried (MgSO₄) and filtrated. Solvents were removed under reduced pressure to give crude product. Purification of the crude on silica gel column afforded the title compound. MS calcd. for [M+H]⁺ C₂₇H₃₃N₃O₅S: 497.2; found: 496.2.

### Example 201

### 4-(3-(2-(Methylsulfonyl)-1,2,3,4-tetrahydroisoquinolin-6-yloxy)propyl)benzonitrile

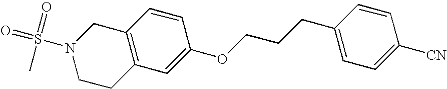

To a reaction vessel was added 6-(3-(4-bromophenyl)propoxy)-2-(methylsulfonyl)-1,2,3,4-tetrahydroisoquinoline (84 mg), Zn(CN)₂ (18 mg), Xantphos (102 mg), TMEDA (0.05 mL) and DMF (3 mL). The mixture was heated at 160° C. for 5 min in a microwave. The mixture was cooled to rt, EtOAc (20 mL) was added. The mixture was washed with brine, dried (MgSO₄) and filtered. Solvents were removed under reduced pressure to give crude product. Purification of the crude on silica gel gave the title compound. MS calcd. for [M+H]⁺ C₂₆H₃₀N₃O₃S: 371.1; found: 371.1.

### Example 202

### 6-(3-(4-(2H-Tetrazol-5-yl)phenyl)propoxy)-2-(methylsulfonyl)-1,2,3,4-tetrahydroisoquinoline

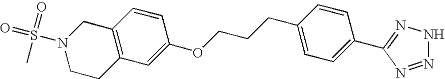

To a reaction vessel was added 4-(3-(2-(methylsulfonyl)-1,2,3,4-tetrahydroisoquinolin-6-yloxy)propyl)benzonitrile (10 mg), NH₄Cl (7 mg), NaN₃ (5 mg) and DMF (2 mL). The mixture was heated in a microwave reaction vessel at 160° C. for 5 min. It was cooled to rt, EtOAc (20 mL) was added and the mixture was washed with water, brine, dried (MgSO₄) and filtered. Solvents were removed under reduced pressure to give crude product. Purification of the crude on silica gel column (EtOAc with 1% HOAc) afforded the title compound. MS calcd. for [M+H]⁺ C₂₆H₃₀N₃O₃S: 414.1; found: 414.1.

### Example 204

### 6-(3-(4-(5-Ethylpyrimidin-2-yl)phenyl)propoxy)-7-fluoro-2-(methylsulfonyl)-1,2,3,4-tetrahydroisoquinoline

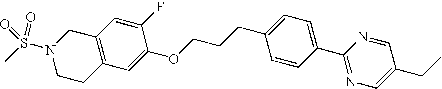

6-(3-(4-(5-Ethylpyrimidin-2-yl)phenyl)propoxy)-7-fluoro-2-(methylsulfonyl)-1,2,3,4-tetrahydroisoquinoline. The title compound was synthesized according to the procedure described for the synthesis of example 146 using 7-fluoro-2-(methylsulfonyl)-1,2,3,4-tetrahydroisoquinolin-6-ol (149e) and mesylate 3-(4-(5-ethylpyrimidin-2-yl)phenyl)propyl methanesulfonate. MS calcd. for [M+H]⁺ C₂₅H₂₉FN₃O₃S: 470.1; found: 470.1.

### Example 206

### tert-Butyl 4-(4-(hydroxyimino)-4-(2-(methylsulfonyl)-1,2,3,4-tetrahydroisoquinolin-6-yl)butyl)piperidine-1-carboxylate

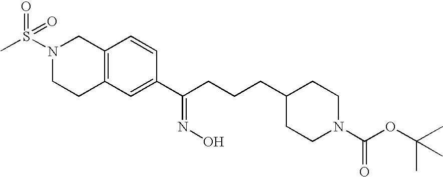

Example 139 (30.0 mg, 0.06 mmol), NaOAc (6.6 mg, 0.08 mmol) and NH₂OH.HCl (5.5 mg, 0.08 mmol) were dissolved in MeOH (0.5 mL) and the mixture was stirred overnight at rt. The mixture was then diluted with MeCN and filtered. The filtrate was purified by reverse-phase HPLC to yield the title compound (Example 206). E-Isomer: ¹H-NMR (400 MHz, CD₃CN) δ=8.84 (s, 1H), 7.39-7.37 (m 2H), 7.08 (d, J=8.0 Hz, 1H), 4.32 (s, 2H), 3.98-3.85 (m, 2H), 3.40 (t, J=6.0 Hz, 2H), 2.90 (t, J=6.0 Hz, 2H), 2.76 (s, 3H), 2.65-2.61 (m, 2H), 2.56-2.44 (m, 2H), 1.54-1.50 (m, 2H), 1.45-1.36 (m, 2H), 1.32 (s, 9H), 1.32-1.21 (m, 1H), 1.21-1.16 (m, 2H), 0.91-0.79 (m 2H); MS calcd. for [M+H]⁺ C₂₄H₃₈N₃O₅S: 480.2; found: 480.2.

### Example 207

### tert-Butyl 4-(4-(methoxyimino)-4-(2-(methylsulfonyl)-1,2,3,4-tetrahydroisoquinolin-6-yl)butyl)piperidine-1-carboxylate

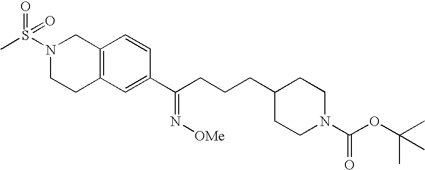

A sample of compound 139 (30.0 mg, 0.06 mmol), NaOAc (6.6 mg, 0.08 mmol) and O-methylhydroxylamine hydrochloride (6.6 mg, 0.08 mmol) were dissolved in MeOH (0.5 mL) and the mixture was stirred overnight at rt. The mixture was then diluted with MeCN and filtered. The filtrate was purified by reverse-phase HPLC to yield the title compound (Example 207). E-isomer: ¹H-NMR (400 MHz, CD₃CN) δ=7.40-7.37 (m 2H), 7.08 (d, J=8.0 Hz, 1H), 4.32 (s, 2H), 3.91-3.88 (m, 2H), 3.82 (s, 3H), 3.41 (t, J=6.0 Hz, 2H), 2.89 (t, J=6.0 Hz, 2H), 2.75 (s, 3H), 2.63-2.59 (m, 2H), 2.56-2.50 (m, 2H), 1.52-1.49 (m, 2H), 1.44-1.36 (m, 2H), 1.32 (s, 9H), 1.32-1.24 (m, 1H), 1.24-1.14 (m, 2H), 0.92-0.82 (m 2H); MS calcd. for [M+H]⁺ C₂₅H₄₀N₃O₅S: 494.2; found: 494.2.

### Example 208

### 1-Methylcyclopropyl 4-(4-hydroxy-4-(2-(methylsulfonyl)-1,2,3,4-tetrahydroisoquinolin-6-yl)butyl)piperidine-1-carboxylate

### Example 209

### 1-Methylcyclopropyl 4-(4-chloro-4-(2-(methylsulfonyl)-1,2,3,4-tetrahydroisoquinolin-6-yl)butyl)piperidine-1-carboxylate

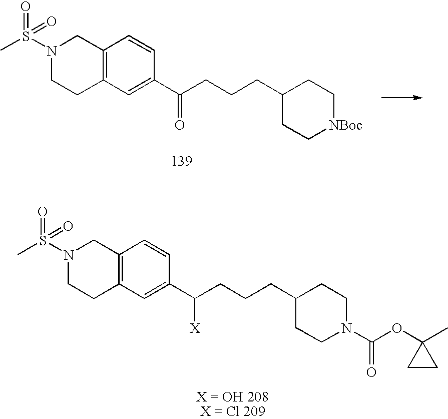

Following the procedure described for Example 129, Example 139 (34 mg, 0.07 mmol) was converted to the corresponding alcohol. Boc deprotection was then performed using the same procedure described for the preparation of 51a, and conversion to the title compound (Example 208) was achieved following the procedure described for Example 60. The mixture was diluted with MeCN and filtered. The filtrate was purified by reverse-phase HPLC to yield the title compounds (Example 208 and Example 209). Compound 208: ¹H-NMR (400 MHz, CD₃CN) δ=7.07-6.99 (m, 3H), 4.46-4.42 (m, 1H), 4.28 (s, 2H), 4.96-4.75 (m, 2H), 3.38 (t, J=6.0 Hz, 2H), 2.85 (t, J=6.0 Hz, 2H), 2.74 (s, 3H), 2.64-2.54 (m, 2H), 1.58-1.45 (m, 4H), 1.38 (s, 3H), 1.33-1.21 (m, 2H), 1.20-1.09 (m, 3H), 0.91-0.81 (m, 2H), 0.70-0.67 (m, 2H), 0.50-0.47 (m, 2H); MS calcd. for [M+H]⁺ C₂₄H₃₇N₂O₅S: 465.2; found: 465.2.

Compound 209: ¹H-NMR (400 MHz, CD₃CN) δ=7.17-7.15 (m, 2H), 7.06 (d, J=8.0 Hz, 1H), 4.87-4.83 (m, 1H), 4.30 (s, 2H), 4.98-4.72 (m, 2H), 3.39 (t, J=6.0 Hz, 2H), 2.86 (t, J=6.0 Hz, 2H), 2.74 (s, 3H), 2.56 (br. s, 2H), 2.04-1.90 (m, 2H), 1.54-1.44 (m, 2H), 1.38 (s, 3H), 1.33-1.24 (m, 1H), 1.20-1.12 (m, 4H), 0.90-0.81 (m, 2H), 0.70-0.68 (m, 2H), 0.50-0.47 (m, 2H); MS calcd. for [M+H]⁺ C₂₄H₃₆ClN₂O₄S: 483.2; found: 483.2.

### Example 214

### 6-(3-(4-(5-Ethylpyrimidin-2-yl)phenyl)propoxy)-1,2,3,4-tetrahydroisoquinoline

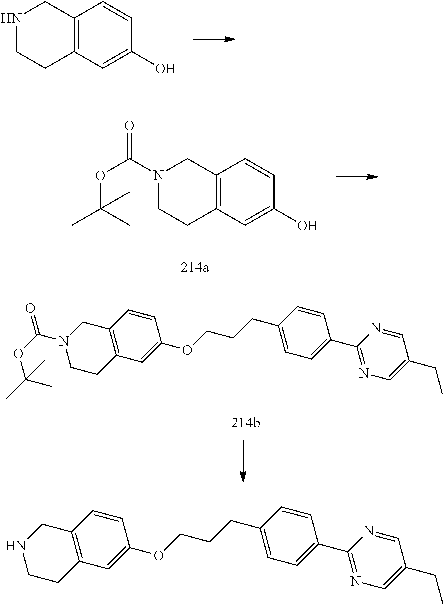

Step A tert-Butyl 6-hydroxy-3,4-dihydroisoquinoline-2(1H)-carboxylate (214a) To a suspension of 1,2,3,4-tetrahydroisoquinolin-6-ol (1.14 gram, HBr salt) in dichloromethane (30 mL) was added Et₃N (0.24 mL) at 0° C. The mixture was stirred for 10 min before the addition of di-tert-butyl dicarbonate (1.1 g) in one portion. The mixture was then stirred at rt overnight. Water (2 mL) was added followed by the addition of dichloromethane (50 mL). The mixture was washed with aqueous HCl (1 N, 10 mL), brine, dried and filtered. Solvent was removed under reduced pressure to give the crude product. The crude was purified on silica gel (EtOAc:Hexanes=1:1) to yield the desired product. MS calcd. for [M+H]⁺ C₁₄H₂₀NO₃: 250.1; found: 250.1.

Step B tert-Butyl 6-(3-(4-(5-ethylpyrimidin-2-yl)phenyl)propoxy)-3,4-dihydroisoquinoline-2(1H)-carboxylate (214b) To a solution of tert-butyl 6-hydroxy-3,4-dihydroisoquinoline-2(1H)-carboxylate (250 mg, 1 mmol) in DMF (5 mL) was added Cs₂CO₃ (600 mg, 1.9 mmol) and 3-(4-(5-ethylpyrimidin-2-yl)phenyl)propyl methanesulfonate (360 mg, 1.15 mmol). The mixture was stirred overnight. Water (5 mL) was added and the mixture was extracted with chloroform (3×10 mL). The organics were combined, washed with brine, dried and filtered. Solvents were removed under reduced pressure to provide the crude product. The crude was purified on silica gel (EtOAc: Hexanes=1:1) to yield the desired product. MS calcd. for [M+H]⁺ C₂₉H₃₆N₃O₃: 474.2; found: 474.2.

Step C 6-(3-(4-(5-Ethylpyrimidin-2-yl)phenyl)propoxy)-1,2,3,4-tetrahydroisoquinoline (214) tert-Butyl 6-(3-(4-(5-ethylpyrimidin-2-yl)phenyl)propoxy)-3,4-dihydroisoquinoline-2(1H)-carboxylate (410 mg, 0.82 mmol) was dissolved in dioxane (2 mL). A solution of HCl in dioxane (4 N, 2 mL) was added and the mixture was stirred for 20 h. Solvent was removed under reduced pressure and the remainder was dried under high vacuum to afford the desired product as HCl salt. MS calcd. for [M+H]⁺ C₂₄H₂₈N₃O: 374.2; found: 374.2.

### Example 215

### 6-(3-(4-(5-Ethylpyrimidin-2-yl)phenyl)propoxy)-2-(vinylsulfonyl)-1,2,3,4-tetrahydroisoquinoline

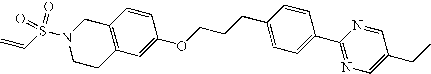

To a suspension of 6-(3-(4-(5-ethylpyrimidin-2-yl)phenyl)propoxy)-1,2,3,4-tetrahydroisoquinoline hydrochloride (50 mg, 0.12 mmol) in dichloromethane (2 mL) was added Et₃N (0.1 mL, 0.78 mmol). The mixture was cooled to 0° C. and 2-chloroethanesulfonyl chloride (19 mg, 0.12 mmol) was added slowly. After stirring at 0° C. for 4 h and rt for 2 h, water (2 mL) was added followed by the addition of chloroform (5 ml). The organics were separated, washed with brine, dried and filtered. Solvents were removed to provide the crude product. The crude was purified on silica gel column (EtOAc:Hexanes=1:2) to afford the desired product. MS calcd. for [M+H]⁺ C₂₆H₃₀N₃O₃S: 464.2; found: 464.2.

### Example 216

### 1-Methylcyclopropyl 4-(3-(2-(methylsulfonyl)-1,2,3,4-tetrahydroisoquinolin-6-yloxy)propyl)piperazine-1-carboxylate

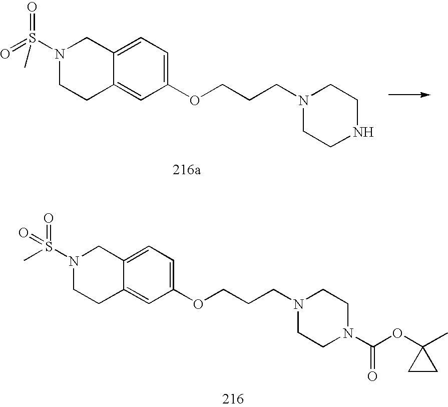

Step A 2-(Methylsulfonyl)-6-(3-(piperazin-1-yl)propoxy)-1,2,3,4-tetrahydroisoquinoline (216a) Trifluoroacetic acid (8 mL) was added at rt to a solution of 261 (500 g, 1.1 mmol) in CH₂Cl₂ (32 mL). The mixture was stirred at rt for 30 mins. The solvents were evaporated and the residue was diluted with chloroform and then neutralized with sat. NaHCO₃. The aqueous was extracted with chloroform-(3×10 mL). The combined organics were dried over Na₂SO₄ and concentrated in vacuo to afford 216a as an off white solid. ¹H-NMR (400 MHz, CDCl₃) δ 6.99 (dd, 1H, J=8.4 Hz), 6.75 (dd, 1H, J=2.4, 8.4 Hz), 6.67 (d, 1H, J=2.0 Hz), 4.40 (s, 2H), 3.99 (t, 2H, J=6.0 Hz), 3.54 (t, 2H, J=6.0 Hz), 3.04 (t, 4H, J=4.8 Hz), 2.94 (t, 2H, J=6.0 Hz), 2.59-2.55 (m, 6H), 1.96 (quint, 2H, J=6.4 Hz); MS calcd. for [M+H]⁺ C₁₇H₂₇N₃O₃S: 354.2; found: 354.1.

Step B To a solution of 216a (20 mg, 0.06 mmol) and triethylamine (16 uL, 0.11 mmol) in CH₂Cl₂ (5 mL) was added 1-methylcyclopropyl 4-nitrophenyl carbonate (14 mg, 0.06 mmol) at 0° C. The ice water bath was removed and the resulting mixture was stirred at rt for 18 h. The solvent was removed under reduced pressure and the crude was purified by flash column chromatography (EtOAc/hexane) to afford the title compound 216. MS calcd. for [M+H]⁺ C₂₂H₃₃N₃O₅S: 452.2; found: 452.2.

### Example 217

### 6-(3-(4-(5-Ethylpyrimidin-2-yl)piperazin-1-yl)propoxy)-2-(methylsulfonyl)-1,2,3,4-tetrahydroisoquinoline

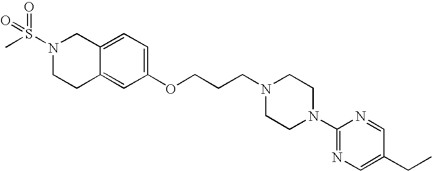

To a microwave reaction vessel was charged with 216a (20 mg, 0.06 mmol), 2-chloro-5-ethyl pyrimidine (20 μL, 0.17 mmol), K₂CO₃ (70 mg, 0.5 mmol) and 1,4-dioxane (1 mL). The vessel was sealed and heated at 160° C. for 20 min under microwave irradiation, then cooled to rt. The mixture was diluted with EtOAc and filtered. The filtrate was concentrated and purified by silica gel column chromatography (EtOAc/hexane) to yield the title compound 217 as a white solid. ¹H-NMR (400 MHz, CDCl₃) δ 8.18 (s, 2H), 7.00 (d, 1H, J=8.4 Hz), 6.77 (dd, 1H, J=2.4, 8.4 Hz), 6.69 (d, 1H, J=2.4 Hz), 4.40 (s, 2H), 4.02 (t, 2H, J=6.0 Hz), 3.80 (t, 4H, J=4.8 Hz), 3.54 (t, 2H, J=6.0 Hz), 2.94 (t, 2H, J=6.0 Hz), 2.83 (s, 3H), 2.58-2.51 (m, 6H), 2.46 (q, 2H, J=7.6 Hz), 2.00 (quint, 2H, J=6.4 Hz), 1.19 (t, 3H, J=7.6 Hz); MS calcd. for [M+H]⁺ C₂₃H₃₃N₅O₃S: 460.2; found: 460.2.

### Example 218

### 2-(Methylsulfonyl)-6-(3-(3-(pyrimidin-2-yloxy)phenyl)propoxy)-1,2,3,4-tetrahydroisoquinoline

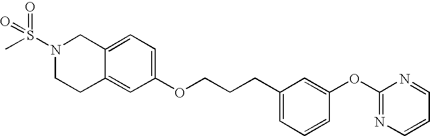

A mixture of 260 (25 mg, 0.069 mmol), 2-chloropyrimidine (20 mg, 0.17 mmol), and Cs₂CO₃ (50 mg, 0.15 mmol) in 1,4-dioxane (2 mL) was heated at 120° C. overnight. The mixture was cooled to rt, then diluted with EtOAc and filtered. The filtrate was concentrated in vacuo and the crude was purified by silica gel column chromatography (EtOAc/hexane) to afford the title compound 218. MS calcd. for [M+H]⁺ C₂₅H₂₉N₃O₄S: 468.2; found: 468.2.

### Example 219

### 2-(Methylsulfonyl)-6-(3-(4-(pyrimidin-2-yloxy)phenyl)propoxy)-1,2,3,4-tetrahydroisoquinoline

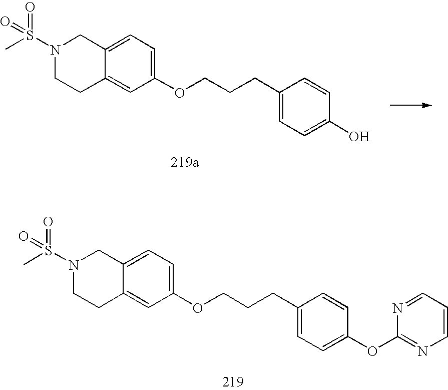

Step A 4-(3-(2-(Methylsulfonyl)-1,2,3,4-tetrahydroisoquinolin-6-yloxy)propyl)phenol (219a) To a solution of 234 (250 mg, 0.55 mmol) in ethyl acetate (50 mL) was added Pd/C (10 wt %, 100 mg). The mixture was stirred under a hydrogen atmosphere for 30 minutes and then filtered through a pad of Celite. Removal of the solvents under reduced pressure afforded intermediate 219a as a white solid. ¹H-NMR (400 MHz, CDCl₃) δ 7.07 (d, 2H, J=8.4 Hz), 6.99 (d, 1H, J=8.4 Hz), 6.77-6.74 (m, 3H), 6.66 (d, 1H, J=2.4 Hz), 4.57 (s, 1H), 4.40 (s, 2H), 3.92 (t, 2H, J=6.4 Hz), 3.54 (t, 2H, J=5.6 Hz), 2.94 (t, 2H, J=6.0 Hz), 2.83 (s, 3H), 2.73 (t, 2H, J=7.2 Hz), 2.05 (quint, 2H, J=6.0 Hz); MS calcd. for [M+H]⁺ C₁₉H₂₃NO₄S: 362.1; found: 361.8.

Step B Example 219 was prepared by analogous methods described for example 218 from derivative 219a and 2-chloropyrimidine. ¹H-NMR (400 MHz, CDCl₃) δ 8.57 (d, 2H, J=4.8 Hz), 7.27 (d, 2H, J=8.4 Hz), 7.14-7.11 (m, 2H), 7.03 (t, 1H, J=4.8 Hz), 7.00 (d, 1H, J=8.4 Hz), 6.77 (dd, 1H, J=2.4, 8.4 Hz), 6.68 (d, 1H, J=2.4 Hz), 4.40 (s, 2H), 3.97 (t, 2H, J=6.4 Hz), 3.55 (t, 2H, J=6.0 Hz), 2.94 (t, 2H, J=6.0 Hz), 2.85-2.81 (m, 5H), 2.15-2.09 (m, 2H); MS calcd. for [M+H]⁺ C₂₃H₂₅N₃O₄S: 440.2; found: 440.1.

### Example 220

### tert-Butyl 4-(4,5-dihydroxy-4-(2-(methylsulfonyl)-1,2,3,4-tetrahydroisoquinolin-6-yl)pentyl)piperidine-1-carboxylate

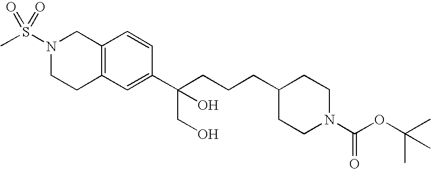

Sodium hydride (24 mg, 1.0 mmol) was suspended in DMSO (1 mL) and the mixture was cooled to 5° C. Trimethylsulfoxonium iodide (202 mg, 0.92 mmol) was added in one portion and the resulting solution was stirred 1 h at 5° C. Example 139 (400 mg, 0.86 mmol) was added in DMSO (3.5 mL) and the solution heated to 50° C. for 24 h. The solution was cooled, diluted with H₂O, and extracted with dichloromethane (3×10 mL). The combined organic layers were dried over Na₂SO₄, and concentrated in vacuo. Purification the crude by flash chromatography (SiO₂, gradient elution with 2% to 5% MeOH in dichloromethane), followed by reversed-phase HPLC (water-acetonitrile gradient with TFA as ion-pairing reagent) and lyophilization afforded Example 220 as a white powder. ¹H-NMR (400 MHz, CD₃CN) δ=7.23 (m, 2H), 7.10 (d, J=8.5 Hz, 1H), 4.38 (s, 2H), 3.95 (d, J=12.8 Hz, 2H), 3.55 (d, J=5.8 Hz, 2H), 3.48 (t, J=5.8 Hz, 2H), 2.94 (t, J=5.8 Hz, 2H), 2.83 (s, 3H), 2.61 (br s, 2H), 1.71 (dd, J=9.0, 7.5 Hz, 2H), 1.52 (d, J=12.3 Hz, 2H), 1.39 (s, 9H), 1.30 (m, 2H), 1.14 (m, 2H), 0.91 (m, 2H); MS calcd. for [M+Na]⁺ C₂₅H₄₀NaN₂O₆S: 519.3; found: 519.3.

### Example 221

### N,N-Dimethyl-2-(5-(4-(3-(2-(methylsulfonyl)-1,2,3,4-tetrahydroisoquinolin-6-yloxy yloxy)propyl)piperidin-1-yl)-2H-tetrazol-2-yl)ethanamine

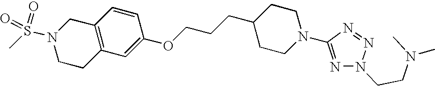

The title compound was prepared in a manner similar to Example 124 from Example 123 using appropriate starting materials.

### Example 222

### 6-(1-(4-(5-Ethylpyrimidin-2-yl)phenyl)pyrolidin-3-yloxy)-2-(methylsulfonyl)-1,2,3,4-tetrahydroisoquinoline

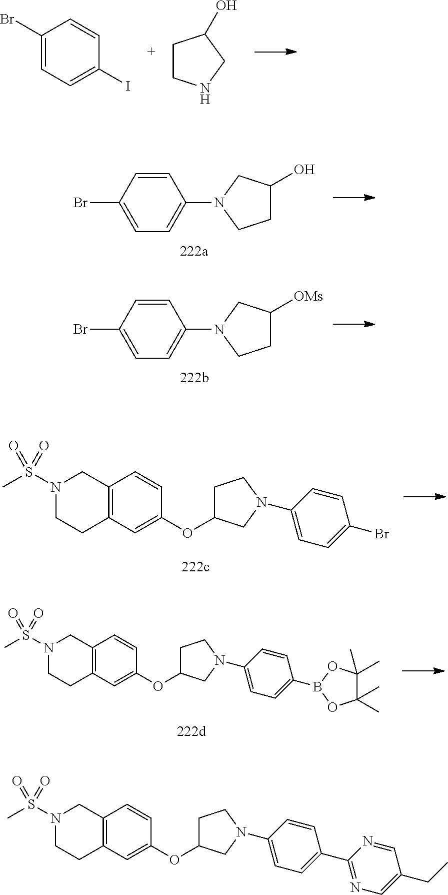

Step A 1-(4-Bromophenyl)pyrrolidin-3-ol (222a) A microwave reaction vessel was charged with 1-bromo-4-iodobenzene (1.2 g, 4.25 mmol), pyrrolidin-3-ol (0.68 g, 7.8 mmol), Cs₂CO₃ (1 g, 3 mmol), pyrrolidine-2-carboxylic acid (0.05 g, 0.43 mmol) and DMF (25 mL). The mixture was irradiated in microwave at 160° C. for 30 min. It was cooled to rt, diluted with EtOAc (60 mL), washed with brine, dried and filtered. Solvents were removed to give crude product. The crude was purified on silica gel (EtOAc:Hexanes=1:2) to yield the desired product. MS calcd. for [M+H]⁺ C₁₀H₁₃BrNO: 242.0; found: 242.0.

Step B 1-(4-Bromophenyl)pyrrolidin-3-yl methanesulfonate (222b) To a solution of 1-(4-bromophenyl)pyrrolidin-3-ol (0.42 g, 1.74 mmol) in dichloromethane (10 mL) was added Et₃N (0.2 g, 2 mmol) followed by MsCl (0.20 g, 1.75 mmol) at 0° C. The mixture was stirred at 0° C. for 3 h, and then water (1 ml) was added to quench the reaction. The organics were washed with brine, dried, filtered. Solvents were removed under reduced pressure to provide the crude product. The crude was purified on silica gel (EtOAc:Hexanes=1:3) to yield the desired product. MS calcd. for [M+H]⁺ C₁₁H₁₅BrNO₃S: 320.0; found: 320.0.

Step C 6-(1-(4-Bromophenyl)pyrrolidin-3-yloxy)-2-(methylsulfonyl)-1,2,3,4-tetrahydroisoquinoline (222c) A mixture of 1-(4-bromophenyl)pyrrolidin-3-yl methanesulfonate (0.16 g, 0.5 mmol), Cs₂O₃ (0.2 g, 0.61 mmol), 2-(methylsulfonyl)-1,2,3,4-tetrahydroisoquinolin-6-ol (0.12 g, 0.53 mmol) and DMF (2 mL) was heated at 60° C. for 4 h under N₂. The mixture was cooled down to rt, diluted with EtOAc (20 mL) and water (5 mL). The organics were separated, washed with brine, dried, and filtered. The solvents were removed to give crude product. The crude was purified on silica gel (EtOAc:Hexanes=1:2) to give desired product. MS calcd. for [M+H]⁺ C₂₀H₂₄BrN₂O₃S: 451.0; found: 451.0.

Step D 2-(Methylsulfonyl)-6-(1-(4-(4,4,5,5-tetramethyl-1,3,2-dioxaborolan-2-yl)phenyl)pyrrolidin-3-yloxy)-1,2,3,4-tetrahydroisoquinoline (222d) A mixture of 6-(1-(4-bromophenyl)pyrrolidin-3-yloxy)-2-(methylsulfonyl)-1,2,3,4-tetrahydroisoquinoline (200 mg, 0.44 mmol), 4,4,4′,4′,5,5,5′,5′-octamethyl-2,2′-bi(1,3,2-dioxaborolane) (150 mg, 0.59 mmol), KOAc (180 mg, 1.85 mmol), dppf (10 mg) and DMSO (5 mL) was degassed and heated at 80° C. for 3 h. It was cooled to rt, and EtOAc (20 mL) was added. The mixture was washed with brine, dried and filtered. The solvents were removed under reduced pressure to give the crude product. The crude was purified on silica gel (EtOAc:Hexanes=1:3) to give the desired product. MS calcd. for [M+H]⁺ C₂₆H₃₆BN₂O₅S: 499.2; found: 499.2.

Step E 6-(1-(4-(5-Ethylpyrimidin-2-yl)phenyl)pyrrolidin-3-yloxy)-2-(methylsulfonyl)-1,2,3,4-tetrahydroisoquinoline (222) A mixture of 2-(methylsulfonyl)-6-(1-(4-(4,4,5,5-tetramethyl-1,3,2-dioxaborolan-2-yl)phenyl)pyrrolidin-3-yloxy)-1,2,3,4-tetrahydroisoquinoline (20 mg, 0.04 mmol), Na₂CO₃ (1 N, 1 mL), 5-ethyl-2-chloropyrimidine (20 mg, 0.14 mmol), (PPh₃)₄Pd (2 mg) and dioxane (3 mL) was heated at 160° C. in microwave for 10 min. It was cooled to rt, and EtOAc (10 mL) was added. The mixture was washed with brine, dried and filtered. The solvents were removed to afford the crude product. The crude was purified on silica gel (EtOAc:Hexanes=1:1) to give desired product. MS calcd. for [M+H]⁺ C₂₆H₃₁N₄O₃S: 479.2; found: 479.2.

### Example 226

### 6-(3-(4-(5-Ethylpyrimidin-2-yl)-3-fluorophenyl)propoxy)-2-(methylsulfonyl)-1,2,3,4-tetrahydroisoquinoline

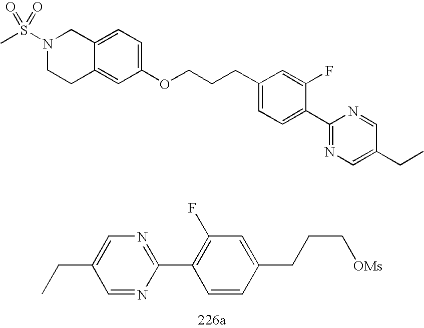

Step A 3-(4-(5-Ethylpyrimidin-2-yl)-3-fluorophenyl)propyl methanesulfonate (226a) The intermediate 226a was prepared in a manner similar to example 249 from 1-bromo-2-fluoro-4-iodobenzene. ¹H-NMR (400 MHz, CDCl₃) δ 8.71 (s, 2H), 7.98 (t, 1H, J=8.0 Hz), 7.07 (dq, 2H, J=1.6, 8.0 Hz), 4.25 (t, 2H, J=6.4 Hz), 3.02 (s, 3H), 2.82 (t, 2H, J=7.2 Hz), 2.71 (q, 2H, J=7.6 Hz), 2.15-2.08 (m, 2H), 1.33 (t, 3H, J=7.6 Hz); MS calcd. for [M+H]⁺ C₁₆H₁₉FN₂O₃S: 339.1; found: 338.8.

Step B The title compound 226 was synthesized according to the procedure described for the synthesis of example 146 from phenol 3 and mesylate 226a. ¹H-NMR (400 MHz, CDCl₃) δ 8.63 (s, 2H), 7.88 (t, 1H, J=8.0 Hz), 7.05-6.96 (m, 2H), 6.93 (d, 1H, J=8.4 Hz), 6.70 (dd, 1H, J=2.8, 8.8 Hz), 6.59 (d, 1H, J=2.4 Hz), 4.33 (s, 2H), 3.87 (t, 2H, J=6.0 Hz), 3.48 (t, 2H, J=6.0 Hz), 2.87 (t, 2H, J=5.6 Hz), 2.80 (t, 2H, J=7.2 Hz), 2.63 (q, 2H, J=7.6 Hz), 2.06 (quint, 2H, J=6.4 Hz), 1.26 (t, 3H, J=7.6 Hz); MS calcd. for [M+H]⁺ C₂₅H₂₈FN₃O₃S: 470.2; found: 470.2.

Examples 223-225 were synthesized by analogous methods from the corresponding phenols and mesylate 226a.

### Example 227

### 2-(5-(4-(3-(2-(Methylsulfonyl)-1,2,3,4-tetrahydroisoquinolin-6-yloxy)propyl)piperidin-1-yl)-2H-tetrazol-2-yl)ethanamine

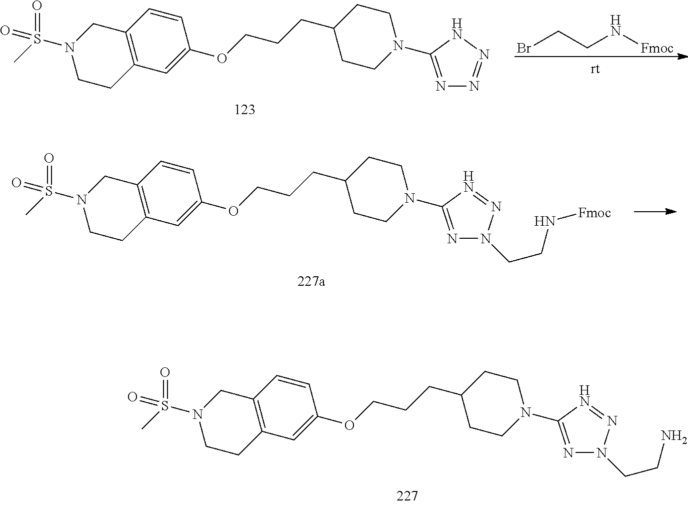

A mixture of Example 123 (21 mg, 0.05 mmol), (9H-fluoren-9-yl)methyl 2-bromoethylcarbamate (36 mg, 0.1 mmol) and K₂CO₃ (20 mg, 0.15 mmol) in DMF (1 mL) was stirred at rt overnight. Piperidine (0.5 mL) was added and the mixture was stirred for another hour. The reaction mixture was purified by HPLC to afford the product as a white solid. ¹H-NMR (400 MHz, CD₃CN) δ=7.97 (br s, 2H), 7.04 (d, J=8.1 Hz, 1H), 6.76 (dd, J=2.8, 8.4 Hz, 1H), 6.72 (d, J=2.4 Hz, 1H), 4.75 (t, J=5.6 Hz, 2H), 4.31 (s, 2H), 4.02 (m, 2H), 3.95 (t, J=6.4 Hz, 2H), 3.49 (t, J=5.6 Hz, 2H), 3.45 (t, J=6.0 Hz, 2H), 2.93-2.86 (m, 4H), 2.80 (s, 3H), 1.80-1.76 (m, 4H), 1.53 (m, 1H), 1.43-1.38 (m, 2H), 1.24 (ddd, J=4.0, 12.4, 24.2 Hz, 2H); MS calcd. for [M+H]⁺ C₂₁H₃₄N₇O₃S: 464.2; found: 464.2.

### Example 228

### Methyl 2-(5-(4-(3-(2-(methylsulfonyl)-1,2,3,4-tetrahydroisoquinolin-6-yloxy)propyl)piperidin-1-yl)-2H-tetrazol-2-yl)acetate

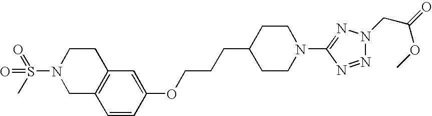

The title compound was prepared in a manner similar to Example 124 from Example 123 using appropriate starting materials.

### Example 229

### 6-(3-(1-(2-(2-Methoxyethyl)-2H-tetrazol-5-yl)piperidin-4-yl)propoxy)-2-(methylsulfonyl)-1,2,3,4-tetrahydroisoquinoline

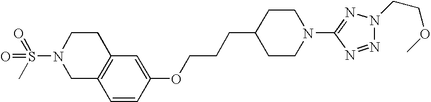

A mixture of Example 123 (42 mg, 0.1 mmol), bromoethanol methyl ether (12 uL, 0.12 mmol) and KOH (7 mg, 0.12 mmol) in 1-propanol (0.6 mL) was stirred in a seal vial at 100° C. overnight. The reaction mixture was purified by HPLC to afford 229 as a white solid. ¹H-NMR (400 MHz, CD₃CN) δ=7.04 (d, J=6.3 Hz, 1H), 6.76 (dd, J=2.8, 8.4 Hz, 1H), 6.72 (d, J=1.8 Hz, 1H), 4.53 (t, J=5.2 Hz, 2H), 4.31 (s, 2H), 3.99-3.93 (m, 4H), 3.81 (t, J=4.8 Hz, 2H), 3.45 (t, J=6.0 Hz, 2H), 3.26 (s, 3H), 2.92-2.83 (m, 4H), 2.81 (s, 3H), 1.80-1.76 (m, 4H), 1.51 (m, 1H), 1.40 (m, 2H), 1.24 (ddd, J=4.4, 12.8, 24.8 Hz, 2H); MS calcd. for [M+H]⁺ C₂₂H₃₅N₆O₄S: 479.2; found: 479.2.

### Example 230

### 2-(5-(4-(3-(2-(Methylsulfonyl)-1,2,3,4-tetrahydroisoquinolin-6-yloxy)propyl)piperidin-1-yl)-2H-tetrazol-2-yl)ethanol

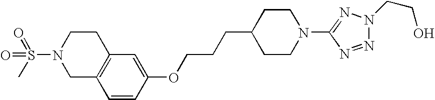

The title compound was prepared in a manner similar to Example 229 from Example 123 using appropriate starting materials.

### Example 231

### 6-(3-(1-Ethylpyrimidin-2-yl)piperidin-4-yl)propoxy)-2-(2-(methylsulfonyl)ethyl)-1,2,3,4-tetrahydroisoquinoline

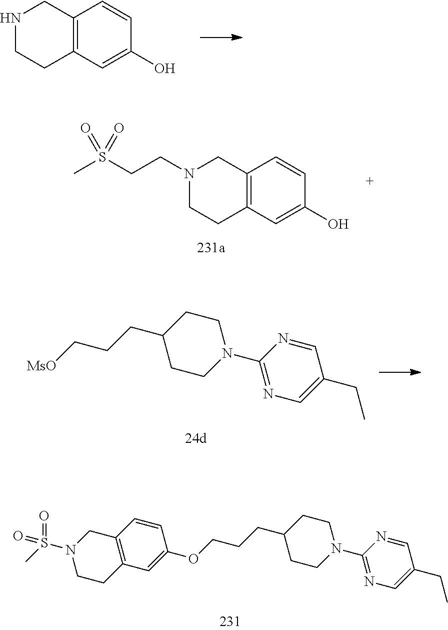

Step A 2-(2-(Methylsulfonyl)ethyl)-1,2,3,4-tetrahydroisoquinolin-6-ol To a suspension of 1,2,3,4-tetrahydroisoquinolin-6-ol hydrobromide (460 mg, 2 mmol) in ethanol was added NaH (80 mg, 2 mmol). The mixture was stirred for 10 min and methylsulfonylethene (2.5 mmol) was added and the mixture was stirred for additional 5 min. White precipitate was collected via filtration and air dried to give the desired product. MS calcd. for [M+H]⁺ C₁₂HR₈NO₃S: 256.1; found: 256.1.

Step B 6-(3-(1-(5-Ethylpyrimidin-2-yl)piperidin-4-yl)propoxy)-2-(2-(methylsulfonyl)ethyl)-1,2,3,4-tetrahydroisoquinoline A mixture of 2-(2-(methylsulfonyl)ethyl)-1,2,3,4-tetrahydroisoquinolin-6-ol (25 mg, 0.1 mmol), 24d (33 mg, 0.1 mmol), Cs₂CO₃ (65 mg, 0.2 mmol) in DMF (2 mL) was stirred for 5 h at rt. CHCl₃ (10 mL) was added and the organics were washed with water and brine. The organics were dried, filtered and the solvents were removed under reduced pressure. The crude thus obtained was purified on silica gel (EtOAc:Hexanes=2:1) to afford the desired product. MS calcd. for [M+H]⁺ C₂₆H₃₉N₄O₃S: 487.2; found: 487.2.

Example 232 was synthesized following the analogous method of the synthesis of example 231.

### Example 233

### 6-(3-(4′-Butylbiphenyl-4-yl)propoxy)-2-(methylsulfonyl)-1,2,3,4-tetrahydroisoquinoline

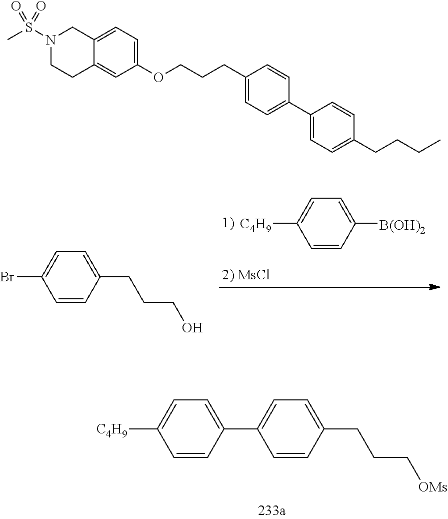

Step A 3-(4′-Butylbiphenyl-4-yl)propyl methanesulfonate (233a) The intermediate 233a was prepared in a manner similar to example 167 (Step C) from 3-(4-bromophenyl)propan-1-ol and example 146 (Step D). ¹H-NMR (400 MHz, CDCl₃) δ 7.53-7.48 (m, 4H), 7.25 (d, 4H, J=9.2 Hz), 4.26 (t, 2H, J=6.4 Hz), 3.01 (s, 3H), 2.79 (t, 2H, J=7.6 Hz), 2.65 (t, 2H, J=7.6 Hz), 2.15-2.08 (m, 2H), 1.67-1.60 (m, 2H), 1.44-1.34 (m, 2H), 0.95 (t, 3H, J=7.2 Hz).

Step B The title compound 233 was synthesized according to the procedure described for the synthesis of example 146 from phenol 3 and mesylate 233a. MS calcd. for [M+H]⁺ C₂₉H₃₅NO₃S: 478.2; found: 477.8.

### Example 234

### 6-(3-(4-(Benzyloxy)phenyl)propoxy)-2-(methylsulfonyl)-1,2,3,4-tetrahydroisoquinoline

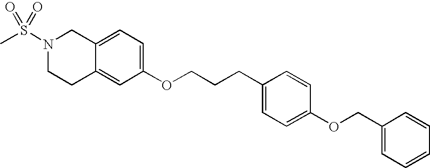

Example 234 was prepared by analogous methods described in example 146 (Step G) from the corresponding 3-(4-(benzyloxy)phenyl)propyl methanesulfonate (which was prepared by analogous methods described in example 146 (Step D) from the corresponding 3-[4-(benzyloxy)phenyl)-1-propanol) and phenol 3). ¹H-NMR (400 MHz, CDCl₃) δ 7.44-7.30 (m, 5H), 7.12 (d, 2H, J=8.8 Hz), 6.99 (d, 1H, J=8.4 Hz), 6.90 (d, 2H, J=8.4 Hz), 6.76 (dd, 1H, J=2.4, 8.4 Hz), 6.66 (d, 1H, J=2.4 Hz), 5.29 (s, 2H), 4.40 (s, 2H), 3.92 (t, 2H, J=6.4 Hz), 3.54 (t, 2H, J=6.0 Hz), 2.93 (t, 2H, J=6.0 Hz), 2.83 (s, 3H), 2.74 (t, 2H, J=7.6 Hz), 2.06 (quint, 2H, J=6.4 Hz); MS calcd. for [M+H]⁺ C₂₆H₂₉NO₄S: 452.2; found: 451.8.

### Example 235

### 2-(Methylsulfonyl)-6-(3-(1-(2-(2-(pyrrolidin-1-yl)ethyl)-2H-tetrazol-5-yl)piperidin-4-yl)propoxy)-1,2,3,4-tetrahydroisoquinoline

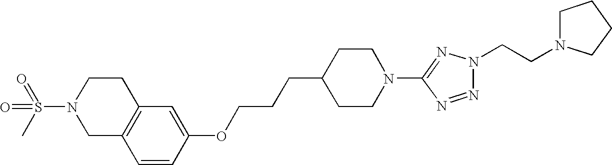

A mixture of Example 123 (16 mg, 0.038 mmol), 1-(2-chloroethyl)pyrrolidine hydrochloride (38 mg, 0.22 mmol) and Cs₂CO₃ (124 mg, 0.38 mmol) in DMF (1 mL) was stirred in a seal vial at 50° C. overnight. The reaction mixture filtered through a syringe filter and purified by HPLC to afford 235 as a white solid. MS calcd. for [M+H]⁺ C₂₅H₄₀N₇O₃S: 518.3, found 518.2 (M+1): ¹H-NMR (400 MHz, CD₃CN) δ=7.04 (d, J=7.6 Hz, 1H), 6.76 (dd, J=2.4, 8.4 Hz, 1H), 6.72 (d, J=2.4 Hz, 1H) 4.83 (t, J=6.4 Hz, 2H), 4.32 (s, 2H), 4.31 (s, 2H), 3.99 (m, 2H), 3.95 (t, J=6.4 Hz, 2H), 3.67 (t, J=6.0 Hz, 2H), 3.45 (t, J=6.0, 2H), 2.92-2.85 (m, 4H), 2.81 (s, 3H), 2.20 (m, 8H) 1.80-1.74 (m, 4H), 1.51 (m, 1H), 1.43-1.37 (m, 2H), 1.3 (dd, J=4.0, 12.4, 24.4 Hz, 2H); MS calcd. for [M+1]⁺ C₂₅H₄₀N₇O₃S 518.3; found: 518.2.

### Example 237

### 4-(2-(5-(4-(3-(2-(Methylsulfonyl)-1,2,3,4-tetrahydroisoquinolin-6-yloxy)propyl)piperidin-1-yl)-2H-tetrazol-2-yl)ethyl)morpholine

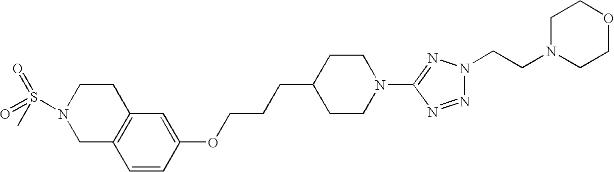

The title compound was prepared in a manner similar to Example 235 from Example 123 using appropriate starting materials.

### Example 239

### N,N-Dimethyl-3-(5-(4-(3-(2-(methylsulfonyl)-1,2,3,4-tetrahydroisoquinolin-6-yloxy)propyl)piperidin-1-yl)-2H-tetrazol-2-yl)propan-1-amine

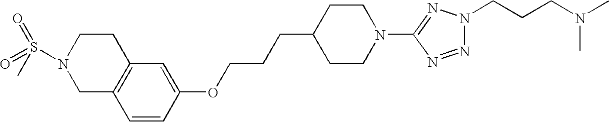

The title compound was prepared in a manner similar to Example 235 from Example 123 using appropriate starting materials.

### Example 240

### 4-(3-(2-(Methylsulfonyl)-1,2,3,4-tetrahydroisoquinolin-6-yloxy)propyl)phenyl dimethylcarbamate

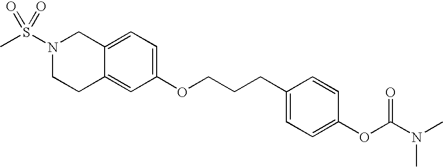

To a mixture of 219a (5 mg, 0.014 mmol) and K₂CO₃ (10 mg, 0.07 mmol) in anhydrous DMF (2 mL) was added dimethylcarbamyl chloride (4 uL, 0.04 mmol) at rt. The mixture was stirred at rt for 1 h, and then extracted with EtOAc. The organics were washed with water (3×5 mL), brine, dried over Na₂SO₄ and filtered. Removal of the solvent under reduced pressure and purification of the crude by silica gel column chromatography (EtOAc/hexanes) afforded the title compound 240. ¹H-NMR (400 MHz, CDCl₃) δ 7.18 (d, 2H, J=8.4 Hz), 7.03-6.98 (m, 3H), 6.76 (dd, 1H, J=2.8, 8.4 Hz), 6.66 (d, 1H, J=2.4 Hz), 4.40 (s, 2H), 3.92 (t, 2H, J=6.0 Hz), 3.54 (t, 2H, J=6.0 Hz), 3.09 (brs, 3H), 3.01 (brs, 3H), 2.94 (t, 2H, J=6.0 Hz), 2.79 (t, 2H, J=7.2 Hz), 2.11-2.05 (m, 2H); MS calcd. for [M+H]⁺ C₂₂H₂₈N₂O₅S: 433.2; found: 432.8.

### Example 243

### N,N-Diethyl-2-(5-(4-(3-(2-(methylsulfonyl)-1,2,3,4-tetrahydroisoquinolin-6-yloxy)propyl)piperidin-1-yl)-2H-tetrazol-2-yl)ethanamine

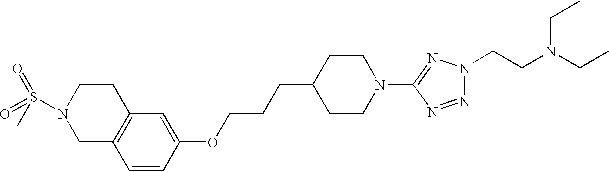

The title compound was prepared in a manner similar to Example 235 from Example 123 using appropriate starting materials.

### Example 244

### 2-(Methylsulfonyl)-6-(3-(1-(2-(2-(piperidin-1-yl)ethyl)-2H-tetrazol-5-yl)piperidin-4-yl)propoxy)-1,2,3,4-tetrahydroisoquinoline

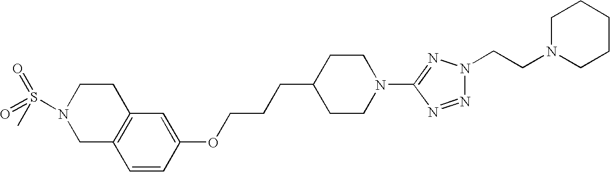

The title compound was prepared in a manner similar to Example 235 from Example 123 using appropriate starting materials.

### Example 245

### 6-(3-(1-(2-(2-(4-Isopropylypiperazin-1-yl)ethyl)-2H-tetrazol-5-yl)piperidin-4-yl)propoxy)-2-(methylsulfonyl)-1,2,3,4-tetrahydroisoquinoline

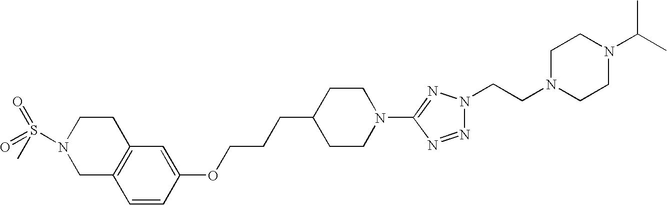

The title compound was prepared in a manner similar to Example 235 from Example 123 using appropriate starting materials.

### Example 246

### 1-Methylcyclopropyl 4-(2-(3-(2-(methylsulfonyl)-1,2,3,4-tetrahydroisoquinolin-6-yl)-5,6-dihydro-1,4-dithiin-2-yl)ethyl)piperidine-1-carboxylate

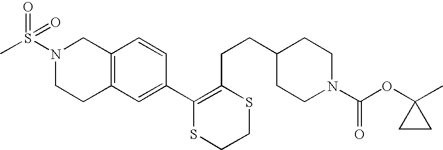

Intermediate 246b: 1-Methylcyclopropyl 4-(3-(2-(2-(methylsulfonyl)-1,2,3,4-tetrahydroisoquinolin-6-yl)-1,3-dithiolan-2-yl)propyl)piperidine-1-carboxylate

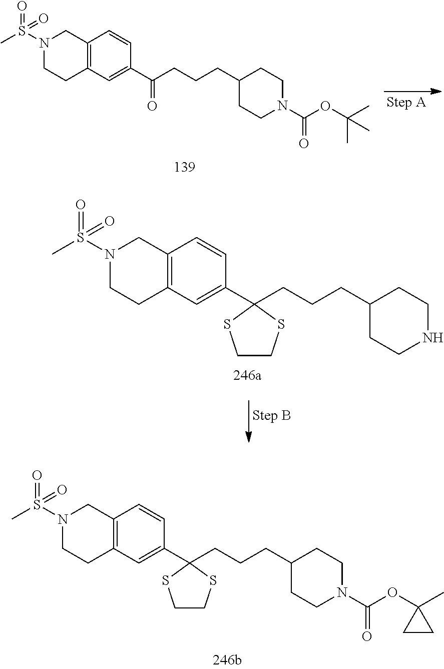

Step A 2-(Methylsulfonyl)-6-(2-(3-(piperidin-4-yl)propyl)-1,3-dithiolan-2-yl)-1,2,3,4-tetrahydroisoquinoline (246a) was prepared from Example 139 (260 mg, 0.56 mmol) according to the procedure described for the preparation of 75a. MS calcd. for [M+H]⁺ C₂₁H₃₃N₂O₂S₃: 441.2 found: 441.1. The aqueous phase was then extracted with EtOAc. The combined organics were dried (Na₂SO₄) and concentrated in vacuo to afford additional 246a.

Step B 1-Methylcyclopropyl 4-(3-(2-(2-(methylsulfonyl)-1,2,3,4-tetrahydroisoquinolin-6-yl)-1,3-dithiolan-2-yl)propyl)piperidine-1-carboxylate (246b) was prepared from 246a (115 mg, 0.26 mmol) according to the procedure described for Example 60. The compound was used in the next step without further purification. MS calcd. for [M+H]⁺ C₂₆H₃₉N₂O₄S₃: 539.2; found: 538.7.

Compound 246b (73 mg, 0.13 mmol) was dissolved in CH₂Cl₂ (0.1 mL). DMSO (30 μL, 0.42 mmol) and WCl₆ (43 mg, 0.11 mmol) were then added and the mixture stirred for 2 h at room temperature. The solvent was evaporated and the crude purified by flash chromatography (hexane/EtOAc) to afford Example 246. ¹H-NMR (600 MHz, CDCl₃) δ=7.10-7.06 (m, 3H), 4.47 (s, 2H), 4.10-3.83 (m, 2H), 3.58 (t, J=6.0 Hz, 2H), 3.28 (s, 4H), 2.98 (t, J=6.0 Hz, 2H), 2.88 (s, 3H), 2.65-2.57 (m, 2H), 2.16-2.13 (m, 2H), 1.54 (s, 3H), 1.48-1.43 (m, 2H), 1.42-1.39 (m, 2H), 1.26-1.22 (m, 1H), 1.00-0.88 (m, 2H), 0.86-0.83 (m, 2H), 0.63-0.60 (m, 2H); MS calcd. for [M+H]⁺ C₂₆H₃₇N₂O₄S₃: 537.2; found: 537.2.

### Example 247

### 2-(Methylsulfonyl)-6-(3-(4-(pyrazin-2-yloxy)phenyl)propoxy)-1,2,3,4-tetrahydroisoquinoline

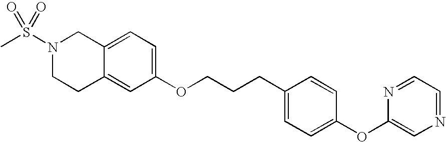

To a reaction tube was charged with 4-(3-(2-(methylsulfonyl)-1,2,3,4-tetrahydroisoquinolin-6-yloxy)propyl)phenol (219a) (20 mg, 0.055 mmol), iodopyrazine (6 uL, 0.066 mmol), CuI (11 mg, 0.055 mmol), N,N-dimethylglycine (6 mg, 0.055 mmol), Cs₂CO₃ (36 mg, 0.11 mmol) and 1,4-dioxane (1 mL). The mixture was degassed and stirred at 120° C. overnight. It was then cooled to rt, filtered and rinsed with ethyl acetate. Solvents were removed under reduced pressure and the crude was purified by silica gel flash column chromatography (EtOAc/hexanes) to afford the title compound 247 as a white solid. ¹H-NMR (400 MHz, CDCl₃) δ 8.41 (d, 1H, J=1.2 Hz), 8.25 (d, 1H, J=2.8 Hz), 8.12 (dd, 1H, J=1.2, 2.8 Hz), 7.27 (d, 2H, J=7.6 Hz), 7.08 (d, 2H, J=8.4 Hz), 7.00 (d, 1H, J=8.8 Hz), 6.77 (dd, 1H, J=2.4, 8.4 Hz), 6.68 (d, 1H, J=2.4 Hz), 4.40 (s, 2H), 3.97 (t, 2H, J=6.4 Hz), 3.55 (t, 2H, J=6.0 Hz), 2.95 (t, 2H, J=6.0 Hz), 2.85-2.81 (m, 5H), 2.15-2.08 (m, 2H); MS calcd. for [M+H]⁺ C₂₃H₂₅N₃O₄S: 440.2; found: 439.8.

Examples 248, 252-257 were synthesized by analogous methods from derivative 219a and appropriate heteroaryl bromides or iodides.

### Example 249

### 6-(3-(4-(5-Ethylpyrimidin-2-yl)-3-methylphenyl)propoxy)-2-(methylsulfonyl)-1,2,3,4-tetrahydroisoquinoline

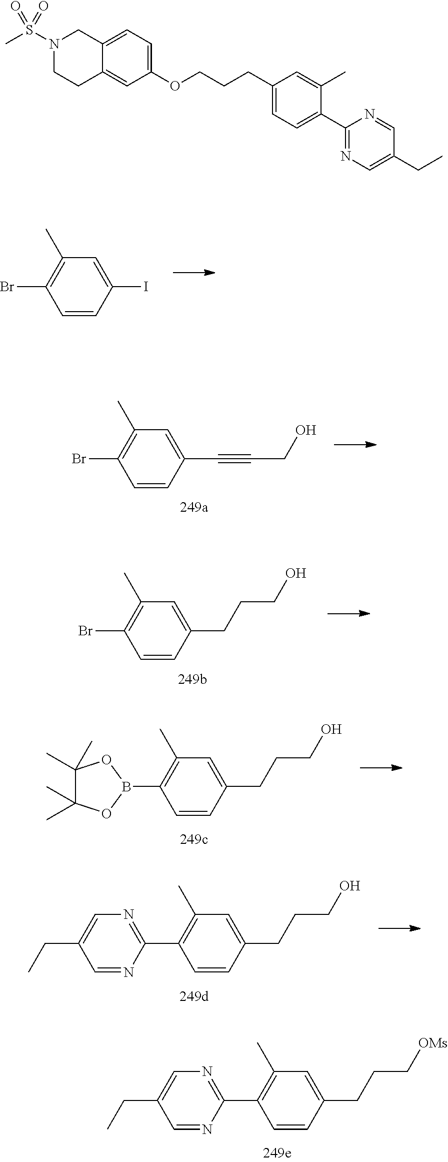

Step A 3-(4-Bromo-3-methylphenyl)propan-1-ol (249b) Intermediate 249b was prepared in a manner similar to example 266 (Step A and B) from 2-bromo-5-iodotoluene. 249a: ¹H-NMR (400 MHz, CDCl₃) δ 7.48 (d, 1H, J=5.6 Hz), 7.31 (d, 1H, J=1.2 Hz), 7.11 (dd, 1H, J=1.2, 6.4 Hz), 4.48 (s, 2H), 2.37 (s, 3H). 249b: ¹H-NMR (400 MHz, CDCl₃) δ 7.42 (d, 2H, J=8.0 Hz), 7.07 (d, 1H, J=2.0 Hz), 6.88 (dd, 1H, J=2.0, 8.0 Hz), 3.67 (t, 2H, J=6.4 Hz), 2.64 (t, 2H, J=7.2 Hz), 2.37 (s, 3H), 1.95 (m, 2H).

Step B 3-(4-(5-ethylpyrimidin-2-yl)-3-methylphenyl)propan-1-ol (249d) Compound 249d was prepared in a manner similar to example 167 (Step B and C) from 249b. MS calcd. for [M+H]⁺ C₁₆H₂₀N₂O₃S: 257.2; found: 257.2.

Step C 3-(4-(5-Ethylpyrimidin-2-yl)-3-methylphenyl)propyl methanesulfonate (249e) Intermediate 249e was prepared by analogous methods described in example 146 (Step D) from the corresponding hydroxyl 249d. ¹H-NMR (400 MHz, CDCl₃) δ 8.69 (s, 2H), 7.72 (d, 1H, J=8.0 Hz), 7.13-7.11 (m, 2H), 4.24 (t, 2H, J=6.4 Hz), 3.00 (s, 3H), 2.77 (t, 2H, J=7.6 Hz), 2.71 (q, 2H, J=7.6 Hz), 2.52 (s, 3H), 2.10 (quint, 2H, J=6.8 Hz), 1.34 (t, 3H, J=7.6 Hz); MS calcd. for [M+H]⁺ C₁₇H₂₂N₂O₃S: 335.1; found: 335.1.

Step D 6-(3-(4-(5-Ethylpyrimidin-2-yl)-3-methylphenyl)propoxy)-2-(methylsulfonyl)-1,2,3,4-tetrahydroisoquinoline The title compound 249 was synthesized according to the procedure described for the synthesis of example 146 from phenol 3 and mesylate 249e. ¹H-NMR (400 MHz, CDCl₃) δ 8.73 (s, 2H), 7.74 (d, 1H, J=5.2 Hz), 7.16-7.14 (m, 2H), 6.99 (d, 1H, J=5.6 Hz), 6.77 (dd, 1H, J=1.6, 5.6 Hz), 6.66 (d, 1H, J=1.6 Hz), 4.40 (s, 2H), 3.94 (t, 2H, J=4.0 Hz), 3.55 (t, 2H, J=4.0 Hz), 2.94 (t, 2H, J=3.6 Hz), 2.84-2.81 (m, 5H), 2.73 (q, 2H, J=5.2 Hz), 2.52 (s, 3H), 2.12 (quint, 2H, J=4.4 Hz), 1.35 (t, 3H, J=5.2 Hz); MS calcd. for [M+H]⁺ C₂₆H₃₁N₃O₃S: 466.2; found: 466.2.

### Example 258

### 6-((7-(5-Ethylpyrimidin-2-yl)-5,6,7,8-tetrahydroimidazo[1,2-a]pyrazin-2-yl)methoxy)-2-(methylsulfonyl)-1,2,3,4-tetrahydroisoquinoline

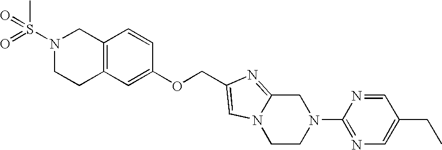

Intermediate 258c: 2-(Chloromethyl)-7-(5-ethylpyrimidin-2-yl)-5,6,7,8-tetrahydroimidazo[1,2-a]pyrazine

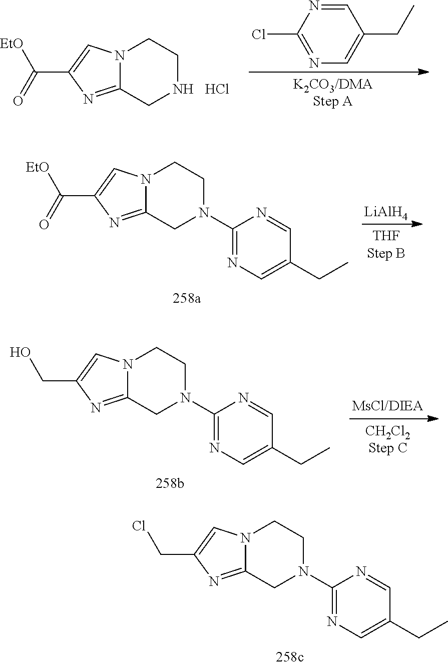

Step A Ethyl 7-(5-ethylpyrimidin-2-yl)-5,6,7,8-tetrahydroimidazo[1,2-a]pyrazine-2-carboxylate 258a was prepared from commercially available ethyl 5,6,7,8-tetrahydroimidazo[1,2-a]pyrazine-2-carboxylate hydrochloride (500 mg, 2.16 mmol) and 2-chloro-5-ethylpyrimidine (0.32 mL, 2.60 mmol) according to the procedure described for 24c. ¹H-NMR (400 MHz, CDCl₃) δ=8.24 (s, 2H), 7.58 (s, 1H), 5.06 (s, 2H), 4.37 (q, J=7.1 Hz, 2H), 4.28 (m, 2H), 4.13 (m, 2H), 2.50 (q, J=7.6 Hz, 2H), 1.39 (t, J=7.1 Hz, 3H), 1.20 (t, J=7.6 Hz, 3H); MS calcd. for [M+H]⁺ C₁₅H₂₀N₅O₂: 302.1; found: 302.2.

Step B Ethyl 7-(5-ethylpyrimidin-2-yl)-5,6,7,8-tetrahydroimidazo[1,2-a]pyrazine-2-carboxylate (258a) (91 mg, 0.30 mmol) was dissolved in THF (5 mL) and the mixture was cooled to 0° C. A solution of LiAlH₄ in THF (1N, 0.46 mL, 0.45 mmol) was added and the mixture was stirred for 10 min at 0° C., then the reaction was quenched by dropwise addition of H₂O. The mixture was extracted with EtOAc (20 mL), washed with brine (10 mL), dried (MgSO₄), filtered and concentrated to provide (7-(5-ethylpyrimidin-2-yl)-5,6,7,8-tetrahydroimidazo[1,2-a]pyrazin-2-yl)methanol (258b). The compound was used in the next step without further purification. MS calcd. for [M+H]⁺ C₁₃H₁₈N₅O: 260.1; found: 260.1.

Step C (7-(5-Ethylpyrimidin-2-yl)-5,6,7,8-tetrahydroimidazo[1,2-a]pyrazin-2-yl)methanol (258b) (55 mg, 0.21 mmol) was dissolved in DCM (5 mL), then diisopropylethylamine (73 μL, 0.42 mmol) and methanesulfonyl chloride (39 μL, 0.25 mmol) were added and the mixture was stirred at rt for 1 h. The mixture was diluted with sat. aq. NaHCO₃ (10 mL) and extracted with DCM (10 mL). The organic layer was combined, washed with brine, dried (MgSO₄), filtered, and concentrated to provide 258c. MS calcd. for [M+H]⁺ C₁₃H₁₇ClN₅: 278.1; found: 278.2.

Intermediate 3 (16 mg, 0.07 mmol), 258c (20 mg, 0.07 mmol), and Cs₂CO₃ were heated in ACN at 80° C. for 12 h. The mixture was cooled, filtered, concentrated, and purified by reversed-phase HPLC (water-acetonitrile gradient with TFA as ion-pairing reagent) to afford Example 258 as a white solid. ¹H-NMR (400 MHz, CDCl₃) δ=8.30 (s, 2H), 7.10 (s, 1H), 7.03 (d, J=8.4 Hz, 1H), 6.83 (dd, J=2.4, 8.4 Hz, 1H), 6.78 (d, J=2.4 Hz, 1H), 5.36 (s, 2H), 5.19 (s, 2H), 4.42 (s, 2H), 4.38 (m, 2H), 4.21 (m, 2H), 3.5 (m, 2H), 2.97 (m, 2H), 2.86 (s, 3H), 2.55 (q, J=7.6 Hz, 2H), 1.24 (t, J=7.6 Hz, 3H); MS calcd. for [M+H]⁺ C₂₃H₂₉N₆O₃S: 469.2; found: 469.2.

### Example 259

### 3-(3-(2-(Methylsulfonyl)-1,2,3,4-tetrahydroisoquinolin-6-yloxy)propyl)phenyl methanesulfonate

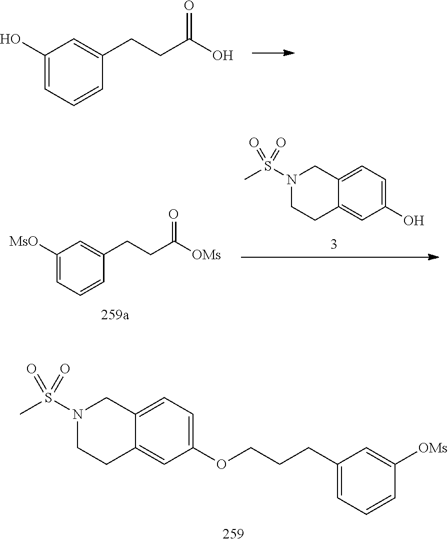

Step A 3-(3-(Methylsulfonyloxy)phenyl)propyl methanesulfonate (259a) To a solution of 3-(3-hydroxyphenyl)propionic acid (2 g, 12 mmol) in anhydrous THF (20 mL) was added dropwise a solution of BH₃ in THF (1 M, 24 mL, 24 mmol) at −10° C. After the completion, the mixture was warmed up to rt and stirred overnight. It was then cooled back to 0° C. and water was slowly added. The mixture was extracted with EtOAc. Organics were combined, washed with sat. aq. NaHCO₃, dried (Na₂SO₄) and filtered. Removal of the solvents under reduced pressure afforded crude 3-(3-hydroxypropyl)phenol.

Crude 3-(3-hydroxypropyl)phenol was dissolved in dichloromethane. The solution was cooled to 0° C. and Et₃N (2 mL) was added followed by addition of methanesulfonyl chloride (1.4 g). The mixture was stirred for 3 h and then quenched with water. The organics were washed with brine, dried and filtered. Solvents were removed under reduce pressure and the crude was purified on silica gel column to afford 259a. ¹H-NMR (400 MHz, CDCl₃) δ 7.38-7.34 (m, 1H), 7.18-7.13 (m, 3H), 4.23 (t, 2H, J=6.0 Hz), 3.16 (s, 3H), 3.01 (s, 3H), 2.80 (t, 2H, J=7.2 Hz), 2.12-2.06 (m, 2H)); MS calcd. for [M+H]⁺ C₁₁H₁₆O₆S₂: 309.0; found: 309.0.

Step B Example 259 was prepared by analogous methods described in example 146 (Step G) from the corresponding dimesylate 259a and phenol 3. MS calcd. for [M+H]⁺ C₂₀H₂₅NO₆S₂: 440.1; found: 440.0.

### Example 260

### 4-(3-(2-(Methylsulfonyl)-1,2,3,4-tetrahydroisoquinolin-6-yloxy)propyl)phenol

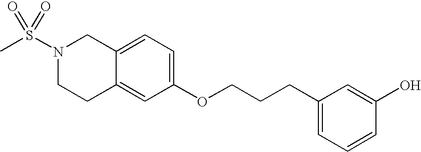

Example 260 was prepared by analogous methods described in Example 1 (Step B) from Example 259. MS calcd. for [M+H]⁺ C₁₉H₂₃NO₄S: 362.1; found: 362.1.

### Example 261

### tert-Butyl 4-(3-(2-(methylsulfonyl)-1,2,3,4-tetrahydroisoquinolin-6-yloxy)propyl)piperazine-1-carboxylate

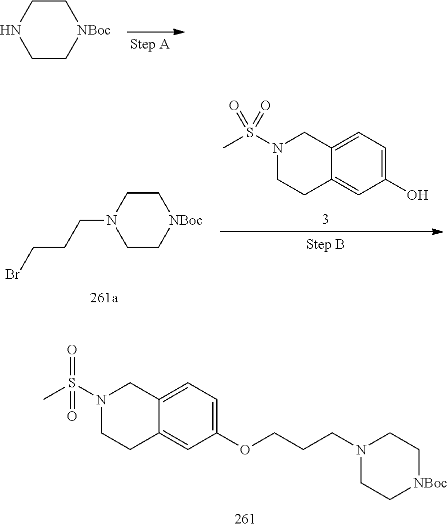

Step A A mixture of 1-Boc-piperazine (0.5 g, 2.7 mmol), 1,3-dibromopropane (2.75 mL, 27 mmol) and K₂CO₃ (1.9 g, 13.5 mmol) in 1,4-dioxane (20 mL) was heated at 60° C. overnight. The salts were filtered, and the filtrate was concentrated in vacuo. Purification of the crude by silica gel flash column chromatography (EtOAc/hexanes) gave the desired intermediate 261a as a solid. ¹H-NMR (400 MHz, CDCl₃) δ 3.47 (t, 2H, J=6.4 Hz), 3.43 (brs, 4H), 2.49 (t, 2H, J=6.4 Hz), 2.39 (br s, 4H), 2.07-2.02 (m, 2H), 1.46 (s, 9H); MS calcd. for [M+H]⁺ C₁₂H₂₃BrN₂O₂: 307.1; found: 307.0.

Step B A reaction vessel was charged with 2-(methylsulfonyl)-1,2,3,4-tetrahydroisoquinolin-6-ol (3) (300 mg, 1.3 mmol), 261a (419 mg, 1.4 mmol), Cs₂CO₃ (845 mg, 2.6 mmol) and acetonitrile (10 mL). The mixture was stirred at 80° C. for 42 h. It was filtered and rinsed with CH₂Cl₂. The organics were combined, and the solvents were removed under reduced pressure to give crude product. The crude was purified by silica gel flash column chromatography (EtOAc/hexanes) to afford the title compound 261 as a white solid. MS calcd. for [M+H]⁺ C₂₂H₃₅N₃O₅S: 454.2; found: 454.2.

### Example 262

### 4-(5-Ethylpyrimidin-2-yl)-1-(3-(2-(methylsulfonyl)-1,2,3,4-tetrahydroisoquinolin-6-yloxy)propyl)piperazin-2-one

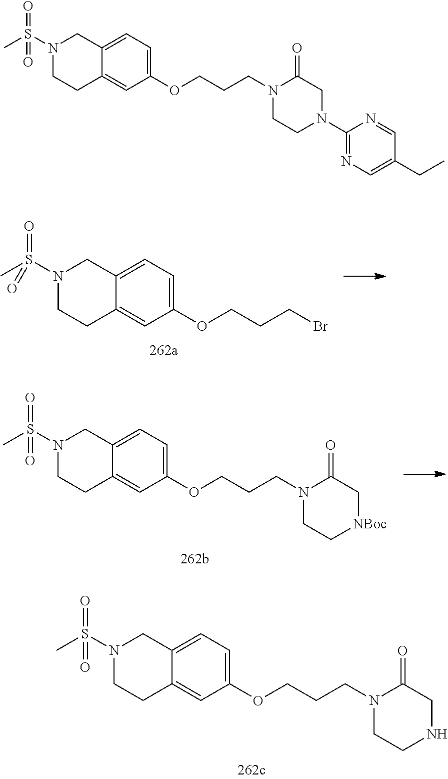

Step A 6-(3-Bromopropoxy)-2-(methylsulfonyl)-1,2,3,4-tetrahydroisoquinoline (262a) A mixture of phenol 3 (0.5 g, 2.2 mmol), 1,3-dibromopropane (2.2 mL, 22 mmol) and K₂CO₃ (0.9 g, 6.6 mmol) in DMF (10 mL) was stirred at rt overnight. The mixture was diluted with EtOAc and water. The mixture was extracted with EtOAc and organics were combined, washed with sat. aqueous NH₄Cl, water, brine, dried (Na₂SO₄), filtered. Solvents were removed under reduced pressure and the crude was purified by silica gel column chromatography (EtOAc/hexanes) to afford 262a. ¹H-NMR (400 MHz, CDCl₃) δ 7.01 (d, 1H, J=8.8 Hz), 6.77 (dd, 1H, J=2.8, 8.4 Hz), 6.69 (d, 1H, J=2.4 Hz), 4.40 (s, 2H), 4.09 (t, 2H, J=6.0 Hz), 3.60 (t, 2H, J=6.4 Hz), 3.55 (t, 2H, J=6.0 Hz), 2.95 (t, 2H, J=6.0 Hz), 2.83 (s, 3H), 2.31 (quint, 2H, J=6.4 Hz); MS calcd. for [M+H]⁺ C₁₃H₁₈BrNO₃S: 348.0; found: 348.0.

Step B tert-Butyl 4-(3-(2-(methylsulfonyl)-1,2,3,4-tetrahydroisoquinolin-6-yloxy)propyl)-3-oxopiperazine-1-carboxylate (262b) To a solution of 1-Boc-3-oxopiperazine (260 mg, 1.3 mmol) in DMF (10 mL) was added NaH (60% in mineral oil, 66 mg, 1.74 mmol) at 0° C. The mixture was stirred for 30 min at 0° C. Then 262a (300 mg, 0.86 mmol) was added in one portion and the resulting mixture was stirred at rt overnight. The mixture was diluted with EtOAc and then water was slowly added. The mixture was extracted with EtOAc and organics were combined, washed with sat. aq NH₄Cl, water, brine, dried (Na₂SO₄) and filtered. Solvents were removed under reduced pressure and the crude was purified by silica gel column chromatography (EtOAc/hexanes) to afford 262b as a white solid. MS calcd. for [M+H]⁺ C₂₂H₃₃N₃O₆S: 468.2; found: 412.1 [M-tBu].

Step C 1-(3-(2-(Methylsulfonyl)-1,2,3,4-tetrahydroisoquinolin-6-yloxy)propyl)piperazin-2-one (262c) Compound 262b was treated with 25% TFA in a similar manner to that described in example 215 to afford 262c as a solid. ¹H-NMR (400 MHz, CDCl₃) δ 6.99 (dd, 1H, J=8.4 Hz), 6.75 (dd, 1H, J=2.8, 8.4 Hz), 6.67 (d, 1H, J=2.8 Hz), 4.40 (s, 2H), 3.98 (t, 2H, J=6.4 Hz), 3.58-3.52 (m, 6H), 3.36 (t, 2H, J=5.2 Hz), 3.08 (t, 2H, J=5.6 Hz), 2.94 (t, 2H, J=6.0 Hz), 2.83 (s, 3H), 2.07 (quint, 2H, J=6.4 Hz); MS calcd. for [M+H]⁺ C₁₇H₂₅N₃O₄S: 368.2; found: 368.1.

Step D The title compound 262 was synthesized according to the procedure described for the synthesis of example 216. ¹H-NMR (400 MHz, CDCl₃) δ 8.21 (s, 2H), 6.98 (d, 1H, J=8.4 Hz), 6.73 (dd, 1H, J=2.4, 8.4 Hz), 6.66 (d, 1H, J=2.4 Hz), 4.38 (d, 2H, J=8.0 Hz), 4.37 (s, 2H), 4.03-3.97 (m, 4H), 3.63 (t, 2H, J=6.8 Hz), 3.54 (t, 2H, J=6.0 Hz), 3.47 (t, 2H, J=5.6 Hz), 2.93 (t, 2H, J=5.6 Hz), 2.83 (s, 3H), 2.49 (q, 2H, J=7.6 Hz), 2.09 (quint, 2H, J=6.4 Hz), 1.20 (t, 3H, J=7.6 Hz); MS calcd. for [M+H]⁺ C₂₃H₃₁N₅O₄S: 474.2; found: 474.1.

### Example 263

### tert-Butyl 4-(5-hydroxy-4-(2-(methylsulfonyl)-1,2,3,4-tetrahydroisoquinolin-6-yl)pentyl)piperidine-1-carboxylate

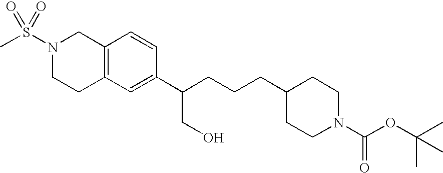

Example 220 (20 mg, 0.04 mmol) was dissolved in EtOH (10 mL) and acetic acid (1 mL). The solution was subjected to hydrogenolysis at 70° C. (70 atm, H-Cube®, with 10% palladium black on charcoal as catalyst). Evaporation, purification of the crude by reversed-phase HPLC and lyophilization of the product afforded Example 263 as a white solid. ¹H-NMR (400 MHz, CD₃CN) δ=7.06 (m, 3H), 4.47 (m, 1H), 4.36 (s, 2H), 3.95 (d, J=12.6 Hz, 2H), 3.57 (dd, J=6.5, 1.8 Hz, 2H), 3.47 (t, J=5.0 Hz, 2H), 2.93 (t, J=6.2 Hz, 2H), 2.82 (s, 3H), 2.63 (br s, 2H), 1.60-1.79 (m, 2H), 1.54 (m, 2H), 1.39 (s, 9H), 1.30 (m, 2H), 1.18 (m, 4H), 0.91 (m, 2H); MS calcd. for [M+Na]⁺ C₂₅H₄₀NaN₂O₅S: 503.3; found: 503.3.

### Example 264

### 6-(4-(1-(5-Ethylpyrimidin-2-yl)piperidin-4-yloxy)pyridin-2-yl)-2-(methylsulfonyl)-1,2,3,4-tetrahydroisoquinoline

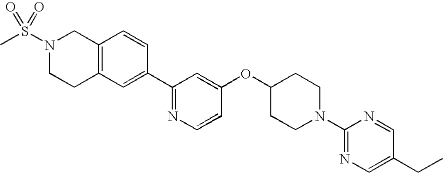

Intermediate 264c: 2-(4-(2-Chloropyridin-4-yloxy)piperidin-1-yl)-5-ethylpyrimidine

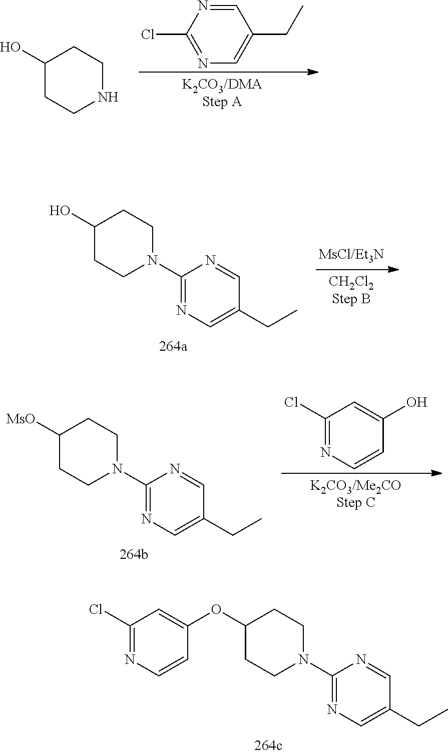

Step A 1-(5-ethylpyrimidin-2-yl)piperidin-4-ol (264a) was prepared from commercially available piperidin-4-ol (2.03 g, 20 mmol) and 2-chloro-5-ethylpyrimidine (2.43 mL, 20 mmol) similar to the procedure described for 24c, using K₂CO₃ (4.15 g, 30 mmol) as a base and heating to 180° C. for 10 min under microwave irradiation. ¹H-NMR (400 MHz, CDCl₃) δ=8.19 (s, 2H), 4.42 (m, 2H), 3.96 (m, 1H), 3.28 (m, 2H), 2.48 (q, J=7.5 Hz, 2H), 1.98 (m, 2H), 1.55 (m, 2H), 1.21 (t, J=7.5 Hz, 3H); MS calcd. for [M+H]⁺ C₁₁H₁₇N₃O: 208.1; found: 208.2.

Step B 1-(5-ethylpyrimidin-2-yl)piperidin-4-yl methanesulfonate (264b) was prepared from 264a (2.08 g, 10 mmol) according to the procedure described for 26b. The compound was used in the next step without further purification. ¹H-NMR (400 MHz, CD₃CN) δ=8.23 (s, 2H), 4.90 (m, 1H), 4.20 (m, 2H), 3.54 (m, 2H), 3.08 (s, 3H), 2.48 (q, J=7.5 Hz, 2H), 2.04 (m, 2H), 1.78 (m, 2H), 1.19 (t, J=7.5 Hz, 3H); MS calcd. for [M+H]⁺ C₁₂H₁₉N₃O₃S: 286.1; found: 286.1.

Step C A pressure vial was charged with 2-chloro-4-pyridinoyl (64 mg, 0.5 mmol), 264b (144 mg, 0.5 mmol), K₂CO₃ (103 mg, 0.75 mmol), and acetone (2.5 mL). The vial was sealed and heated to 130° C. for 15 min under microwave irradiation, then cooled to rt. The mixture was diluted with H₂O (20 mL), extracted with CH₂Cl₂ (3×20 mL), and the combined organics was washed with brine, dried (MgSO₄), and concentrated in vacuo. Purification of the crude by flash chromatography (EtOAc/hexanes=20% to 75%) afforded 2-(4-(2-chloropyridin-4-yloxy)piperidin-1-yl)-5-ethylpyrimidine (264c) as a pale yellow oil. ¹H-NMR (400 MHz, CD₃CN) δ=8.23 (s, 2H), 8.19 (d, J=5.8 Hz, 1H), 7.02 (d, J=2.2 Hz, 1H), 6.92 (dd, J=5.8, 2.2 Hz, 1H), 4.77 (m, 1H), 4.23 (m, 2H), 3.55 (m, 2H), 2.48 (q, J=7.5, 2H), 2.05 (m, 2H), 1.70 (m, 2H), 1.19 (t, J=7.5 Hz, 3H); MS calcd. for [M+H]⁺ C₁₆H₁₉ClN₄O: 319.1; found: 319.1.

Intermediate 264d: 2-(Methylsulfonyl)-6-(4,4,5,5-tetramethyl-1,3,2-dioxaborolan-2-yl)-1,2,3,4-tetrahydroisoquinoline

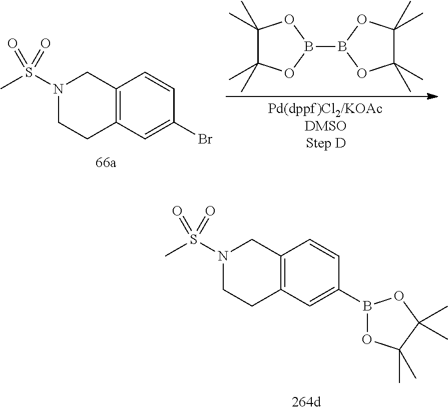

Step D 66a (2.04 g, 7 mmol), bis(pinacolato)diboron (1.88 g, 7.4 mmol), and potassium acetate (2.06 g, 21 mmol) were suspended in DMSO (50 mL) and the solution was degassed by vacuum/nitrogen purges. Pd(dppf)Cl₂ (250 mg, 5 mol %) was added, and the mixture was heated to 80° C. for 3 h and then cooled to rt. The solution was diluted with H₂O (100 mL) and extracted with EtOAc (3×50 mL). The combined organics were washed sequentially with H₂O (50 mL), 1N HCl (50 mL), and brine (20 mL), dried (MgSO₄), and concentrated in vacuo. Purification of the crude by flash chromatography (EtOAc/hexanes=30%) afforded 2-(methylsulfonyl)-1,2,3,4-tetrahydroisoquinolin-6-ylboronic acid pinacol ester (264d) as an off-white crystalline powder. ¹H-NMR (400 MHz, DMSO-D₆) δ=7.50 (s, 1H), 7.48 (d, J=7.5 Hz, 1H), 7.20 (d, J=7.5 Hz, 1H), 4.38 (s, 2H), 3.43 (t, J=6.0 Hz, 2H), 2.94 (s, 3H), 2.92 (t, J=6.0 Hz, 2H), 1.29 (s, 12H); MS calcd. for [M+H]⁺ C₁₆H₂₄BNO₄S: 338.2; found: 338.1.

A pressure vial was charged with 264c (86 mg, 0.27 mmol), 264d (103 mg, 0.31 mmol), Pd(dppf)Cl₂ (20 mg, 10 mol %), dioxane (2.7 mL), and a solution of degassed aqueous Cs₂CO₃ (2M, 0.27 mL). The mixture was heated to 150° C. for 20 min under microwave irradiation, cooled to rt, and partitioned between EtOAc (20 mL) and 2N Na₂CO₃ (10 mL). The aqueous layer was extracted with EtOAc (2×10 mL), and the combined organics was washed with brine, dried (MgSO₄), filtered and concentrated in vacuo. The resulting syrup was taken up in EtOAc, filtered through silica gel, and purified by reverse-phase HPLC (water-acetonitrile gradient with TFA as ion-pairing reagent). Repurification of the crude by flash chromatography (EtOAc/hexanes=50% to 80%) afforded Example 264 as a white solid. ¹H-NMR (400 MHz, CD₃CN) δ=8.62 (d, J=6.5 Hz, 1H), 8.23 (s, 2H), 7.81 (s, 1H), 7.78 (d, J=8.0 Hz, 1H), 7.50 (d, J=2.6 Hz, 1H), 7.34 (d, J=8.0 Hz, 1H), 7.19 (dd, J=6.5, 2.6 Hz, 1H), 5.01 (m, 1H), 4.49 (s, 2H), 4.23 (m, 2H), 3.60 (m, 2H), 3.54 (t, J=5.8 Hz, 2H), 3.05 (t, J=5.8 Hz, 2H), 2.86 (s, 3H), 2.47 (q, J=7.7 Hz, 2H), 2.10 (m, 2H), 1.77 (m, 2H), 1.17 (t, J=7.7 Hz, 3H); MS calcd. for [M+H]⁺ C₂₆H₃₁N₅O₃S: 494.2; found: 494.2.

### Example 265

### 2-(Methylsulfonyl)-6-(3-(6-phenylpyridin-3-yl)propoxy)-1,2,3,4-tetrahydroisoquinoline

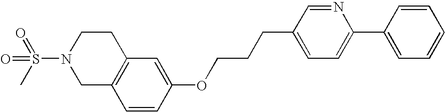

Intermediate 265c: 6-(3-(6-Chloropyridin-3-yl)propoxy)-2-(methylsulfonyl)-1,2,3,4-tetrahydroisoquinoline

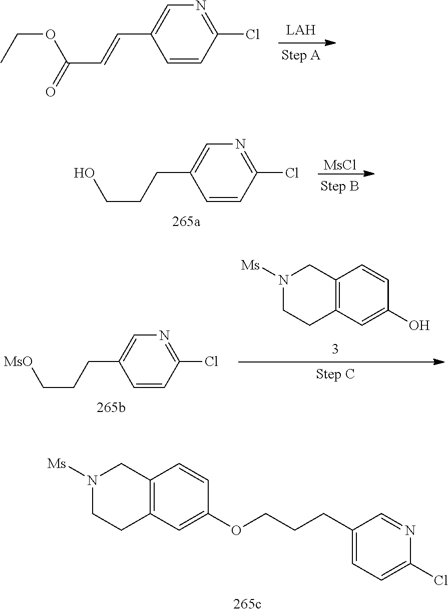

Step A A solution of (E)-ethyl 3-(6-chloropyridin-3-yl)acrylate (500 mg, 2.36 mmol) in anhydrous ether (10 mL) was added slowly (30 min) to a stirring solution of LiAlH₄ (1M in ether, 15 mL) at 0° C. After addition, the reaction mixture was stirred at 0° C. for 10 min and then rt for 50 min. The reaction mixture was diluted with ether (25 mL) and cooled in ice bath. The reaction was quenched with slow addition of water (0.2 mL) and then 1N NaOH (4×0.2 mL). The resulting mixture was stirred at rt for 15 min, followed by addition of MgSO₄. The mixture was stirred for another 15 min and filtered. The filter cake was washed with ether and the filtrate was concentrated in vacuo to give an oily residue. The crude was purified by flash chromatography (EtOAc/hexanes=50-100%) to afford 265a as a light yellow liquid. ¹H-NMR (400 MHz, CD₃CI) δ=8.63 (d, J=1.6 Hz, 1H), 7.85 (dd, J=2.0, 8.0 Hz, 1H), 7.32 (dd, J=0.8, 8.4 Hz, 1H), 4.51 (t, J=5.2 Hz, 1H), 2.13 (t, J=6.0 Hz, 1H), 1.62 (s, 2H); MS calcd. for [M+H]⁺ C₈H₁₁ClNO: 172.1; found: 172.0.

Step B To a solution of the alcohol 265a (60 mg, 0.35 mmol) and TEA (0.1 mL, 0.72 mmol) in DCM (5 mL) was added slowly MsCl (28 uL, 0.37 mmol) in 1 mL DCM at 0° C. After stirring at 0° C. for 2 h, the reaction was quenched with water (10 mL) and the resulting mixture was extracted with EtOAc (3×25 mL). The EtOAc extracts were combined, washed with brine (5 mL), dried (MgSO₄) and filtered. The solvents were removed to afford crude 265b. The crude was used directly in next step without further purification. MS calcd. for [M+H]⁺ C₉H₁₃ClNO₃S: 250.0; found: 250.0.

Step C A mixture of 2-(methylsulfonyl)-1,2,3,4-tetrahydroisoquinolin-6-ol (3) (35 mg, 0.15 mmol), mesylate 265b (41 mg, 0.17 mmol) and Cs₂CO₃ (73 mg, 0.22 mmol) in DMF (0.5 mL) was stirred at rt overnight. The solvent was evaporated to give a dark residue. Water (5 mL) was added and the mixture was extracted with EtOAc (4×15 mL). The EtOAc extracts were combined, washed with brine (3 mL), dried (Na₂SO₄), and filtered. The solvents were removed to afford as an off white solid. The solid was purified by flash column (EtOAc/hexanes=0-40%) to give the desired product as a white solid. ¹H-NMR (400 MHz, CD₃CI) δ=8.26 (br s, 1H), 7.52 (m, 1H), 7.27 (br s, 1H), 7.00 (d, J=8.4 Hz, 1H), 6.74 (dd, J=2.4, 8.4 Hz, 1H), 6.65 (d, J=2.0 Hz, 1H), 4.40 (s, 2H), 3.93 (t, J=5.6 Hz, 2H), 3.54 (t, J=6.0 Hz, 2H), 2.94 (t, J=7.2 Hz, 2H), 2.83 (s, 3H), 2.83 (m, 2H), 2.08 (quintet, J=6.4 Hz, 2H); MS calcd. for [M+H]⁺ C₁₈H₂₂ClN₂O₃S: 381.1; found: 381.0.

A mixture of 265c (10 mg, approx 0.021 mmol), phenylboronic acid (5 mg, 0.04), Pd₂ dba₃ (1 mg, 0.001 mmol), dicyclohexyl(2′,6′-dimethoxybiphenyl-3-yl)phosphine (1 mg, 0.002 mmol) and K₂CO₃ (22 mg, 0.16 mmol) in MeCN/H₂O (0.6 mL/0.3 mL) was degassed and then sealed in a pressure vial. The mixture was heated to 160° C. for 15 min under microwave irradiation. The reaction mixture was cooled to rt, purified by HPLC to give the Example 265 as a white powder. MS calcd. for [M+H]⁺ C₂₄H₂₇N₂O₃S: 423.2; found: 423.1.

### Example 266

### 2-(Methylsulfonyl)-6-(3-(5-phenylpyridin-2-yl)propoxy)-1,2,3,4-tetrahydroisoquinoline

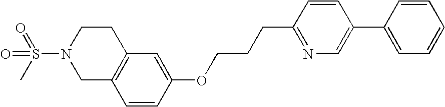

Intermediate 266b: 2-(3-(5-Bromopyridin-2-yl)propoxy)-6-(methylsulfonyl)-5,6,7,8-tetrahydropyrido[4,3-d]pyrimidine

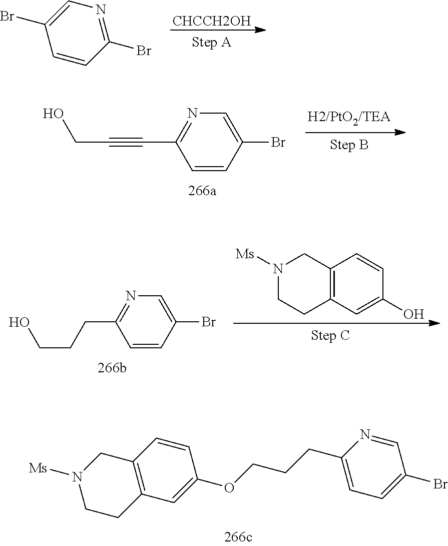

Step A A suspension of 2,5-dibromopyridine (4.68 g, 20 mmol), propargyl alcohol (1.18 g, 21 mmol), CuI (190 mg, 1.0 mmol), (PPh₃)₂PdCl₂(700 mg, 1.0 mmol) and TEA (14 mL, 50 mmol) in anhydrous ACN (100 mL) was degassed and sealed in a vial. After stirring at rt overnight, the mixture was filtered through a celite plug and the plug was washed with EtOAc. The filtrate was concentrated. The dark residue was taken up with water (25 mL) and extracted with EtOAc (3×50 mL). The EtOAc extracts were combined, washed with brine (15 mL), dried over MgSO₄ and concentrated. The crude was purified by flash chromatography (EtOAc/hexanes=20-50%) to afford the 266a as a yellow solid. ¹H-NMR (400 MHz, CD₃Cl) δ=8.63 (d, J=1.6 Hz, 1H), 7.85 (dd, J=2.0, 8.0 Hz, 1H), 7.32 (dd, J=0.8, 8.4 Hz, 1H), 4.51 (t, J=5.2 Hz, 1H), 2.13 (t, J=6.0 Hz, 1H), 1.62 (s, 2H); MS calcd. for [M+H]⁺ C₈H₇BrNO: 212.0; found: 211.9.

Step B A suspension of compound 266a (1.80 g, 8.5 mmol), PtO₂ (0.77 mL, 5.5 mmol) and TEA (0.77 mL, 5.5 mmol) in ethanol (35 mL) was stirred under hydrogen (1 atm) at rt for 7 h. The reaction mixture was filtered through a celite plug and the plug was washed with EtOAc. The filtered was concentrated to give a dark residue. The crude was purified by flash chromatography (EtOAc/hexanes=50-100%) to afford compound 266b as an amber colored oil. ¹H-NMR (400 MHz, CD₃Cl) δ=8.56 (d, J=2.4 Hz, 1H), 7.73 (dd, J=2.4, 8.4 Hz, 1H), 7.09 (d, J=8.0 Hz, 1H), 3.69 (t, J=6.0 Hz, 2H), 3.11 (br s, 1H), 2.91 (t, J=6.8 Hz, 2H), 1.98 (quintet, J=6.4 Hz, 2H); MS calcd. for [M+H]⁺ C₈H, BrNO: 216.0; found: 216.0.

Step C To a mixture of compound 3 (50 mg, 0.2 mmol), 266b (73 mg, 0.33 mmol) and PPh₃ (115 mg, 0.44 mmol) in THF (2 mL) at 0° C. was added DEAD (58 mg, 0.33 mmol). The reaction mixture was then stirred at rt overnight. Additional PPh₃ (70 mg, 0.27 mmol) and DEAD (78 mg, 0.45 mmol) were added and the reaction was continued for another 7 h. The reaction mixture was then purified by HPLC to give 266c as a greenish solid. MS calcd. for [M+H]⁺ C₁₈H₂₂BrN₂O₃S: 425.1; found: 425.0.

The title compound was prepared in a manner similar to Example 265. ¹H-NMR (400 MHz, acetone-d₆) δ=8.96 (d, J=2.0 Hz, 1H), 8.29 (dd, J=2.0, 8.0 Hz, 1H), 7.78-7.75 (m, 2H), 7.67 (d, J=8.4 Hz, 1H), 7.55 (m, 2H), 7.47 (m, 1H), 7.06 (d, J=8.4 Hz, 1H), 6.77 (dd, J=2.8, 8.4 Hz, 1H), 6.73 (d, J=2.4 Hz, 1H), 4.34 (s, 2H), 4.09 (t, J=6.4 Hz, 2H), 3.48 (t, J=6.0 Hz, 2H), 3.17 (t, J=7.4 Hz, 2H), 2.91 (t, J=6.0 Hz, 2H), 2.87 (s, 3H), 2.30 (m, 2H); MS calcd. for [M+H]⁺ C₂₄H₂₇N₂O₃S: 423.2; found: 423.1.

### Example 267

### 4-(5-(3-(2-(Methylsulfonyl)-1,2,3,4-tetrahydroisoquinolin-6-yloxy)propyl)pyridin-2-yl)morpholine

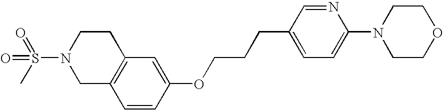

A mixture of compound 265c (10 mg, approx 0.021 mmol), morpholine (100 mg, 1.15 mmol) and Cs₂CO₃ (25 mg, 0.078 mmol) in anhydrous dioxane (1 mL) was stirred at 150° C. in a sealed pressure vial for 2 days. The reaction mixture was cooled, purified by HPLC to afford compound 267 as a white solid. MS calcd. for [M+H]⁺ C₂₂H₃₀N₃O₄S: 432.2; found: 432.1.

### Example 268

### 6-(3-(1-(1H-Benzo[d]imidazol-2-yl)piperidin-4-yl)propoxy)-2-(methylsulfonyl)-1,2,3,4-tetrahydroisoquinoline

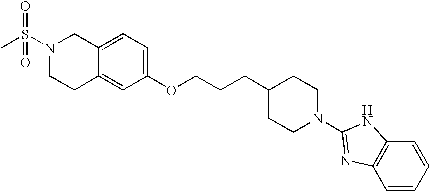

A mixture of 2-(methylsulfonyl)-6-(3-(piperidin-4-yl)propoxy)-1,2,3,4-tetrahydroisoquinoline 77d (HCl salt, 30 mg, 0.078 mmol), 2-chloro-1H-benzo[d]imidazole (24 mg, 0.15 mmol), diisopropylethylamine (86 uL, 0.52 mmol), and CuI (2 mg, 0.0012 mmol) in 1,4-dioxane (1 mL) was degassed. The vial was sealed and heated at 120° C. overnight. After cooling to rt, the reaction mixture was diluted with chloroform, and organics were washed with brine, dried and filtered. Solvents were removed and the crude was purified by mass trigger prep HPLC to afford the title compound 268 as a white solid. ¹H-NMR (400 MHz, CDCl₃+CD₃OD) δ 7.36 (br s, 1H), 7.12 (br s, 1H), 7.03 (br s, 1H), 6.96 (d, 2H, J=8.4 Hz), 6.72 (dd, 1H, J=2.8, 8.4 Hz), 6.64 (d, 1H, J=2.4 Hz), 4.36 (s, 2H), 4.02 (d, 2H, J=12.4 Hz), 3.90 (t, 2H, J=6.4 Hz), 3.50 (t, 2H, J=6.0 Hz), 3.01-2.94 (m, 2H), 2.91 (t, 2H, J=6.0 Hz), 2.80 (s, 3H), 1.82-1.75 (m, 4H), 1.57-1.46 (m, 1H), 1.43-1.38 (m, 2H), 1.34-1.24 (m, 2H); MS calcd. for [M+H]⁺ C₂₅H₃₂N₄O₃S: 469.2; found: 469.1.

### Example 269

### 6-(3-(1-(1-Methyl-1H-benzo[d]imidazol-2-yl)piperidin-4-yl)propoxy)-2-(methylsulfonyl)-1,2,3,4-tetrahydroisoquinoline

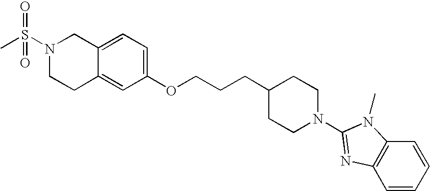

To a solution of Example 268 (10 mg, 0.021 mmol) in DMF (1 mL) was added NaH (60% in mineral oil, 20 mg, 0.55 mmol) at 0° C. The mixture was stirred for 30 min at 0° C. Then iodomethane (10 uL, 0.16 mmol) was added and the resulting mixture was stirred at rt overnight. It was cooled to 0° C. and water was slowly added until gas evolution ceased. The mixture was extracted with EtOAc and organics were combined, washed with sat. aq NH₄Cl, water and brine. The organics were dried (Na₂SO₄), filtered, concentrated under reduced pressure. The crude thus obtained was purified by silica gel column chromatography (EtOAc/hexanes) to afford 269 as a white solid. MS calcd. for [M+H]⁺ C₂₆H₃₄N₄O₃S: 483.2; found: 483.1.

### Example 270

### 4-(3-(2-(Methylsulfonyl)-1,2,3,4-tetrahydroisoquinolin-6-yloxy)propyl)-1-(pyridin-2-yl)piperazin-2-one

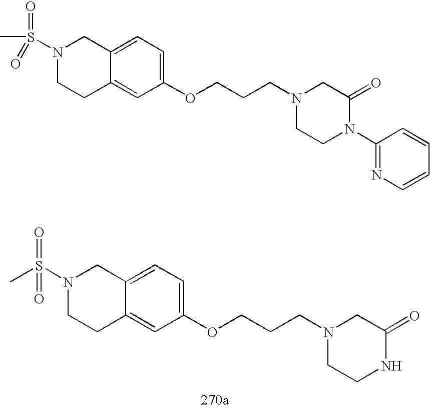

Step A 4-(3-(2-(Methylsulfonyl)-1,2,3,4-tetrahydroisoquinolin-6-yloxy)propyl)piperazin-2-one (270a) A suspension of 262a (300 mg, 0.86 mmol), piperazin-2-one (95 mg, 0.95 mmol) and K₂CO₃ (236 mg, 1.7 mmol) in anhydrous ACN (10 mL) was heated in a sealed vessel at 80° C. overnight. The salts were filtered from the solution while it was hot. The filtrate was cooled to rt, and crystals were formed upon standing. The solids were filtered to afford 270a. The mother liquor was concentrated in vacuo and the crude was purified by flash column chromatography (MeOH/CH₂Cl₂=10%) to afford additional 270a. ¹H-NMR (400 MHz, CDCl₃) δ 7.00 (d, 1H, J=8.8 Hz), 6.76 (dd, 1H, J=2.4, 8.4 Hz), 6.68 (d, 1H, J=2.4 Hz), 5.78 (br s, 1H), 4.40 (s, 2H), 4.01 (t, 2H, J=6.0 Hz), 3.54 (t, 2H, J=6.0 Hz), 3.40-3.36 (m, 2H), 3.17 (s, 2H), 2.94 (t, 2H, J=5.6 Hz), 2.83 (s, 3H), 2.69 (t, 2H, J=5.2 Hz), 2.61 (t, 2H, J=6.8 Hz), 1.97 (quint, 2H, J=6.4 Hz); MS calcd. for [M+H]⁺ C₁₇H₂₅N₃O₄S: 368.2; found: 368.1.

Step B To a degassed mixture of 270a (20 mg, 0.054 mmol), 2-bromopyridine (6 uL, 0.065 mmol), and Cs₂CO₃ (53 mg, 0.16 mmol) in 1,4-dioxane (1 mL) was added Pd₂(dba)₃ (5 mg, 0.005 mmol) and xantphos (10 mg, 0.017 mmol). The vial was sealed and heated at 110° C. for 1 h. The mixture was cooled to rt, filtered and the filtrate was concentrated in vacuo. The crude product was purified by silica gel column chromatography (EtOAc/hexanes) to afford the title compound 270. MS calcd. for [M+H]⁺ C₂₂H₂₈N₄O₄S: 445.2; found: 445.1.

### Example 271

### 2-(1-(5-Ethylpyrimidin-2-yl)piperidin-4-yloxy)-3-(2-(methylsulfonyl)-1,2,3,4-tetrahydroisoquinolin-6-yloxy)propan-1-ol

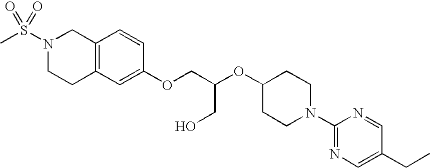

Intermediate 271d: 3-(2-(Methylsulfonyl)-1,2,3,4-tetrahydroisoquinolin-6-yloxy)-2-(piperidin-4-yloxy)propan-1-ol

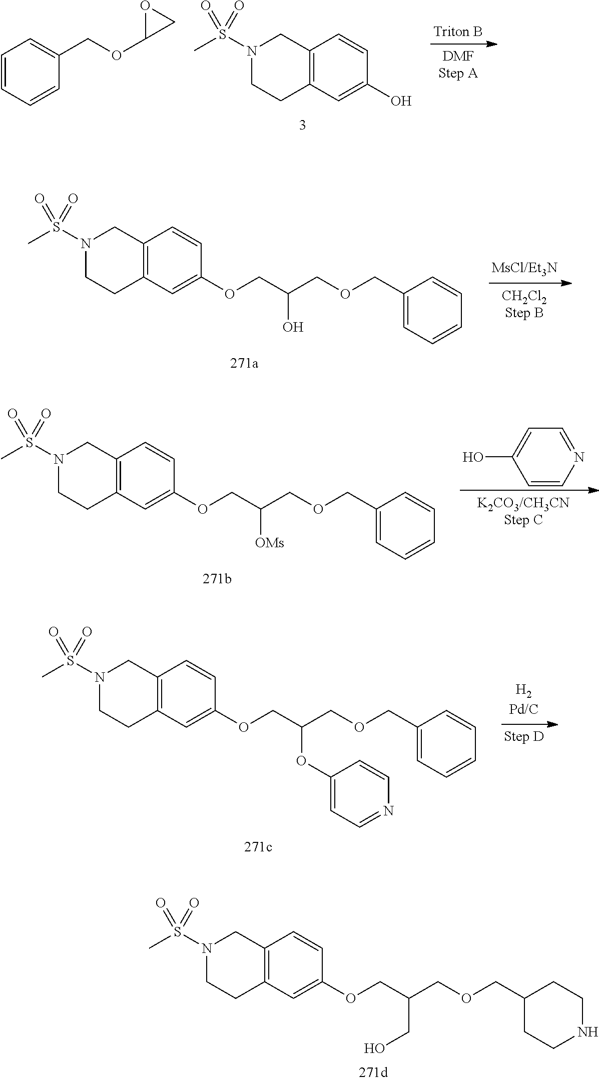

Step A To a solution of 3 (229 mg, 1.0 mmol) in DMF (4 mL) was added benzyl glycidyl ether (166 mg, 1.0 mmol) and benzyltrimethylammonium hydroxide (40% aqueous, 15 μL). The solution was heated to 155° C. overnight and cooled to rt. The solution was diluted with methanol (3 mL) and concentrated in vacuo to a thick yellow oil. The crude product was dissolved in ethyl acetate (30 mL), washed with saturated NaHCO₃, water, and brine, dried over MgSO₄ and filtered. Removal of the solvent in vacuo afforded 1-(benzyloxy)-3-(2-(methylsulfonyl)-1,2,3,4-tetrahydroisoquinolin-6-yloxy)propan-2-ol (271a) as a yellow solid. ¹H-NMR (400 MHz, CDCl₃) δ=7.36 (m, 5H), 7.03 (d, J=8.4 Hz, 1H), 6.80 (dd, J=8.4, 2.7 Hz, 1H), 6.71 (d, J=2.7 Hz, 1H), 4.61 (s, 2H), 4.42 (s, 2H), 4.21 (m, 1H), 4.04 (m, 2H), 3.68 (m, 2H), 3.57 (t, J=6.0 Hz, 2H), 2.96 (t, J=6.0 Hz, 2H), 2.85 (s, 3H), 2.54 (d, J=5.0 Hz, 1H); MS calcd. for [M+H]⁺ C₂₀H₂₅NO₅S: 392.2; found: 392.1.

Step B 1-(Benzyloxy)-3-(2-(methylsulfonyl)-1,2,3,4-tetrahydroisoquinolin-6-yloxy)propan-2-yl methanesulfonate (271b) was prepared from 271a (373 mg, 0.95 mmol) according to the procedure described for 26b. The compound was used in the next step without further purification. ¹H-NMR (400 MHz, CDCl₃) δ=7.33 (m, 5H), 7.01 (d, J=8.4 Hz, 1H), 6.74 (dd, J=8.4, 2.7 Hz, 1H), 6.67 (d, J=2.7 Hz, 1H), 5.06 (m, 1H), 4.59 (d, J=4.0 Hz, 2H), 4.39 (s, 2H), 4.20 (m, 2H), 3.81 (m, 2H), 3.54 (t, J=6.0 Hz, 2H), 3.09 (s, 3H), 2.94 (t, J=6.0 Hz, 2H), 2.83 (s, 3H); MS calcd. for [M+H]⁺ C₂₁H₂₇NO₇S₂: 470.1; found 470.1.

Step C 271b and 4-hydroxypyridine (133 mg, 1.4 mmol) were treated according to the procedure described for 264c, using acetonitrile (5 mL) as the solvent and heating to 80° C. for 10 min. The crude product was purified by reverse-phase HPLC (water/acetonitrile) to afford 6-(3-(benzyloxy)-2-(pyridin-4-yloxy)propoxy)-2-(methylsulfonyl)-1,2,3,4-tetrahydroisoquinoline (271c) as a white solid. MS calcd. for [M+H]⁺ C₂₅H₂₈N₂O₅S: 469.2; found: 469.2.

Step D 271c (90 mg, 0.2 mmol) was hydrogenated according to the procedure for Example 263, using a mixture of EtOH (10 mL), EtOAc (5 mL) and HOAc (0.5 mL) as the solvent, to afford 271d as a colorless oil. MS calcd. for [M+H]⁺ C₁₈H₂₈N₂O₅S: 385.2; found: 385.2.

271d (21 mg, 0.05 mmol) and 2-chloro-5-ethylpyrimidine (7 uL, 0.05 mmol) were treated as described for 24c, using K₂CO₃ (25 mg, 0.2 mmol) as a base and heating to 170° C. for 10 min under microwave irradiation, to afford Example 271 as a white solid. ¹H-NMR (400 MHz, CD₃CN)=8.28 (s, 2H), 7.05 (d, J=8.3 Hz, 1H), 6.80 (m, 2H), 4.32 (s, 2H), 4.13 (m, 2H), 4.04 (m, 2H), 3.97 (m, 2H), 3.85 (m, 2H), 3.65 (m, 2H), 3.49 (m, 2H), 3.45 (t, J=6.0 Hz, 2H), 2.91 (t, J=6.0 Hz, 2H), 2.80 (s, 3H), 2.50 (q, J=7.6 Hz, 2H), 1.57 (m, 2H), 1.17 (t, J=7.6 Hz, 3H); MS calcd. for [M+H]⁺ C₂₄H₃₄N₄O₅S: 491.2; found: 491.2.

### Example 272

### 1-Methylcyclopropyl 4-(4-(2-(methylsulfonyl)-1,2,3,4-tetrahydroisoquinolin-6-yl)-3-morpholino-4-oxobutyl)piperidine-1-carboxylate

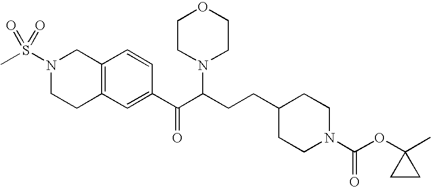

Intermediate 272a: 1-Methylcyclopropyl 4-(4-(2-(methylsulfonyl)-1,2,3,4-tetrahydroisoquinolin-6-yl)-4-oxo-3-(tosyloxy)butyl)piperidine-1-carboxylate

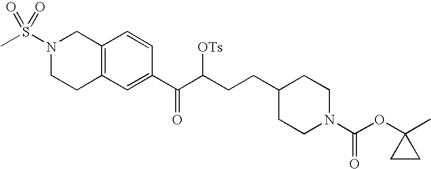

Example 141 (100 mg, 0.22 mmol) was suspended in acetonitrile (1 mL), treated successively with iodobenzene (2.5 μL, 0.02 mmol), toluenesulfonic acid (45 mg, 0.23 mmol) and m-chloroperbenzoate (70%, 58 mg, 0.23 mmol) and stirred at 50° C. overnight. The mixture was then poured into sat. aq. NaHCO₃ and extracted with CH₂Cl₂. The organic layer was dried (Na₂SO₄) and concentrated in vacuo to afford 1-methylcyclopropyl 4-(4-(2-(methylsulfonyl)-1,2,3,4-tetrahydroisoquinolin-6-yl)-4-oxo-3-(tosyloxy)butyl)piperidine-1-carboxylate (272a). The crude mixture was used in the next step without further purification: MS calcd. for [M+H]⁺ C₃₁HIN₂O₈S₂: 633.2, found 633.2.

Intermediate 272a (63 mg, 0.1 mmol) was dissolved in CH₂Cl₂ (0.25 mL) and treated with morpholine (0.25 mL). The mixture was stirred at rt overnight, then diluted with MeCN and filtered. The filtrate was purified by reverse-phase HPLC to yield the title compound (Example 272): ¹H-NMR (400 MHz, CD₃CN) δ=7.75-7.71 (m, 2H), 7.28 (d, J=8.0 Hz, 1H), 4.94 (t, J=6.0 Hz, 1H), 4.41 (s, 2H), 3.86-3.67 (m, 6H), 3.44 (t, J=6.0 Hz, 2H), 3.30 (br. s, 2H), 3.18 (br. s, 2H), 2.98-2.94 (m, 2H), 2.78 (s, 3H), 2.52 (br. s, 2H), 1.98-1.91 (m, 2H), 1.50-1.40 (m, 1H), 1.36 (s, 3H), 1.39-1.31 (m, 1H), 1.27-1.17 (m, 1H), 1.10-0.95 (m 2H), 0.86-0.70 (m, 2H), 0.68-0.65 (m, 2H), 0.49-0.46 (m, 2H); MS calcd. for [M+H]⁺ C₂₈H₄₂N₃O₆S: 548.3, found: 548.3.

By repeating the procedures described in the above examples, using appropriate starting materials, the following compounds of Formula I, as identified in Table 1, were obtained.

TABLE 1

| Example # | Structure | NMR and/or ESMS |
|---|---|---|
| 1 | 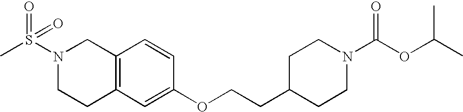 | MS calcd. for [M + H]⁺C₂₁H₃₃N₂O₅S: 425.2; found: 425.2 |
| 2 | 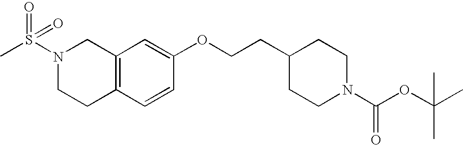 | MS calcd. for [M + H]⁺ C₂₂H₃₅N₂O₅S: 439.2; found: 439.2 |
| 3 | 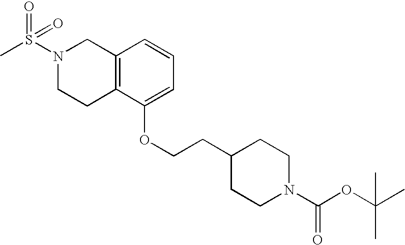 | MS calcd. for [M + H]⁺ C₂₂H₃₅N₂O₅S: 439.2; found: 439.2 |
| 4 | 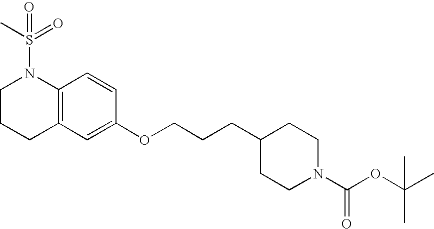 | MS calcd. for [M + H]⁺ C₂₂H₃₆N₂O₅S: 453.2; found: 453.2 |
| 5 | 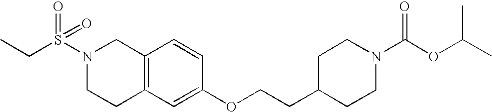 | MS calcd. for [M + H]⁺C₂₂H₃₅N₂O₅S: 439.2; found: 439.2 |
| 6 | 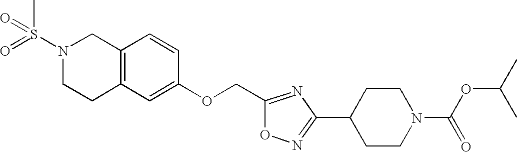 | MS calcd. for [M + H]⁺ C₂₂H₃₁N₄O₆S: 479.2; Found: 479.2 |
| 7 | 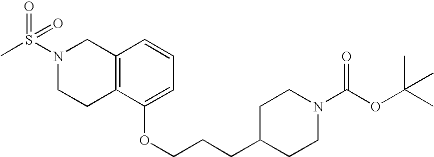 | MS calcd. for [M + H]⁺ C₂₂H₃₅N₂O₅S: 453.2; found: 453.2 |
| 8 | 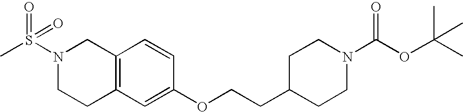 | H¹ NMR (CDCl₃): δ 7.00 (d, 1H), 6.75 (dd, 1H), 6.67 (d, 1H), 4.40 (s, 2H), 4.10 (brs, 2H), 3.99 (t, 2H), 3.55 (t, 2H), 2.94 (t, 2H), 2.83 (s, 3H), 2.71 (t, 2H), 1.73-1.70 (m, 5H), 1.46 (s, 9H), 1.22-1.12 (m, 2H). MS calcd. for [M + H]⁺ C₂₂H₃₅N₂O₅S: 439.2; found: 439.2 |
| 9 | 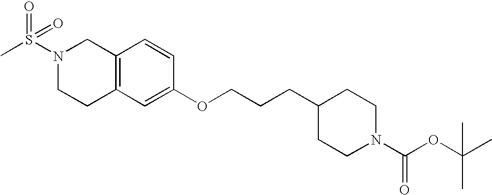 | MS calcd. for [M + H]⁺ C₂₂H₃₅N₂O₅S: 453.23; found: 453.2 |
| 10 | 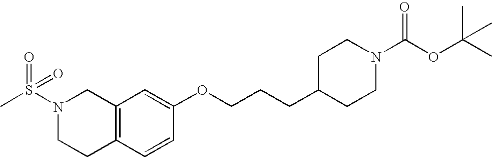 | MS calcd. for [M + H]⁺ C₂₂H₃₅N₂O₅S: 453.2; found: 453.2 |
| 11 | 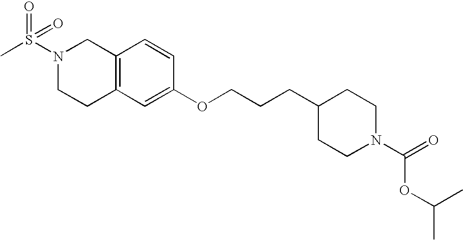 | H¹ NMR (CDCl₃): δ 6.99 (d, 1H), 6.75 (dd, 1H), 6.66 (d, 1H), 4.94-4.88 (m, 1H), 4.40 (s, 2H), 4.13 (brs, 2H), 3.92 (t, 2H), 3.54 (t, 2H), 2.94 (t, 2H), 2.82 (s, 3H), 1.82- 1.77 (m, 2H), 1.69 (d, 2H), 1.49-1.38 (m, 3H), 1.24 (s, 3H), 1.23 (s, 3H), 1.15-1.09 (m, 2H). MS calcd. for [M + H]⁺ C₂₂H₃₅N₂O₅S: 439.2; found: 439.2 |
| 12 | 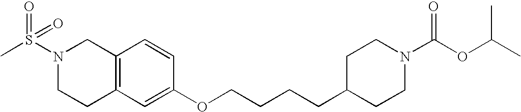 | MS calcd. for [M + H]⁺ C₂₂H₃₅N₂O₅S: 453.23; found: 453.2 |
| 13 | 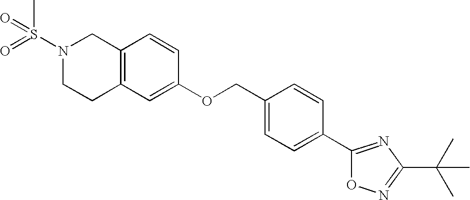 | MS calcd. for [M + H]⁺ C₂₃H₂₈N₃O₄S: 442.2; found: 442.2 |
| 14 | 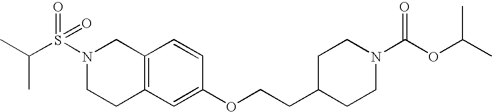 | MS calcd. for [M + H]⁺ C₂₂H₃₅N₂O₅S: 453.23; found: 453.2 |
| 15 | 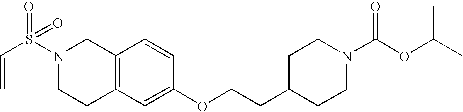 | MS calcd. for [M + H]⁺ C₂₂H₃₃N₂O₅S: 437.2; found: 437.2. |
| 16 | 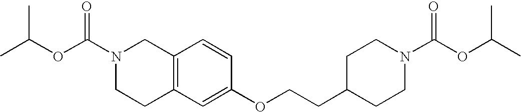 | MS calcd. for [M + H]⁺ C₂₄H₃₇N₂O₅: 433.2; found: 433.2 |
| 17 | 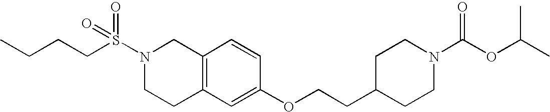 | MS calcd. for [M + H]⁺ C₂₄H₃₉N₂O₅S: 467.2; found: 467.2 |
| 18 | 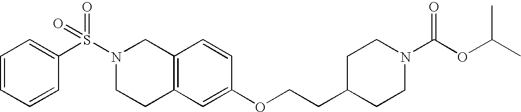 | MS calcd. for [M + H]⁺ C₂₆H₃₅N₂O₅S: 487.2; found: 487.2 |
| 19 | 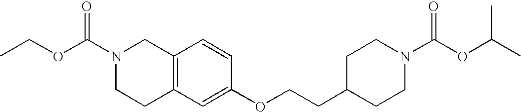 | MS calcd. for [M + H]⁺ C₂₃H₃₅N₂O₅: 419.2; found: 419.2 |
| 20 | 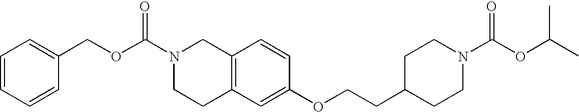 | MS calcd. for [M + H]⁺ C₂₈H₃₇N₂O₅ 481.2; found: 481.2 |
| 21 | 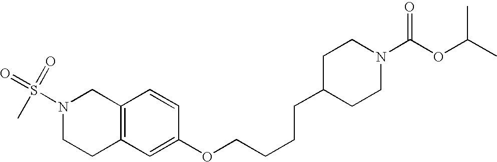 | MS calcd. for [M + H]⁺ C₂₃H₃₇N₂O₅S 453.2; found: 453.2 |
| 22 | 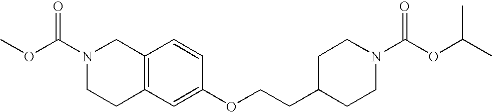 | MS calcd. for [M + H]⁺ C₂₂H₃₃N₂O_{5:} 405.2; found: 405.2 |
| 23 | 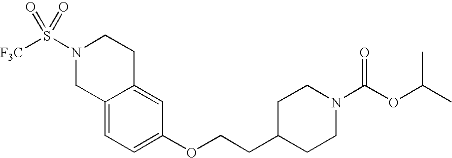 | MS calcd. for [M + H]⁺ C₂₁H₃₀F₃N₂O₅S: 479.2; found: 479.2 |
| 24 | 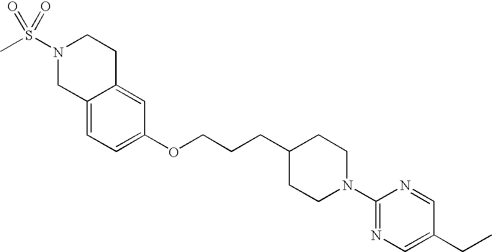 | ¹H-NMR (400 MHz, CDCl₃) δ 8.16 (2 H, s); 6.98 (1 H, d, J = 8.4 Hz); 6.74 (1 H, dd, J = 2.8 Hz, J = 8.4 Hz); 6.66 (1 H, d, J = 2.4 Hz); 4.71 (1 H, brs); 4.67 (1 H, brs); 4.38 (2 H, s); 3.92 (2 H, t, J = 6.4 Hz); 3.53 (2 H, t, = 6.0 Hz); 2.93 (2 H, t, J = 6.0 Hz); 2.86 (2 H, dt, J = 2.0 Hz, J = 13.2 Hz); 2.81 (3 H, s); 2.44 (2 H, q, J = 7.6 Hz); 1.80 (3 H, m); 1.55 (2 H, m); 1.42 (2 H, m); 1.37 (2 H, m); 1.36 (3 H, t, J = 7.6 Hz). MS calcd. for [M + H]⁺ C₂₄H₃₅N₄O₃S: 459.2; found: 459.2 |
| 25 | 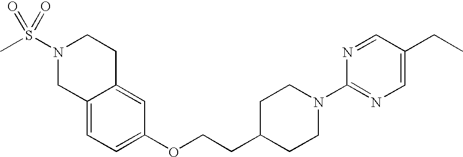 | MS calcd. for [M + H]⁺ C₂₃H₃₃N₄O₃S: 445.2; found: 445.2 |
| 26 | 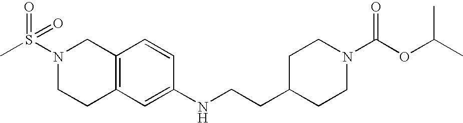 | ¹H-NMR (400 MHz, CD₃CN) δ = 7.20 (d, J = 8.4 Hz, 1H), 7.09-7.05 (m, 2H), 5.26 (br s, 1H), 4.83 (septet, J = 6.0 Hz, 1H), 4.40 (s, 2H), 4.05 (d, J = 12.4 Hz, 2H), 3.50 (t, J = 6.0 Hz, 2H), 3.31 (t, J = 7.2 Hz, 2H), 2.96 (t, J = 6.0 Hz, 2H), 2.86 (s, 3H), 2.80-2.67 (m, 2H), 1.73- 1.52 (m, 5H), 1.22 (d, J = 6.0 Hz, 6H), 1.15-1.00 (m, 2H). MS calcd. for [M + H]⁺ C₂₁H₃₄N₃O₄S: 424.2; found: 424.2. |
| 27 | 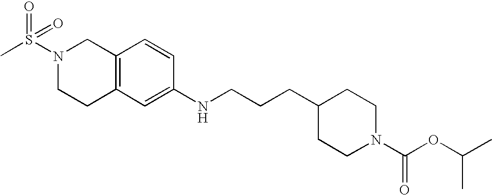 | ¹H-NMR (400 MHz, CD₃CN) δ = 7.18 (d, J = 8.4 Hz, 1H), 7.07 (d, J = 8.4 Hz, 1H), 7.05 (s, 1H), 4.83 (septet, J = 6.0 Hz, 1H), 4.40 (s, 2H), 4.05 (br. d, J = 12.8 Hz, 2H), 3.50 (t, J = 6.0 Hz, 2H), 3.25 (t, J = 7.6 Hz, 2H), 2.96 (t, J = 6.0 Hz, 2H), 2.86 (s, 3H), 2.77-2.68 (m, 2H), 1.73-1.64 (m, 3H), 1.55-1.40 (m, 2H), 1.36-1.28 (m, 2H), 1.21 (d, J = 6.0 Hz, 6H), 1.02 (ddd, J = 12.4, 12.4, 4.0 Hz, 2H); MS calcd. for [M + H]⁺ C₂₂H₃₆N₃O₄S: 438.2; found: 438.3 |
| 28 | 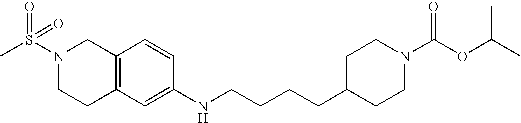 | ¹H-NMR (400 MHz, CD₃CN) δ = 7.15 (d, J = 8.4 Hz, 1H), 7.00 (d, J = 8.4 Hz, 1H), 6.97 (s, 1H), 4.83 (septet, J = 6.4 Hz, 1H), 4.38 (s, 2H), 4.05 (br. d, J = 13.2 Hz, 2H), 3.49 (t, J = 6.0 Hz, 2H), 3.23 (t, J = 7.6 Hz, 2H), 2.94 (t, J = 6.0 Hz, 2H), 2.85 (s, 3H), 2.78-2.68 (m, 2H), 1.70-1.62 (m, 4H), 1.46-1.36 (m, 3H), 1.30-1.24 (m, 2H), 1.21 (d, J = 6.4 Hz, 6H), 1.02 (ddd, J = 12.4, 12.4, 4.0 Hz, 2H); MS calcd. for [M + H]⁺ C₂₄H₃₉N₂O₄S: 451.3; found: 451.2 |
| 29 | 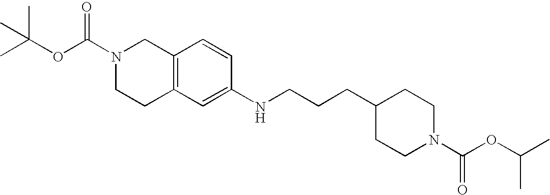 | ¹H-NMR (400 MHz, CD₃CN) δ = 7.04 (d, J = 8.4 Hz, 1H), 6.81 (dd, J = 8.4, 2.4 Hz, 1H), 6.76 (s, 1H), 4.83 (septet, J = 6.4 Hz, 1H), 4.47 (s, 2H), 4.04 (br. d, J = 13.2 Hz, 2H), 3.58 (t, J = 6.0 Hz, 2H), 3.15 (t, J = 7.6 Hz, 2H), 2.76 (t, J = 6.0 Hz, 2H), 2.76-2.68 (m, 2H), 1.69-1.61 (m, 4H), 1.47 (s, 9H), 1.47-1.40 (m, 1H), 1.34-1.28 (m, 2H), 1.21 (d, J = 6.4 Hz, 6H), 1.02 (ddd, J = 12.8, 12.4, 4.0 Hz, 2H); MS calcd. for [M + 2H- Boc]⁺ C₂₁H₃₄N₃O₂: 360.2; found: 360.1 |
| 30 | 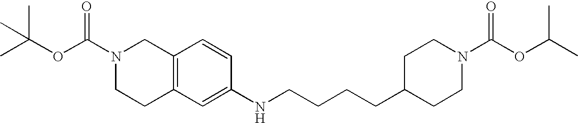 | ¹H-NMR (400 MHz, CDCl₃) δ = 6.93 (d, J = 8.0 Hz, 1H), 6.48 (dd, J = 8.0, 2.4 Hz, 1H), 6.39 (d, J = 2.4 Hz, 1H), 4.93 (septet, J = 6.0 Hz, 1H), 4.48 (s, 2H), 4.20-4.10 (m, 2H), 3.62 (br. s, 2H), 3.11 (t, J = 7.2 Hz, 2H), 2.82-2.68 (m, 4H), 1.69- 1.60 (m, 4H), 1.50 (s, 9H), 1.45-1.39 (m, 3H), 1.32-1.27 (m, 2H), 1.25 (d, J = 6.0 Hz, 6H), 1.15- 1.05 (m, 2H); MS calcd. for [M + 2H- Boc]⁺ C₂₂H₃₆N₃O₂: 374.3; found: 374.1 |
| 31 | 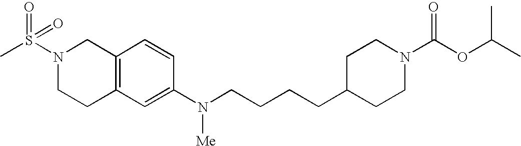 | ¹H-NMR (400 MHz, CD₃CN) δ = 7.00 (d, J = 8.4 Hz, 1H), 6.82 (d, J = 8.4 Hz, 1H), 6.77 (s, 1H), 4.71 (septet, J = 6.4 Hz, 1H), 4.25 (s, 2H), 3.92 (br. d, J = 13.6 Hz, 2H), 3.38 (t, J = 6.0 Hz, 2H), 3.25 (t, J = 7.6 Hz, 2H), 2.87 (s, 3H), 2.84 (t, J = 6.0 Hz, 2H), 2.74 (s, 3H), 2.66-2.50 (m, 2H), 1.83- 1.78 (m, 2H), 1.54 (br. d, J = 12.0 Hz, 2H), 1.44- 1.36 (m, 2H), 1.33-1.18 (m, 3H), 1.21 (d, J = 6.0 Hz, 6H), 0.89 (ddd, J = 12.8, 12.8, 4.0 Hz, 2H); MS calcd. for [M + H]⁺ C₂₄H₄₀N₃O₄S: 466.3; found: 466.2 |
| 32 | 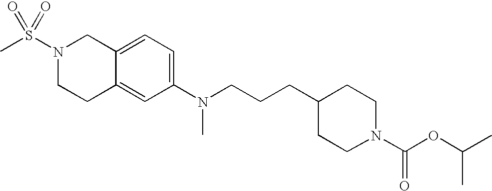 | ¹H-NMR (400 MHz, CD₃CN) δ = 6.97 (d, J = 8.4 Hz, 1H), 6.62 (dd, J = 8.4, 2.8 Hz, 1H), 6.51 (d, J = 2.8 Hz, 1H), 4.82 (septet, J = 6.4 Hz, 1H), 4.29 (s, 2H), 4.07-4.03 (m, 2H), 3.46 (t, J = 6.0 Hz, 2H), 3.30 (t, J = 7.6 Hz, 2H), 2.90 (t, J = 6.0 Hz, 2H), 2.89 (s, 3H), 2.82 (s, 3H), 2.82-2.70 (m, 2H), 1.70-1.65 (m, 2H), 1.60-1.53 (m, 2H), 1.30-1.24 (m, 3H), 1.21 (d, J = 6.4 Hz, 6H), 1.02 (ddd, J = 12.8, 12.8, 4.0 Hz, 2H); MS calcd. for [M + H]⁺ C₂₃H₃₈N₃O₄S: 452.3; found: 452.2. |
| 33 | 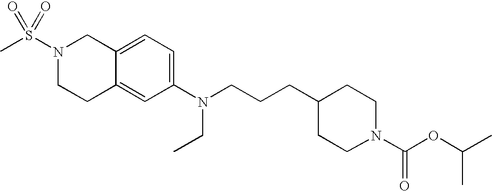 | ¹H-NMR (400 MHz, CD₃CN) δ 6.97 (d, J = 8.4 Hz, 1H), 6.62 (dd, J = 8.4, 2.8 Hz, 1H), 6.53 (d, J = 2.8 Hz, 1H), 4.82 (septet, J = 6.0 Hz, 1H), 4.29 (s, 2H), 4.04 (d, J = 11.6 Hz, 2H), 3.46 (t, J = 6.0 Hz, 2H), 3.37 (q, J = 6.8 Hz, 2H), 3.25 (t, J = 7.6 Hz, 2H), 2.90 (t, J = 6.0 Hz, 2H), 2.83 (s, 3H), 2.76-2.67 (m, 2H), 1.70- 1.65 (m, 2H), 1.60-1.53 (m, 2H), 1.30-1.25 (m, 3H), 1.21 (d, J = 6.0 Hz, 6H), 1.10 (t, J = 6.8 Hz, 3H), 1.03 (ddd, J = 12.8, 12.8, 4.0 Hz, 2H); MS calcd. for [M + H]⁺ C₂₄H₄₀N₃O₄S: 466.3; found: 466.2. |
| 34 | 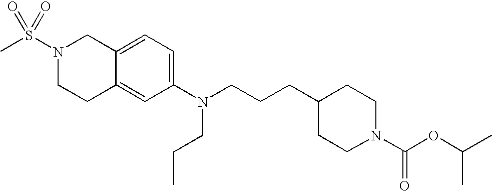 | ¹H-NMR (400 MHz, CD₃CN) δ = 6.94 (d, J = 8.4 Hz, 1H), 6.57 (dd, J = 8.4, 2.8 Hz, 1H), 6.47 (d, J = 2.8 Hz, 1H), 4.82 (septet, J = 6.0 Hz, 1H), 4.28 (s, 2H), 4.04 (d, J = 11.2 Hz, 2H), 3.46 (t, J = 6.0 Hz, 2H), 3.28-3.21 (m, 4H), 2.89 (t, J = 6.0 Hz, 2H), 2.82 (s, 3H), 2.76-2.67 (m, 2H), 1.70- 1.65 (m, 2H), 1.60-1.54 (m, 2H), 1.30-1.25 (m, 3H), 1.21 (d, J = 6.0 Hz, 6H), 1.08-0.98 (m, 2H), 0.92 (t, J = 7.2 Hz, 3H); MS calcd. for [M + H]⁺ C₂₅H₄₂N₃O₄S: 480.3; found: 480.2. |
| 35 | 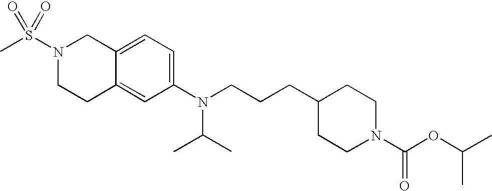 | ¹H-NMR (400 MHz, CD₃CN) δ = 6.96 (d, J = 8.4 Hz, 1H), 6.62 (dd, J = 8.4, 2.8 Hz, 1H), 6.51 (d, J = 2.8 Hz, 1H), 4.82 (septet, J = 6.0 Hz, 1H), 4.31 (s, 2H), 4.06-4.01 (m, 2H), 3.50-3.45 (m, 2H), 3.19-3.12 (m, 1H), 2.93-2.88 (m, 2H), 2.83 (s, 3H), 2.76-2.68 (m, 2H), 1.72-1.64 (m, 2H), 1.55-1.50 (m, 2H), 1.30- 1.26 (m, 3H), 1.22 (d, J = 6.0 Hz, 3H), 1.21 (d, J = 6.4 Hz, 6H), 1.17 (d, J = 6.8 Hz, 3H), 1.09-0.98 (m, 2H); MS calcd. for [M + H]⁺ C₂₅H₄₂N₃O₄S: 480.3; found: 480.2. |
| 36 | 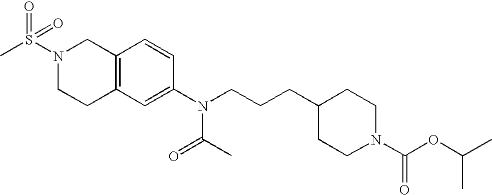 | ¹H-NMR (400 MHz, CD₃CN) δ = 7.15 (d, J = 8.8 Hz, 1H), 7.02-7.00 (m, 2H), 4.71 (septet, J = 6.4 Hz, 1H), 4.31 (s, 2H), 3.92 (br. d, J = 13.2 Hz, 2H), 3.56 (t, J = 7.6 Hz, 2H), 3.41 (t, J = 6.0 Hz, 2H), 2.88 (t, J = 6.0 Hz, 2H), 2.77 (s, 3H), 2.70- 2.55 (m, 4H), 1.74 (s, 3H), 1.52 (br. d, J = 10.8 Hz, 2H), 1.43-1.35 (m, 3H), 1.10 (d, J = 6.4 Hz, 6H), 0.89 (ddd, J = 12.4, 12.4, 4.0 Hz, 2H); MS calcd. for [M + H]⁺ C₂₄H₃₈N₃O₅S: 480.3; found: 480.2 |
| 37 | 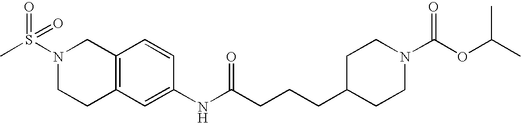 | ¹H-NMR (400 MHz, CDCl₃) δ = 7.52 (br. s, 1H), 7.23 (d, J = 8.4 Hz, 1H), 7.07-7.05 (m, 2H), 4.92 (septet, J = 6.4 Hz, 1H), 4.44 (s, 2H), 4.14 (br. s, 2H), 3.57 (t, J = 6.0 Hz, 2H), 2.99 (t, J = 6.0 Hz, 2H), 2.86 (s, 3H), 2.72 (t, J = 12.8 Hz, 2H), 2.37 (t, J = 7.2 Hz, 2H), 1.81-1.66 (m, 4H), 1.48- 1.41 (m, 1H), 1.37-1.31 (m, 2H), 1.25 (d, J = 6.4 Hz, 6H), 1.18-1.08 (m, 2H); MS calcd. for [M + H]⁺ C₂₃H₃₆N₃O₅S: 466.2; found: 466.2 |
| 38 | 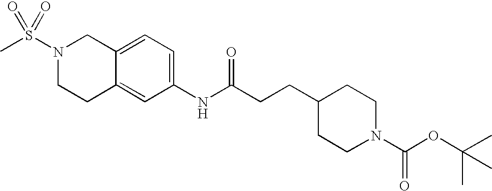 | ¹H-NMR (400 MHz, CDCl₃) δ = 7.51 (br. s, 1H), 7.24-7.22 (m, 1H), 7.08-7.05 (m, 2H), 4.41 (s, 2H), 4.11 (br. s, 2H), 3.57 (t, J = 6.0 Hz, 2H), 2.99 (t, J = 6.0 Hz, 2H), 2.86 (s, 3H), 2.73-2.67 (m, 2H), 2.41 (t, J = 7.6 Hz, 2H), 1.75-1.68 (m, 4H), 1.58-1.49 (m, 1H), 1.48 (s, 9H), 1.15 (ddd, J = 12.4, 12.4, 4.4 Hz, 2H); MS calcd. for [M + 2H- Boc]⁺ C₁₈H₂₈N₃O₃S: 366.2; found: 366.2. |
| 39 | 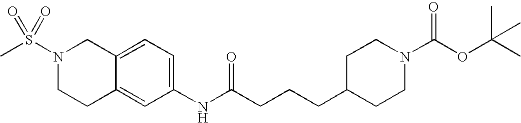 | ¹H-NMR (400 MHz, CDCl₃) δ = 7.51 (br. s, 1H), 7.24-7.22 (m, 1H), 7.08-7.02 (m, 2H), 4.41 (s, 2H), 4.10 (br. s, 2H), 3.57 (t, J = 6.0 Hz, 2H), 2.99 (t, J = 6.0 Hz, 2H), 2.86 (s, 3H), 2.69 (br. t, J = 12.4 Hz, 2H), 2.37 (t, J = 7.6 Hz, 2H), 1.81-1.66 (m, 4H), 1.47 (s, 9H), 1.47-1.39 (m, 1H), 1.36- 1.31 (m, 2H), 1.11 (ddd, J = 12.4, 12.4, 4.4 Hz, 2H); MS calcd. for [M + 2H- Boc]⁺ C₁₉H₃₀N₃O₃S: 380.2; found: 380.2. |
| 40 | 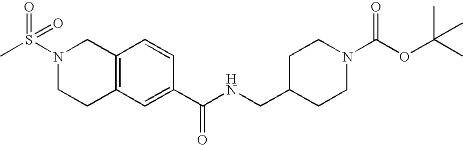 | ¹H-NMR (400 MHz, CDCl₃) δ = 7.53 (s, 1H), 7.51 (d, J 8.0 Hz, 1H), 7.14 (d, J = 8.0, 1H), 6.96 (br. t, 1H), 4.36 (s, 2H), 3.95 (br. d, J = 13.2, 2H), 3.43 (t, J = 6.0 Hz, 2H), 3.14 (t, J = 6.4 Hz, 2H), 2.92 (t, J = 6.0 Hz, 2H), 2.76 (s, 3H), 2.61 (br. s, 2H), 1.67-1.59 (m, 3H), 1.33 (s, 9H), 1.00 (ddd, J = 12.4, 12.4, 4.4 Hz, 2H); MS calcd. for [M + 2H- Boc]⁺ C₁₇H₂₆N₃O₃S: 352.1; found: 352.1 |
| 41 | 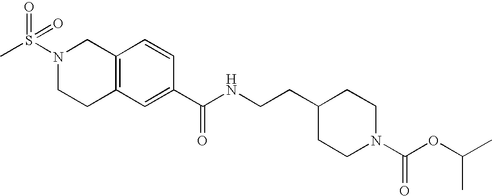 | ¹H-NMR (400 MHz, CD₃CN) δ = 7.53 (s, 1H), 7.50 (d, J = 8.0 Hz, 1H), 7.13 (d, J = 8.0, 1H), 6.88 (br. t, 1H), 4.72 (septet, J = 6.4 Hz, 1H), 4.36 (s, 2H), 3.95 (br. d, J = 13.2, 2H), 3.42 (t, J = 6.0 Hz, 2H), 3.29 (q, J = 6.0 Hz, 2H), 2.92 (t, J = 6.0 Hz, 2H), 2.76 (s, 3H), 2.63 (br. t, 2H), 1.64 (br. d, J = 12.8, 2H), 1.50-1.39 (m, 3H), 1.11 (d, J = 6.4 Hz, 6H), 0.98 (ddd, J = 12.8, 12.4, 4.4 Hz, 2H); MS calcd. for [M + H]⁺ C₂₂H₃₄N₃O₅S: 452.2; found: 452.2 |
| 42 | 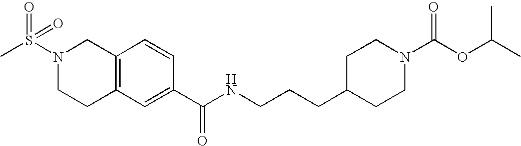 | ¹H-NMR (400 MHz, CD₃CN) δ = 7.63 (s, 1H), 7.61 (d, J = 8.0 Hz, 1H), 7.24 (d, J = 8.0, 1H), 7.01 (br. t, 1H), 4.82 (septet, J = 6.4 Hz, 1H), 4.46 (s, 2H), 4.05 (br. d, J = 13.2, 2H), 3.53 (t, J = 6.0 Hz, 2H), 3.33 (q, J = 6.8 Hz, 2H), 3.02 (t, J = 6.0 Hz, 2H), 2.86 (s, 3H), 2.73 (br. t, 2H), 1.70 (br. d, J = 12.8, 2H), 1.65-1.57 (m, 2H), 1.53-1.42 (m, 1H), 1.34-1.28 (m, 2H), 1.21 (d, J = 6.4 Hz, 6H), 1.04 (ddd, J = 12.4, 12.4, 4.4 Hz, 2H); MS calcd. for [M + H]⁺ C₂₃H₃₆N₃O₅S: 466.2; found: 466.2 |
| 43 | 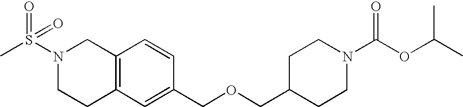 | ¹H-NMR (400 MHz, CD₃CN) δ = 7.08-7.03 (m, 3H), 4.72 (septet, J = 6.4 Hz, 1H), 4.34 (s, 2H), 4.30 (s, 2H), 3.97 (d, J = 12.8 Hz, 2H), 3.40 (t, J = 6.0 Hz, 2H), 3.23 (d, J = 6.4 Hz, 2H), 2.86 (t, J = 6.0 Hz, 2H), 2.74 (s, 3H), 2.65 (br. t, 2H), 1.74-1.66 (m, 1H), 1.62 (br. d, J = 13.2 Hz, 2H), 1.11 (d, J = 6.4 Hz, 6H), 1.01 (ddd, J = 12.4, 12.4, 4.4 Hz, 2H); MS calcd. for [M + H]⁺ C₂₁H₃₃N₂O₅S: 425.2; found: 425.2 |
| 44 | 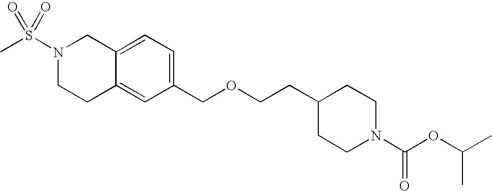 | ¹H-NMR (400 MHz, CD₃CN) δ = 7.08-7.03 (m, 3H), 4.72 (septet, J = 6.4 Hz, 1H), 4.33 (s, 2H), 4.30 (s, 2H), 3.93 (br. d, J = 13.2 Hz, 2H), 3.41 (t, J = 6.4 Hz, 2H), 3.40 (t, J = 6.0 Hz, 2H), 2.86 (t, J = 6.0 Hz, 2H), 2.74 (s, 3H), 2.62 (br. t, 2H), 1.57-1.47 (m, 3H), 1.46-1.40 (m, 2H), 1.11 (d, J = 6.4 Hz, 6H), 1.00-0.90 (m, 2H); MS calcd. for [M + H]⁺ C₂₂H₃₅N₂O₅S: 439.2; found: 439.2. |
| 45 | 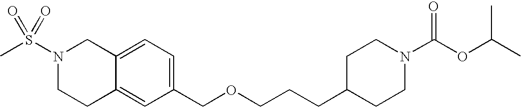 | ¹H-NMR (400 MHz, CD₃CN) δ = 7.08-7.03 (m, 3H), 4.72 (septet, J = 6.4 Hz, 1H), 4.33 (s, 2H), 4.30 (s, 2H), 3.94 (d, J = 12.8 Hz, 2H), 3.40 (t, J = 6.0 Hz, 2H), 3.36 (t, J = 6.4 Hz, 2H), 2.86 (t, J = 6.0 Hz, 2H), 2.74 (s, 3H), 2.61 (br. t, 2H), 1.57 (br. d, J = 13.2 Hz, 2H), 1.54- 1.46 (m, 2H), 1.37-1.26 (m, 1H), 1.22-1.17 (m, 2H), 1.11 (d, J = 6.4 Hz, 6H), 0.92 (ddd, J = 12.4, 12.2, 4.4 Hz, 2H); MS calcd. for [M + H]⁺ C₂₃H₃₇N₂O₅S: 453.2; found: 453.2. |
| 46 | 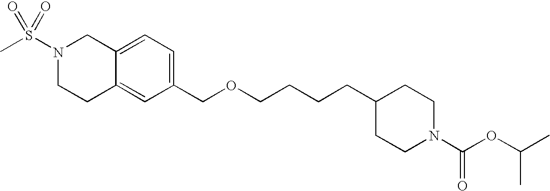 | ¹H-NMR (400 MHz, CD₃CN) δ = 7.08-7.03 (m, 3H), 4.72 (septet, J = 6.4 Hz, 1H), 4.33 (s, 2H), 4.30 (s, 2H), 3.97-3.92 (m, 2H), 3.40 (t, J = 6.0 Hz, 2H), 3.36 (t, J = 6.4 Hz, 2H), 2.86 (t, J = 6.0 Hz, 2H), 2.74 (s, 3H), 2.61 (br. t, 2H), 1.56 (br. d, J = 12.0 Hz, 2H), 1.50- 1.43 (m, 2H), 1.39-1.24 (m, 3H), 1.19-1.13 (m, 2H), 1.11 (d, J = 6.4 Hz, 6H), 0.91 (ddd, J = 12.4, 12.2, 4.4 Hz, 2H); MS calcd. for [M + H]⁺ C₂₄H₃₉N₂O₅S: 467.2; found: 467.2. |
| 47 | 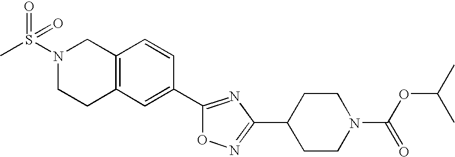 | ¹H-NMR (400 MHz, CDCl₃) δ = 7.99-7.96 (m, 2H), 7.28 (d, J = 8.4 Hz, 1H), 4.97 (septet, J = 6.4 Hz, 1H), 4.56 (s, 2H), 4.29-4.18 (m, 2H), 3.63 (t, J = 6.0 Hz, 2H), 3.12-3.00 (m, 5H), 2.91 (s, 3H), 2.11-2.08 (m, 2H), 1.93- 1.83 (m, 2H), 1.29 (d, J = 6.0 Hz, 6H); MS calcd. for [M + H]⁺ C₂₁H₂₉N₄O₅S: 449.2; found: 449.2 |
| 48 | 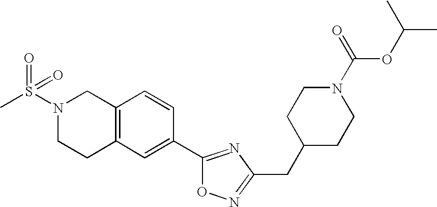 | ¹H-NMR (400 MHz, CD₃CN) δ = 7.96 (s, 1H), 7.94 (d, J = 8.0 Hz, 1H), 7.39 (d, J = 8.0 Hz, 1H), 4.83 (septet, J = 6.4 Hz, 1H), 4.51 (s, 2H), 4.08 (br. d, J = 13.6 Hz, 2H), 3.56 (t, J = 6.0 Hz, 2H), 3.09 (t, J = 6.0 Hz, 2H), 2.88 (s, 3H), 2.82-2.73 (m, 2H), 2.73 (d, J = 6.8 Hz, 2H), 2.08-1.95 (m, 1H), 1.75 (br. d, J = 13.2 Hz, 2H), 1.30-1.16 (m, 2H), 1.22 (d, J = 6.4 Hz, 6H); MS calcd. for [M + H]⁺ C₂₂H₃₁N₄O₅S: 463.2; found: 463.2 |
| 49 | 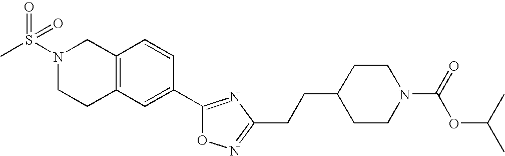 | MS calcd. for [M + H]⁺ C₂₃H₃₃N₄O₅S: 477.2; found: 477.2 |
| 50 | 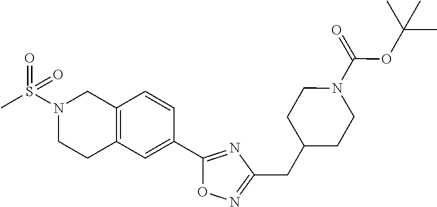 | ¹H-NMR (400 MHz, CDCl₃) δ = 7.91-7.87 (m, 2H), 7.19 (d, J = 8.4 Hz, 1H), 4.47 (s, 2H), 4.04 (br. s, 2H), 3.54 (t, J = 6.0 Hz, 2H), 3.01 (t, J = 6.0 Hz, 2H), 2.81 (s, 3H), 2.71-2.62 (m, 2H), 2.67 (d, J = 7.2 Hz, 2H), 2.01- 1.90 (m, 1H), 1.67 (br. d, J = 12.4 Hz, 2H), 1.38 (s, 9H), 1.25-1.14 (m, 2H); MS calcd. for [M + 2H- Boc]⁺ C₁₈H₂₅N₄O₃S: 377.1; found: 377.1 |
| 51 | 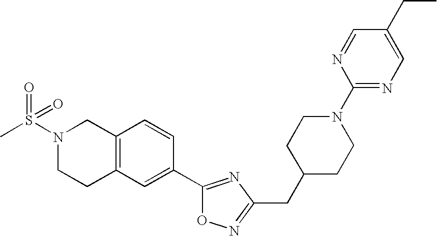 | ¹H-NMR (400 MHz, CD₃CN) δ = 8.20 (s, 2H), 7.97 (s, 1H), 7.95 (d, J = 8.0 Hz, 1H), 7.39 (d, J = 8.0 Hz, 1H), 4.70 (br, d, J = 13.6 Hz, 2H), 4.52 (s, 2H), 3.56 (t, J = 6.0 Hz, 2H), 3.09 (t, J = 6.0 Hz, 2H), 2.91-2.83 (m, 2H), 2.88 (s, 3H), 2.75 (d, J = 6.8 Hz, 2H), 2.46 (q, J = 7.6 Hz, 2H), 2.16-2.12 (m, 1H), 1.84-1.78 (m, 2H), 1.27 (ddd, J = 12.4, 12.4, 4.4 Hz, 2H), 1.18 (t, J = 7.6 Hz, 3H); MS calcd. for [M + H]⁺ C₂₄H₃₁N₆O₃S: 483.1; found: 482.9 |
| 52 | 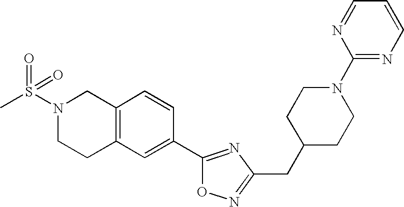 | ¹H-NMR (400 MHz, CD₃CN) δ = 8.32 (d, J = 5.2 Hz, 2H), 7.86 (s, 1H), 7.85 (d, J = 8.0 Hz, 1H), 7.29 (d, J = 8.0 Hz, 1H), 6.60 (t, J = 5.2 Hz, 1H), 4.52 (br. d, J = 13.6 Hz, 2H), 4.41 (s, 2H), 3.46 (t, J = 6.0 Hz, 2H), 3.00-2.93 (m, 4H), 2.78 (s, 3H), 2.67 (d, J = 6.8 Hz, 2H), 2.15-2.06 (m, 1H), 1.83- 1.77 (m, 2H), 1.26 (ddd, J = 12.8, 12.8, 4.4 Hz, 2H); MS calcd. for [M + H]⁺ C₂₄H₃₁N₆O₃S: 483.1; found: 482.9. |
| 53 | 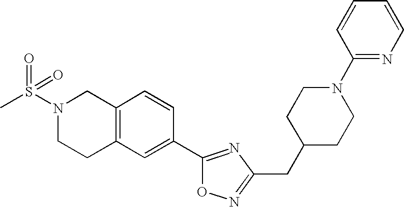 | ¹H-NMR (400 MHz, CD₃CN) δ = 7.90 (dd, J = 6.4, 1.6 Hz, 1H), 7.86 (s, 1H), 7.84 (d, J = 8.0 Hz, 1H), 7.79 (ddd, J = 9.0, 6.4, 1.6 Hz, 1H), 7.29 (d, J = 8.0 Hz, 1H), 7.06 (d, J = 9.0 Hz, 1H), 6.75 (t, J = 7.2 Hz, 1H), 4.41 (s, 2H), 4.08 (br. d, J = 13.6 Hz, 2H), 3.45 (t, J = 6.0 Hz, 2H), 3.15-3.08 (m, 2H), 2.98 (J = 6.0 Hz, 2H), 2.78 (s, 3H), 2.68 (d, J = 7.2 Hz, 2H), 2.19-2.08 (m, 1H), 1.87-1.82 (m, 2H), 1.35 (ddd, J = 13.2, 12.4, 4.0 Hz, 2H); MS calcd. for [M + H]⁺ C₂₃H₂₈N₅O₃S: 454.2; found: 454.2. |
| 54 | 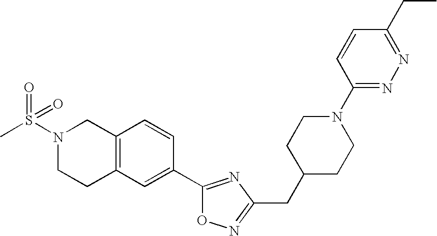 | ¹H-NMR (400 MHz, CD₃CN) δ = 7.86 (s, 1H), 7.83 (d, J = 8.0 Hz, 1H), 7.61-7.55 (m, 2H), 7.28 (d, J = 8.0 Hz, 1H), 4.41 (s, 2H), 4.14 (br. d, J = 13.6 Hz, 2H), 3.45 (t, J = 6.0 Hz, 2H), 3.11-3.04 (m, 2H), 2.98 (t, J = 6.0 Hz, 2H), 2.78 (q, J = 7.2 Hz, 2H), 2.77 (s, 3H), 2.67 (d, J = 7.2 Hz, 2H), 2.20-2.07 (m, 1H), 1.88- 1.83 (m, 2H), 1.32 (ddd, J = 12.5, 12.4, 4.0 Hz, 2H), 1.17 (t, J = 7.2 Hz, 3H); MS calcd. for [M + H]⁺ C₂₄H₃₁N₆O₃S: 483.2; found: 483.2. |
| 55 | 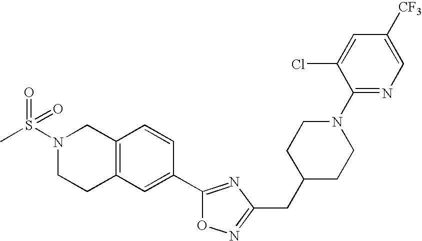 | ¹H-NMR (400 MHz, CD₃CN) δ = 8.34 (m, 1H), 7.86-7.81 (m, 3H), 7.28 (d, J = 8.0 Hz, 1H), 4.40 (s, 2H), 3.96 (br. d, J = 13.2 Hz, 2H), 3.45 (t, J = 6.0 Hz, 2H), 2.98 (t, J = 6.0 Hz, 2H), 2.82 (td, J = 13.2, 2.0 Hz, 2H), 2.77 (s, 3H), 2.69 (d, J = 6.8 Hz, 2H), 2.04-1.96 (m, 1H), 1.80-1.76 (m, 2H), 1.39 (ddd, J = 12.2, 12.0, 3.6 Hz, 2H); MS calcd. for [M + H]⁺ C₂₄H₂₆ClF₃N₅O₃S: 556.1; found: 556.1. |
| 56 | 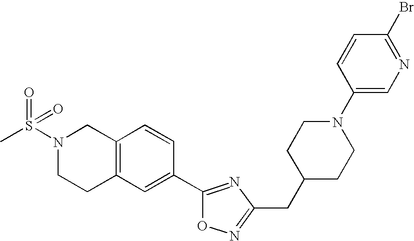 | ¹H-NMR (400 MHz, CD₃CN) δ = 7.92 (d, J = 3.2 Hz, 1H), 7.85 (s, 1H), 7.83 (d, J = 8.0 Hz, 1H), 7.28 (d, J = 8.0 Hz, 1H), 7.22 (d, J = 8.8 Hz, 1H), 7.11 (dd, J = 8.8, 3.2 Hz, 1H), 4.40 (s, 2H), 3.62 (br. d, J = 12.8 Hz, 2H), 3.45 (t, J = 6.0 Hz, 2H), 2.98 (t, J = 6.0 Hz, 2H), 2.77 (s, 3H), 2.72-2.65 (m, 4H), 1.97-1.88 (m, 1H), 1.78-1.72 (m, 2H), 1.34 (ddd, J = 12.2, 12.0, 4.0 Hz, 2H); MS calcd. for [M + H]⁺ C₂₃H₂₇BrN₅O₃S: 532.1; found: 532.1. |
| 57 | 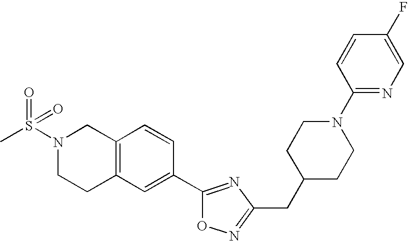 | ¹H-NMR (400 MHz, CD₃CN) δ = 8.00 (d, J = 2.8 Hz, 1H), 7.96 (s, 1H), 7.95 (d, J = 8.0 Hz, 1H), 7.80 (ddd, J = 10.0, 7.2, 2.8 Hz, 1H), 7.39 (d, J = 8.0 Hz, 1H), 7.18 (dd, J = 10.0, 4.0 Hz, 1H), 4.52 (s, 2H), 4.10 (br. d, J = 13.6 Hz, 2H), 3.56 (t, J = 6.0 Hz, 2H), 3.22 (td, J = 13.2, 2.4 Hz, 2H), 3.09 (d, J = 6.0 Hz, 2H), 2.89 (s, 3H), 2.79 (d, J = 7.2 Hz, 2H), 2.27-2.16 (m, 1H), 1.95-1.90 (m, 2H), 1.46 (ddd, J = 13.2, 12.4, 4.0 Hz, 2H); MS calcd. for [M + H]⁺ C₂₃H₂₇FN₅O₃S: 472.2; found: 472.2. |
| 58 | 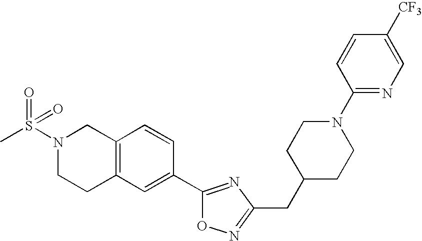 | ¹H-NMR (400 MHz, CDCl₃) δ = 8.39 (m, 1H), 7.98-7.97 (m, 2H), 7.62 (dd, J = 8.8, 2.4 Hz, 1H), 7.28 (d, J = 8.0 Hz, 1H), 6.66 (d, J = 8.0 Hz, 1H), 4.56 (s, 2H), 4.44 (br. d, J = 13.2 Hz, 2H), 3.63 (t, J = 6.0 Hz, 2H), 3.11 (d, J = 6.0 Hz, 2H), 2.99-2.92 (m, 2H), 2.91 (s, 3H), 2.79 (d, J = 7.2 Hz, 2H), 2.26-2.14 (m, 1H), 1.92- 1.88 (m, 2H), 1.40 (ddd, J = 12.4, 12.4, 4.0 Hz, 2H); MS calcd. for [M + H]⁺ C₂₄H₂₇F₃N₅O₃S: 522.2; found: 522.2 |
| 59 | 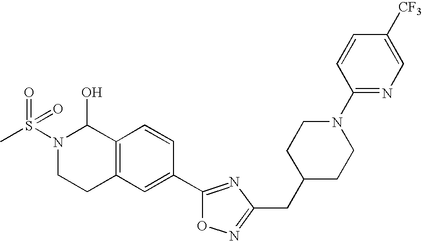 | ¹H-NMR (400 MHz, CD₃CN) δ = 8.28 (m, 1H), 7.90-7.88 (m, 2H), 7.75 (dd, J = 9.6, 2.4 Hz, 1H), 7.48 (d, J = 8.0 Hz, 1H), 6.94 (d, J = 8.0 Hz, 1H), 6.09 (s, 1H), 4.25 (br. d, J = 16.8 Hz, 2H), 3.77-3.72 (m, 1H), 3.40-3.33 (m, 1H), 3.05-2.94 (m, 4H), 2.92 (s, 3H), 2.68 (d, J = 6.8 Hz, 2H), 2.18-2.07 (m, 1H), 1.85-1.80 (m, 2H), 1.30 (ddd, J = 12.8, 12.4, 4.0 Hz, 2H); MS calcd. for [M + H]⁺ C₂₄H₂₇F₃N₅O₄S: 538.2; found: 538.2. |
| 60 | 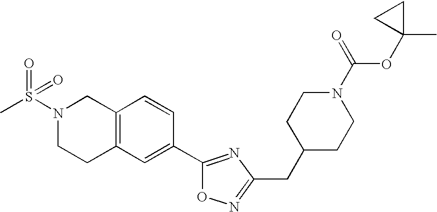 | ¹H-NMR (400 MHz, CDCl₃) δ = 7.88-7.86 (m, 2H), 7.19 (d, J = 8.0 Hz, 1H), 4.47 (s, 2H), 4.10- 3.92 (m, 2H), 3.54 (t, J = 6.0 Hz, 2H), 3.01 (t, J = 6.0 Hz, 2H), 2.81 (s, 3H), 2.69-2.62 (m, 2H), 2.67 (d, J = 6.8 Hz, 2H), 2.01- 1.90 (m, 1H), 1.69-1.65 (m, 2H), 1.47 (s, 3H), 1.25-1.13 (m, 2H), 0.80- 0.77 (m, 2H), 0.56-0.53 (m, 2H); MS calcd. for [M + Na]⁺ C₂₃H₃₀NaN₄O₅S: 497.1; found: 497.1 |
| 61 | 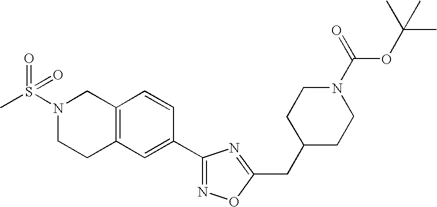 | ¹H-NMR (400 MHz, CD₃CN) δ = 7.79-7.77 (m, 2H), 7.23 (d, J = 8.4 Hz, 1H), 4.39 (s, 2H), 3.95 (br. d, J = 12.8 Hz, 2H), 3.45 (t, J = 6.0 Hz, 2H), 2.97 (t, J = 6.0 Hz, 2H), 2.82 (t, J = 7.2 Hz, 2H), 2.77 (s, 3H), 2.70- 2.58 (m, 2H), 2.03-1.95 (m, 1H), 1.68-1.62 (m, 2H), 1.33 (s, 9H), 1.12 (ddd, J = 12.0, 12.0, 4.0 Hz, 2H); MS calcd. for [M + 2H- Boc]⁺ C₁₈H₂₅N₄O₃S: 377.1; found: 377.1 |
| 62 | 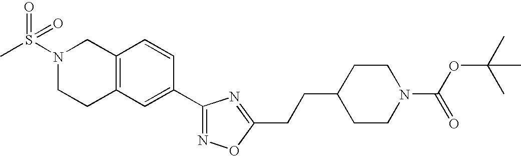 | ¹H-NMR (400 MHz, CD₃CN) δ = 7.78-7.76 (m, 2H), 7.23 (d, J = 8.4 Hz, 1H), 4.39 (s, 2H), 3.94 (br. d, J = 13.2 Hz, 2H), 3.45 (t, J = 6.0 Hz, 2H), 2.96 (t, J = 6.0 Hz, 2H), 2.90 (t, J = 8.0 Hz, 2H), 2.77 (s, 3H), 2.60 (br. s, 2H), 1.73-1.67 (m, 2H), 1.64-1.61 (m, 2H), 1.52-1.38 (m, 1H), 1.33 (s, 9H), 1.00 (ddd, J = 12.8, 12.4, 4.4 Hz, 2H); MS calcd. for [M + 2H- Boc]⁺ C₁₉H₂₇N₄O₃S: 391.2; found: 391.1. |
| 63 | 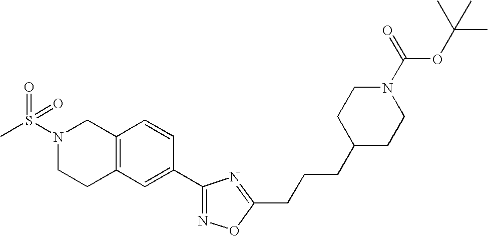 | ¹H-NMR (400 MHz, CD₃CN) δ = 7.78-7.76 (m, 2H), 7.23 (d, J = 8.8 Hz, 1H), 4.39 (s, 2H), 3.92 (br. d, J = 12.4 Hz, 2H), 3.44 (t, J = 6.0 Hz, 2H), 2.97 (t, J = 6.0 Hz, 2H), 2.85 (t, J = 7.6 Hz, 2H), 2.77 (s, 3H), 2.59 (br. s, 2H), 1.83-1.74 (m, 2H), 1.60-1.56 (m, 2H), 1.42-1.35 (m, 1H), 1.33 (s, 9H), 1.29-1.24 (m, 2H), 0.93 (ddd, J = 12.8, 12.4, 4.4 Hz, 2H); MS calcd. for [M + 2H- Boc]⁺ C₂₀H₂₉N₄O₃S: 405.2; found: 405.2. |
| 64 | 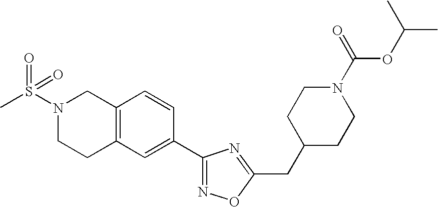 | ¹H-NMR (400 MHz, CDCl₃) δ = 7.82-7.80 (m, 2H), 7.14 (d, J = 8.0 Hz, 1H), 4.42 (s, 2H), 4.14 (septet, J = 6.0 Hz, 1H), 3.53 (t, J = 6.0 Hz, 2H), 3.47 (br. d, J = 12.0 Hz, 2H), 2.99 (t, J = 6.0 Hz, 2H), 2.90 (t, J = 7.2 Hz, 2H), 2.85-2.82 (m, 2H), 2.80 (s, 3H), 2.21-2.12 (m, 1H), 1.99-1.96 (m, 2H), 1.85-1.75 (m, 2H), 1.25 (d, J = 6.0 Hz, 2H); MS calcd. for [M + 2H- Boc]⁺ C₁₈H₂₅N₄O₃S: 377.1; found: 377.1 |
| 65 | 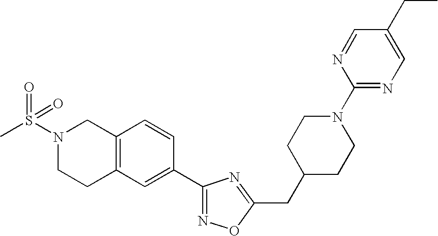 | ¹H-NMR (400 MHz, DMSO-d₆) δ = 8.23 (s, 2H), 7.84-7.81 (m, 2H), 7.39 (d, J = 8.0 Hz, 1H), 4.62 (br. d, J = 13.2 Hz, 2H), 4.45 (s, 2H), 3.47 (t, J = 6.0 Hz, 2H), 3.04-2.98 (m, 4H), 2.98 (s, 3H), 2.90-2.83 (m, 2H), 2.42 (q, J = 7.6 Hz, 2H), 2.21- 2.11 (m, 1H), 1.77-1.73 (m, 2H), 1.23 (ddd, J = 12.4, 12.4, 4.4 Hz, 2H), 1.12 (t, J = 7.6 Hz, 3H); MS calcd. for [M + H]⁺ C₂₄H₃₁N₆O₃S: 483.1; found: 483.2 |
| 66 | 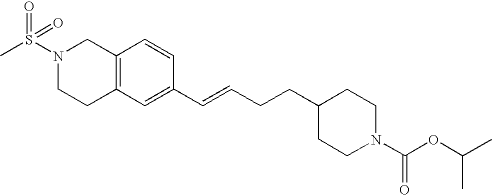 | ¹H-NMR (400 MHz, CD₃CN) δ 7.14-7.09 (m, 2H), 6.99 (d, J = 8.0 Hz, 1H), 6.29 (d, J = 16.0 Hz, 1H), 6.20 (dt, J = 16.0, 6.8 Hz, 1H), 4.72 (septet, J = 6.0 Hz, 1H), 4.28 (s, 2H), 3.98-3.93 (m, 2H), 3.39 (t, J = 6.0 Hz, 2H), 2.84 (t, J = 6.0 Hz, 2H), 2.74 (s, 3H) 2.65 (br. s, 2H), 2.17-1.10 (m, 2H), 1.62 (br. d, J = 13.2 Hz, 2H), 1.43-1.35 (m, 1H), 1.34-1.28 (m, 2H), 1.11 (d, J = 6.0 Hz, 6H), 1.04-0.91 (m, 2H); MS calcd. for [M + H]⁺ C₂₃H₃₅N₂O₄S: 435.2; found: 435.2 |
| 67 | 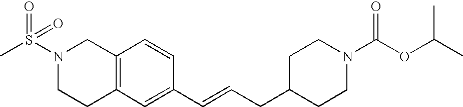 | ¹H-NMR (400 MHz, CDCl₃) δ = 7.21 (dd, J = 8.0, 1.2 Hz, 1H), 7.14 (s, 1H), 7.05 (d, J = 8.0 Hz, 1H), 6.35 (d, J = 16.0 Hz, 1H), 6.19 (dt, J = 16.0, 7.2 Hz, 1H), 4.93 (septet, J = 6.0 Hz, 1H), 4.46 (s, 2H), 4.15 (br. s, 2H), 3.58 (t, J = 6.0 Hz, 2H), 2.98 (t, J = 6.0 Hz, 2H), 2.86 (s, 3H) 2.74 (br. t, J = 12.0 Hz, 2H), 2.18 (t, J = 6.8 Hz, 2H), 1.73 (br. d, J = 12.8 Hz, 2H), 1.63-1.54 (m, 1H), 1.25 (d, J = 6.0 Hz, 6H), 1.22-1.12 (m, 2H); MS calcd. for [M + H]⁺ C₂₂H₃₃N₂O₄S: 421.2; found: 421.2. |
| 68 | 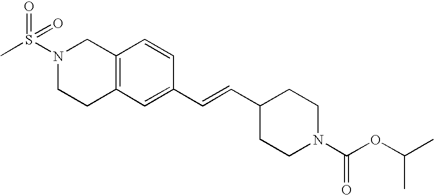 | ¹H-NMR (400 MHz, CDCl₃) δ = 7.22 (dd, J = 7.6, 1.2 Hz, 1H), 7.15 (s, 1H), 7.05 (d, J = 8.4 Hz, 1H), 6.36 (d, J = 16.0 Hz, 1H), 6.15 (dd, J = 16.0, 6.8 Hz, 1H), 4.95 (septet, J = 6.0 Hz, 1H), 4.46 (s, 2H), 4.21 (br. s, 2H), 3.58 (t, J = 6.0 Hz, 2H), 2.98 (t, J = 6.0 Hz, 2H), 2.85 (s, 3H) 2.87-2.80 (m, 2H), 2.38-2.27 (m, 1H), 1.78 (br. d, J = 12.8 Hz, 2H), 1.45-1.36 (m, 2H), 1.26 (d, J = 6.0 Hz, 6H); MS calcd. for [M + H]⁺ C₂₁H₃₁N₂O₄S: 407.2; found: 407.2. |
| 69 | 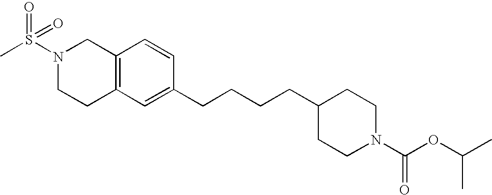 | ¹H-NMR (400 MHz, CD₃CN) δ = 6.99-6.93 (m, 3H), 4.72 (septet, J = 6.0 Hz, 1H), 4.27 (s, 2H), 3.93 (br. d, J = 12.4 Hz, 2H), 3.38 (t, J = 6.0 Hz, 2H), 2.83 (t, J = 6.0 Hz, 2H), 2.74 (s, 3H) 2.61 (br. t, 2H), 2.48 (t, J = 7.6 Hz, 2H), 1.56-1.44 (m, 4H), 1.36-1.15 (m, 5H), 1.10 (d, J = 6.0 Hz, 6H), 0.99- 0.85 (m, 2H); MS calcd. for [M + H]⁺ C₂₃H₃₇N₂O₄S: 437.2; found: 437.2 |
| 70 | 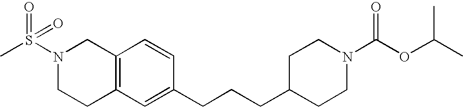 | ¹H-NMR (400 MHz, CD₃CN) δ = 6.99-6.93 (m, 3H), 4.72 (septet, J = 6.0 Hz, 1H), 4.27 (s, 2H), 3.93 (br. d, J = 13.2 Hz, 2H), 3.38 (t, J = 6.0 Hz, 2H), 2.83 (t, J = 6.0 Hz, 2H), 2.73 (s, 3H) 2.61 (br. t, 2H), 2.46 (t, J = 8.0 Hz, 2H), 1.58-1.46 (m, 4H), 1.40-1.29 (m, 1H), 1.20- 1.14 (m, 2H), 1.10 (d, J = 6.0 Hz, 6H), 0.91 (ddd, J 12.8, 12.4, 4.4 Hz, 2H); MS calcd. for [M + H]⁺ C₂₂H₃₅N₂O₄S: 423.2; found: 423.2. |
| 71 | 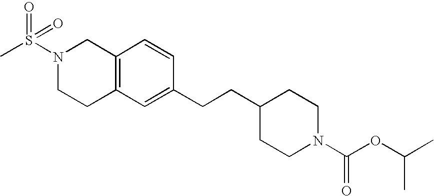 | ¹H-NMR (400 MHz, CDCl₃) δ = 7.03-6.98 (m, 3H), 4.93 (septet, J = 6.4 Hz, 1H), 4.45 (s, 2H), 4.15 (br. s, 2H), 3.57 (t, J = 6.0 Hz, 2H), 2.97 (t, J = 6.0 Hz, 2H), 2.86 (s, 3H) 2.73 (br. t, J = 12.4 Hz, 2H), 2.61 (t, J = 8.0 Hz, 2H), 1.75-1.72 (br. d, J = 12.8 Hz, 2H), 1.59-1.54 (m, 2H), 1.51-1.40 (m, 1H), 1.25 (d, J = 6.4 Hz, 6H), 1.21-1.10 (m, 2H); MS calcd. for [M + H]⁺ C₂₁H₃₃N₂O₄S: 409.2; found: 409.2. |
| 72 | 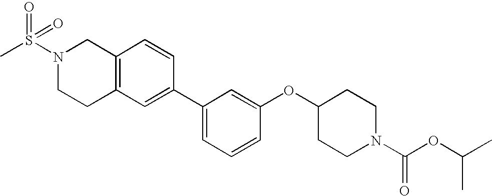 | ¹H-NMR (400 MHz, CD₃CN) δ = 7.40-7.38 (m, 2H), 7.27 (t, J = 8.0 Hz, 1H), 7.16-7.10 (m, 3H), 6.87-6.85 (m, 1H), 4.75 (septet, J = 6.4 Hz, 1H), 4.58-4.52 (m, 1H), 4.36 (s, 2H), 3.71-3.65 (m, 2H), 3.44 (t, J = 6.0 Hz, 2H), 3.23-3.17 (m, 2H), 2.94 (t, J = 6.0 Hz, 2H), 2.77 (s, 3H), 1.91- 1.88 (m, 2H), 1.60-1.52 (m, 2H), 1.13 (d, J = 6.4 Hz, 6H); MS calcd. for [M + H]⁺ C₂₅H₃₃N₂O₅S: 473.2; found: 473.2 |
| 73 | 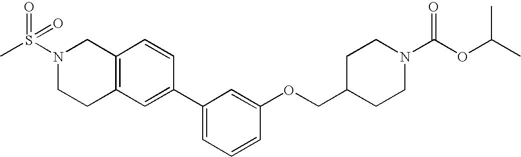 | ¹H-NMR (400 MHz, CD₃CN) δ = 7.40-7.37 (m, 2H), 7.28-7.25 (m, 1H), 7.15-7.08 (m, 3H), 6.82 (dd, J = 8.4, 2.4 Hz, 1H), 4.75 (septet, J = 6.4 Hz, 1H), 4.36 (s, 2H), 4.03 (br. d, J = 12.8 Hz, 2H), 3.83 (d, J = 6.4 Hz, 2H), 3.44 (t, J = 6.0 Hz, 2H), 2.94 (t, J = 6.0 Hz, 2H), 2.76 (s, 3H), 2.71 (br. t, 2H), 1.96-1.88 (m, 1H), 1.73 (br. d, J = 12.8 Hz, 2H), 1.20-1.10 (m, 2H), 1.13 (d, J = 6.4 Hz, 6H); MS calcd. for [M + H]⁺ C₂₆H₃₅N₂O₅S: 487.2; found: 487.2 |
| 74 | 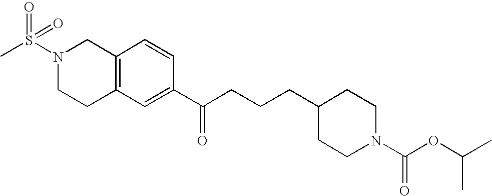 | ¹H-NMR (400 MHz, CDCl₃) δ = 7.81-7.78 (m 2H), 7.21 (d, J = 8.0 Hz, 1H), 4.92 (septet, J = 6.4 Hz, 1H), 4.52 (s, 2H), 4.14 (br. s, 2H), 3.61 (t, J = 6.0 Hz, 2H), 3.07 (t, J = 6.0 Hz, 2H), 2.89 (s, 3H), 2.72 (br. t, J = 12.4 Hz, 2H), 1.81-1.69 (m, 4H), 1.49-1.42 (m, 1H), 1.37- 1.31 (m, 2H), 1.25 (d, J = 6.4 Hz, 6H), 1.16-1.07 (m 2H); MS calcd. for [M + H]⁺ C₂₃H₃₅N₂O₅S: 451.2; found: 451.2 |
| 75 | 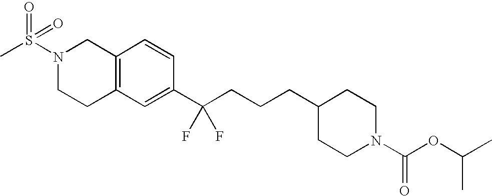 | ¹H-NMR (400 MHz, CD₃CN) δ = 7.36-7.34 (m 2H), 7.26 (d, J = 8.4 Hz, 1H), 4.82 (septet, J = 6.0 Hz, 1H), 4.46 (s, 2H), 4.03 (br. d, J = 12.8 Hz, 2H), 3.53 (t, J = 6.0 Hz, 2H), 3.01 (t, J = 6.0 Hz, 2H), 2.87 (s, 3H), 2.70 (br. t, 2H), 2.23-2.11 (m, 2H), 1.62 (br. d, J = 12.4 Hz, 2H), 1.46-1.35 (m, 3H), 1.29-1.25 (m, 2H), 1.21 (d, J = 6.0 Hz, 6H), 0.99 (ddd, J = 13.2, 12.8, 4.4, 2H); ¹⁹F-NMR (376 MHz, CD₃CN) δ = - 94.585; MS calcd. for [M + H]⁺ C₂₃H₃₅F₂N₂O₄S: 473.2; found: 473.2 |
| 76 | 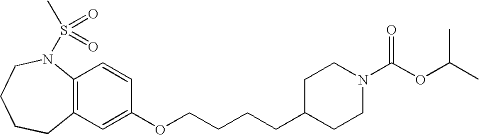 | ¹H-NMR (400 MHz, CD₃CN) δ = 7.13 (d, J = 8.4 Hz, 1H), 6.70 (d, J = 2.8 Hz, 1H), 6.64 (dd, J = 8.8, 3.2 Hz, 1H), 4.72 (septet, J = 6.0 Hz, 1H), 3.95 (br. d, J = 13.2 Hz, 2H), 3.88 (t, J = 6.4 Hz, 2H), 3.42 (br. s, 2H), 2.94 (s, 3H), 2.73-2.70 (m, 2H), 2.63 (br. t, 2H), 1.80- 1.75 (m, 2H), 1.70-1.57 (m, 6H), 1.42-1.30 (m, 3H), 1.24-1.18 (m, 2H), 1.11 (d, J = 6.0 Hz, 6H), 0.93 (ddd, J = 12.8, 12.4, 4.0 Hz, 2H); MS calcd. for [M + H]⁺ C₂₄H₃₉N₂O₅S: 467.2; found: 467.2 |
| 77 | 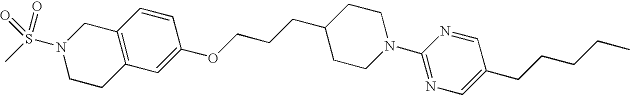 | ¹H NMR (400 MHz, CD₃CN) δ 8.16 (s, 2H), 7.04 (d, J = 6.2 Hz, 1H), 6.75 (dd, J = 1.8, 6.3 Hz, 1H), 6.72 (d, J = 2.1 Hz, 1H), 4.65 (m, 2H), 4.31 (s, 2H), 3.94 (t, J = 5.1 Hz, 2H), 3.45 (t, J = 4.5 Hz, 2H), 2.91 (t, J = 4.2 Hz, 2H), 2.82 (dt, J = 1.8, 9.9 Hz, 2H), 2.81 (s, 3H), 2.40 (t, J = 5.7 Hz, 2H), 1.80 (m, 4H), 1.53 (m, 3H), 1.41-1.25 (m, 6H), 1.09 (ddd, J = 3.3, 9.6, 18.6 Hz, 2H), 0.89 (t, J = 5.1 Hz, 3H; MS calcd. for [M + H]⁺ C₂₇H₄₁N₄O₃S: 501.3; found: 501.2. |
| 78 | 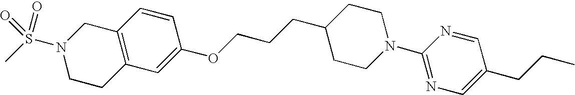 | ¹H NMR (400 MHz, CD₃CN) δ 8.16 (s, 2H), 7.04 (d, J = 6.3 Hz, 1H), 6.76 (dd, J = 2.1, 6.3 Hz, 1H), 6.72 (d, J = 2.1 Hz, 1H), 4.65 (dt, J = 9.9, 1.5 Hz, 2H), 4.31 (s, 2H), 3.94 (t, J = 5.1 Hz, 2H), 3.45 (t, J = 4.5 Hz, 2H), 2.91 (t, J = 4.5 Hz, 2H), 2.82 (dt, J = 1.8, 9.9 Hz, 2H), 2.81 (s, 3H), 2.38 (t, J = 5.4 Hz, 2H), 1.78 (m, 4H), 1.86 (m, 3H), 1.38 (m, 2H), 1.09 (ddd, J = 3.0, 9.6, 18.3 Hz, 2H), 0.91 (t, J = 5.4 Hz, 3H); MS calcd. for [M + H]⁺ C₂₅H₃₇N₄O₃S: 473.3; found: 473.2. |
| 79 | 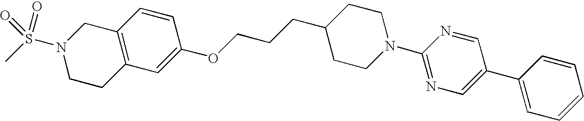 | ¹H NMR (400 MHz, CD₃CN) δ 8.59 (s, 2H), 7.58 (m, 2H), 7.46 (m, 2H), 7.35 (m, 1H), 7.04 (d, J = 6.3 Hz, 1H), 6.76 (dd, J = 2.1, 6.3 Hz, 1H), 6.73 (d, J = 1.8 Hz, 1H), 4.76 (m, 2H), 4.32 (s, 2H), 3.95 (t, J = 5.1 Hz, 2H), 3.45 (t, J = 4.5 Hz, 2H), 2.91 (m, 2H), 2.81 (s, 3H), 1.80 (m, 4H), 1.63 (m, 1H), 1.40 (m, 2H), 1.14 (ddd, J = 3.0, 9.6, 18.3 Hz, 2H); MS calcd. for [M + H]⁺ C₂₈H₃₅N₄O₃S: 507.2; found: 507.2. |
| 80 | 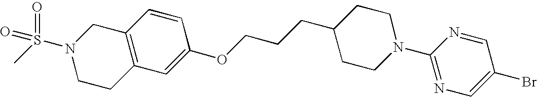 | ¹H NMR (400 MHz, CD₃CN) δ 8.31 (s, 2H), 7.04 (d, J = 6.3 Hz, 1H), 6.75 (dd, J = 2.1, 6.3 Hz, 1H), 6.72 (d, J = 1.8 Hz, 1H), 4.62 (m, 2H), 4.31 (s, 2H), 3.94 (t, J = 4.8 Hz, 2H), 3.45 (t, J = 4.5 Hz, 2H), 2.92-2.82 (m, 4H), 2.81 (s, 3H), 1.82- 1.74 (m, 4H), 1.59 (m, 1H), 1.38 (m, 2H), 1.10 (ddd, J = 3.0, 9.6, 18.3 Hz, 2H); MS calcd. for [M + H]⁺ C₂₂H₂₉BrN₄O₃S: 509.1; found: 509.0. |
| 81 | 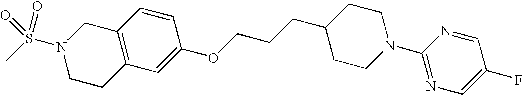 | ¹H NMR (400 MHz, CD₃CN) δ 8.23 (s, 2H), 7.04 (d, J = 6.3 Hz, 1H), 6.75 (dd, J = 1.8, 6.3 Hz, 1H), 6.72 (d, J = 1.8 Hz, 1H), 4.61 (m, 2H), 4.31 (s, 2H), 3.94 (t, J = 4.8 Hz, 2H), 3.45 (t, J = 4.5 Hz, 2H), 2.92-2.81 (m, 4H), 2.81 (s, 3H), 1.80- 1.76 (m, 4H), 1.57(m, 1H), 1.42-1.36 (m, 2H), 1.10 (ddd, J = 3.3, 9.6, 18.6 Hz, 2H); MS calcd. for [M + H]⁺ C₂₂H₃₀FN₄O₃S: 449.2; found: 449.2. |
| 82 | 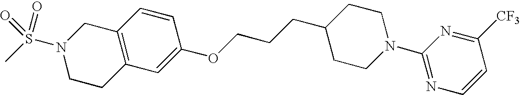 | ¹H NMR (400 MHz, CD₃CN) δ 8.53 (d, J = 2.4 Hz, 1H), 7.04 (d, J = 4.2 Hz, 1H), 6.80 (d, J = 2.4 Hz, 1H), 6.76 (dd, J = 1.2, 4.2 Hz, 1H), 6.20 (d, J = 0.9 Hz, 1H), 4.71 (m, 2H), 4.32 (s, 2H), 3.95 (t, J = 3.3 Hz, 2H), 3.45 (t, J = 3.0 Hz, 2H), 2.95-2.90 (m, 4H), 2.81 (s, 3H), 1.80 (m, 4H), 1.63 (m, 1H), 1.40 (m, 2H), 1.12 (ddd, J = 2.1, 6.6, 12.3 Hz, 2H); MS calcd. for [M + H]⁺ C₂₃H₃₀F₃N₄O₃S: 499.2; found: 499.2. |
| 83 | 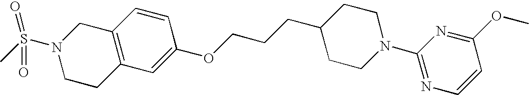 | ¹H NMR (400 MHz, CD₃CN) δ 8.12 (d, J = 5.1 Hz, 1H), 7.04 (d, J = 6.3 Hz, 1H), 6.75 (dd, J = 1.8, 6.3 Hz, 1H), 6.72 (d, J = 2.1 Hz, 1H), 6.22 (d, J = 5.1 Hz, 1H), 4.31 (s, 2H), 3.98 (s, 3H), 3.94 (t, J = 5.1 Hz, 2H), 3.45 (t, J = 5.1 Hz, 2H), 3.45 (t, J = 5.1 Hz, 2H), 3.09 (m, 2H), 2.90 (t, J = 4.5 Hz, , 2H), 2.81 (s, 3H), 1.87 (m, 2H), 1.77 (m, 2H), 1.68 (m, 1H), 1.41 (m, 2H), 1.22 (ddd, J = 3.3, 9.9, 18.9 Hz, 2H); MS calcd. for [M + H]⁺ C₂₃H₃₃N₄O₄S: 461.2; found: 461.2. |
| 84 | 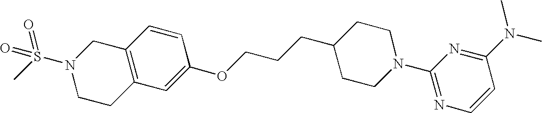 | ¹H NMR (400 MHz, CD₃CN) δ 7.74 (d, J = 5.7 Hz, 1H), 7.04 (d, J = 6.3 Hz, 1H), 6.76 (dd, J = 1.8, 6.3 Hz, 1H), 6.72 (d, J = 1.8 Hz, 1H), 6.20 (d, J = 5.7 Hz, 1H), 4.40 (m, 2H), 4.31 (s, 2H), 3.95 (t, J = 4.8 Hz, 2H), 3.45 (t, J = 4.5 Hz, 2H), 3.19 (s, 3H), 3.12 (s, 3H), 3.03 (m, 2H), 2.91 (t, J = 4.5 Hz, 2H), 2.81 (s, 3H), 1.84 (m, 2H), 1.77 (m, 2H), 1.64 (m 1H), 1.40 (m, 2H), 1.19 (ddd, J = 3.0, 9.6, 18.9 Hz, 2H); MS calcd. for [M + H]⁺ C₂₄H₃₆N₅O₃S: 474.3; found: 474.2. |
| 85 | 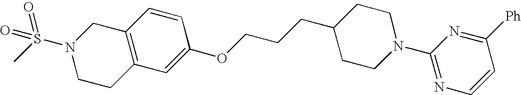 | ¹H NMR (400 MHz, CD₃CN) δ 8.36 (d, J = 4.2 Hz, 1H), 8.15 (m, 2H), 7.55 (m, 3H), 7.16 (d, J = 4.2 Hz, 1H), 7.05 (d, J = 6.3 Hz, 1H), 6.76 (dd, J = 2.1, 6.3 Hz, 1H), 6.75 (d, J = 1.8 Hz, 1H), 4.77 (m, 2H), 4.31 (s, 2H), 3.96 (t, J = 5.1 Hz 2H), 3.45 (t, J = 4.5, 2H), 3.04 (dt, J = 1.5, 10.2 Hz, 2H), 2.91 (t, J = 4.5 Hz, 2H), 2.81 (s, 3H), 1.87 (m, 2H), 1.81 (m, 2H), 1.68 (m 1H), 1.42 (m, 2H), 1.21 (ddd, J = 3.3, 9.9, 18.9 Hz, 2H); MS calcd. for [M + H]⁺ C₂₈H₃₅N4O₃S: 507.2; found: 507.2. |
| 86 | 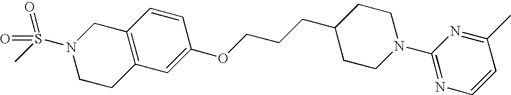 | MS calcd. for [M + H]⁺ C₂₃H₃₃N₄O₃S: 445.2; found: 445.2. |
| 87 | 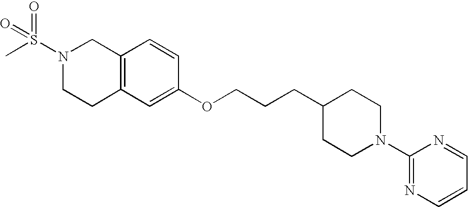 | ¹H NMR (400 MHz, CD₃CN) δ 8.27 (d, J = 3.3 Hz, 1H), 7.04 (d, J = 6.3 Hz, 1H), 6.76 (dd, J = 1.8, 6.3 Hz, 1H), 6.72 (d, J = 2.1 Hz, 1H), 6.48 (t, J = 3.6 Hz, 1H), 4.70 (dt, J = 6.6, 1.8 Hz, 2H), 4.31 (s, 2H), 3.94 (t, J = 4.8 Hz, 2H), 3.45 (t, J = 4.5 Hz, 2H), 2.91 (t, J = 4.5 Hz, 2H), 2.84 (dt, J = 2.1, 9.9 Hz, 2H), 2.81 (s, 3H), 1.78 (m, 4H), 1.59 (m, 1H), 1.38 (m, 2H), 1.09 (ddd, J = 3.0, 9.3, 18.3 Hz, 2H); MS calcd. for [M + H]⁺ C₂₂H₃₁N₄O₃S: 431.2; found: 431.2. |
| 88 | 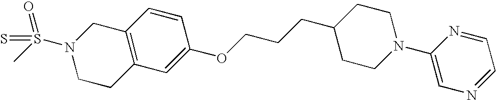 | ¹H NMR (400 MHz, CD₃CN) δ 8.17 (d, J = 1.2, 2.1 Hz, 1H), 7.99 (dd, J = 1.2 Hz, 1H), 7.73 (d, J = 1.8 Hz, 1H), 7.04 (d, J = 6.3 Hz, 1H), 6.75 (dd, J = 1.8, 6.3 Hz, 1H), 6.72 (d, J = 1.8 Hz, 1H), 4.34 (m, 2H), 4.31 (s, 2H), 3.94 (t, J = 4.8 Hz, 2H), 3.45 (t, J = 4.5 Hz, 2H), 2.91 (t, J = 4.5 Hz, 2H), 2.85 (dt, J = 1.5, 9.3 Hz, 2H), 2.81 (s, 3H), 1.84- 1.74 (m, 4H), 1.60 (m, 1H), 1.40 (m, 2H), 1.17 (ddd, J = 3.3, 9.9, 18.6 Hz, 2H); MS calcd. for [M + H]⁺ C₂₂H₃₁N₄O₃S: 431.2; found: 431.2. |
| 89 | 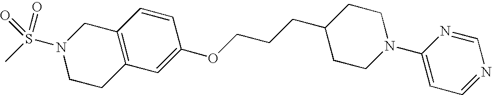 | ¹H NMR (400 MHz, CD₃CN) δ 8.44 s, 1H), 8.10 (d, J = 4.8 Hz, 1H), 7.04 (d, J = 6.3 Hz, 1H), 6.75 (dd, J = 1.8, 6.3 Hz, 1H), 6.72 (d, J = 1.8, 6.3 Hz, 1H), 6.62 (dd, J = 0.9, 4.8 Hz, 1H), 4.39 (m, 2H), 4.31 (s, 2H), 3.94 (t, J = 4.8 Hz, 2H), 3.45 (t, J = 4.5 Hz, 2H), 2.92-2.82 (m, 4H), 2.81 (s, 3H), 1.28 (m, 4H), 1.62 (m, 1H), 1.38 (m, 2H), 1.21 (ddd, J = 3.0, 9.6, 18.6 Hz, 2H); MS calcd. for [M + H]⁺ C₂₂H₃₁N₄O₃S: 431.2; found: 431.2. |
| 90 | 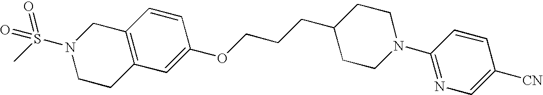 | ¹H NMR (400 MHz, CDCl₃) δ 8.37 (dd, J = 0.6, 1.8 Hz, 1H), 7.65 (dd, J = 1.8, 6.6 Hz, 1H), 7.40 (d, J = 6.6 Hz, 1H), 6.77- 6.70 (m, 3H), 4.43 (d, J = 10.2 Hz, 2H), 4.31 (s, 2H), 3.94 (t, J = 4.8 Hz, 2H), 3.45 (t, J = 4.5 Hz, 2H), 2.90 (m, 4H), 2.8 (s, 3H), 1.78 (m, 4H), 1.63 (m, 1H), 1.39 (m, 2H), 1.13 (ddd, J = 3.3, 9.6, 18.6 Hz, 2H); MS calcd. for [M + H]⁺ C₂₄H₃₁N₄O₃S: 455.2; found: 455.2. |
| 91 | 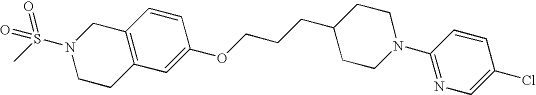 | ¹H NMR (400 MHz, CDCl₃) δ 8.04 (d, J = 2.1 Hz, 1H), 7.45 (dd, J = 1.8, 6.6 Hz, 1H), 7.04 (d, J = 2.1 Hz, 1H), 6.77-6.70 (m, 3H), 4.31 (s, 2H), 4.24 (d, J = 9.9 Hz, 2H), 3.94 (t, J = 4.8 Hz, 2H), 3.45 (t, J = 4.5 Hz, 2H), 2.90 (t, J = 4.5 Hz, 2H), 2.83-2.79 (m, 5H), 1.81- 1.74 (m, 4H), 1.53 (m, 1H), 1.41-1.36 (m, 2H), 1.15 (ddd, J = 3.0, 9.6, 18.6 Hz, 2H); MS calcd. for [M + H]⁺ C₂₃H₃₁ClN₃O₃S: 464.2; found: 464.1. |
| 92 | 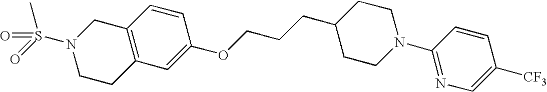 | ¹H NMR (400 MHz, CDCl₃) δ 8.35 (d, J = 0.3 Hz, 1H), 7.67 (dd, J = 2.1, 6.9 Hz, 1H), 7.04 (d, J = 6.3 Hz, 1H), 6.79-6.72 (m, 3H), 4.41 (d, J = 9.9 Hz, 2H), 4.31 (s, 2H), 3.94 (t, J = 5.1 Hz, 2H), 3.45 (t, J = 4.5 Hz, 2H), 2.93-2.86 (m, 2H), 2.81 (s, 3H), 1.82-1.74 (m, 2H), 1.60 (m, 1H), 1.42-1.36 (m, 2H), 1.14 (ddd, J = 3.0, 9.6, 18.6 Hz, 2H); MS calcd. for [M + H]⁺ C₂₄H₃₁F₃N₃O₃S: 498.2; found: 498.2. |
| 93 | 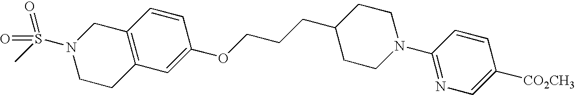 | ¹H NMR (400 MHz, CD₃CN) δ 8.67 (d, J = 1.8 Hz, 1H), 7.94 (dd, J = 1.8, J = 6.9 Hz, 1H), 7.04 (d, J = 8.7 Hz, 1H), 6.75 (dd, J = 2.1, 6.3 Hz, 1H), 6.71 (d, J = 6.9 Hz, 1H), 4.45 (d, J = 9.9 Hz, 2H), 4.31 (s, 2H), 3.94 (t, J = 5.1 Hz, 2H), 3.80 (s, 3H), 3.45 (t, J = 4.5 Hz, 2H), 2.91 (m, 4H), 2.81 (s, 3H), 1.82- 1.74 (m, 4H), 1.62 (m, 1H), 1.39 (m, 2H), 1.14 (ddd, J = 3.0, 9.6, 18.6 Hz, 2H); MS calcd. for [M + H]⁺ C₂₅H₃₄N₃O₅S: 488.2; found: 488.2. |
| 94 | 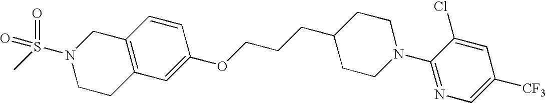 | ¹H NMR (400 MHz, CD₃CN) δ 8.42 (dd, J = 0.6, 1.5 Hz, 1H), 7.89 (d, J = 1.5 Hz, 1H), 7.04 (d, J = 6.3 Hz, 1H), 6.76 (dd, J = 2.1, 6.3 Hz, 1H), 6.73 (d, J = 1.8 Hz, 1H), 4.31 (s, 2H), 4.03 (m, 2H), 3.95 (t, J = 4.8 Hz, 2H), 3.45 (t, J = 4.5 Hz, 2H), 2.92-2.84 (m, 4H), 2.81 (s, 3H), 1.85-1.75 (m, 4H), 1.56 (m, 1H), 1.42 (m, 2H), 1.31 (ddd, J = 3.3, 9.9, 18.9 Hz, 2H); MS calcd. for [M + H]⁺ C₂₄H₃₀ClF₃N₃O₃S: 532.2; found: 532.1. |
| 95 | 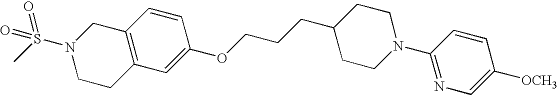 | ¹H NMR (400 MHz, CD₃CN) δ 7.76 (dd, J = 1.2, 3.6 Hz, 1H), 7.12 (dd, J = 1.2, 6.0 Hz, 1H), 7.04 (d, J = 6.3 Hz, 1H), 6.81 (dd, J = 3.6, 6.0 Hz, 1H), 6.76 (dd, J = 2.1, 6.3 Hz, 1H), 6.72 (d, J = 1.8 Hz, 1H), 4.32 (s, 2H), 3.95 (t, J = 5.1 Hz, 2H), 3.91 (m, 2H), 3.80 (s, 3H), 3.45 (t, J = 4.5 Hz, 2H), 2.91 (t, J = 4.5 Hz, 2H), 2.81 (s, 3H), 2.67 (dt, J = 1.5, 9.3, 2H), 1.81-1.75 (m, 4H), 1.47 (m, 1H), 1.42 (m, 2H), 1.28 (ddd, J = 2.7, 9.3, 18.6 Hz, 2H); MS calcd. for [M + H]⁺ C₂₄H₃₄N₃O₄S: 460.2; found: 460.2. |
| 96 | 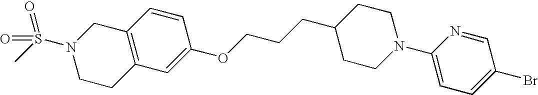 | ¹H NMR (400 MHz, CD₃CN) δ 8.13 (m, 1H), 7.58 (m, 1H), 7.04 (d, J = 6.3 Hz, 1H), 6.75 (dd, J = 2.1, 6.3 Hz, 1H), 6.71 (m, 2H), 4.31 (s, 2H), 4.23 (m, 2H), 3.94 (t, J = 4.8 Hz, 2H), 3.45 (t, J = 4.5 Hz, 2H), 2.91 (t, J = 4.5 Hz, 2H), 2.82 (m, 2H), 2.81 (s, 3H), 1.81-1.74 (m, 4H), 1.55 (m, 1H), 1.41-1.36 (m, 2H), 1.55 (ddd, J = 3.0, 9.3, 18.6 Hz, 2H); MS calcd. for [M + H]⁺ C₂₃H₃₁BrN₃O₃S: 508.1; found: 508.1. |
| 97 | 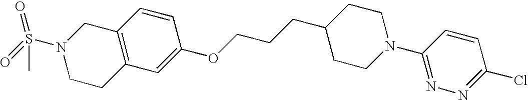 | ¹H NMR (400 MHz, CDCl₃) δ 7.26 (d, J = 2.4 Hz, 1H), 7.02 (dd, J = 7.2, 17.7 Hz, 2H), 6.75 (dd, J = 1.8, 6.3 Hz, 1H), 6.66 (d, J = 1.5 Hz, 1H), 4.41 (m, 4H), 3.93 (t, J = 4.8 Hz, 2H), 3.54 (t, J = 4.5 Hz, 2H), 3.04 (t, J = 9.6 Hz, 2H), 2.94 (t, J = 4.5 Hz, 2H), 2.83 (s, 3H), 1.91-1.78 (m, 4H), 1.63 (m, 1H), 1.45 (m, 2H), 1.30 (ddd, J = 3.3, 9.6, 18.9 Hz, 2H); MS calcd. for [M + H]⁺ C₂₂H₃₀ClN₄O₃S: 465.2; found: 465.2. |
| 98 | 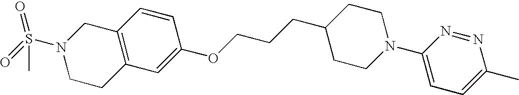 | ¹H NMR (400 MHz, CD₃CN) δ 7.62 (s, 2H), 7.04 (d, J = 6.2, 1H), 6.76 (dd, J = 1.8, 6.3 Hz, 1H), 6.72 (d, J = 1.8 Hz, 1H), 4.31 (s, 2H), 4.23 (m, 2H), 3.95 (t, J = 4.8 Hz, 2H), 3.45 (t, J = 4.5 Hz, 2H), 3.10 (dt, J = 1.8, 9.9 Hz, 2H), 2.91 (t, J = 4.5 Hz, 2H), 2.81 (s, 3H), 1.88 (m, 2H), 1.77 (m, 2H), 1.68 (m, 1H), 1.41 (m, 2H), 1.24 (ddd, J = 3.0, 9.9, 18.9 Hz, 2H); MS calcd. for [M + H]⁺ C₂₃H₃₃N₄O₃S: 445.2; found: 445.2. |
| 99 | 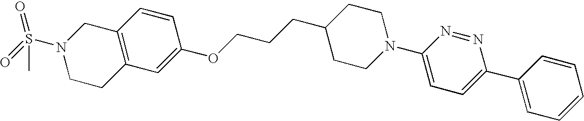 | MS calcd. for [M + H]⁺ C₂₈H₃₅N₄O₃S: 507.2, found: 507.2. |
| 100 | 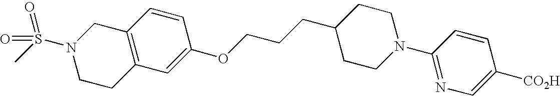 | ¹H NMR (400 MHz, DMSO-d₆) δ 8.60 (d, J = 1.8 Hz, 1H), 7.88 (dd, J = 1.8, 6.9 Hz, 1H), 7.07 (d, J = 6.3 Hz, 1H), 6.84 (d J = 6.9 Hz, 1H), 6.76 (m, 2H), 4.46 (d, J = 9.9 Hz, 2H), 4.27 (s, 2H), 3.93 (t, J = 4.8 Hz, 2H), 3.38 (t, J = 4.5 Hz, 2H), 2.92 (s, 3H), 2.86 (m, 4H), 1.73 (m, 4H), 1.59 (m, 1H), 1.35 (m, 2H), 1.07 (ddd, J = 3.0, 9.6, 18.6 Hz, 2H); MS calcd. for [M + H]⁺ C₂₄H₃₂N₃O₅S: 474.2; found: 474.2. |
| 101 | 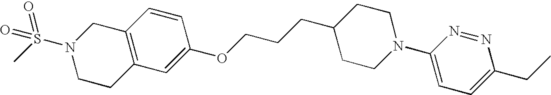 | ¹H NMR (400 MHz, CD₃CN) δ 7.47 (dd, J = 7.2, 15.6 Hz, 2H), 6.96 (d, J = 6.3 Hz, 1H), 6.67 (dd, J = 2.1, 6.3 Hz, 1H), 6.64 (d, J = 1.8 Hz, 1H), 4.22 (s, 2H), 4.19 (m, 2H), 3.86 (t, J = 4.8 Hz, 2H), 3.36 (t, J = 4.5 Hz, 2H), 2.98 (dt, J = 1.8, 9.9 Hz, 2H), 2.80 (m, 4H), 2.72 (2, 3H), 1.78 (m, 2H), 1.69 (m, 2H), 1.58 (m, 1H), 1.33 (m, 2H), 1.19- 1.13 9 (m, 5H); MS calcd. for (M + H]⁺ C₂₄H₃₅N₄O₃S: 459.2; found: 459.2. |
| 102 | 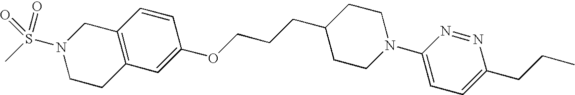 | ¹H NMR (400 MHz, CD₃CN) δ 7.65 (d, J = 0.6 Hz, 1H), 7.04 (d, J = 6.3 Hz, 1H), 6.76 (dd, J = 2.1, 6.3 Hz, 1H), 6.72 (d, J = 1.8 Hz, 1H), 4.31 (m, 4H), 3.95 (t, J = 5.1 Hz, 2H), 3.72-3.50 (m, 2H), 3.44 (t, J = 4.5 Hz, 2H), 3.11 (dt, J = 1.8, 10.2 Hz, 2H), 2.91 (m, 4H), 2.81 (s, 3H), 1.89 (m, 2H), 1.81-1.66 (m, 5H), 1.42 (m, 2H), 1.25 (m, 2H), 0.96 (t, J = 5.7 Hz, 5H); MS calcd. for [M + H]⁺ C₂₅H₃₇N₄O₃S: 473.3; found: 473.2. |
| 103 | 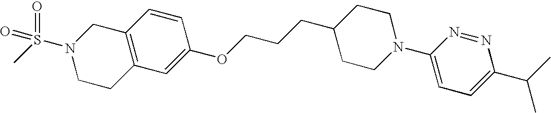 | ¹H NMR (400 MHz, CD₃CN) δ 7.19 (d, J = 6.9 Hz, 1H), 7.03 (m, 2H), 6.66 (dd, J = 2.1, 6.3 Hz, 1H), 6.72 (d, J = 1.8 Hz, 1H), 4.31 (m, 4H), 3.95 (t, J = 4.8 Hz, 2H), 3.45 (t, J = 4.5 Hz, 2H), 3.08 (septet, J = 5.1 Hz, 1H), 2.92-2.83 (m, 4H), 2.81 (s, 3H), 1.82-1.75 (m, 4H), 1.57 (m, 1H), 1.43-1.37 (m, 2H), 1.26 (d, J = 5.1 Hz, 6H), 1.26- 1.16 (m, 2H); MS calcd. for [M + H]⁺ C₂₅H₃₇N₄O₃S: 473.3; found: 473.2. |
| 104 | 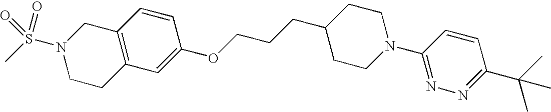 | ¹H NMR (400 MHz, CD₃CN) δ 7.36 (d, J = 7.2 Hz, 1H), 7.04 (d, J = 6.0 Hz, 1H), 7.03 (d, J = 7.2 Hz, 1H), 6.76 (dd, J = 2.1, 6.3 Hz, 1H), 6.73 (d, J = 1.8 Hz, 1H), 4.35 (m, 2H), 4.31 (s, 2H), 3.95 (t, J = 4.8 Hz, 2H), 3.45 (t, J = 4.5 Hz, 2H), 2.92-2.82 (m, 4H), 2.81 (s, 3H), 1.79 (m, 4H), 1.58 (m, 1H), 1.40 (m, 2H), 1.33 (s, 9H), 1.33 (m, 2H), 1.20 (ddd, J = 3.0, 9.0, 18.3 Hz, 2H); MS calcd. for [M + H]⁺ C₂₆H₃₉N₄O₃S: 487.3; found: 486.9. |
| 105 | 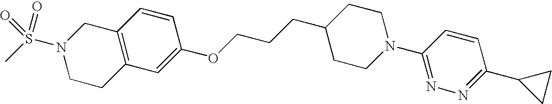 | ¹H NMR (400 MHz, CD₃CN) δ 7.06 (d, J = 6.9 Hz, 1H), 7.04 (d, J = 6.3 Hz, 1H), 6.98 (d, J = 6.9 Hz, 1H), 6.76 (dd, J = 2.1, 6.3 Hz, 1H), 6.72 (d, J = 2.1 Hz, 1H), 4.31 (s, 2H), 4.27 (m, 2H), 3.94 (t, J = 4.8 Hz, 2H), 3.45 (t, J = 4.5 Hz, 2H), 2.91 (t, J = 4.5 Hz, 2H), 2.84 (m, 2H), 2.81 (s, 3H), 2.05 (m, 1H), 1.82-1.76 (m, 4H), 1.57 (m, 1H), 1.42-1.37 (m, 2H), 1.19 (ddd, J = 3.3, 9.6, 18.6 Hz, 2H), 0.97-0.87 (m, 4H); MS calcd. for [M + H]⁺ C₂₅H₃₅N₄O₃S: 471.2; found: 471.2. |
| 106 | 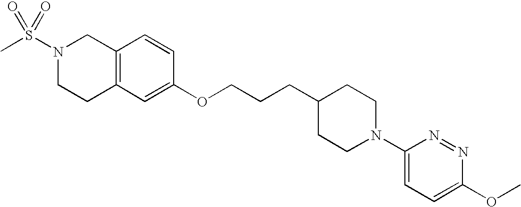 | ¹H NMR (400 MHz, CD₃CN) δ 7.51 (d, J = 7.5 Hz, 1H), 7.21 (d, J = 7.5 Hz, 1H), 7.05 (d, J = 6.3 Hz, 1H), 6.76 (dd, J = 1.8, 6.3 Hz, 1H), 6.72 (d, J = 1.8 Hz, 1H), 4.31 (s, 2H), 4.18 (m, 2H), 3.95 (t, J = 2.4 Hz, 2H), 3.92 (s, 3H), 3.45 (t, J = 4.5 Hz, 2H), 3.11 (dt, J = 1.8, 9.9 Hz, 2H), 2.91 (t, J = 4.5 Hz, 2H), 2.81 (s, 3H), 1.88 (m, 2H), 1.78 (m, 2H), 1.66 (m, 1H), 1.42m, 2H), 1.28 (ddd, J = 3.0, 9.9, 18.6 Hz, 2H); MS calcd. for [M + H]⁺ C₂₃H₃₃N₄O₄S: 461.2; found: 461.2. |
| 107 | 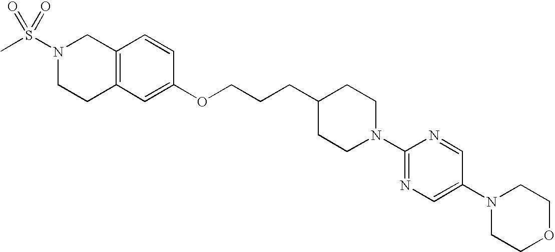 | ¹H NMR (400 MHz, CD₃CN) δ 8.09 (s, 2H), 7.04 (d, J = 6.3 Hz, 1H), 6.75 (dd, J = 2.1, 6.3 Hz, 1H), 6.72 (d, J = 0.9 Hz, 1H), 4.58 (m, 2H), 4.31 (s, 2H), 3.94 (m, 2H), 3.75 (t, J = 3.6 Hz, 4H), 3.45 (t, J = 4.5 Hz, 2H), 2.97 (t, J = 3.3 Hz, 4H), 2.91 (t, J = 4.5 Hz, 2H), 2.81 (s, 3H), 2.80 (m, 2H), 1.77 (m, 4H), 1.55 (m, 1H), 1.40 (m, 2H), 1.10 (ddd, J = 3.3, 9.6, 18.3 Hz, 2H); MS calcd. for [M + H]⁺ C₂₆H₃₈N₅O₄S: 516.3; found: 516.2. |
| 108 | 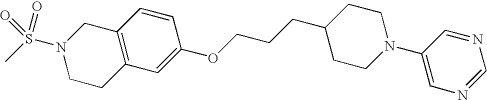 | ¹H NMR (400 MHz, CD₃CN) δ 8.51 (s, 1H), 8.38 (s, 2H), 7.04 (d, H = 6.3, 1H), 6.76 (dd, J = 2.1, 6.2, Hz, 1H), 6.73 (d, J = 1.8 Hz, 1H), 4.31 (s, 2H), 4.95 (t, J = 5.1 Hz, 2H), 3.78 (m, 2H), 3.45 (t, J = 4.5 Hz, 2H), 2.91 (t, J = 4.2 Hz, 2H), 2.81 (s, 3H), 2.77 (dd, J = 2.1, 9.0 Hz, 2H), 1.85-1.75 (m, 4H), 1.50 (m, 1H), 1.40 (m, 2H), 1.35 (m, 2H); MS calcd. for [M + H]⁺ C₂₂H₃₁N₄O₃S: 431.2; found: 431.1. |
| 109 | 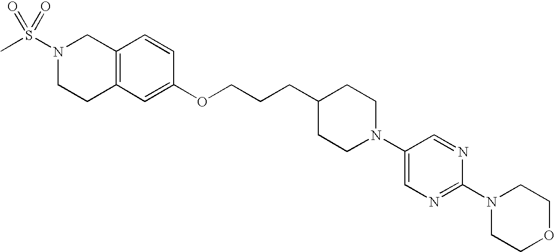 | ¹H NMR (400 MHz, CD₃CN) δ 8.84 (s, 2H), 7.05 (d, J = 6.3 Hz, 1H), 6.78 (dd, J = 2.1, 6.3 Hz, 1H), 6.74 (d, J = 1.8 Hz, 1H), 4.32 (s, 2H), 3.97 (t, J = 4.8 Hz, 2H), 3.79 (t, J = 3.3 Hz, 4H), 3.69 (t, J = 3.9 Hz, 4H), 3.55 (m, 2H), 3.46 (t, J = 4.5 Hz, 2H), 3.37 (dd, J = 3.3, 9.0 Hz, 2H), 2.92 (t, J = 4.5 Hz, 2H), 2.81 (s, 3H), 2.00 (m, 4H), 1.78 (m, 2H), 1.70 (m, 1H), 1.49 (m, 2H); MS calcd. for [M + H]⁺ C₂₆H₃₈N₅O₄S: 516.3; found: 516.2. |
| 110 | 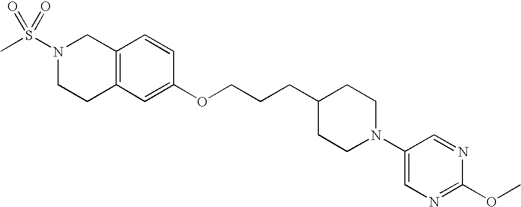 | ¹H NMR (400 MHz, CD₃CN) δ 8.21 (s, 2H), 7.04 (d, J = 6.3 Hz, 1H), 6.76 (dd, J = 1.8, 6.0 Hz, 1H), 6.73 (d, J = 1.8 Hz, 1H), 4.31 (s, 2H), 3.95 (t, J = 4.8 Hz, 2H), 3.86 (s, 3H), 3.54 (m, 2H), 3.45 (t, J = 4.5 Hz, 2H), 2.91 (t, J = 4.5 Hz, 2H), 2.81 (s, 3H), 2.66 (dt, J = 1.8, 9.0 Hz, 2H), 1.80 (m, 4H), 1.42 (m, 2H), 1.31 (m, 2H); MS calcd. for [M + H]⁺ C₂₃H₃₃N₄O₄S: 461.2; found: 461.2. |
| 111 | 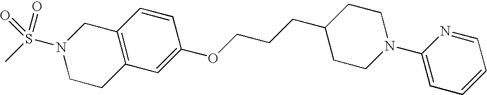 | ¹H NMR (400 MHz, CD₃CN) δ 8.04 (d, J = 4.5 Hz, 1H), 7.83 (t, J = 5.7 Hz, 1H), 7.11 (d, J = 6.9 Hz, 1H), 7.04 (d, J = 6.3 Hz, 1H), 6.81-6.73 (m, 3H), 4.31 (s, 2H), 4.19 (d, J = 9.9 Hz, 2H), 3.95 (t, J = 4.8 Hz, 2H), 3.45 (t, J = 4.5 Hz, 2H), 3.13 (t, J = 9.3 Hz, 2H), 2.91 (t, J = 4.5 Hz, 2H), 2.81 (s, 3H), 1.88 (m, 2H), 1.78 (m, 2H), 1.67 (m, 2H), 1.40 (m, 2H), 1.28 (m, 2H); MS calcd. for [M + H]⁺ C₂₃H₃₃N₄O₄S: 430.2; found: 430.1. |
| 112 | 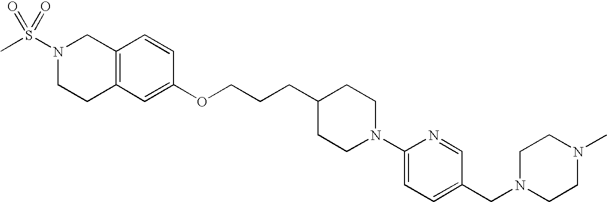 | ¹H NMR (400 MHz, CD₃CN) δ 7.97 (s, 1H), 7.85 (dd, J = 1.5, 6.9 Hz, 1H), 7.13 (d, J = 7.2 Hz, 1H), 7.04 (d, J = 6.3 Hz, 1H), 6.75 (dd, J = 1.8, 6.3 Hz, 1H), 6.72 (d, J = 1.8 Hz, 1H), 4.31 (s, 2H), 4.17 (m, 2H), 3.94 (t, J = 4.8 Hz, 2H), 3.74 (s, 2H), 3.45 (t, J = 4.5 Hz, 2H), 3.16 (dt, J = 1.8, 10.2 Hz, 2H), 2.91 (t, J = 4.2 Hz, 2H), 2.81 (s, 3H), 2.77 (s, 3H), 1.92 (m, 2H), 1.77 (m, 2H), 1.69 (m, 1H), 1.41 (m, 2H), 1.25 (ddd, J = 2.7, 9.6, 18.9 Hz, 2H); MS calcd. for [M + H]⁺ C₂₉H₄₄N₅O₃S: 542.3; found: 542.3. |
| 113 | 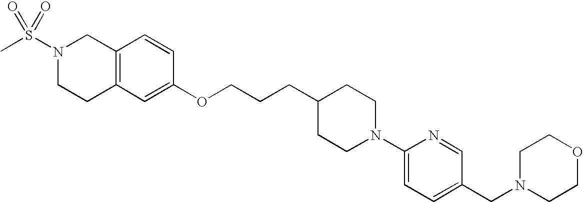 | ¹H NMR (400 MHz, CD₃CN) δ 8.14 (d, J = 1.2 Hz, 1H), 7.85 (dd, J = 1.5, 7.2 Hz, 1H), 7.05 (dd, J = 6.0, 7.2 Hz, 1H), 6.75 (dd, J = 2.1, 6.3 Hz, 1H), 6.72 (d, J = 1.8 Hz, 1H), 4.31 (s, 2H), 4.23 (m, 2H), 4.12 (s, 2H), 3.94 (t, J = 4.8 Hz, 2H), 3.45 (t, J = 4.8 Hz, 2H), 3.11 (dt, J = 1.8, 10.2 Hz, 2H), 2.91 (t, J = 4.5 Hz, 2H), 2.81 (s, 3H), 1.89 (m, 2H), 1.77 (m, 2H), 1.67 (m, 1H), 1.40 (m, 2H), 1.23 (ddd, J = 3.0, 9.6, 18.9 Hz, 2H); MS calcd. for [M + H]⁺ C₂₈H₄₁N₄O₄S: 529.3; found: 529.2. |
| 114 | 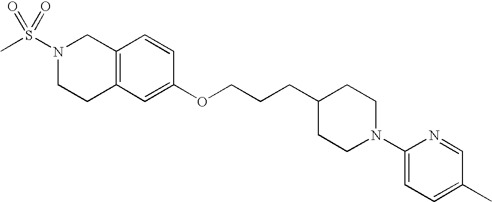 | ¹H NMR (400 MHz, CD₃CN) δ 7.86 (d, J = 0.6 Hz, 1H), 7.72 (dd, J = 1.5, 6.9 Hz, 1H), 7.05 (t, J = 6.9 Hz, 1H), 6.75 (dd, J = 1.8, 6.3 Hz, 1H), 6.72 (d, J = 1.8 Hz, 1H), 4.31 (s, 2H), 4.17 (m, 2H), 3.94 (t, J = 4.8 Hz, 2H), 3.45 (t, J = 4.5 Hz, 2H), 3.11 (dt, J = 1.8, 9.0 Hz, 2H), 2.90 (t, J = 4.5 Hz, 2H), 2.81 (s, 3H), 1.86 (m, 2H), 1.77 (m, 2H), 1.64 (m, 1H), 1.40 (m, 2H), 1.25 (ddd, J = 3.0, 9.9, 18.9 Hz, 2H); MS calcd. for [M + H]⁺ C₂₄H₃₄N₃O₃S: 444.2; found: 444.2. |
| 115 | 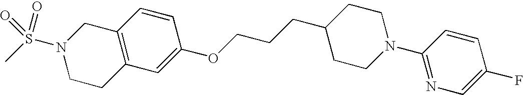 | ¹H NMR (400 MHz, CD₃CN) δ 7.99 (d, J = 2.4 Hz, 1H), 7.32 (m, 1H), 7.04 (d, J = 6.6 Hz, 1H), 6.76 (dd, J = 2.1, 6.3 Hz, 1H), 6.72 (dd, J = 2.4, 4.5 Hz, 1H), 4.31 (s, 2H), 4.18 (m, 2H), 3.94 (t, J = 5.1 Hz, 2H), 3.45 (t, J = 4.5 Hz, 2H), 2.91 (t, J = 4.5 Hz, 2H), 2.81 (s, 3H), 2.78 (dt, J = 2.1, 9.6 Hz, 2H), 1.80-1.75 (m, 4H), 1.51 (m, 1H), 1.39 (m, 2H), 1.19 (ddd, J = 3.0, 9.0, 18.6 Hz, 2H); MS calcd. for [M + H]⁺ C₂₃H₃₁FN₃O₃S: 448.2; found: 448.2. |
| 116 | 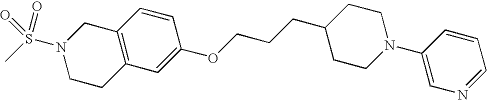 | ¹H NMR (400 MHz, CD₃CN) δ 8.29 (s, 1H), 8.03 (d, J = 2.7 Hz, 1H), 7.81 (d, J = 6.3 Hz, 1H), 7.64 (dd, J = 3.9, 6.0 Hz, 1H), 7.04 (d, J = 6.6 Hz, 1H), 7.75 (d, J = 6.6 Hz, 1H), 6.73 (s, 1H), 4.31 (s, 2H), 3.95 (t, J = 4.8 Hz, 2H), 3.85 (d, J = 9.0 Hz, 2H), 3.45 (t, 4.5 Hz, 2H), 2.92 (m, 4H), 2.81 (s, 3H), 1.81 (m, 4H), 1.58 (m, 1H), 1.41 (m, 2H), 1.28 (m, 2H); MS calcd. for [M + H]⁺ C₂₃H₃₂N₃O₃S: 430.2; found: 430.1. |
| 117 | 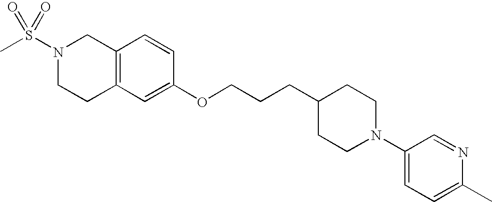 | ¹H NMR (400 MHz, CD₃CN) δ 8.15 (d, J = 2.1 Hz, 1H), 7.80 (dd, J = 2.1, 6.6 Hz, 1H), 7.48 (d, J = 6.9 Hz 1H), 7.04 (d, J = 6.3 Hz, 1H), 6.75 (dd, J = 1.8, 6.3 Hz, 1H), 6.72 (d, J = 1.8, 1H), 4.31 (s, 2H), 3.94 (t, J = 4.8 Hz, 2H), 3.78 (m, 2H), 3.45 (t, J = 4.5 Hz, 2H), 2.92-2.82 (m, 4H), 2.81 (s, 3H), 2.58 (s, 3H), 1.90-1.74 (m, 4H), 1.53 (m, 1H), 1.42 (m, 2H), 1.26 (ddd, J = 3.0, 9.6, 18.6 Hz, 2H); MS calcd. for [M + H]⁺ C₂₄H₃₄N₃O₃S: 444.2; found: 444.2. |
| 118 | 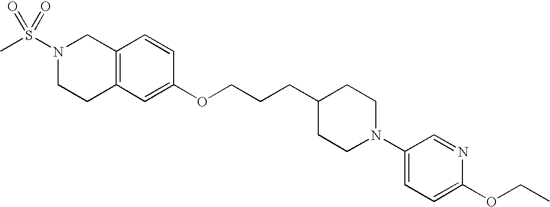 | ¹H NMR (400 MHz, CD₃CN) δ 8.00 (d, J = 1.8 Hz, 1H), 7.57 (dd, J = 2.4, 6.6 Hz, 1H), 7.05 (d, J = 6.3 Hz, 1H), 6.76 (dd, J = 1.8, 6.3 Hz, 1H), 6.73 (m, 2H), 4.32 (s, 2H), 4.28 (dd, J = 5.4, 10.8 Hz, 2H), 3.95 (t, J = 4.8 Hz, 2H), 3.53 (m, 2H), 3.45 (t, J = 4.5 Hz, 2H), 2.93-2.85 (m, 4H), 2.81 (s, 3H), 1.91 (m, 2H), 1.79 (m, 2H), 1.50-1.44 (m, 5H), 1.32 (t, J = 5.4 Hz, 2H); MS calcd. for [M + H]⁺ C₂₅H₃₆N₃O₄S: 474.2; found:.474.2. |
| 119 | 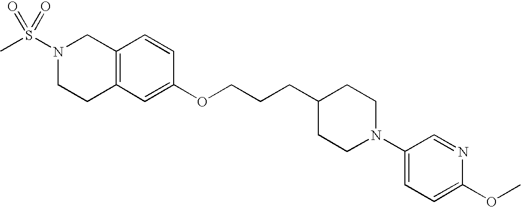 | ¹H NMR (400 MHz, CD₃CN) δ 7.97 (d, J = 1.8 Hz, 1H), 7.53 (dd, J = 2.1, 6.9 Hz, 1H), 7.05 (d, J = 6.3 Hz, 1H), 6.77 (dd, J = 1.8, 6.3 Hz, 1H), 6.73 (m, 2H), 4.32 (s, 2H), 3.95 (t, J = 4.8 Hz, 2H), 3.84 (s, 3H), 3.52 (m, 2H), 3.45 (t, J = 4.5 Hz, 2H), 2.91 (t, J = 4.5 Hz, 2H), 2.82 (m, 2H), 2.81 (s, 3H), 2.10 (m, 1H), 1.88 (m, 2H), 1.78 (m, 2H), 1.44 (m, 4H); MS calcd. for [M + H]⁺ C₂₄H₃₄N₃O₄S: 460.2; found: 460.2. |
| 120 | 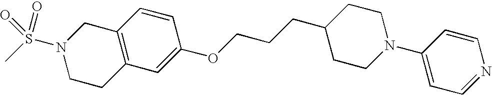 | ¹H NMR (400 MHz, CD₃CN) δ 8.03 (d, J = 5.4 Hz, 2H), 7.04 (d, J = 6.3 Hz, 1H), 6.95 (d, J = 5.4 Hz, 2H), 7.75 (dd, J = 1.8, 6.3 Hz, 1H), 4.31 (s, 2H), 4.12 (d, J = 10.2 Hz, 2H), 3.95 (t, J = 4.8 Hz, 2H), 3.45 (t, J = 4.5 Hz, 2H), 3.13 (dd, J = 1.8, 10.2 Hz, 2H), 2.91 (t, J = 4.5 Hz, 2H), 2.81 (s, 3H), 1.90 (m, 2H), 1.81-1.69 (m, 3H), 1.41 (m, 2H), 1.22 (ddd, J = 2.7, 9.6, 18.9 Hz, 2H); MS calcd. for [M + H]⁺ C₂₃H₃₂N₃O₃S: 430.2; found: 430.1. |
| 121 | 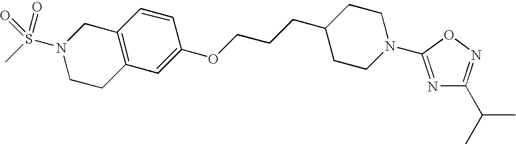 | ¹H NMR (400 MHz, CD₃CN) δ 7.04 (d, J = 6.3 Hz, 1H), 6.77 (dd, J = 1.8, 6.3 Hz, 1H), 6.72 (d, J = 2.1 Hz, 1H), 4.31 (s, 2H), 4.03 (m, 2H), 3.94 (t, J = 4.8 Hz, 2H), 3.45 (t, J = 4.5 Hz, 2H), 3.05 (dt, J = 2.1, 9.6 Hz, 2H), 2.91 (t, J = 4.5 Hz, 2H), 2.82 (m, 1H), 2.81 (s, 3H), 1.76 (m, 4H), 1.53 (m, 1H), 1.41 (m, 2H), 1.21 (d, J = 5.1 Hz, 6H), 1.25- 1.15 (m, 2H); MS calcd. for [M + H]⁺ C₂₃H₃₅N₄O₄S: 463.2; found: 463.2. |
| 122 | 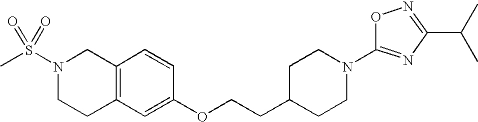 | ¹H NMR (400 MHz, CD₃CN) δ 7.05 (d, J = 6.3 Hz, 1H), 6.76 (dd, J = 2.1, 6.3 Hz, 1H), 6.73 (d, J = 1.8 Hz, 1H), 4.31 (s, 2H), 4.03 (m, 4H), 3.54 (t, J = 4.5 Hz, 2H), 3.06 (dt, J = 1.8, 9.6 Hz, 2H), 2.91 (t, J = 4.2 Hz, 2H), 2.83 (m, 1H), 2.81 (s, 3H); 1.83-1.70 (m, 5H), 1.28 (m, 2H), 1.21 (d, J = 5.1 Hz, 6H); MS calcd. for [M + H]⁺ C₂₂H₃₃N₄O₄S: 449.2; found 449.1 |
| 123 | 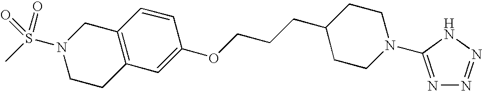 | ¹NMR (400 MHz, CD₃CN) δ 7.04 (d, J = 6.3 Hz, 1H), 6.77-6.72 (m, 2H), 4.31 (s, 2H), 3.94 (t, J = 5.1 Hz, 2H), 3.86 (m, 2H), 3.45 (t, J = 4.5 Hz, 2H), 2.97 (dt, J = 2.1, 9.3 Hz, 2H), 2.91 (t, J = 4.8 Hz, 2H), 2.81 (s, 3H), 1.81-1.74 (m, 2H), 1.50 (m, 1H), 1.42-1.35 (m, 2H), 1.25 (ddd, J = 3.3, 9.3, 18.6 Hz, 2H); MS calcd. for [M + H]⁺ C₁₉H₂₉N₆O₃S: 421.2; found: 421.9. |
| 124 | 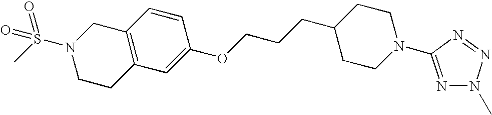 | ¹NMR (400 MHz, CD₃CN) δ 7.04 (d, J = 6.3 Hz, 1H), 6.76-6.72 (m, 2H), 4.31 (s, 2H), 4.09 (s, 3H), 3.99-3.93 (m, 4H), 3.45 (t, J = 4.5 Hz, 2H), 2.92-2.82 (m, 4H), 2.81 (s, 3H), 1.81-1.74 (m, 4H), 1.50 (m, 1H), 1.42-1.37 (m, 2H), 1.23 (ddd, J = 3.3, 9.3, 18.3 Hz, 2H); MS calcd. for [M + H]⁺ C₂₀H₃₁N₆O₃S: 435.2; found: 434.9. |
| 125 | 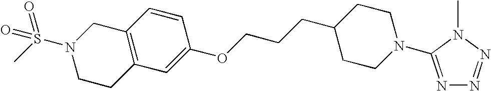 | ¹NMR (400 MHZ, CD₃CN) δ 7.04 (d, J = 6.3 Hz, 1H), 6.77-6.72 (m, 2H), 4.31 (s, 2H), 3.95 (t, J = 4.8 Hz, 2H), 3.81 (s, 3H), 3.58 (m, 2H), 3.45 (t, J = 4.5, 2H), 2.96 (dd, J = 1.8, 9.3 Hz, 2H), 2.91 (t, J = 4.2 Hz, 2H), 2.81 (s, 3H); MS calcd. for [M + H]⁺ C₂₀H₃₁N₆O₃S: 435.2; found: 434.9. |
| 126 | 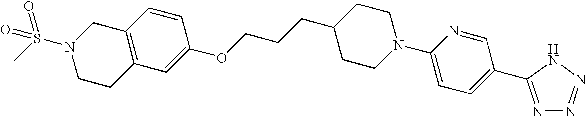 | MS calcd. for [M + H]⁺ C₂₄H₃₂N₇O₃S: 498.2; found: 498.2. |
| 127 | 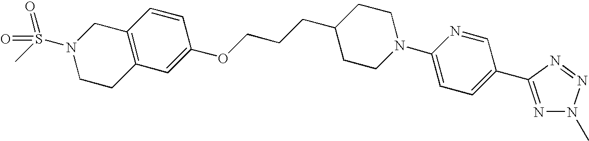 | ¹H NMR (400 MHz, CD₃CN) δ 8.80 (d, J = 1.8 Hz, 1H), 8.13 (dd, J = 1.8, 7.7 Hz, 1H), 7.04 (d, J = 6.3 Hz, 1H), 6.90 (d, J = 6.9 Hz, 1H), 6.76 (dd, J = 1.8, 6.3 Hz, 1H), 6.73 (d, J = 1.8 Hz, 1H), 4.40 (m, 2H), 4.32 (s, 3H), 4.31 (s, 2H), 3.95 (t, J = 4.8 Hz, 2H), 3.45 (t, J = 4.5 Hz, 2H), 2.98-2.89 (m, 4H), 2.81 (s, 3H), 1.85-1.75 (m, 4H), 1.62 (m, 1H), 1.42-1.37 (m, 2H), 1.20 (ddd, J = 3.3, 9.3, 18.6 Hz, 2H); MS calcd. for [M + H]⁺ C₂₅H₃₄N₇O₃S: 512.2; found: 512.2. |
| 128 | 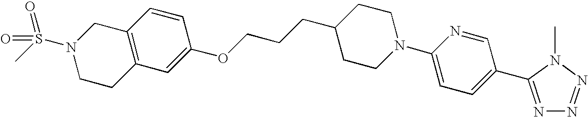 | ¹H NMR (400 MHz, CD₃CN) δ 8.51 (d, J = 1.5 Hz, 1H, 7.87 (dd, J = 1.8, 6.6 Hz, 1H), 7.04 (d, J = 6.3 Hz, 1H), 6.90 (d, J = 6.9 Hz, 3H), 6.77-6.73 (m, 2H), 4.45 (m, 2H), 4.32 (s, 3H), 4.10 (s, 2H), 3.95 (t, J = 5.1 Hz, 2H), 3.45 (t, J = 4.5 Hz, 2H), 2.96-2.90 (m, 4H), 2.81 (s, 3H), 1.81-1.77 (m, 4H), 1.62 (m, 1H), 1.44-1.38 (m, 2H), 1.18 (ddd, J = 3.0, 9.6, 18.6 Hz, 2H); MS calcd. for [M + H]⁺ C₂₅H₃₄N₇O₃S: 512.2; found: 512.2. |
| 129 | 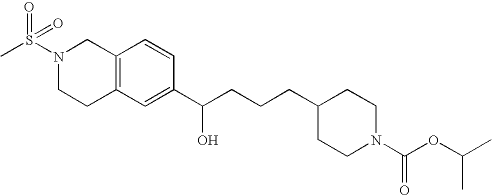 | ¹H-NMR (400 MHz, CD₃CN) δ = 7.19-7.16 (m, 2H), 7.12 (d, J = 8.0 Hz, 1H), 4.82 (septet, J = 6.4 Hz, 1H), 4.58-4.54 (m, 1H), 4.40 (m, 2H), 4.03 (br. d, 2H), 3.50 (t, J = 6.0 Hz, 2H), 3.18 (d, J = 4.4 Hz, 1H), 2.96 (t, J = 6.0 Hz, 2H), 2.85 (s, 3H), 2.74-2.66 (m, 2H), 1.68- 1.57 (m, 4H), 1.46-1.34 (m, 2H), 1.30-1.23 (m, 3H), 1.21 (d, J = 6.4 Hz, 6H), 1.05-0.94 (m 2H); MS calcd. for [M + H]⁺ C₂₃H₃₇N₂O₅S: 453.2; found: 453.2 |
| 130 | 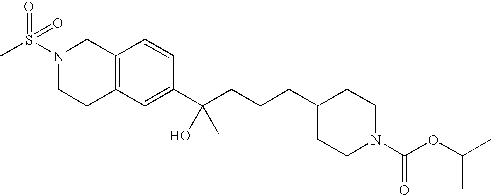 | ¹H-NMR (400 MHz, CD₃CN) δ = 7.26-7.23 (m 2H), 7.10 (d, J = 8.4 Hz, 1H), 4.81 (septet, J = 6.4 Hz, 1H), 4.38 (s, 2H), 4.04-3.96 (br. t, 2H), 3.48 (t, J = 6.0 Hz, 2H), 2.96 (t, J = 6.0 Hz, 2H), 2.85 (s, 3H), 2.73-2.60 (m, 2H), 1.77-1.63 (m, 2H), 1.58-1.55 (br. d, J = 12.8 Hz, 2H), 1.44 (s, 3H), 1.38-1.25 (m, 2H), 1.19 (d, J = 6.4 Hz, 6H), 1.17- 1.12 (m 2H), 1.08-1.00 (m, 1H), 0.98-0.87 (m, 2H); MS calcd. for [M + H]⁺ C₂₄H₃₉N₂O₅S: 467.2; found: 467.2 |
| 131 | 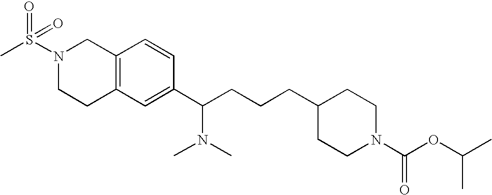 | ¹H-NMR (400 MHz, CD₃CN) δ = 7.40 (s, 1H) 7.38 (d, J = 8.0 Hz, 1H), 7.24 (d, J = 8.0 Hz, 1H), 4.70 (septet, J = 6.4 Hz, 1H), 4.39 (s, 2H), 4.26- 4.22 (m, 1H), 3.93-3.90 (br. d, 2H), 3.46 (t, J = 6.0 Hz, 2H), 2.94 (t, J = 6.0 Hz, 2H), 2.83 (s, 3H), 2.76 (br. s, 3H), 2.61 (br. s, 3H), 2.57-2.47 (m, 2H), 2.23-2.11 (m, 2H), 1.48- 1.42 (m, 2H), 1.30-1.22 (m, 2H), 1.19-1.15 (m, 2H), 1.08 (d, J = 6.4 Hz, 6H), 1.04-0.97 (m 1H), 0.89-0.76 (m, 2H); MS calcd. for [M + H]⁺ C₂₅H₄₂N₃O₄S: 480.3; found: 480.2 |
| 132 | 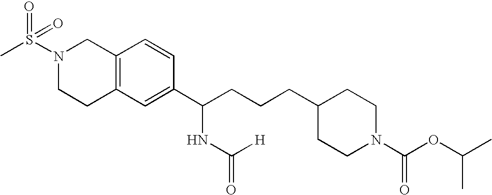 | ¹H-NMR (400 MHz, CD₃CN) δ = 7.98 (s, 1H) 7.05-6.99 (m, 3H), 6.73 (d, J = 8.0 Hz, 0.8H), 6.65 (d, J = 8.0 Hz, 0.2H), 4.73-4.61 (m, 2H), 4.28 (s, 2H), 3.95-3.87 (m, 2H), 3.38 (t, J = 6.0 Hz, 2H), 2.84 (t, J = 6.0 Hz, 2H), 2.73 (s, 3H), 2.64- 2.55 (m, 2H), 1.63-1.48 (m, 4H), 1.30-1.24 (m, 2H), 1.18-1.13 (m, 3H), 1.09 (d, J = 6.4 Hz, 6H), 0.93-0.83 (m, 2H); MS calcd. for [M + H]⁺ C₂₄H₃₈N₃O₅S: 480.2; found: 480.2 |
| 133 | 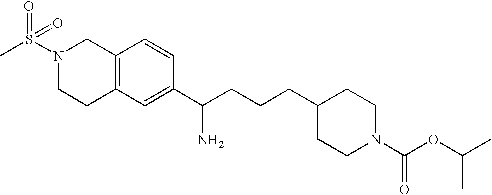 | ¹H-NMR (400 MHz, CD₃CN) δ = 7.68 (br. s, 3H), 7.18 (m, 2H), 7.11 (d, J = 8.0 Hz, 1H), 4.70 (septet, J = 6.4 Hz, 1H), 4.32 (m, 2H), 4.15-4.11 (m, 1H), 3.90 (br. d, J = 12.4 Hz, 2H), 3.40 (t, J = 6.0 Hz, 2H), 2.86 (t, J = 6.0 Hz, 2H), 2.75 (s, 3H), 2.60-2.50 (m, 2H), 1.49- 1.45 (m, 2H), 1.28-1.02 (m, 7H), 1.09 (d, J = 6.4 Hz, 6H), 0.91-0.78 (m 2H); MS calcd. for [M + H]⁺ C₂₃H₃₈N₃O₄S: 451.2; found: 451.2 |
| 134 | 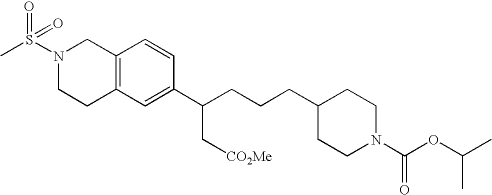 | ¹H-NMR (400 MHz, CD₃CN) δ = 7.10-7.07 (m, 2H), 7.05 (s, 1H), 4.81 (septet, J = 6.4 Hz, 1H), 4.38 (s, 2H), 4.00 (br. d, J = 12.8 Hz, 2H), 3.54 (s, 3H), 3.49 (t, J = 6.0 Hz, 2H), 3.06-2.98 (m, 1H), 2.94 (t, J = 6.0 Hz, 2H), 2.85 (s, 3H), 2.74-2.61 (m, 2H), 2.65 (dd, J = 15.2, 6.4 Hz, 1H), 2.54 (dd, J = 15.2, 8.8 Hz, 1H), 1.64-1.54 (m, 4H), 1.37-1.30 (m, 2H), 1.20 (d, J = 6.4 Hz, 6H), 1.18- 1.12 (m 2H), 1.01-0.88 (m, 3H); MS calcd. for [M + H]⁺ C₂₆H₄₁N₂O₆S: 509.2; found: 509.2 |
| 135 | 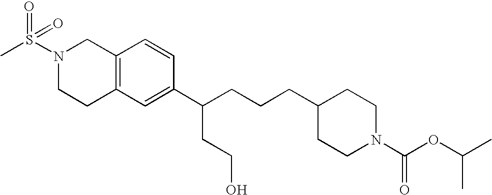 | ¹H-NMR (400 MHz, CD₃CN) δ = 7.00 (d, J = 8.0 Hz, 1H), 6.96-6.94 (m, 2H), 4.70 (septet, J = 6.4 Hz, 1H), 4.29 (s, 2H), 3.94-3.90 (m, 2H), 3.41 (t, J = 6.0 Hz, 2H), 3.34-3.21 (m, 3H), 3.16-3.11 (m, 1H), 2.85 (t, J = 6.0 Hz, 2H), 2.79 (s, 3H), 2.67- 2.59 (m, 2H), 1.80-1.72 (m, 1H), 1.67-1.58 (m, 1H), 1.54-1.43 (m, 4H), 1.25-1.15 (m, 3H), 1.12- 1.10 (m, 2H), 1.08 (d, J = 6.4 Hz, 6H), 0.91-0.76 (m, 2H); MS calcd. for [M + H]⁺ C₂₅H₄₁N₂O₅S: 481.2; found: 481.2 |
| 136 | 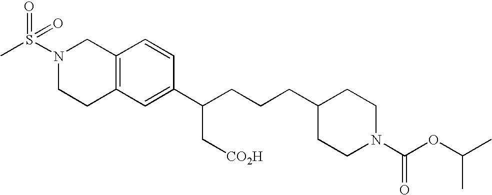 | ¹H-NMR (400 MHz, CD₃CN) δ = 7.00-6.96 (m, 3H), 4.68 (septet, J = 6.4 Hz, 1H), 4.26 (s, 2H), 3.91-3.88 (br. d, 2H), 3.38 (t, J = 6.0 Hz, 2H), 2.96- 2.88 (m, 1H), 2.82 (t, J = 6.0 Hz, 2H), 2.76 (s, 3H), 2.62-2.48 (m, 2H), 2.52 (dd, J = 15.6, 7.2 Hz, 1H), 2.42 (dd, J = 15.6, 8.0 Hz, 1H), 1.61-1.44 (m, 4H), 1.26-1.13 (m, 3H), 1.12- 1.07 (m, 2H), 1.05 (d, J = 6.4 Hz, 6H), 0.88-0.74 (m, 2H); MS calcd. for [M + H]⁺ C₂₅H₃₉N₂O₆S: 495.2; found: 495.2 |
| 137 | 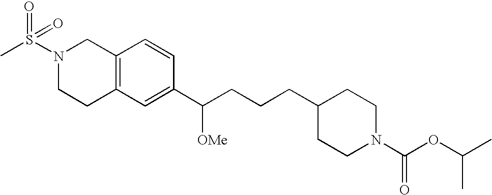 | ¹H-NMR (400 MHz, CD₃CN) δ = 7.06-7.03 (m, 2H), 7.01 (s, 1H), 4.70 (septet, J = 6.4 Hz, 1H), 4.30 (s, 2H), 3.97 (dd, J = 7.2, 6.0 Hz, 1H), 3.91 (br. s, 2H), 3.43-3.34 (m, 2H), 3.02 (s, 3H), 2.86 (t, J = 6.0 Hz, 2H), 2.75 (s, 3H), 2.65-2.50 (m, 2H), 1.65-1.57 (m, 1H), 1.55-1.48 (m, 2H), 1.48-1.40 (m, 1H), 1.32- 1.21 (m, 2H), 1.15-1.10 (m, 3H), 1.09 (d, J = 6.4 Hz, 6H), 0.91-0.80 (m 2H); MS calcd. for [M + H]⁺ C₂₄H₃₉N₂O₅S: 467.2; found: 467.2 |
| 138 | 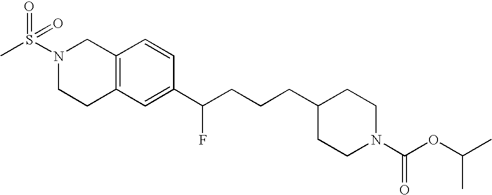 | ¹H-NMR (400 MHz, CD₃CN) δ = 7.11-7.06 (m, 3H), 5.33 (ddd, J = 48.0, 8.0, 5.2 Hz, 1H), 4.71 (septet, J = 6.4 Hz, 1H), 4.31 (s, 2H), 3.92 (br. d, J = 12.8 Hz, 2H), 3.40 (t, J = 6.0 Hz, 2H), 2.87 (t, J = 6.0 Hz, 2H), 2.74 (s, 3H), 2.65-2.54 (m, 2H), 1.72-1.61 (m, 1H), 1.57-1.50 (m, 2H), 1.41-1.22 (m, 3H), 1.20- 1.13 (m, 3H), 1.10 (d, J = 6.4 Hz, 6H), 0.94-0.84 (m 2H); ¹⁹F-NMR (376 MHz, CD₃CN) δ = −172.817; MS calcd. for [M + H]⁺ C₂₃H₃₆FN₂O₄S: 455.2; found: 455.2 |
| 139 | 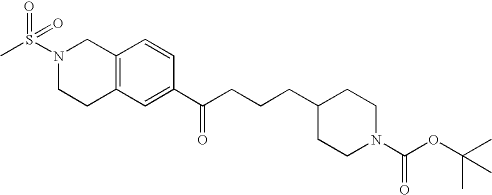 | ¹H-NMR (400 MHz, CDCl₃) δ = 7.81-7.78 (m 2H), 7.21 (d, J = 8.0 Hz, 1H), 4.53 (s, 2H), 4.10 (br. s, 2H), 3.61 (t, J = 6.0 Hz, 2H), 3.07 (t, J = 6.0 Hz, 2H), 2.96 (t, J = 7.2 Hz, 2H), 2.89 (s, 3H), 2.74-2.63 (m, 2H), 1.81- 1.74 (m, 2H), 1.72-1.67 (m, 2H), 1.47 (s, 9H), 1.44-1.39 (m, 1H), 1.36- 1.31 (m, 2H), 1.17-1.06 (m 2H); MS calcd. for [M + H]⁺ C₂₄H₃₇N₂O₅S: 465.2; found: 465.2 |
| 140 | 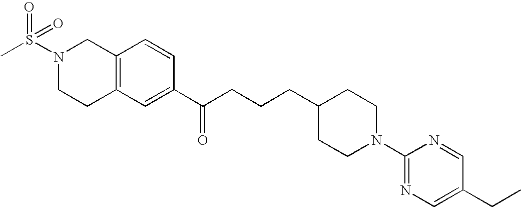 | ¹H-NMR (400 MHz, CDCl₃) δ = 8.17 (s, 2H), 7.79 (d, J = 8.0 Hz, 1H), 7.78 (s, 1H), 7.20 (d, J = 8.0 Hz, 1H), 4.69 (d, J = 13.2 Hz, 2H), 4.52 (s, 2H), 3.61 (t, J = 6.0 Hz, 2H), 3.07 (t, J = 4.8 Hz, 2H), 2.97 (t, J = 7.2 Hz, 2H), 2.88 (s, 3H), 2.85 (td, J = 2.4, 12.4 Hz, 2H), 2.46 (q, J = 7.6 Hz, 2H), 1.80 (m, 4H), 1.57 (m, 1H), 1.36 (m, 2H), 1.23 (m, 4H); MS calcd. for [M + H]⁺ C₂₅H₃₅N₄O₃S: 471.2; found: 471.2 |
| 141 | 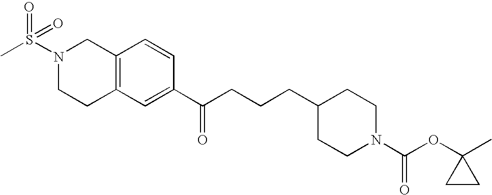 | ¹H-NMR (400 MHz, CDCl₃) δ = 7.78 (m, 2H), 7.20 (d, J = 8.0 Hz, 1H), 4.52 (s, 2H), 3.60 (t, J = 6.0 Hz, 2H), 3.07 (t, J = 6.0 Hz, 2H), 2.95 (t, J = 7.2 Hz, 2H), 2.88 (s, 3H), 2.71 (m, 2H), 1.75 (m, 3H), 1.67 (s, 2H), 1.55 (s, 3H), 1.43 (m, 1H), 1.32 (m, 2H), 1.25 (d, J = 6.4 Hz, 1H), 1.10 (m, 2H), 0.87 (t, J = 6.4 Hz, 2H), 0.63 (t, J = 6.4 Hz, 2H); MS calcd. for [M + H]⁺ C₂₄H₃₅N₂O₅S: 463.2; found: 463.2 |
| 142 | 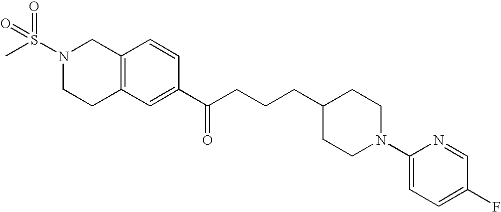 | ¹H-NMR (400 MHz, CDCl₃) δ = 7.96 (d, J = 3.2 Hz, 1H), 7.71 (m, 2H), 7.16 (m, 1H), 7.11 (d, J = 8.0 Hz, 1H), 6.54 (dd, J = 3.2, 9.6 Hz, 1H), 4.44 (s, 2H), 4.07 (d, J = 12.8 Hz, 2H), 3.52 (t, J = 6.0 Hz, 2H), 2.98 (t, J = 6.0 Hz, 2H), 2.88 (t, J = 7.2 Hz, 2H), 2.80 (s, 3H), 2.70 (td, J = 2.4, 12.4 Hz, 2H), 1.71 (m, 4H), 1.43 (m, 1H), 1.20 (m, 4H); MS calcd. for [M + H]⁺ C₂₄H₃₀FN₃O₃S: 460.2; found: 460.2 |
| 143 | 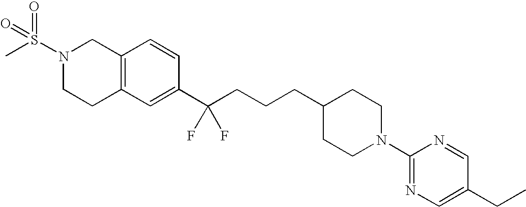 | ¹H-NMR (400 MHz, CDCl₃) δ = 8.11 (s, 2H), 7.22 (d, J = 8.0 Hz, 1H), 7.07 (d, J = 8.0 Hz, 1H), 4.59 (d, J = 13.2 Hz, 2H), 4.41 (s, 2H), 3.51 (t, J = 6.0 Hz, 2H), 2.95 (t, J = 4.8 Hz, 2H), 2.79 (s, 3H), 2.76 (td, J = 2.4, 12.4 Hz, 2H), 2.39 (q, J = 7.6 Hz, 2H), 2.02 (m, 2H), 1.67 (m, 8H), 1.39 (m, 3H), 1.21 (m, 2H), 1.11 (t, J = 7.6 Hz, 3H), 1.05 (m, 1H); MS calcd. for [M + H]⁺ C₂₅H₃₅F₂N₄O₂S: 493.2; found: 493.2. |
| 144 | 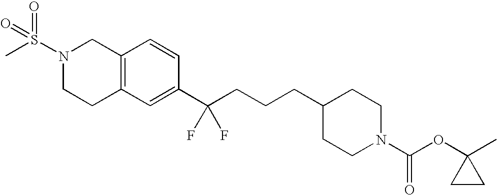 | ¹H-NMR (400 MHz, CDCl₃) δ = 7.28 (m, 2H), 7.16 (d, J = 8.0 Hz, 1H), 4.50 (s, 2H), 4.13 (m, 2H), 3.60 (t, J = 6.0 Hz, 2H), 3.03 (t, J = 6.0 Hz, 2H), 2.88 (s, 3H), 2.70 (m, 2H), 2.09 (m, 2H), 1.70 (s, 2H), 1.65 (s, 1H), 1.62 (s, 2H), 1.55 (s, 1H), 1.44 (m, 3H), 1.30 (m, 2H), 1.25 (s, 2H), 1.24 (s, 2H), 1.07 (m, 2H), 0.86 (t, J = 6.4 Hz, 1H), 0.64 (t, J = 6.4 Hz, 1H); MS calcd. for [M + H]⁺ C₂₄H₃₅F₂N₂O₄S: 485.2; found: 485.2. |
| 145 | 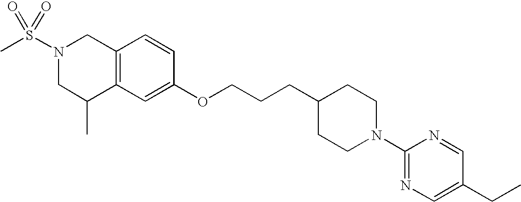 | MS calcd. for [M + H]⁺ C₂₅H₃₆N₄O₃S: 473.3; found: 473.8. |
| 146 | 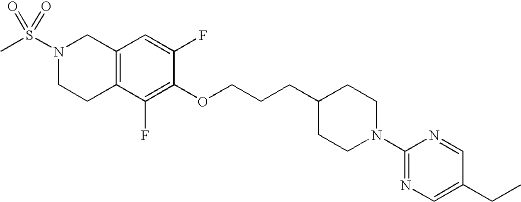 | MS calcd. for [M + H]⁺ C₂₄H₃₂F₂N₄O₃S: 495.2; found: 495.2. |
| 147 | 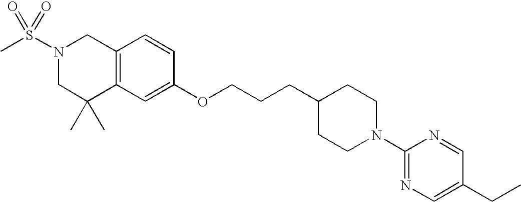 | MS calcd. for [M + H]⁺ C₂₆H₃₈N₄O₃S: 487.3; found: 487.8 |
| 148 | 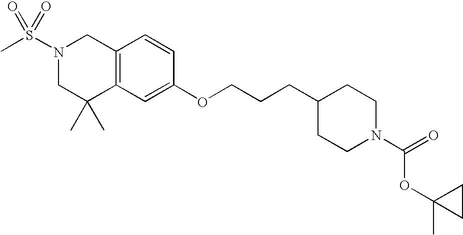 | MS calcd. for [M + H]⁺ C₂₅H₃₈N₂O₅S: 479.3; found: 479.8 |
| 149 | 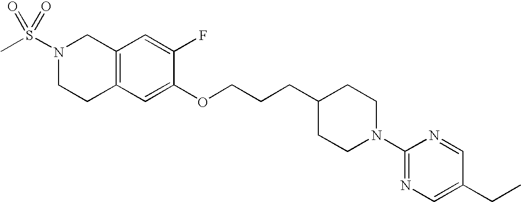 | MS calcd. for [M + H]⁺ C₂₄H₃₃FN₄O₃S: 477.2; found: 477.8. |
| 150 | 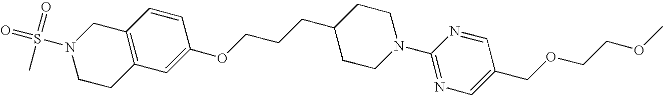 | MS calcd. for [M + H]⁺ C₂₆H₃₈N₄O₅S: 519.26; found: 519.2 |
| 151 | 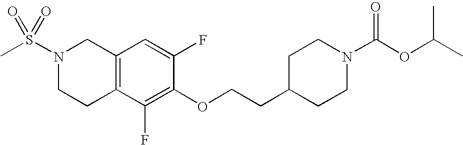 | MS calcd. for [M + H]⁺ C₂₁H₃₀F₂N₂O₅S: 461.2; found: 461.8. |
| 152 | 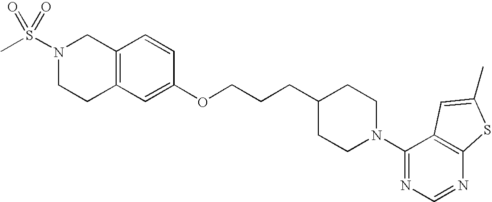 | MS calcd. for [M + H]⁺ C₂₅H₃₂N₄O₃S₂: 501.2; found: 501.2 |
| 153 | 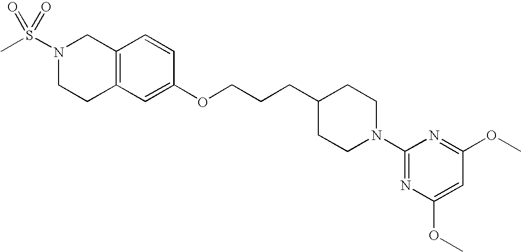 | MS calcd. for [M + H]⁺ C₂₄H₃₄N₄O₅S: 491.2; found: 491.2. |
| 154 | 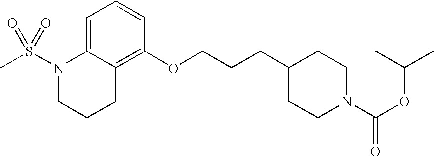 | MS calcd. for [M + H]⁺ C₂₂H₃₄N₂O₅S: 439.2; found: 439.2 |
| 155 | 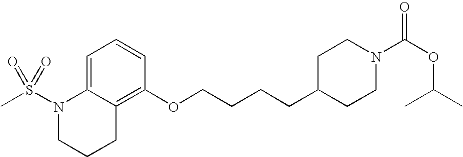 | MS calcd. for [M + H]⁺ C₂₃H₃₆N₂O₅S: 453.2; found: 453.2 |
| 156 | 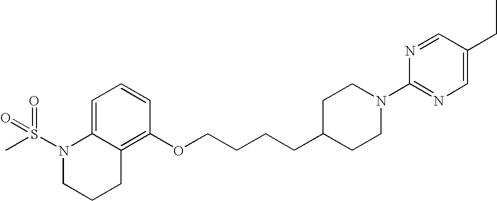 | MS calcd. for [M + H]⁺ C₂₅H₃₆N₄O₃S: 472.2; found: 472.2 |
| 157 | 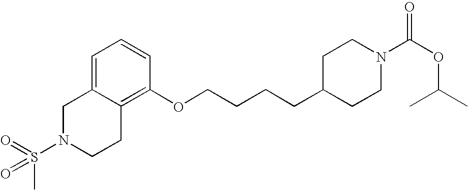 | MS calcd. for [M + H]⁺ C₂₃H₃₆N₂O₅S: 452.2; found: 452.2 |
| 158 | 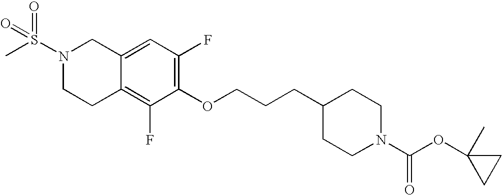 | MS calcd. for [M + H]⁺ C₂₃H₃₂F₂N₂O₅S: 487.2; found: 486.8. |
| 159 | 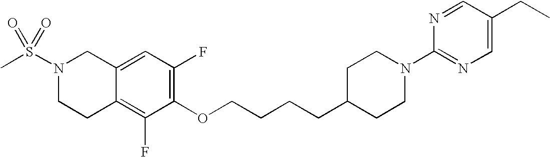 | MS calcd. for [M + H]⁺ C₂₅H₃₄F₂N₄O₃S: 509.2; found: 509.8. |
| 160 | 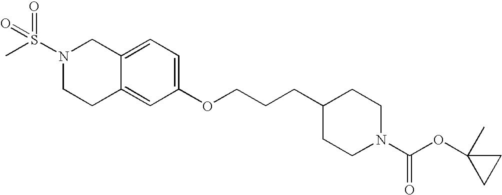 | MS calcd. for [M + H]⁺ C₂₃H₃₄N₂O₅S: 451.2; found: 451.2. |
| 161 | 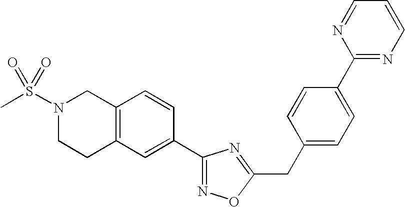 | MS calcd. for [M + H]⁺ C₂₃H₂₁N₅O₃S: 448.1; found 448.1. |
| 162 | 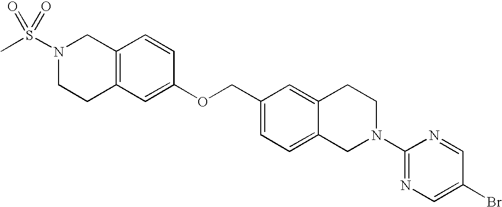 | MS Calcd for [M + H]⁺ C₂₄H₂₆BrN₄O₃S: 529.1; Found: 529.1 |
| 163 | 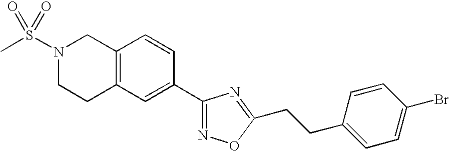 | ¹H NMR (CDCl₃) δ 7.89 (1 H, dd, J = 1.6 Hz, J = 8 Hz), 7.87 (1 H, s), 7.42 (2 H, d, J = 8 Hz), 7.21 (1H, d, J = 8 Hz), 7.11 (2H, d, J = 8 Hz), 4.51 (2 H, s), 3.60 (2 H, t, J = 2 Hz), 3.24 (2 H, m), 3.21 (2 H, m), 3.07 (2 H, t, J = 2 Hz), 2.86 (3H, s). MS Calcd for [M + H]⁺ C₂₀H₂₁BrN₃O₃S: 462.0; Found: 462.0 |
| 164 | 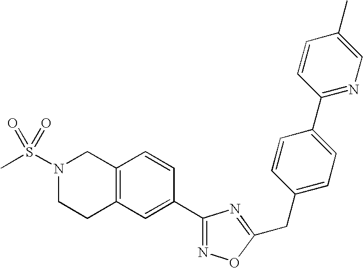 | MS Calcd for [M + H]⁺ C₂₅H₂₅N₄O₃S: 461.1; Found: 461.2 |
| 165 | 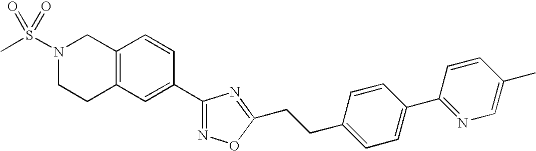 | ¹H NMR (CDCl₃) δ 8.50 (1 H, brs), 7.91 (2 H, d, J = 8 Hz), 7.89 (2 H, brs), 7.60 (1 H, d, J = 8 Hz), 7.54 (1 H, dd, J = 8 Hz, J = 2 Hz), 7.34 (2 H, d, J = 8 Hz), 7.21 (1 H, d, J = 8 Hz), 4.51 (2 H, s), 3.60 (2 H, t, J = 6 Hz), 3.28 (4 H, m), 3.06 (2 H, t, J = 6 Hz), 2.86 (3H, s), 2.36 (3H, s) MS Calcd for [M + H]⁺ C₂₆H₂₇N₄O₃S: 475.2; Found: 475.2 |
| 166 | 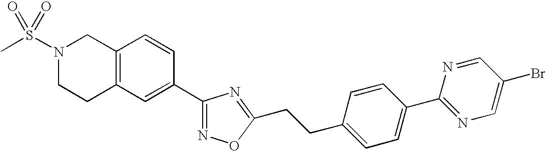 | ¹H NMR (CDCl₃) δ 8.81 (2 H, s), 8.34 (2 H, d, J = 8 Hz), 7.90 (1 H, d, J = 8 Hz), 7.88 (1 H, s), 7.38 (2 H, d, J = 8 Hz), 7.21 (1 H, d, J = 8 Hz), 4.51 (2 H, s), 3.60 (2 H, t, J = 6 Hz), 3.25-3.35 (4 H, m), 3.06 (2 H, t, J = 6 Hz), 2.86 (3 H, s) MS Calcd for [M + H]⁺ C₂₄H₂₃BrN₅O₃S: 540.1; Found: 540.1 |
| 167 | 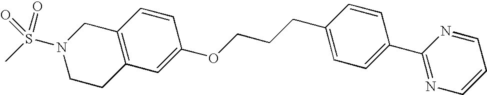 | ¹H NMR (CDCl₃) δ 8.79 (2 H, d, J = 4.8 Hz), 8.35 (2 H, d, J = 8 Hz), 7.33 (2 H, d, J = 8 Hz), 7.17 (1 H, t, J = 4.9 Hz), 6.99 (1 H, d, J = 8 Hz), 6.76 (1 H, dd, J = 8 Hz, J = 2.7 Hz), 6.66 (1 H, d, J = 2.5 Hz), 4.40 (2 H, s), 3.94 (2 H, t, J = 6.4 Hz), 3.54 (2 H, t, J = 6.0 Hz), 2.86-2.94 (4 H, m), 2.83 (3 H, s), 2.14 (2 H, m). MS Calcd for [M + H]⁺ C₂₃H₂₆N₃O₃S: 424.2; Found: 424.2 |
| 168 | 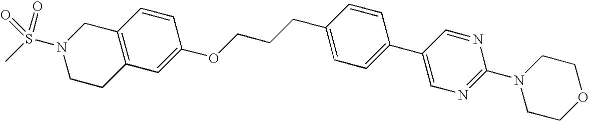 | ¹H NMR (CDCl₃) δ 8.54 (2 H, s), 7.40 (2 H, d, J = 8 Hz), 7.28 (2 H, J = 8 Hz), 7.00 (1 H, d, J = 8 Hz), 6.77 (1 H, dd, J = 8 Hz, J = 2.8 Hz), 6.67 (1 H, J = 2.4 Hz), 4.39 (2 H, s), 3.95 (2 H, t, J = 6.2 Hz), 3.84 (4 H, m), 3.78 (4 H, m), 3.53 (2 H, t, J = 6.2 Hz), 2.93 (2 H, t, J = 6 Hz), 2.84 (2 H, t, J = 7.8 Hz), 2.82 (3 H, s), 2.11 (2 H, m). MS Calcd for [M + H]⁺ C₂₇H₃₂N₄O₄S: 508.2; Found: 508.2 |
| 169 | 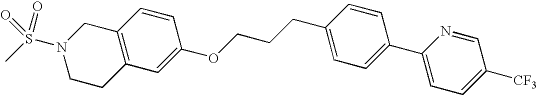 | ¹HNMR (CDCl₃) δ 8.91 (2 H, brs), 7.94-7.98 (3 H, m), 7.81 (1 H, d, J = 8.4 Hz), 7.34 (2 H, d, J = 8.4 Hz), 6.99 (1 H, d, J = 8.4 Hz), 6.76 (1 H, dd, J = 8.4 Hz, J = 2.4 Hz), 6.66 (1 H, d, J = 2.8 Hz), 4.39 (2 H, s), 3.94 (2 H, t, J = 6.4 Hz), 3.53 (2 H, t, J = 6.0 hz), 2.93 (2 H, t, J = 6.0 Hz), 2.90 (2 H, t, J = 7.6 hz), 2.82 (3 H, s), 2.13 (2 H, m). MS Calcd for [M + H]⁺ C₂₅H₂₆F₃N₂O₃S: 491.1; Found: 491. |
| 170 | 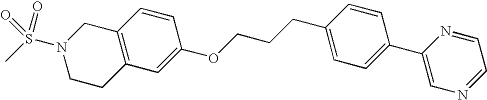 | ¹H NMR (CDCl₃) δ 9.00 (1 H, d, J = 1.6 Hz), 8.60 (1 H, m), 8.48 (1 H, d, J = 2.8 Hz), 7.93 (2 H, d, J = 8 Hz), 7.34 (2 H, d, J = 8 Hz), 6.99 (1 H, d, J = 8.4 Hz), 7.75 (1 H, dd, J = 8.4 Hz, J = 2.8 Hz), 6.66 (1 H, d, J = 2.8 Hz), 4.38 (2 H, s), 3.94 (2 H, t, J = 6.2 Hz), 3.53 (2 H, t, J = 6.0 Hz), 2.92 (2 H, t, J = 6.0 Hz), 2.87 (2 H, t, J = 7.4 Hz), 2.81 (3 H, s), 2.13 (2 H, m). MS Calcd for [M + H]⁺ C₂₃H₂₆N₃O₃S: 424.2; Found: 424.2 |
| 171 | 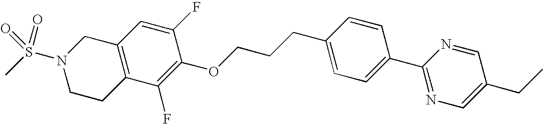 | MS calcd. for [M + H]⁺ C₂₅H₂₇F₂N₃O₃S: 488.2; found 488.1. |
| 172 | 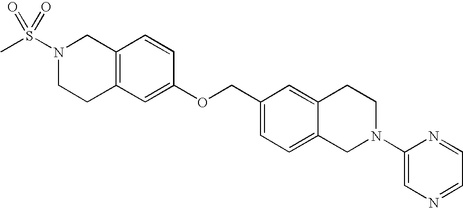 | ¹H NMR (CDCl₃) δ 8.18 (1 H, d, J = 1.6 Hz), 8.09 (1 H, m), 7.85 (1 H, d, J = 2.4 Hz), 7.24-7.28 (3 H, m), 7.01 (1 H, d, J = 8.6 Hz), 6.84 (1 H, dd, J = 8.4 Hz, J = 2.4 hz), 6.76 (1 H, d, J = 2.4 H), 5.01 (2 H, s), 4.74 (2H, s), 4.40 (2H, s), 3.87 (2H, t, J = 6 Hz), 3.54 (2H, t, J = 6.0 Hz), 3.00 (2H, t, J = 6 Hz), 2.95 (2 H, t, J = 6.0 Hz), 2.83 (3 H, s). MS Calcd for [M + H]⁺ C₂₄H₂₇N₄O₃S: 451.2; found: 451.2 |
| 173 | 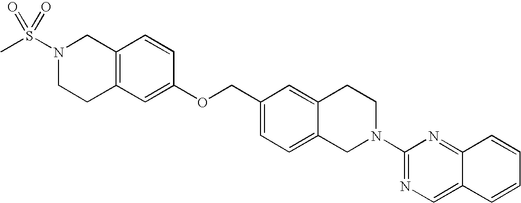 | MS Calcd for [M + H]⁺ C₂₈H₂₉N₄O₃S: 501.2; found: 501.2 |
| 174 | 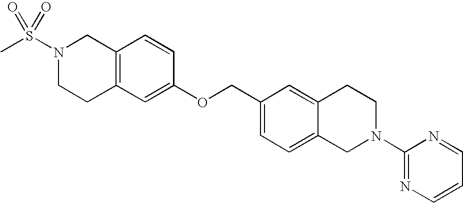 | ¹H NMR (CDCl₃) δ 8.36 (2 H, d, J = 4.8 Hz), 7.20- 7.25 (3 H, m), 7.00 (1 H, d, J = 8.4 Hz), 6.83 (1 H, dd, J = 8.4 Hz, J = 2.8 Hz), 6.76 (1 H, d, J = 2.4 hz), 6.51 (1 H, t, J = 4.8 Hz), 5.00 (2 H, s), 4.91 (2 H, s), 4.40 (2 H, s), 4.06 (2 H, t, J = 6.0 Hz), 3.54 (2 H, t, J = 6.0 Hz), 2.90- 2.97 (4 H, m), 2.82 (3 H, s). MS Calcd for [M + H]⁺ C₂₄H₂₇N₄O₃S: 451.2; found: 451.2 |
| 175 | 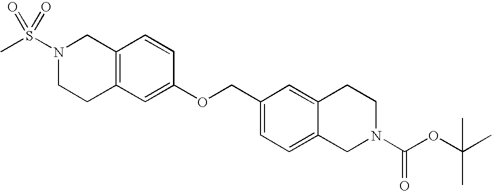 | MS Calcd for [M + H]⁺ C₂₅H₃₃N₂O₅S: 472.2; found: 473.2 |
| 176 | 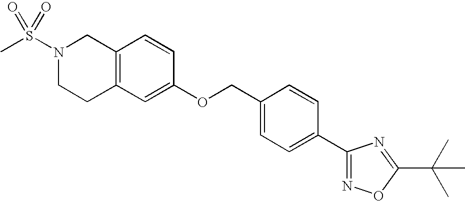 | MS Calcd for [M + H]⁺ C₂₃H₂₈N₃O₄S: 442.2; found: 442.2 |
| 177 | 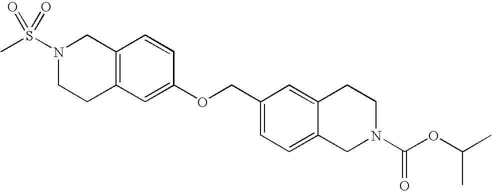 | ¹H NMR (CDCl₃) δ 7.20- 7.24 (2 H, m), 7.13 (1 H, d, J = 7.6 Hz), 7.00 (1 H, d, J = 8.4 Hz), 6.83 (1 H, dd, J = 8.4 Hz, J = 2.4 Hz), 6.75 (1 H, d, J = 6.4 Hz), 4.98 (2 H, s), 4.61 (2 H, brs), 4.39 (2 H, s), 3.68 (2H, brs), 3.54 (2 H, t, J = 6 Hz), 2.94 (2 H, t, J = 6.0 Hz), 2.85 (1 H, m), 2.82 (3 H, s), 1.27 (6 H, d, J = 6.4 Hz). MS Calcd for [M + H]⁺ C₂₄H₃₁N₂O₅S: 459.2; found: 459.2 |
| 178 | 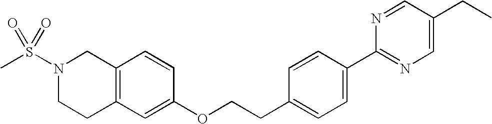 | MS calcd. for [M + H]⁺ C₂₄H₂₇N₃O₃S: 438.2, found 438.1. |
| 179 | 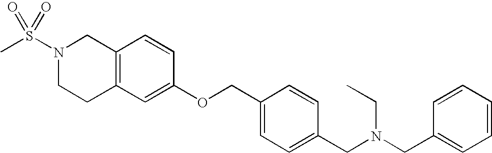 | ¹H-NMR (400 MHz, CDCl₃) δ = 7.59-7.40 (m, 9H), 7.02 (d, J = 8.4 Hz, 2H), 6.84 (dd, J = 8.4, 2.5 Hz, 2H), 6.77 (d, J = 2.5 Hz, 2H), 5.10 (s, 2H), 4.41 (s, 2H), 4.44-4.36 (m, 2H), 4.22-4.12 (m, 2H), 3.55 (t, J = 6.0 Hz, 2H), 3.04 (q, J = 7.2 Hz, 2H), 2.96 (t, J = 6.0 Hz, 2H), 2.85 (s, 3H), 1.40 (t, J = 7.2 Hz, 3H); MS calcd. for [M + H]⁺ C₂₇H₃₃N₂O₃S: 465.2; found: 465.2. |
| 180 | 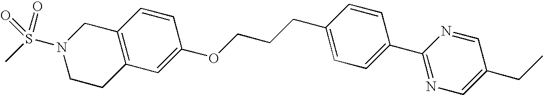 | MS calcd. for [M + H]⁺ C₂₅H₂₉N₃O₃S: 452.2; found 452.1. |
| 182 | 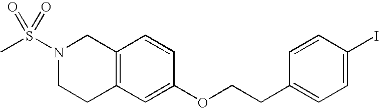 | MS Calcd for [M + H]⁺ C₁₈H₂₁INO₃S: 458.0, found: 458.0 |
| 182 | 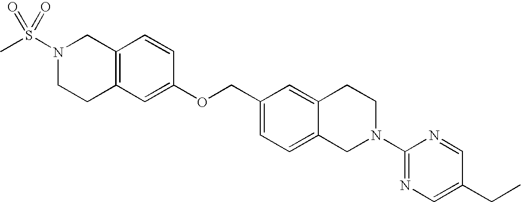 | MS calcd. for [M + H]⁺ C₂₆H₃₀N₄O₃S: 479.2; found 479.1. |
| 183 | 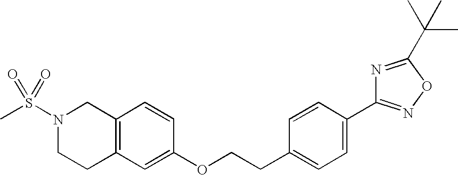 | MS Calcd for [M + H]⁺ C₂₄H₂₉N₃O₄S: 455.2; found: 455.2 |
| 184 | 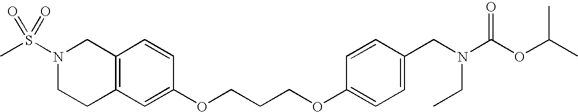 | ¹H-NMR (400 MHz, CDCl₃) δ = 7.18 (br s, 2H), 7.01 (d, J = 8.4 Hz, 1H), 6.87 (d, J = 8.8 Hz, 2H), 6.79 (dd, J = 2.8, 8.4 Hz, 1H), 6.71 (d, J = 2.4 Hz, 1H), 4.99 (m, 1H), 4.42 (s, 4H), 4.16 (t, J = 6.0 Hz, 4H), 3.56 (t, J = 6.0 Hz, 2H), 3.24 (m, 2H), 2.95 (t, J = 6.0 Hz, 2H), 2.84 (s, 3H), 2.26 (m, 2H), 1.28 (s, 3H), 1.27 (s, 3H), 1.07 (s, 3H); MS calcd. for [M + H]⁺ C₂₆H₃₇N₂O₆S: 505.22 found: 505.2. |
| 185 | 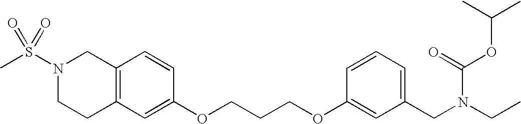 | ¹H-NMR (400 MHz, CDCl₃) δ = 7.24 (t, J = 8.0 Hz, 1H), 7.01 (d, J = 8.8 Hz, 1H), 6.81 (m, 4H), 6.71 (d, J = 2.4 Hz, 1H), 4.99 (m, 1H), 4.42 (m, 4H), 4.16 (t, J = 6.0 Hz, 4H), 3.56 (t, J = 6.0 Hz, 2H), 3.24 (m, 2H), 2.95 (t, J = 6.0 Hz, 2H), 2.84 (s, 3H), 2.26 (m, 2H), 1.59 (s, 8H), 1.28 (s, 6H), 1.09 (s, 3H); MS calcd. for [M + H]⁺ C₂₆H₃₇N₂O₆S: 505.2; found: 505.2. |
| 186 | 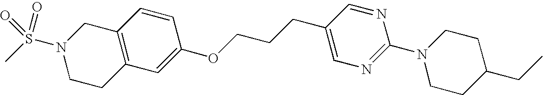 | MS calcd. for M + H⁺ C₂₃H₃₃N₅O₃S : 460.2; found 460.1. |
| 187 | 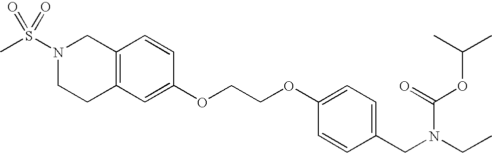 | ¹H-NMR (400 MHz, CDCl₃) δ = 7.05 (m, 2H), 6.92 (d, J = 8.4 Hz, 2H), 6.85 (m, 2H), 6.76 (d, J = 2.4 Hz, 1H), 5.00 (m, 1H), 4.43 (m, 5H), 4.31 (s, 3H), 4.04 (q, J = 7.2 Hz, 1H), 3.57 (m, 3H), 3.24 (m, 2H), 2.97 (t, J = 6.0 Hz, 2H), 2.85 (s, 3H), 1.60 (s, 6H), 1.28 (s, 3H), 1.27 (s, 3H), 1.07 (m, 3H); MS calcd. for [M + H]⁺ C₂₅H₃₅N₂O₆S: 491.2; found: 491.2. |
| 188 | 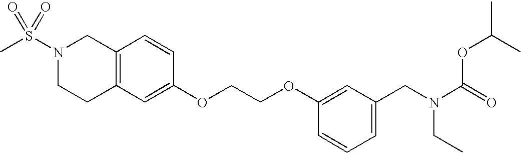 | ¹H-NMR (400 MHz, CDCl₃) δ = 7.25 (t, J = 8.0 Hz, 1H), 7.04 (d, J = 8.4 Hz, 1H), 6.85 (m, 4H), 6.76 (d, J = 2.4 Hz, 1H), 5.00 (m, 1H), 4.46 (s, 2H), 4.43 (s, 2H), 4.32 (s, 3H), 3.56 (t, J = 6.0 Hz, 2H), 3.26 (m, 2H), 2.98 (t, J = 6.0 Hz, 2H), 2.85 (s, 3H), 1.28 (s, 6H), 1.09 (s, 3H); MS calcd. for [M + H]⁺ C₂₅H₃₅N₂O₆S: 491.2; found: 491.2. |
| 189 | 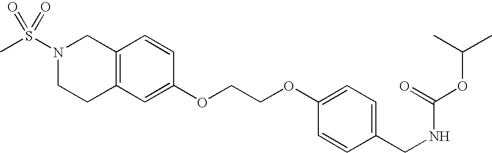 | ¹H-NMR (400 MHz, CDCl₃) δ = 7.24 (d, J = 8.4 Hz, 2H), 7.08 (d, J = 8.8 Hz, 1H), 7.03 (d, J = 8.4 Hz, 1H), 6.93 (d, J = 8.4 Hz, 2H), 6.84 (dd, J = 2.4, 8.4 Hz, 1H), 6.76 (d, J = 2.4 Hz, 1H), 4.97 (m, 1H), 4.87 (s, 1H), 4.43 (s, 3H), 4.32 (2, 5H), 3.56 (m, 2H), 2.97 (m, 2H), 2.86 (s, 3H), 1.71 (s, 4H), 1.26 (s, 3H), 1.25 (s, 3H). MS calcd. for [M + H]⁺ C₂₃H₃₁N₂O₆S: 463.2; found: 463.2. |
| 190 | 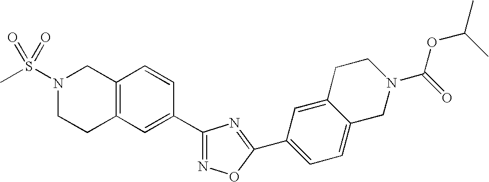 | MS calcd. for [M + H]⁺ C₂₅H₂₈N₄O₅S: 497.2; found 497.2. |
| 191 | 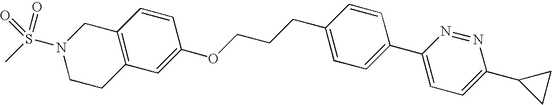 | MS Calcd for [M + H]⁺ C₂₆H₃₀N₃O₃S: 464.6; found: 464.6 |
| 192 | 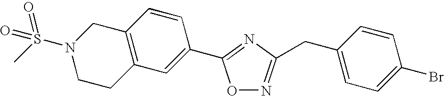 | MS Calcd for [M + H]⁺ C₂₆H₃₀N₃O₃S: 448.3; found: 448.3 |
| 193 | 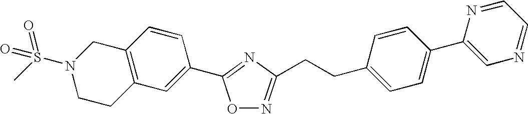 | MS Calcd for [M + H]⁺ C₂₆H₂₉N₃O₃S: 462.1; found: 462.1 |
| 194 | 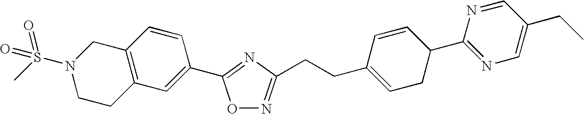 | MS Calcd for [M + H]⁺ C₂₆H₃₀N₃O₃S: 492.2; found: 492.2 |
| 195 | 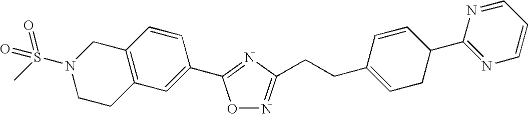 | MS Calcd for [M + H]⁺ C₂₆H₂₉N₃O₃S: 462.1; found: 462.1 |
| 196 | 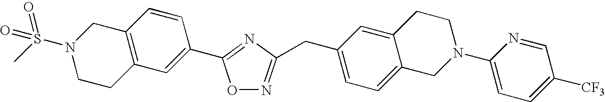 | MS Calcd for [M + H]⁺ C₂₆H₃₀N₃O₃S: 570.1; found: 570.1 |
| 197 | 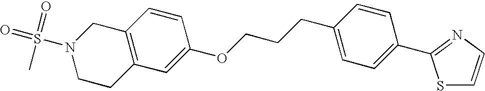 | MS Calcd for [M + H]⁺ C₂₆H₃₀N₃O₃S: 429.1; found: 429.1 |
| 198 | 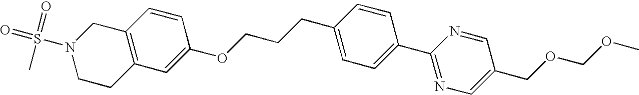 | MS Calcd for [M + H]⁺ C₂₆H₃₁N₃O₅S: 497.2; found: 497.8 |
| 199 | 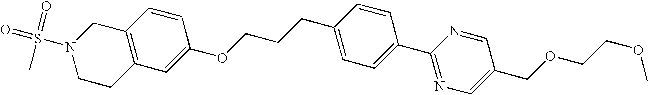 | MS Calcd for [M + H]⁺ C₂₇H₃₃N₃O₅S: 511.2; found: 511.7 |
| 200 | 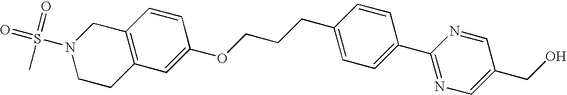 | MS Calcd for [M + H]⁺ C₂₄H₂₇N₃O₄S: 453.1; found: 453.8 |
| 201 | 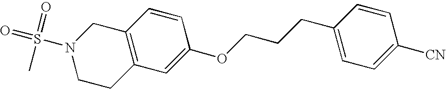 | MS Calcd for [M + H]⁺ C₂₆H₃₀N₃O₃S: 371.1; found: 371.1 |
| 202 | 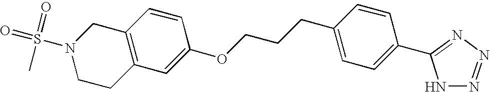 | MS Calcd for [M + H]⁺ C₂₆H₃₀N₃O₃S: 414.1; found: 414.1 |
| 203 | 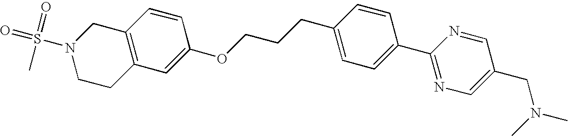 | MS Calcd for [M + H]⁺ C₂₆H₃₃N₄O₃S: 481.2, found: 481.2 |
| 204 | 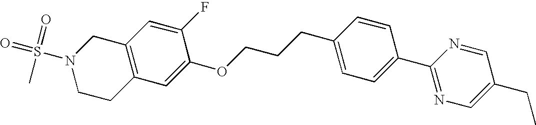 | MS Calcd for [M + H]⁺ C₂₅H₂₈FN₃O₃S: 470.1; found: 470.1 |
| 205 | 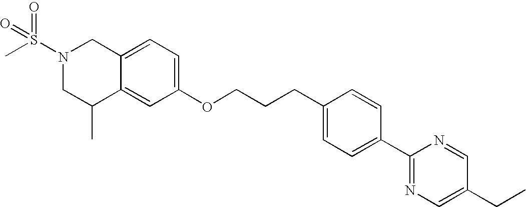 | MS Calcd for [M + H]⁺ C₂₆H₃₁N₃O₃S: 466.2; found: 466.2. |
| 206 | 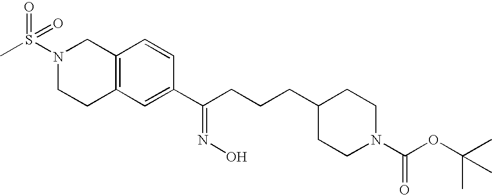 | ¹H-NMR (400 MHz, CD₃CN) δ = 8.84 (s, 1H), 7.39-7.37 (m 2H), 7.08 (d, J = 8.0 Hz, 1H), 4.32 (s, 2H), 3.98-3.85 (m, 2H), 3.40 (t, J = 6.0 Hz, 2H), 2.90 (t, J = 6.0 Hz, 2H), 2.76 (s, 3H), 2.65-2.61 (m, 2H), 2.56-2.44 (m, 2H), 1.54-1.50 (m, 2H), 1.45-1.36 (m, 2H), 1.32 (s, 9H), 1.32-1.21 (m, 1H), 1.21-1.16 (m, 2H), 0.91-0.79 (m 2H); MS calcd. for [M + H]⁺ C₂₄H₃₈N₃O₅S: 480.2; found: 480.2. |
| 207 | 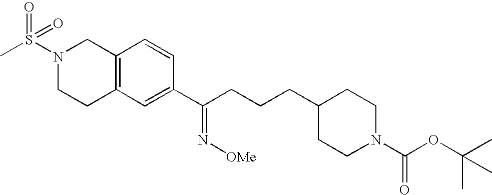 | ¹H-NMR (400 MHz, CD₃CN) δ = 7.40-7.37 (m 2H), 7.08 (d, J = 8.0 Hz, 1H), 4.32 (s, 2H), 3.91- 3.88 (m, 2H), 3.82 (s, 3H), 3.41 (t, J = 6.0 Hz, 2H), 2.89 (t, J = 6.0 Hz, 2H), 2.75 (s, 3H), 2.63- 2.59 (m, 2H), 2.56-2.50 (m, 2H), 1.52-1.49 (m, 2H), 1.44-1.36 (m, 2H), 1.32 (s, 9H), 1.32-1.24 (m, 1H), 1.24-1.14 (m, 2H), 0.92-0.82 (m 2H); MS calcd. for [M + H]⁺ C₂₅H₄₀N₃O₅S: 494.2; found: 494.2. |
| 208 | 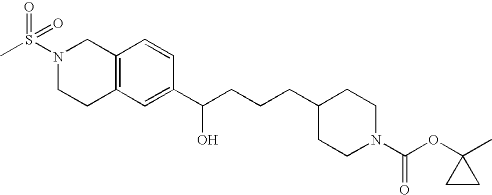 | ¹H-NMR (400 MHz, CD₃CN) δ = 7.07-6.99 (m, 3H), 4.46-4.42 (m, 1H), 4.28 (s, 2H), 4.96- 4.75 (m, 2H), 3.38 (t, J = 6.0 Hz, 2H), 2.85 (t, J = 6.0 Hz, 2H), 2.74 (s, 3H), 2.64-2.54 (m, 2H), 1.58- 1.45 (m, 4H), 1.38 (s, 3H), 1.33-1.21 (m, 2H), 1.20-1.09 (m, 3H), 0.91- 0.81 (m, 2H), 0.70-0.67 (m, 2H), 0.50-0.47 (m, 2H); MS calcd. for [M + H]⁺ C₂₄H₃₇N₂O₅S: 465.2; found: 465.2. |
| 209 | 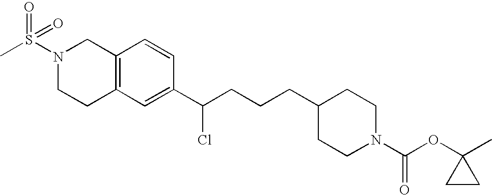 | ¹H-NMR (400 MHz, CD₃CN) δ = 7.17-7.15 (m, 2H), 7.06 (d, J = 8.0 Hz, 1H), 4.87-4.83 (m, 1H), 4.30 (s, 2H), 4.98- 4.72 (m, 2H), 3.39 (t, J = 6.0 Hz, 2H), 2.86 (t, J = 6.0 Hz, 2H), 2.74 (s, 3H), 2.56 (br. s, 2H), 2.04-1.90 (m, 2H), 1.54-1.44 (m, 2H), 1.38 (s, 3H), 1.33- 1.24 (m, 1H), 1.20-1.12 (m, 4H), 0.90-0.81 (m, 2H), 0.70-0.68 (m, 2H), 0.50-0.47 (m, 2H); MS calcd. for [M + H]⁺ C₂₄H₃₆ClN₂O₄S: 483.2; found: 483.2. |
| 210 | 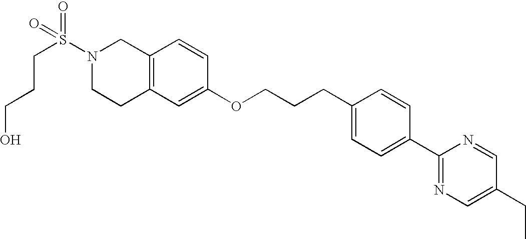 | MS calcd. for [M + H]⁺ C₂₇H₃₄N₃O₄S: 496.2; found: 496.2. |
| 211 | 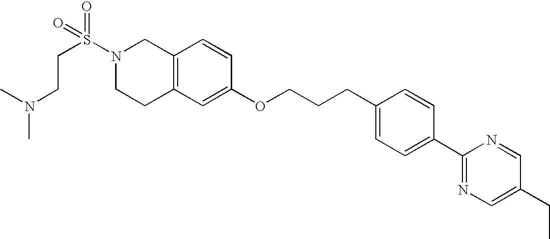 | MS calcd. for [M + H]⁺ C₂₈H₃₇N₄O₃S: 509.2; found: 509.2 |
| 212 | 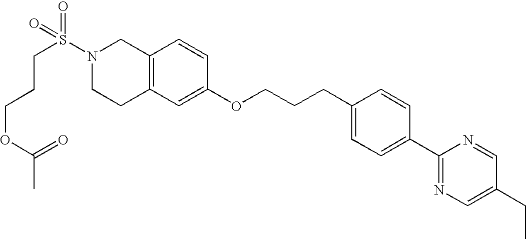 | MS calcd. for [M + H]⁺ C₂₉H₃₆N₃O₅S: 538.2; found: 538.2 |
| 213 | 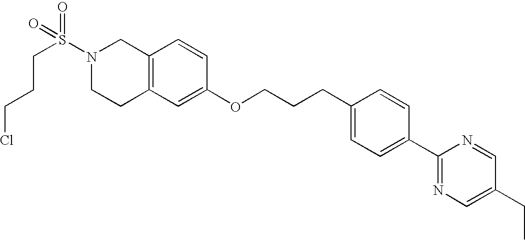 | MS calcd. for [M + H]⁺ C₂₇H₃₃ClN₃O₃S: 514.1; found: 514.1 |
| 214 | 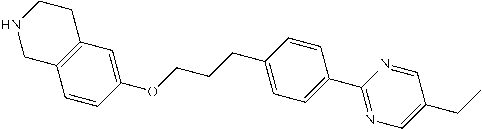 | MS calcd. for [M + H]⁺ C₂₄H₂₈N₃O: 374.2; found: 374.2. |
| 215 | 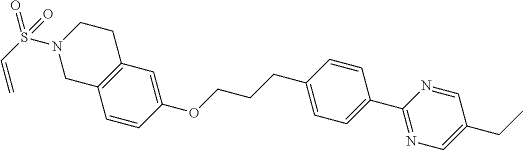 | MS calcd. for [M + H]⁺ C₂₆H₃₀N₃O₃S: 464.2; found: 464.2. |
| 216 | 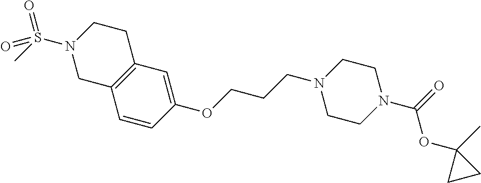 | MS calcd. for [M + H]⁺ C₂₂H₃₃N₃O₅S: 452.2; found: 452.2. |
| 217 | 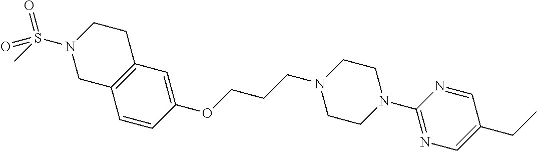 | ¹H-NMR (400 MHz, CDCl₃) δ 8.18 (s, 2H), 7.00 (d, 1H, J = 8.4 Hz), 6.77 (dd, 1H, J = 2.4, 8.4 Hz), 6.69 (d, 1H, J = 2.4 Hz), 4.40 (s, 2H), 4.02 (t, 2H, J = 6.0 Hz), 3.80 (t, 4H, J = 4.8 Hz), 3.54 (t, 2H, J = 6.0 Hz), 2.94 (t, 2H, J = 6.0 Hz), 2.83 (s, 3H), 2.58-2.51 (m, 6H), 2.46 (q, 2H, J = 7.6 Hz), 2.00 (quint, 2H, J = 6.4 Hz), 1.19 (t, 3H, J = 7.6 Hz); MS calcd. for [M + H]⁺ C₂₃H₃₃N₅O₃S: 460.2; found: 460.2. |
| 218 | 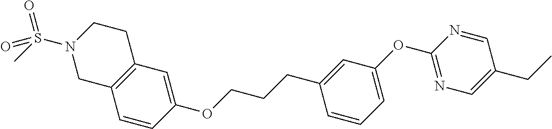 | MS calcd. for [M + H]⁺ C₂₅H₂₉N₃O₄S: 468.2; found: 468.2. |
| 219 | 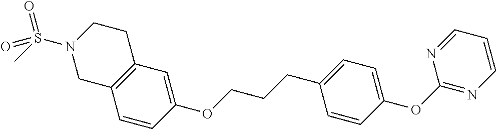 | ¹H-NMR (400 MHz, CDCl₃) δ 8.57 (d, 2H, J = 4.8 Hz), 7.27 (d, 2H, J = 8.4 Hz), 7.14-7.11 (m, 2H), 7.03 (t, 1H, J = 4.8 Hz), 7.00 (d, 1H, J = 8.4 Hz), 6.77 (dd, 1H, J = 2.4, 8.4 Hz), 6.68 (d, 1H, J = 2.4 Hz), 4.40 (s, 2H), 3.97 (t, 2H, J = 6.4 Hz), 3.55 (t, 2H, J = 6.0 Hz), 2.94 (t, 2H, J = 6.0 Hz), 2.85- 2.81 (m, 5H), 2.15-2.09 (m, 2H); MS calcd. for [M + H]⁺ C₂₃H₂₅N₃O₄S: 440.2; found: 440.1. |
| 220 | 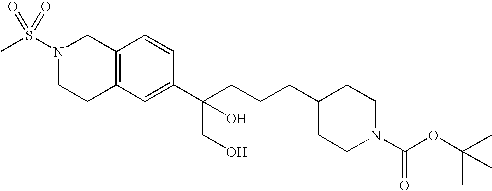 | ¹H-NMR (400 MHz, CD₃CN) δ = 7.23 (m, 2H), 7.10 (d, J = 8.5 Hz, 1H), 4.38 (s, 2H), 3.95 (d, J = 12.8 Hz, 2H), 3.55 (d, J = 5.8 Hz, 2H), 3.48 (t, J = 5.8 Hz, 2H), 2.94 (t, J = 5.8 Hz, 2H), 2.83 (s, 3H), 2.61 (br s, 2H), 1.71 (dd, J = 9.0, 7.5 Hz, 2H), 1.52 (d, 12.3 Hz, 2H), 1.39 (s, 9H), 1.30 (m, 2H), 1.14 (m, 2H), 0.91 (m, 2H); MS calcd. for C₂₅H₄₀N₂O₆S (M + Na⁺) 519.3; found: 519.3. |
| 221 | 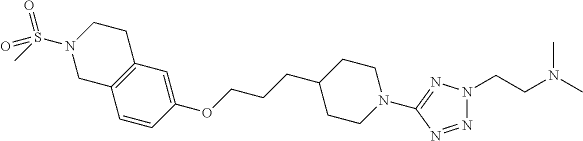 | ¹H-NMR (400 MHz, CD₃CN) δ = 7.04 (d, J = 8.4 Hz, 1H), 6.76 (dd, J = 2.8, 8.4 Hz, 1H), 6.72 (d, J = 2.4 Hz, 1H), 4.49 (t, J = 6.4 Hz, 2H), 4.31 (s, 2H), 4.00-3.93 m, 4H), 3.45 (t, J = 6.0 Hz, 2H), 3.08 (m, 2H), 2.92-2.84 (m, 4H), 2.81 (s, 3H), 2.74 (s, 6H), 2.33 (m, 2H), 1.80-1.76 (m, 4H), 1.50 (m, 1H), 1.43-1.37 (m, 2H), 1.24 (ddd, J = 4.4, 12.8, 24.8 Hz, 2H); MS calcd. for [M + H]⁺ C₂₃H₃₈N₇O₃S: 492.3; found: 492.2. |
| 222 | 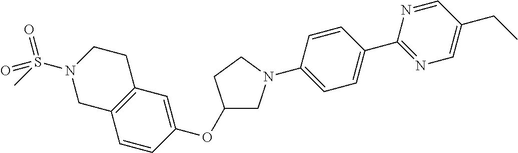 | MS calcd. for [M + H]⁺ C₂₆H₃₁N₄O₃S: 479.2; found: 479.7 |
| 223 | 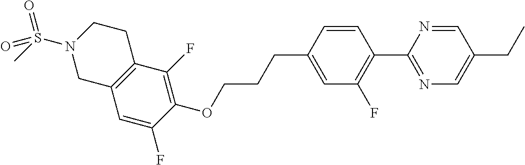 | MS calcd. for [M + H]⁺ C₂₅H₂₆F₃N₃O₃S: 506.2; found: 505.7 |
| 224 | 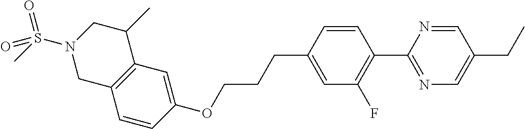 | MS calcd. for [M + H]⁺ C₂₆H₃₀FN₃O₃S: 484.2; found: 483.8 |
| 225 | 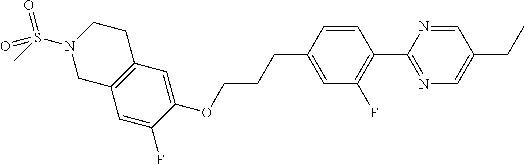 | MS calcd. for [M + H]⁺ C₂₅H₂₇F₂N₃O₃S: 488.2; found: 488.1 |
| 226 | 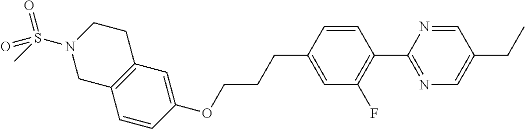 | ¹H-NMR (400 MHz, CDCl₃) δ 8.63 (s, 2H), 7.88 (t, 1H, J = 8.0 Hz), 7.05-6.96 (m, 2H), 6.93 (d, 1H, J = 8.4 Hz), 6.70 (dd, 1H, J = 2.8, 8.8 Hz), 6.59 (d, 1H, J = 2.4 Hz), 4.33 (s, 2H), 3.87 (t, 2H, J = 6.0 Hz), 3.48 (t, 2H, J = 6.0 Hz), 2.87 (t, 2H, J = 5.6 Hz), 2.80 (t, 2H, J = 7.2 Hz), 2.63 (q, 2H, J = 7.6 Hz), 2.06 (quint, 2H, J = 6.4 Hz), 1.26 (t, 3H, J = 7.6 Hz); MS calcd. for [M + H]⁺ C₂₅H₂₈FN₃O₃S: 470.2; found: 470.2. |
| 227 | 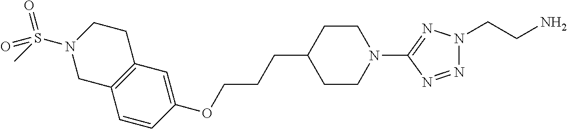 | ¹H-NMR (400 MHz, CD₃CN) δ = 7.97 (br s, 2H), 7.04 (d, J = 8.1 Hz, 1H), 6.76 (dd, J = 2.8, 8.4 Hz, 1H), 6.72 (d, J = 2.4 Hz, 1H), 4.75 (t, J = 5.6 Hz, 2H), 4.31 (s, 2H), 4.02 (m, 2H), 3.95 (t, J = 6.4 Hz, 2H), 3.49 (t, J = 5.6 Hz, 2H), 3.45 (t, J = 6.0 Hz, 2H), 2.93-2.86 (m, 4H), 2.80 (s, 3H), 1.80-1.76 (m, 4H), 1.53 (m, 1H), 1.43-1.38 (m, 2H), 1.24 (ddd, J = 4.0, 12.4, 24.2 Hz, 2H); MS calcd. for [M + H]⁺ C₂₁H₃₄N₇O₃S: 464.2; found: 464.2 |
| 228 | 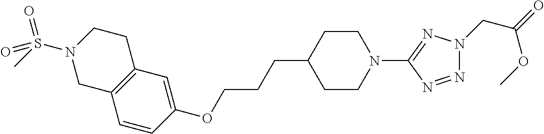 | ¹H-NMR (400 MHz, CD₃CN) δ = 7.04 (d J = 84 Hz, 1H), 6.76 (dd, J = 2.4, 8.4 Hz, 1H), 6.72 (d, J = 2.4 Hz, 1H), 5.26 (s, 2H), 4.31 (s, 2H), 4.00- 3.93 (m, 4H), 3.75 (s, 3H), 3.45 (t, J = 6.0 Hz, 2H), 2.93-2.86 (m, 4H), 2.81 (s, 3H), 2.17 (br s, 2H), 1.82-1.76 (m, 4H), 1.52 (m, 1H), 1.43-1.38 (m, 2H), 1.24 (ddd, J = 4.4, 12.8, 24.4 Hz, 2H); MS calcd. for [M + H]⁺ C₂₂H₃₃N₆O₅S: 493.2; found: 493.2. |
| 229 | 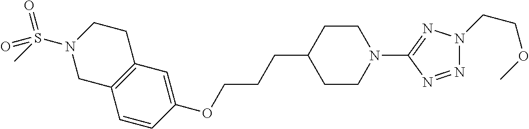 | ¹H-NMR (400 MHz, CD₃CN) δ = 7.04 (d, J = 6.3 Hz, 1H), 6.76 (dd, J = 2.8, 8.4 Hz, 1H), 6.72 (d, J = 1.8 Hz, 1H), 4.53 (t, J = 5.2 Hz, 2H), 4.31 (s, 2H), 3.99-3.93 (m, 4H), 3.81 (t, J = 4.8 Hz, 2H), 3.45 (t, J = 6.0 Hz, 2H), 3.26 (s, 3H), 2.92-2.83 (m, 4H), 2.81 (s, 3H), 1.80-1.76 (m, 4H), 1.51 (m, 1H), 1.40 (m, 2H), 1.24 (ddd, J = 4.4, 12.8, 24.8 Hz, 2H); MS calcd. for [M + H]⁺ C₂₂H₃₅N₆O₄S: 479.2; found: 479.2. |
| 230 | 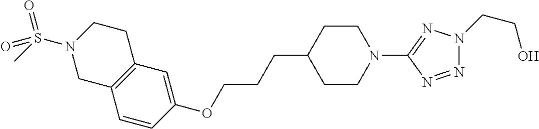 | ¹H-NMR (400 MHz, CD₃CN) δ = 7.04 (d, J = 8.4 Hz, 1H), 6.76 (dd, J = 2.8, 8.4 Hz, 1H), 6.72 (d, J = 2.4 Hz, 1H), 4.45 (t, J = 5.2 Hz, 2H), 4.31 (s, 2H), 4.01-3.92 (m, 6H), 3.45 (t, J = 6.0 Hz, 2H), 3.06 (br s, 1H), 2.92-2.84 (m, 4H), 2.81 (s, 3H), 1.80-1.76 (m, 4H), 1.51 (m, 1H), 1.41 (m, 2H), 1.24 (ddd, J = 4.4, 12.8, 24.8 Hz, 2H); MS calcd. for [M + H]⁺ C₂₁H₃₃N₆O₄S 465.2; found: 465.2. |
| 231 | 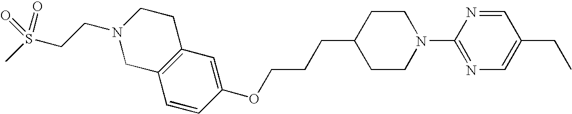 | MS calcd. for [M + H]⁺ C₂₆H₃₉N₄O₃S: 487.2; found: 487.2. |
| 232 | 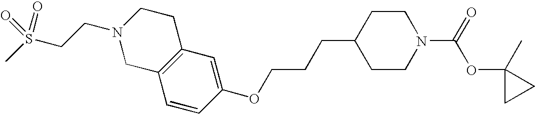 | MS calcd. for [M + H]⁺ C₂₅H₃₉N₂O₅S: 479.2; found: 479.8. |
| 233 | 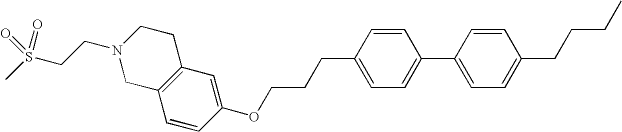 | MS calcd. for [M + H]⁺ C₂₉H₃₅NO₃S: 478.2; found: 477.8. |
| 234 | 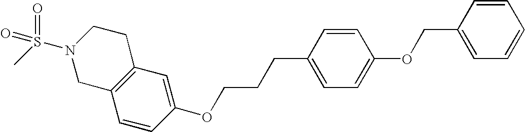 | ¹H-NMR (400 MHz, CDCl₃) δ 7.44-7.30 (m, 5H), 7.12 (d, 2H, J = 8.8 Hz), 6.99 (d, 1H, J = 8.4 Hz), 6.90 (d, 2H, J = 8.4 Hz), 6.76 (dd, 1H, J = 2.4, 8.4 Hz), 6.66 (d, 1H, J = 2.4 Hz), 5.29 (s, 2H), 4.40 (s, 2H), 3.92 (t, 2H, J = 6.4 Hz), 3.54 (t, 2H, J = 6.0 Hz), 2.93 (t, 2H, J = 6.0 Hz), 2.83 (s, 3H), 2.74 (t, 2H, J = 7.6 Hz), 2.06 (quint, 2H, J = 6.4 Hz); MS calcd. for [M + H]⁺ C₂₆H₂₉NO₄S: 452.2; found: 451.8. |
| 235 | 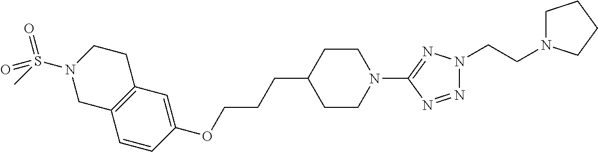 | ¹H-NMR (400 MHz, CD₃CN) δ = 7.04 (d, J = 7.6 Hz, 1H), 6.76 (dd, J = 2.4, 8.4 Hz, 1H), 6.72 (d, J = 2.4 Hz, 1H) 4.83 (t, J = 6.4 Hz, 2H), 4.32 (s, 2H), 4.31 (s, 2H), 3.99 (m, 2H), 3.95 (t, J = 6.4 Hz, 2H), 3.67 (t, J = 6.0 Hz, 2H), 3.45 (t, J = 6.0, 2H), 2.92-2.85 (m, 4H), 2.81 (s, 3H), 2.20 (m, 8H) 1.80-1.74 (m, 4H), 1.51 (m, 1H), 1.43-1.37 (m, 2H), 1.3 (dd, J = 4.0, 12.4, 24.4 Hz, 2H); MS calcd. for C₂₅H₄₀N₇O₃S [M + H]⁺ 518.3; found: 518.2. |
| 236 | 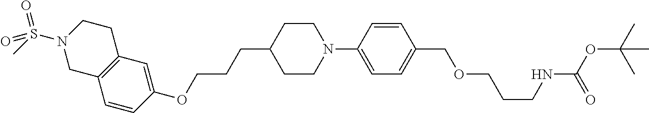 | MS calcd. for [M + H]⁺ C₃₃H₅₀N₃O₆S: 616.3; found: 616.3. |
| 237 | 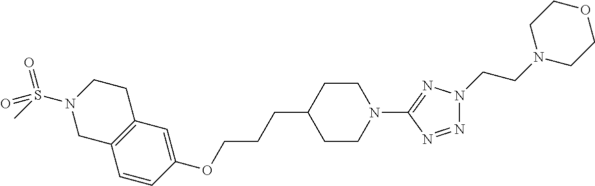 | ¹H-NMR (400 MHz, CD₃CN) δ = 7.04 (d, J = 8.4 Hz, 1H), 6.76 (dd, J = 2.8, 10.4 Hz, 1H), 6.72 (d, J = 2.4 Hz, 1H), 4.82 (t, J = 6.4 Hz, 2H), 4.31 (s, 2H), 4.00-3.93 (m, 4H), 3.81 (t, J = 4.4 Hz, 4H), 3.53 (t, J = 6.0, 2H), 3.45 (t, J = 6.0 Hz, 2H), 3.12 (br s, 4H), 2.92-2.85 (m, 4H), 2.81 (s, 3H), 1.80- 1.76 (m, 4H), 1.52 (m, 1H), 1.43-1.37 (m, 4H), 1.23 (ddd, J = 4.4, 12.8, 24.8 Hz, 2H); MS calcd. for C₂₅H₄₀N₇O₄S [M + H]⁺ 534.3; found: 534.2. |
| 238 | 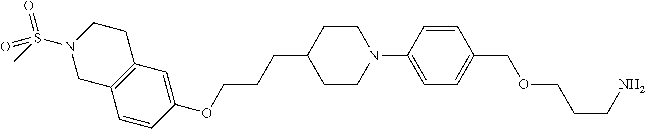 | MS calcd. for [M + H]⁺ C₂₈H₄₂N₃O₄S: 516.3; found: 516.3. |
| 239 | 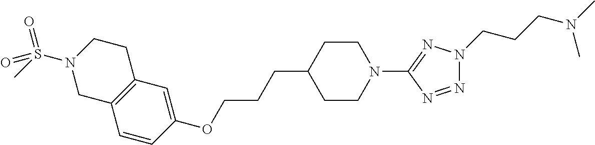 | ¹H-NMR (400 MHz, CD₃CN) δ = 7.04 (d, J = 8.4 Hz, 1H), 6.76 (dd, J = 2.8, 8.4 Hz, 1H), 6.72 (d, J = 2.4 Hz, 1H), 4.83 (t, J = 6.0 Hz, 2H), 4.31 (s, 2H), 3.99 (m, 2H), 3.94 (t, J = 6.8 Hz, 2H), 3.59 (t, J = 6.0 Hz, 2H), 3.45 (t, J = 6.0 Hz, 2H), 2.92-2.85 (m, 4H), 2.81 (s, 3H), 2.79 (s, 6H), 1.80-1.76 (m, 4H), 1.50 (m, 1H), 1.43-1.39 (m, 2H), 1.23 (ddd, J = 4.4, 12.8 Hz, 24.8 Hz, 2H); MS calcd. for C₂₄H₄₀N₇O₃S [M + H]⁺ 506.3; found: 506.2. |
| 240 | 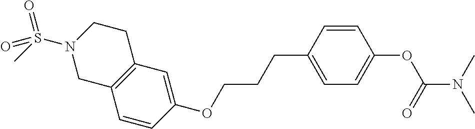 | ¹H-NMR (400 MHz, CDCl₃) δ 7.18 (d, 2H, J = 8.4 Hz), 7.03-6.98 (m, 3H), 6.76 (dd, 1H, J = 2.8, 8.4 Hz), 6.66 (d, 1H, J = 2.4 Hz), 4.40 (s, 2H), 3.92 (t, 2H, J = 6.0 Hz), 3.54 (t, 2H, J = 6.0 Hz), 3.09 (brs, 3H), 3.01 (brs, 3H), 2.94 (t, 2H, J = 6.0 Hz), 2.79 (t, 2H, J = 7.2 Hz), 2.11-2.05 (m, 2H); MS calcd. for [M + H]⁺ C₂₂H₂₈N₂O₅S: 433.2; found: 432.8. |
| 241 | 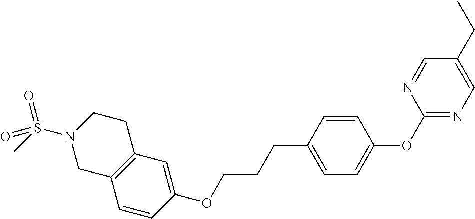 | MS calcd. for [M + H]⁺ C₂₅H₃₀N₃O₄S: 468.2; found: 468.2. |
| 242 | 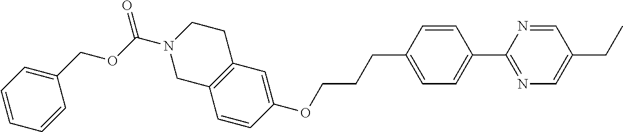 | MS calcd. for [M + H]⁺ C₃₂H₃₄N₃O₃: 508.2; found: 508.2. |
| 243 | 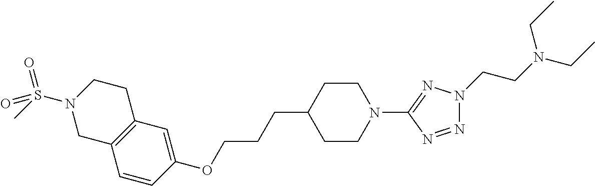 | ¹H-NMR (600 MHz, acetone-d₃) δ = 7.08 (d, J = 5.6 Hz, 1H), 6.79 (dd, J = 0.4, 5.6 Hz, 1H), 6.75 (d, J = 1.6 Hz, 1H), 5.03 (t, J = 4.4 Hz, 2H), 4.34 (s, 2H), 4.04 (m, 2H), 3.99 (t, J = 4.4 Hz, 2H), 3.77 (t, J = 3.6 Hz, 2H), 3.49 (t, J = 4.0 Hz, 2H), 3.30 (m, 4H), 2.94-2.88 (m, 4H), 2.87 (s, 3H), 1.85-1.80 (m, 4H), 1.56 (m, 1H), 1.45 (m, 2H), 1.32 (t, J = 4.8 Hz, 6H), 1.25 (ddd, J = 4.2, 12.6, 24.6 Hz, 2H); MS calcd. for C₂₅H₄₂N₇O₃S [M + 1]⁺ 520.3; found: 520.3. |
| 244 | 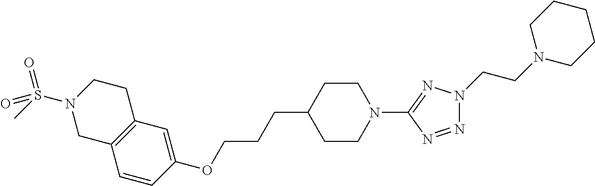 | ¹H-NMR (600 MHz, acetone-d₆) δ = 7.08 (d, J = 5.6 Hz, 1H), 6.79 (dd, J = 1.6, 5.6 Hz, 1H), 6.75 (d, J = 1.6 Hz, 1H), 5.02 (t, J = 4.4 Hz, 2H), 4.34 (s, 2H), 4.03 (m, 2H), 3.99 (t, J = 4.4 Hz, 2H), 3.71 (m, 2H), 3.49 (t, J = 4.0 Hz, 2H), 2.94-2.90 (m, 4H), 2.87 (s, 3H), 1.88 (m, 4H), 1.84-1.80 (m, 4H), 1.56 (m, 1H), 1.47-1.43 (m, 2H), 1.25 (ddd, J = 4.2, 12.6, 24.6 Hz, 2H); MS calcd. for C₂₆H₄₂N₇O₃S [M + 1]⁺ 532.3; found: 532.3. |
| 245 | 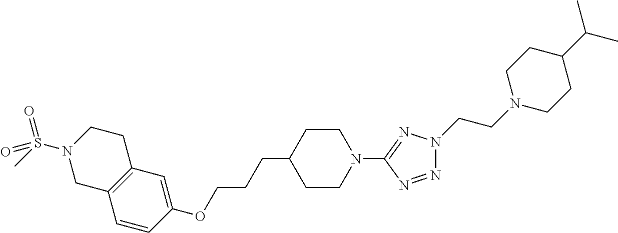 | ¹H-NMR (600 MHz, acetone-d₆) δ = 7.08 (d, J = 5.6 Hz, 1H), 6.79 (dd, J = 2.0, 6.0 Hz, 1H), 6.79 (d, J = 1.6 Hz, 1H), 4.65 (t, J = 4.0 Hz, 2H), 4.34 (s, 2H), 4.20-3.80 (br s, 4H), 4.03 (m, 2H), 3.99 (t, J = 4.4 Hz, 2H), 3.53 (m, 1H), 3.49 (t, J = 1.6 Hz, 2H), 3.16 (br s, 2H), 3.09 (t, J = 4.4 Hz, 2H), 3.06 (br s, 2H), 2.93 (t, J = 4.0 Hz, 2H), 2.88 (m, 2H); 1.82 (m, 4H), 1.55 (m, 1H), 1.45 (m, 2H), 1.37 (d, J = 4.4 Hz, 6H), 1.26 (ddd, J = 4.2, 12.6, 24.6 Hz, 2H); MS calcd. for C₂₈H₄₇N₈O₃S [M + 1]⁺ 575.4; found: 575.3 |
| 246 | 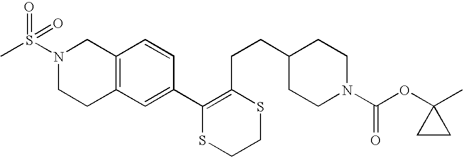 | ¹H-NMR (600 Mhz, CDCl₃) δ = 7.10-7.06 (m, 3H), 4.47 (s, 2H), 4.10- 3.83 (m, 2H), 3.58 (t, J = 6.0 Hz, 2H), 3.28 (s, 4H), 2.98 (t, J = 6.0 Hz, 2H), 2.88 (s, 3H), 2.65-2.57 (m, 2H), 2.16-2.13 (m, 2H), 1.54 (s, 3H), 1.48- 1.43 (m, 2H), 1.42-1.39 (m, 2H), 1.26-1.22 (m, 1H), 1.00-0.88 (m, 2H), 0.86-0.83 (m, 2H), 0.63- 0.60 (m, 2H); MS calcd. for [M + H]⁺ C₂₆H₃₇N₂O₄S₃: 537.2; found: 537.2. |
| 247 | 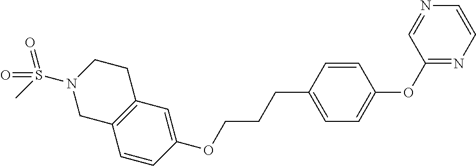 | ¹H-NMR (400 MHz, CDCl₃) δ 8.41 (d, 1H, J = 1.2 Hz), 8.25 (d, 1H, J = 2.8 Hz), 8.12 (dd, 1H, J = 1.2, 2.8 Hz), 7.27 (d, 2H, J = 7.6 Hz), 7.08 (d, 2H, J = 8.4 Hz), 7.00 (d, 1H, J = 8.8 Hz), 6.77 (dd, 1H, J = 2.4, 8.4 Hz), 6.68 (d, 1H, J = 2.4 Hz), 4.40 (s, 2H), 3.97 (t, 2H, J = 6.4 Hz), 3.55 (t, 2H, J = 6.0 Hz), 2.95 (t, 2H, J = 6.0 Hz), 2.85-2.81 (m, 5H), 2.15- 2.08 (m, 2H); MS calcd. for [M + H]⁺ C₂₃H₂₅N₃O₄S: 440.2; found: 439.8. |
| 248 | 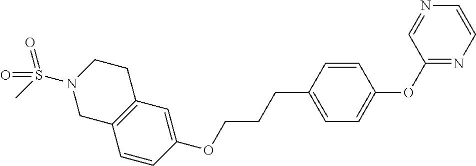 | MS calcd. for [M + H]⁺ C₂₃H₂₅N₃O₄S: 440.2; found: 439.8. |
| 249 | 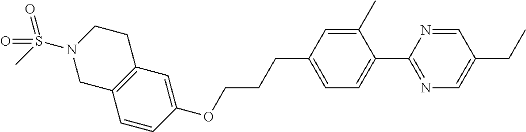 | ¹H-NMR (400 MHz, CDCl₃) δ 8.73 (s, 2H), 7.74 (d, 1H, J = 5.2 Hz), 7.16-7.14 (m, 2H), 6.99 (d, 1H, J = 5.6 Hz), 6.77 (dd, 1H, J = 1.6, 5.6 Hz), 6.66 (d, 1H, J = 1.6 Hz), 4.40 (s, 2H), 3.94 (t, 2H, J = 4.0 Hz), 3.55 (t, 2H, J = 4.0 Hz), 2.94 (t, 2H, J = 3.6 Hz), 2.84-2.81 (m, 5H), 2.73 (q, 2H, J = 5.2 Hz), 2.52 (s, 3H), 2.12 (quint, 2H, J = 4.4 Hz), 1.35 (t, 3H, J = 5.2 Hz); MS calcd. for [M + H]⁺ C₂₆H₃₁N₃O₃S: 466.2; found: 466.2. |
| 250 | 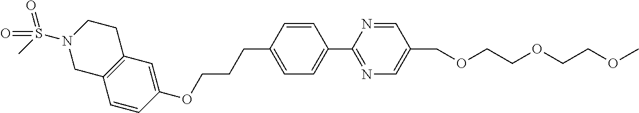 | MS calcd. for [M + H]⁺ C₂₉H₃₈N₃O₆S: 556.2; found: 556.2. |
| 251 | 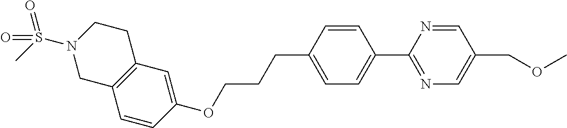 | MS calcd. for [M + H]⁺ C₂₅H₃₀N₃O₄S: 468.2; found: 468.1. |
| 252 | 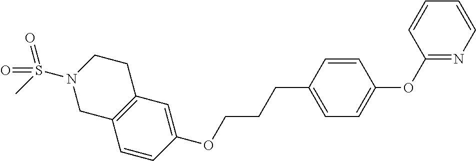 | MS calcd. for [M + H]⁺ C₂₄H₂₆N₂O₄S: 439.2; found: 439.1. |
| 253 | 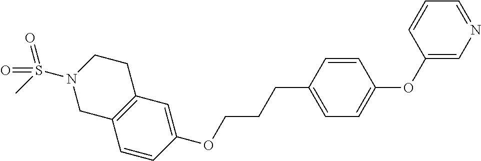 | MS calcd. for [M + H]⁺ C₂₄H₂₆N₂O₄S: 439.2; found: 439.1. |
| 254 | 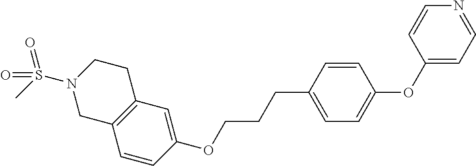 | MS calcd. for [M + H]⁺ C₂₄H₂₆N₂O₄S: 439.2; found: 439.1. |
| 255 | 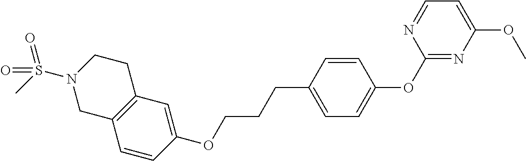 | MS calcd. for [M + H]⁺ C₂₄H₂₇N₃O₅S: 470.2; found: 470.1. |
| 256 | 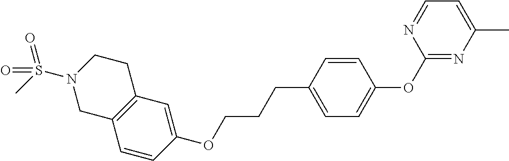 | MS calcd. for [M + H]⁺ C₂₄H₂₇N₃O₄S: 454.2; found: 454.1. |
| 257 | 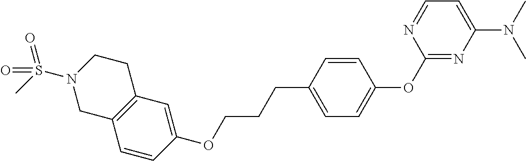 | MS calcd. for [M + H]⁺ C₂₅H₃₀N₄O₄S: 483.2; found: 483.2. |
| 258 | 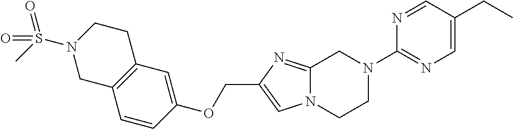 | ¹H-NMR (400 MHz, CDCl₃) δ = 8.30 (s, 2H), 7.10 (s, 1H), 7.03 (d, J = 8.4 Hz, 1H), 6.83 (dd, J = 2.4, 8.4 Hz, 1H), 6.78 (d, J = 2.4 Hz, 1H), 5.36 (s, 2H), 5.19 (s, 2H), 4.42 (s, 2H), 4.38 (m, 2H), 4.21 (m, 2H), 3.5 (m, 2H), 2.97 (m, 2H), 2.86 (s, 3H), 2.55 (q, J = 7.6 Hz, 2H), 1.24 (t, J = 7.6 Hz, 3H); MS calcd. for [M + H]⁺ C₂₃H₂₉N₆O₃S: 469.2; found: 469.2. |
| 259 | 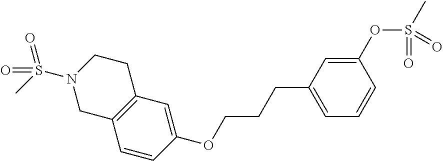 | MS calcd. for [M + H]⁺ C₂₀H₂₅NO₆S₂: 440.1; found: 440.0. |
| 260 | 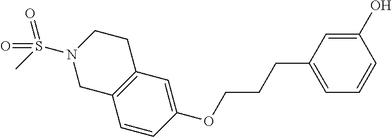 | MS calcd. for [M + H]⁺ C₁₉H₂₃NO₄S: 362.1; found: 362.1. |
| 261 | 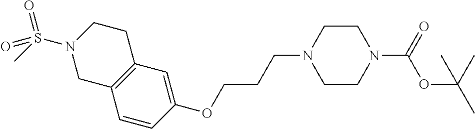 | MS calcd. for [M + H]⁺ C₂₂H₃₅N₃O₅S: 454.2; found. 454.2. |
| 262 | 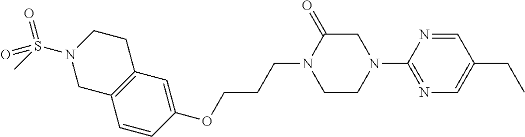 | ¹H-NMR (400 MHz, CDCl₃) δ 8.21 (s, 2H), 6.98 (d, 1H, J = 8.4 Hz), 6.73 (dd, 1H, J = 2.4, 8.4 Hz), 6.66 (d, 1H, J = 2.4 Hz), 4.38 (d, 2H, J = 8.0 Hz), 4.37 (s, 2H), 4.03- 3.97 (m, 4H), 3.63 (t, 2H, J = 6.8 Hz), 3.54 (t, 2H, J = 6.0 Hz), 3.47 (t, 2H, J = 5.6 Hz), 2.93 (t, 2H, J = 5.6 Hz), 2.83 (s, 3H), 2.49 (q, 2H, J = 7.6 Hz), 2.09 (quint, 2H, J = 6.4 Hz), 1.20 (t, 3H, J = 7.6 Hz); MS calcd. for (M + H]⁺ C₂₃H₃₁N₅O₄S: 474.2; found: 474.1. |
| 263 | 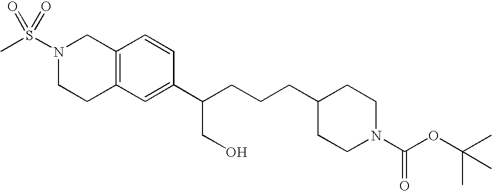 | ¹H-NMR (400 MHz, CD₃CN) δ = 7.06 (m, 3H), 4.47 (m, 1H), 4.36 (s, 2H), 3.95 (d, J = 12.6 Hz, 2H), 3.57 (dd, J = 6.5, 1.8 Hz, 2H), 3.47 (t, J = 5.0 Hz, 2H), 2.93 (t, J = 6.2 Hz, 2H), 2.82 (s, 3H), 2.63 (br s, 2H), 1.60-1.79 (m, 2H), 1.54 (m, 2H), 1.39 (s, 9H), 1.30 (m, 2H), 1.18 (m, 4H), 0.91 (m, 2H); MS calcd. for C₂₅H₄₀N₂O₅S (M + Na⁺) 503.3; found: 503.3. |
| 264 | 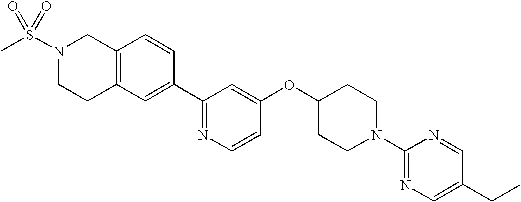 | ¹H-NMR (400 MHz, CD₃CN) δ = 8.62 (d, J = 6.5 Hz, 1H), 8.23 (s, 2H), 7.81 (s, 1H), 7.78 (d, J = 8.0 Hz, 1H), 7.50 (d, J = 2.6 Hz, 1H), 7.34 (d, J = 8.0 Hz, 1H), 7.19 (dd, J = 6.5, 2.6 Hz, 1H), 5.01 (m, 1H), 4.49 (s, 2H), 4.23 (m, 2H), 3.60 (m, 2H), 3.54 (t, J = 5.8 Hz, 2H), 3.05 (t, J = 5.8 Hz, 2H), 2.86 (s, 3H), 2.47 (q, J = 7.7 Hz, 2H), 2.10 (m, 2H), 1.77 (m, 2H), 1.17 (t, J = 7.7 Hz, 3H); MS calcd. for [M + H]⁺ C₂₆H₃₁N₅O₃S: 494.2; found: 494.2. |
| 265 | 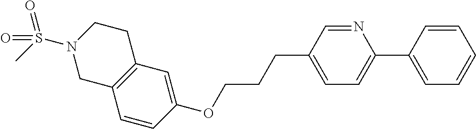 | MS calcd. for [M + H]⁺ C₂₄H₂₇N₂O₃S: 423.2; found: 423.1. |
| 266 | 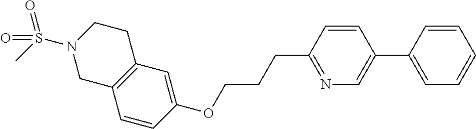 | ¹H-NMR (400 MHz, acetone-d₆) δ = 8.96 (d, J = 2.0 Hz, 1H), 8.29 (dd, J = 2.0, 8.0 Hz, 1H), 7.78- 7.75 (m, 2H), 7.67 (d, J = 8.4 Hz, 1H), 7.55 (m, 2H), 7.47 (m, 1H), 7.06 (d, J = 8.4 Hz, 1H), 6.77 (dd, J = 2.8, 8.4 Hz, 1H), 6.73 (d, J = 2.4 Hz, 1H), 4.34 (s, 2H), 4.09 (t, J = 6.4 Hz, 2H), 3.48 (t, J = 6.0 Hz, 2H), 3.17 (t, J = 7.4 Hz, 2H), 2.91 (t, J = 6.0 Hz, 2H), 2.87 (s, 3H), 2.30 (m, 2H); MS calcd. for [M + H]⁺ C₂₄H₂₇N₂O₃S: 423.2; found: 423.1. |
| 267 | 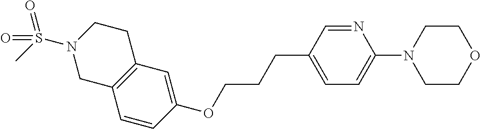 | MS calcd. for [M + H]⁺ C₂₂H₃₀N₃O₄S: 432.2; found: 432.1. |
| 268 | 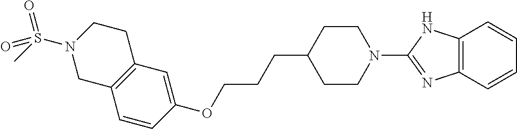 | ¹H-NMR (400 MHz, CDCl₃ + CD₃OD) δ 7.36 (br s, 1H), 7.12 (br s, 1H), 7.03 (br s, 1H), 6.96 (d, 2H, J = 8.4 Hz), 6.72 (dd, 1H, J = 2.8, 8.4 Hz), 6.64 (d, 1H, J = 2.4 Hz), 4.36 (s, 2H), 4.02 (d, 2H, J = 12.4 Hz), 3.90 (t, 2H, J = 6.4 Hz), 3.50 (t, 2H, J = 6.0 Hz), 3.01-2.94 (m, 2H), 2.91 (t, 2H, J = 6.0 Hz), 2.80 (s, 3H), 1.82- 1.75 (m, 4H), 1.57-1.46 (m, 1H), 1.43-1.38 (m, 2H), 1.34-1.24 (m, 2H); MS calcd. for [M + H]⁺ C₂₅H₃₂N₄O₃S: 469.2; found: 469.1. |
| 269 | 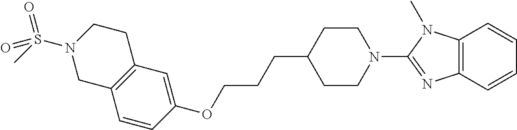 | MS calcd. for [M + H]⁺ C₂₆H₃₄N₄O₃S: 483.2; found: 483.1. |
| 270 | 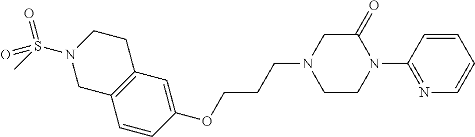 | MS calcd. for [M + H]⁺ C₂₂H₂₈N₄O₄S: 445.2; found: 445.1. |
| 271 | 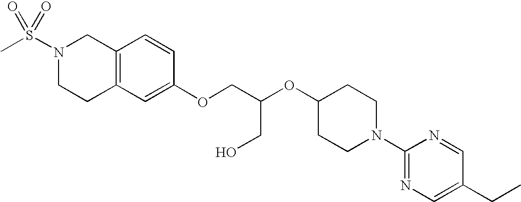 | ¹H-NMR (400 MHz, CD₃CN) = 8.28 (s, 2H), 7.05 (d, J = 8.3 Hz, 1H), 6.80 (m, 2H), 4.32 (s, 2H), 4.13 (m, 2H), 4.04 (m, 2H), 3.97 (m, 2H), 3.85 (m, 2H), 3.65 (m, 2H), 3.49 (m, 2H), 3.45 (t, J = 6.0 Hz, 2H), 2.91 (t, J = 6.0 Hz, 2H), 2.80 (s, 3H), 2.50 (q, J = 7.6 Hz, 2H), 1.57 (m, 2H), 1.17 (t, J = 7.6 Hz, 3H); MS calcd. for [M + H]⁺ C₂₄H₃₄N₄O₅S: 491.2; found: 491.2. |
| 272 | 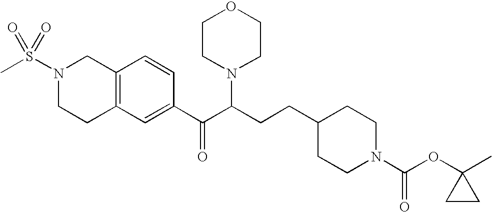 | ¹H-NMR (400 MHz, CD₃CN) δ = 7.75-7.71 (m, 2H), 7.28 (d, J = 8.0 Hz, 1H), 4.94 (t, J = 6.0 Hz, 1H), 4.41 (s, 2H), 3.86-3.67 (m, 6H), 3.44 (t, J = 6.0 Hz, 2H), 3.30 (br. s, 2H), 3.18 (br. s, 2H), 2.98-2.94 (m, 2H), 2.78 (s, 3H), 2.52 (br. s, 2H), 1.98-1.91 (m, 2H), 1.50-1.40 (m, 1H), 1.36 (s, 3H), 1.39-1.31 (m, 1H), 1.27-1.17 (m, 1H), 1.10-0.95 (m 2H), 0.86- 0.70 (m, 2H), 0.68-0.65 (m, 2H), 0.49-0.46 (m, 2H); MS calcd. for [M + H]⁺ C₂₈H₄₂N₃O₆S: 548.3, found: 548.3. |

### Biological Assays

Generation of Stable Cell Line

Flp-In-CHO cells (Invitrogen, Cat.# R758-07) are maintained in Ham's F12 medium supplemented with 10% fetal bovine serum, 1% antibiotic mixture and 2 mM L-glutamine. The cells are transfected with a DNA mixture containing human GPR119 in pcDNA5/FRT vector and the pOG44 vector (1:9) using Fugene6 (Roche), according to the manufacturer's instruction. After 48 h, the medium is changed to medium supplemented with 400 μg/ml hygromycin B to initiate the selection of stably transfected cells.

Cyclic AMP Assay in Stable Cell Line

To test the activity of compounds of the invention, Flp-In-CHO-hGPR119 cells are harvested and resuspended in DMEM plus 3% lipid-depleted fetal bovine serum. Forth μl of cells are plated in 384 well plates at a density of 15,000 cells/well. IBMX (3-isobutyl-1-methyl-xanthine) is added to the cells to a final concentration of 1 mM, followed by the addition of 500 nl of the compound to be tested. The cells are incubated at 37° C. for 30 minutes. Equal volume (20 μl) of the HTRF reagents, anti-cAMP-Cryptate and cAMP-XL665, are added to the cells. The plates are incubated at rt for 1 h and read on a HTRF reader according to the manufacturer's instruction.

Compounds of Formula I, in free form or in pharmaceutically acceptable salt form, produced a concentration-dependent increase in intracellular cAMP level. Compound of the invention show an EC₅₀ of between 1×10⁻⁵ and 1×10⁻¹⁰ M, preferably less than 500 nM, more preferably less than 100 nM.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 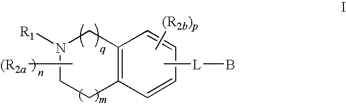
in which:
B is selected from C₆₋₁₀aryl, C₁₋₁₀heteroaryl, C₃₋₁₂cycloalkyl and C₃₋₈heterocycloalkyl; wherein said aryl, heteroaryl, cycloalkyl or heterocycloalkyl of B is substituted with one to three radicals selected from —R₃ and —OXₐR₃; wherein Xₐ is selected from a bond and C₁₋₃alkylene; and wherein any heterocycloalkyl of B can have a CH₂ group replaced with C(O);
n and p are independently selected from 0, 1, 2 and 3;
q is selected from 0, 1 and 2;
m is selected from 1, 2 and 3;
L is —X₁-A—X₂—B₁—X₃—; wherein A and B, are independently selected from a bond, —O—, —S(O)₀₋₂—, —C(O)—, —C(O)O—, —OC(O)—, —NR₄—, —C(O)NR₄—, —C(S)NR₄, —NR₄C(O)—, —CR₄(NR₄C(O)R₄)—, —C(═NOR₄), —CR₄(NR₄R₄)—, —CR₄(OR₄, —CR₄R₄C(O)OR₄—, —N(C(O)R₄)— and —NR₄C(S)—; wherein X₁, X₂ and X₃ are independently selected from a bond, C₁₋₆alkylene, C₂₋₆alkenylene, C₃₋₈cycloalkyl, C₆₋₁₀aryl, C₃₋₈heterocycloalkyl and C₁₋₆heteroarylene; wherein said cycloalkyl, aryl, heterocycloalkyl or heteroaryl of L can be optionally substituted with up to 3 radicals independently selected from hydroxyl, halo, C₁₋₆alkyl, C₁₋₆alkoxy, halo-substituted-C₁₋₆alkyl and halo-substituted-C₁₋₆alkoxy; each R₄ is independently selected from hydrogen, hydroxyl, halo, C₁₋₆alkyl, halo-substituted-C₁₋₆alkyl and halo-substituted-C₁₋₆alkoxy; with the proviso that when A and B are the same moiety, X₂ cannot be a bond; wherein any methylene of L can have the hydrogens replaced by a radical selected from halo, hydroxy, C₁₋₄alkyl, C₁₋₄alkoxy, hydroxy-substituted-C₁₋₄alkyl, —CR₄R₄C(O)OR₄, —X₄OR₄ₐ, —X₄NR₄ₐR₄ₐ, —X₄NR₄ₐX₄OR₄ₐ, —X₄C(O)OR₄ₐ and —X₄C(O)R₄ₐ; wherein X₄ is selected from a bond and C₁₋₄alkylene; R₄ₐ is selected from hydrogen and C₁₋₄alkyl;
R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy, —NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;
R₂ₐ and R_{2b} are independently selected from halo, cyano, hydroxy, C₁₋₄alkyl, amino, nitro, —C(O)OR₅ₑ, —C(O)R₅ₑ and —NR₅ₑR_{5f}; wherein R₅ₑ and R_{5f} are independently selected from hydrogen, C₁₋₆alkyl, C₃₋₁₂cycloalkyl, halo-substituted-C₁₋₆alkyl, halo-substituted-C₁₋₆cycloalkyl, C₆₋₁₀aryl and C₁₋₁₀heteroaryl; wherein said aryl or heteroaryl of R₅ₑ or R_{5f} can be optionally substituted with 1 to 3 radicals independently selected from C₁₋₆alkyl, C₁₋₆alkoxy, halo-substituted-C₁₋₆alkyl and halo-substituted-C₁₋₆alkoxy;
R₃ is selected from hydrogen, C₁₋₁₀heteroaryl, C₆₋₁₀aryl, C₃₋₈heterocycloalkyl, —C(O)OR₆ₐ, —C(O)R₆ₐ, —S(O)₀₋₂R₆ₐ, —C(O)R₇, —C(O)X₅NR₆ₐC(O)OR_{6b}, —C(S)OR₆ₐ, —C(S)R₆ₐ, —C(S)R₇ and —C(S)X₅NR₆ₐC(O)OR_{6b}; wherein X₅ is selected from a bond and C₁₋₆alkylene; R₆ₐ and R_{6b} are independently selected from hydrogen, C₁₋₆alkyl, halo-substituted-C₁₋₆alkyl, C₃₋₁₂cycloalkyl optionally substituted with C₁₋₄alkyl, halo-substituted-C₁₋₆cycloalkyl; R₇ is selected from C₁₋₈alkyl, C₃₋₈cycloalkyl, C₆₋₁₀aryl, C₁₋₁₀heteroaryl, halo-substituted C₁₋₈alkyl, halo-substituted-C₃₋₈cycloalkyl, halo-substituted-C₆₋₁₀aryl and halo-substituted-C₆₋₁₀heteroaryl; wherein said aryl, heteroaryl or heterocycloalkyl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐC(O)R₈ₐ, —X₅ₐR₉, C₁₋₆alkyl, C₁₋₆alkoxy, halo-substituted-C₁₋₆alkyl and halo-substituted-C₁₋₆alkoxy; wherein R₉ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl; wherein said aryl, heteroaryl, cycloalkyl or heterocycloalkyl of R₉ is optionally substituted with 1 to 3 radicals independently selected from halo, C₁₋₄alkyl and C₁₋₄alkoxy; or the pharmaceutically acceptable salts thereof.

2. The compound of claim 1 of Formula Ia: 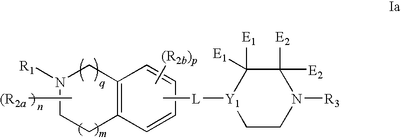
in which:
n and p are independently selected from 0, 1, 2 and 3;
q is selected from 0 and 1;
m is selected from 1, 2 and 3;
E₁ is hydrogen or both E₁ radicals, together with the carbon atom to which they are attached, can form C(═O);
E₂ is hydrogen or both E₂ radicals, together with the carbon atom to which they are attached, can form C(═O);
L is selected from C₁₋₁₀heteroarylene, —X₂OX₃—, —OX₂X₃—, —C(O)X₂—, —X₂X₃—, —OX₂O—, —OX₂C(O)X₃—, —OX₂C(O)OX₃—, —CR₄(NR₄R₄)X₂—, —CR₄(NR₄C(O)R₄)X₂—, —C(═NOR₄)X₂—, —NR₄C(O)X₂—, —C(O)NR₄X₂—, —NR₄X₂—, —N(C(O)R₄)X₂— and —OC(O)NR₄X₂—; wherein X₂ and X₃ are independently selected from a bond, C₁₋₆alkylene, C₂₋₆alkenylene, C₆₋₁₀aryl, C₃₋₈cycloalkyl and C₁₋₁₀heteroarylene; R₄ is selected from hydrogen and C₁₋₆alkyl; wherein any methylene of L can have the hydrogens replaced by a radical selected from halo, hydroxy, C₁₋₄alkyl, C₁₋₄alkoxy, hydroxy-substituted-C₁₋₄alkyl and —CR₄R₄C(O)OR₄;
R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy, —NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;
R₂ₐ and R_{2b} are independently selected from halo, methyl, cyano and nitro;
R₃ is selected from aryl, C₁₋₁₀heteroaryl and —C(O)OR₆ₐ; wherein R₆ₐ is selected from hydrogen, C₁₋₆alkyl and C₃₋₁₂cycloalkyl optionally substituted with C₄alkyl; wherein said heteroaryl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐR₉, C₁₋₆alkyl, C₁₋₆alkoxy and halo-substituted-C₁₋₆alkyl; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl-C₀₋₄alkyl; wherein said aryl, heteroaryl, cycloalkyl or heterocycloalkyl of R₉ is optionally substituted with 1 to 3 radicals independently selected from halo, C₁₋₄alkyl and C₁₋₄alkoxy; and
Y₁ is selected from CH and N.

3. The compound of claim 2 in which L is selected from 3,5-1,2,4-oxadiazolylene, (1,2,4-oxadiazol-5-yl)methoxy, (1,2,4-oxadiazol-5-yl)methyl, (1,2,4-oxadiazol-5-yl)ethyl, (1,2,4-oxadiazol-5-yl)propyl, phenoxy, phenoxy-methyl, —C(O)NHCH₂—, —C(O)NH(CH₂)₂—, —CH₂OCH₂—, —C(O)NH(CH₂)₃—, —CH((CH₂)₂OH)(CH₂)₃—, —CH(CH₂C(O)OCH₃)(CH₂)₃—, —C(O)(CH₂)₃—, —CH(OH)(CH₂)₃—, —CH(Cl)(CH₂)₃—, —C(CH₃)(OH)(CH₂)₃—, —CH(N(CH₃)₂)(CH₂)₃—, —CH(NH₂)(CH₂)₃—, —CH(NHC(O)H)(CH₂)₃—, —CF₂(CH₂)₃—, —O(CH₂)₂—, —(CH₂)₂—, —(CH₂)₃—, —CH₂)₄—, —O(CH₂)₄—, —O(CH₂)₃—, —NH(CH₂)₂—, —NH(CH₂)₃—, —C(═NOCH₃)(CH₂)₃—, —C(═NOH)(CH₂)₃—, —NHC(O)(CH₂)₃—, —NH(CH₂)₄—, —NCH₃(CH₂)₄—, —N(C(O)CH₃)(CH₂)₃—, —NC₂H₅(CH₂)₃—, —NC₃H₇(CH₂)₃—, —O(CH₂)₃O—, —O(CH₂)₂O—, —CH═CH(CH₂)₂—; —CH═CH—; —OCH₂CH(CH₂OH)O—; —C(O)CH(N(CH₂)₂—O—(CH₂)₂)—(CH₂)₂—; —NCH₃(CH₂)₃—; —N(CH(CH₃)₂)(CH₂)₃—; —NHC(O)(CH₂)₂—; —CH₂—O—(CH₂)₂—; —CH₂—O—(CH₂)₃—; —CH₂—O—(CH₂)₄—; —CH═CHCH₂—; —CH(CH₂COOH)(CH₂)₃—; —CH(OCH₃)(CH₂)₃—; —CH(F)(CH₂)₃—; —C(OH)(CH₂OH)(CH₂)₃—; —CH(CH₂OH)(CH₂)₃—;

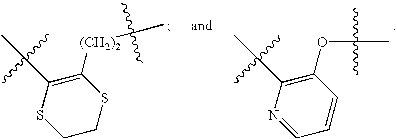

4. The compound of claim 3 in which R₁ is selected from methyl-sulfonyl, butyl-sulfonyl, phenyl-sulfonyl, isopropyl-sulfonyl, ethyl-sulfonyl, ethenyl-sulfonyl, methyl-sulfonyl-ethyl, isopropoxy-carbonyl, benzyloxy-carbonyl, ethoxy-carbonyl, methoxy-carbonyl, t-butoxy-carbonyl and trifluoromethyl-sulfonyl.

5. The compound of claim 4 in which R₃ is selected from t-butoxy-carbonyl, dimethylamino-carbonyl, methyl-sulfonyl, isopropoxy-carbonyl(ethyl)amino-methyl, isopropoxy-carbonyl-amino-methyl, benzyl(ethyl)amino-methyl, piperidinyl, quinazolinyl, isopropoxy-carbonyl, thieno[2,3-d]pyrimidin-4-yl, 4H-1,2,4-triazolyl, cyclopropoxy-carbonyl, (1,2,4-oxadiazol-5-yl), tetrazolyl, thiazolyl, triazolyl, pyrimidinyl, pyrazinyl, pyridinyl, phenyl, benzimidazolyl and pyridazinyl; wherein said cyclopropoxy, quinazolinyl, thieno[2,3-d]pyrimidinyl, thiazolyl, oxadiazolyl, tetrazolyl, pyrimidinyl, pyrazinyl, pyridinyl, phenyl, benzimidazolyl or pyridazinyl can be optionally substituted by 1 to 2 radicals independently selected from halo, cyano, methyl, methoxy-carbonyl, carboxyl, isopropyl, t-butyl, cyclopropyl, morpholino, methyl-piperazinyl-methyl, morpholino-methyl, ethoxy-methoxy-methyl, hydroxy-methyl, methoxy-ethoxy-methyl, methoxy-methoxy-methyl, ethoxy, trifluoromethyl, pentyl, phenyl, methoxy, dimethylamino, dimethylamino-methyl, dimethylamino-ethyl, aminoethyl, methoxy-carbonyl-methyl, methoxy-ethyl, hydroxyl-ethyl, pyrrolidinoethyl, t-butoxycarbonylamino-propoxy-methyl, morpholino-ethyl, aminopropoxy-methyl, dimethylamino-methyl, diethylamino-methyl, isopropyl-piperazino-ethyl, methoxy-ethoxy-ethoxy-methyl, methoxy-methyl, propyl and ethyl.

6. The compound of claim 1 selected from: tert-butyl 4-(2-(2-(methylsulfonyl)-1,2,3,4-tetrahydroisoquinolin-5-yloxy)ethyl)piperidine-1-carboxylate; tert-butyl 4-(3-(2-(methylsulfonyl)-1,2,3,4-tetrahydroisoquinolin-5-yloxy)propyl)piperidine-1-carboxylate; tert-butyl 4-(2-(2-(methylsulfonyl)-1,2,3,4-tetrahydroisoquinolin-6-yloxy)ethyl)piperidine-1-carboxylate; tert-butyl 4-(3-(2-(methylsulfonyl)-1,2,3,4-tetrahydroisoquinolin-6-yloxy)propyl)piperidine-1-carboxylate; tert-butyl 4-(2-(2-(methylsulfonyl)-1,2,3,4-tetrahydroisoquinolin-7-yloxy)ethyl)piperidine-1-carboxylate; tert-butyl 4-(3-(2-(methylsulfonyl)-1,2,3,4-tetrahydroisoquinolin-7-yloxy)propyl)piperidine-1-carboxylate; tert-butyl 4-(3-(1-(methylsulfonyl)-1,2,3,4-tetrahydroquinolin-6-yloxy)propyl)piperidine-1-carboxylate; isopropyl 4-(2-(2-(methylsulfonyl)-1,2,3,4-tetrahydroisoquinolin-6-yloxy)ethyl)piperidine-1-carboxylate; isopropyl 4-(4-(2-(methylsulfonyl)-1,2,3,4-tetrahydroisoquinolin-6-yloxy)butyl)piperidine-1-carboxylate; isopropyl 4-(2-(2-(ethylsulfonyl)-1,2,3,4-tetrahydroisoquinolin-6-yloxy)ethyl)piperidine-1-carboxylate; isopropyl 4-(3-(2-(methylsulfonyl)-1,2,3,4-tetrahydroisoquinolin-6-yloxy)propyl)piperidine-1-carboxylate; isopropyl 4-(5-((2-(methylsulfonyl)-1,2,3,4-tetrahydroisoquinolin-6-yloxy)methyl)-1,2,4-oxadiazol-3-yl)piperidine-1-carboxylate; isopropyl 4-(2-(2-(isopropylsulfonyl)-1,2,3,4-tetrahydroisoquinolin-6-yloxy)ethyl)piperidine-1-carboxylate; isopropyl 4-(2-(2-(vinylsulfonyl)-1,2,3,4-tetrahydroisoquinolin-6-yloxy)ethyl)piperidine-1-carboxylate; isopropyl 6-(2-(1-(isopropoxycarbonyl)piperidin-4-yl)ethoxy)-3,4-dihydroisoquinoline-2(1H)-carboxylate; isopropyl 4-(2-(2-(butylsulfonyl)-1,2,3,4-tetrahydroisoquinolin-6-yloxy)ethyl)piperidine-1-carboxylate; isopropyl 4-(2-(2-(phenylsulfonyl)-1,2,3,4-tetrahydroisoquinolin-6-yloxy)ethyl)piperidine-1-carboxylate; ethyl 6-(2-(1-(isopropoxycarbonyl)piperidin-4-yl)ethoxy)-3,4-dihydroisoquinoline-2(1H)-carboxylate; benzyl 6-(2-(1-(isopropoxycarbonyl)piperidin-4-yl)ethoxy)-3,4-dihydroisoquinoline-2(1H)-carboxylate; isopropyl 4-(4-(2-(methylsulfonyl)-1,2,3,4-tetrahydroisoquinolin-6-yloxy)butyl)piperidine-1-carboxylate; methyl 6-(2-(1-(isopropoxycarbonyl)piperidin-4-yl)ethoxy)-3,4-dihydroisoquinoline-2(1H)-carboxylate; isopropyl 4-(2-(2-(trifluoromethylsulfonyl)-1,2,3,4-tetrahydroisoquinolin-6-yloxy)ethyl)piperidine-1-carboxylate; 6-(3-(1-(5-ethylpyrimidin-2-yl)piperidin-4-yl)propoxy)-2-(methylsulfonyl)-1,2,3,4-tetrahydroisoquinoline; 6-(2-(1-(5-ethylpyrimidin-2-yl)piperidin-4-yl)ethoxy)-2-(methylsulfonyl)-1,2,3,4-tetrahydroisoquinoline; Isopropyl 4-(2-(2-(methylsulfonyl)-1,2,3,4-tetrahydroisoquinolin-6-ylamino)ethyl)-piperidine-1-carboxylate; Isopropyl 4-(3-(2-(methylsulfonyl)-1,2,3,4-tetrahydroisoquinolin-6-ylamino)propyl)-piperidine-1-carboxylate; Isopropyl 4-(3-(2-(methylsulfonyl)-1,2,3,4-tetrahydroisoquinolin-6-ylamino)butyl)-piperidine-1-carboxylate; Tert-butyl 6-(3-(1-(isopropoxycarbonyl)piperidin-4-yl)propylamino)-3,4-dihydroisoquinoline-2(1H)-carboxylate; Tert-butyl 6-(4-(1-(isopropoxycarbonyl)piperidin-4-yl)butylamino)-3,4-dihydroisoquinoline-2(1H)-carboxylate; Isopropyl 4-(3-(methyl(2-(methylsulfonyl)-1,2,3,4-tetrahydroisoquinolin-6-yl)amino)butyl)piperidine-1-carboxylate; isopropyl 4-(3-(methyl(2-(methylsulfonyl)-1,2,3,4-tetrahydroisoquinolin-6-yl)amino)propyl)piperidine-1-carboxylate; isopropyl 4-(3-(ethyl(2-(methylsulfonyl)-1,2,3,4-tetrahydroisoquinolin-6-yl)amino)propyl)piperidine-1-carboxylate; isopropyl 4-(3-((2-(methylsulfonyl)-1,2,3,4-tetrahydroisoquinolin-6-yl)(propyl)amino)propyl)piperidine-1-carboxylate; isopropyl 4-(3-(isopropyl(2-(methylsulfonyl)-1,2,3,4-tetrahydroisoquinolin-6-yl)amino)propyl)piperidine-1-carboxylate; isopropyl 4-(3-(N-(2-(methylsulfonyl)-1,2,3,4-tetrahydroisoquinolin-6-yl)acetamido)propyl)piperidine-1-carboxylate; isopropyl 4-(4-(2-(methylsulfonyl)-1,2,3,4-tetrahydroisoquinolin-6-ylamino)-4-oxobutyl)piperidine-1-carboxylate; tert-butyl 4-(3-(2-(methylsulfonyl)-1,2,3,4-tetrahydroisoquinolin-6-ylamino)-3-oxopropyl)piperidine-1-carboxylate; tert-butyl 4-(4-(2-(methylsulfonyl)-1,2,3,4-tetrahydroisoquinolin-6-ylamino)-4-oxobutyl)piperidine-1-carboxylate; Tert-butyl 4-((2-(methylsulfonyl)-1,2,3,4-tetrahydroisoquinoline-6-carboxamido)methyl)piperidine-1-carboxylate; Isopropyl 4-(2-(2-(methylsulfonyl)-1,2,3,4-tetrahydroisoquinoline-6-carboxamido)ethyl)piperidine-1-carboxylate; Isopropyl 4-(2-(2-(methylsulfonyl)-1,2,3,4-tetrahydroisoquinoline-6-carboxamido)propyl)piperidine-1-carboxylate; Isopropyl 4-(((2-(methylsulfonyl)-1,2,3,4-tetrahydroisoquinolin-6-yl)methoxy)methyl)piperidine-1-carboxylate; isopropyl 4-(2-((2-(methylsulfonyl)-1,2,3,4-tetrahydroisoquinolin-6-yl)methoxy)ethyl)piperidine-1-carboxylate; isopropyl 4-(3-((2-(methylsulfonyl)-1,2,3,4-tetrahydroisoquinolin-6-yl)methoxy)propyl)piperidine-1-carboxylate; isopropyl 4-(4-((2-(methylsulfonyl)-1,2,3,4-tetrahydroisoquinolin-6-yl)methoxy)butyl)piperidine-1-carboxylate; isopropyl 4-(5-(2-(methylsulfonyl)-1,2,3,4-tetrahydroisoquinolin-6-yl)-1,2,4-oxadiazol-3-yl)piperidine-1-carboxylate; Isopropyl 4-((5-(2-(methylsulfonyl)-1,2,3,4-tetrahydroisoquinolin-6-yl)-1,2,4-oxadiazol-3-yl)methyl)piperidine-1-carboxylate; Isopropyl 4-(2-(5-(2-(methylsulfonyl)-1,2,3,4-tetrahydroisoquinolin-6-yl)-1,2,4-oxadiazol-3-yl)ethyl)piperidine-1-carboxylate; Tert-butyl 4-((5-(2-(methylsulfonyl)-1,2,3,4-tetrahydroisoquinolin-6-yl)-1,2,4-oxadiazol-3-yl)methyl)piperidine-1-carboxylate; 3-((1-(5-ethylpyrimidin-2-yl)piperidin-4-yl)methyl)-5-(2-(methylsulfonyl)-1,2,3,4-tetrahydroisoquinolin-6-yl)-1,2,4-oxadiazole; 5-(2-(methylsulfonyl)-1,2,3,4-tetrahydroisoquinolin-6-yl)-3-((1-(pyrimidin-2-yl)piperidin-4-yl)methyl)-1,2,4-oxadiazole; 5-(2-(methylsulfonyl)-1,2,3,4-tetrahydroisoquinolin-6-yl)-3-((1-(pyridin-2-yl)piperidin-4-yl)methyl)-1,2,4-oxadiazole; 3-((1-(6-ethylpyridazin-3-yl)piperidin-4-yl)methyl)-5-(2-(methylsulfonyl)-1,2,3,4-tetrahydroisoquinolin-6-yl)-1,2,4-oxadiazole; 3-((1-(3-chloro-5-(trifluoromethyl)pyridin-2-yl)piperidin-4-yl)methyl)-5-(2-(methylsulfonyl)-1,2,3,4-tetrahydroisoquinolin-6-yl)-1,2,4-oxadiazole; 3-((1-(6-bromopyridin-3-yl)piperidin-4-yl)methyl)-5-(2-(methylsulfonyl)-1,2,3,4-tetrahydroisoquinolin-6-yl)-1,2,4-oxadiazole; 3-((1-(5-fluoropyridin-2-yl)piperidin-4-yl)methyl)-5-(2-(methyl sulfonyl)-1,2,3,4-tetrahydroisoquinolin-6-yl)-1,2,4-oxadiazole; 5-(2-(methylsulfonyl)-1,2,3,4-tetrahydroisoquinolin-6-yl)-3-((1-(5-(trifluoromethyl)pyridin-2-yl)piperidin-4-yl)methyl)-1,2,4-oxadiazole; 2-(methylsulfonyl)-6-(3-((1-(5-(trifluoromethyl)pyridin-2-yl)piperidin-4-yl)methyl)-1,2,4-oxadiazol-5-yl)-1,2,3,4-tetrahydroisoquinolin-1-ol; 1-methylcyclopropyl 4-((5-(2-(methylsulfonyl)-1,2,3,4-tetrahydroisoquinolin-6-yl)-1,2,4-oxadiazol-3-yl)methyl)piperidine-1-carboxylate; Tert-butyl 4-((3-(2-(methylsulfonyl)-1,2,3,4-tetrahydroisoquinolin-6-yl)-1,2,4-oxadiazol-5-yl)methyl)piperidine-1-carboxylate; tert-butyl 4-(2-(3-(2-(methylsulfonyl)-1,2,3,4-tetrahydroisoquinolin-6-yl)-1,2,4-oxadiazol-5-yl)ethyl)piperidine-1-carboxylate; tert-butyl 4-(3-(3-(2-(methylsulfonyl)-1,2,3,4-tetrahydroisoquinolin-6-yl)-1,2,4-oxadiazol-5-yl)propyl)piperidine-1-carboxylate; isopropyl 4-((3-(2-(methylsulfonyl)-1,2,3,4-tetrahydroisoquinolin-6-yl)-1,2,4-oxadiazol-5-yl)methyl)piperidine-1-carboxylate; Isopropyl 4-((3-(2-(methylsulfonyl)-1,2,3,4-tetrahydroisoquinolin-6-yl)-1,2,4-oxadiazol-5-yl)methyl)piperidine-1-carboxylate; 5-((1-(5-ethylpyrimidin-2-yl)piperidin-4-yl)methyl)-3-(2-(methylsulfonyl)-1,2,3,4-tetrahydroisoquinolin-6-yl)-1,2,4-oxadiazole; (E)-isopropyl 4-(4-(2-(methylsulfonyl)-1,2,3,4-tetrahydroisoquinolin-6-yl)but-3-enyl)piperidine-1-carboxylate; (E)-isopropyl 4-(3-(2-(methyl sulfonyl)-1,2,3,4-tetrahydroisoquinolin-6-yl)allyl)piperidine-1-carboxylate; (E)-isopropyl 4-(2-(2-(methylsulfonyl)-1,2,3,4-tetrahydroisoquinolin-6-yl)vinyl)piperidine-1-carboxylate; Isopropyl 4-(4-(2-(methylsulfonyl)-1,2,3,4-tetrahydroisoquinolin-6-yl)butyl)piperidine-1-carboxylate; isopropyl 4-(3-(2-(methylsulfonyl)-1,2,3,4-tetrahydroisoquinolin-6-yl)propyl)piperidine-1-carboxylate; isopropyl 4-(2-(2-(methylsulfonyl)-1,2,3,4-tetrahydroisoquinolin-6-yl)ethyl)piperidine-1-carboxylate; Isopropyl 4-(3-(2-(methylsulfonyl)-1,2,3,4-tetrahydroisoquinolin-6-yl)phenoxy)piperidine-1-carboxylate; Isopropyl 4-((3-(2-(methylsulfonyl)-1,2,3,4-tetrahydroisoquinolin-6-yl)phenoxy)methyl)piperidine-1-carboxylate; Isopropyl 4-(4-(2-(methylsulfonyl)-1,2,3,4-tetrahydroisoquinolin-6-yl)-4-oxobutyl)piperidine-1-carboxylate; Isopropyl 4-(4,4-difluoro-4-(2-(methylsulfonyl)-1,2,3,4-tetrahydroisoquinolin-6-yl)butyl)piperidine-1-carboxylate; Isopropyl 4-(4-(1-(methylsulfonyl)-2,3,4,5-tetrahydro-1H-benzo[b]azepin-7-yloxy)butyl)piperidine-1-carboxylate; 2-(methylsulfonyl)-6-(3-(1-(5-pentylpyrimidin-2-yl)piperidin-4-yl)propoxy)-1,2,3,4-tetrahydroisoquinoline; 2-(methylsulfonyl)-6-(3-(1-(5-propylpyrimidin-2-yl)piperidin-4-yl)propoxy)-1,2,3,4-tetrahydroisoquinoline; 2-(methylsulfonyl)-6-(3-(1-(5-phenylpyrimidin-2-yl)piperidin-4-yl)propoxy)-1,2,3,4-tetrahydroisoquinoline; 6-(3-(1-(5-bromopyrimidin-2-yl)piperidin-4-yl)propoxy)-2-(methylsulfonyl)-1,2,3,4-tetrahydroisoquinoline; 6-(3-(1-(5-fluoropyrimidin-2-yl)piperidin-4-yl)propoxy)-2-(methylsulfonyl)-1,2,3,4-tetrahydroisoquinoline; 2-(methylsulfonyl)-6-(3-(1-(4-(trifluoromethyl)pyrimidin-2-yl)piperidin-4-yl)propoxy)-1,2,3,4-tetrahydroisoquinoline; 6-(3-(1-(4-methoxypyrimidin-2-yl)piperidin-4-yl)propoxy)-2-(methylsulfonyl)-1,2,3,4-tetrahydroisoquinoline; N,N-dimethyl-2-(4-(3-(2-(methylsulfonyl)-1,2,3,4-tetrahydroisoquinolin-6-yloxy)propyl)piperidin-1-yl)pyrimidin-4-amine; 2-(methylsulfonyl)-6-(3-(1-(4-phenylpyrimidin-2-yl)piperidin-4-yl)propoxy)-1,2,3,4-tetrahydroisoquinoline; 6-(3-(1-(4-methylpyrimidin-2-yl)piperidin-4-yl)propoxy)-2-(methylsulfonyl)-1,2,3,4-tetrahydroisoquinoline; 2-(methylsulfonyl)-6-(3-(1-(pyrimidin-2-yl)piperidin-4-yl)propoxy)-1,2,3,4-tetrahydroisoquinoline; 2-(methylsulfonothioyl)-6-(3-(1-(pyrazin-2-yl)piperidin-4-yl)propoxy)-1,2,3,4-tetrahydroisoquinoline; 2-(methylsulfonyl)-6-(3-(1-(pyrimidin-4-yl)piperidin-4-yl)propoxy)-1,2,3,4-tetrahydroisoquinoline; 6-(4-(3-(2-(methylsulfonyl)-1,2,3,4-tetrahydroisoquinolin-6-yloxy)propyl)piperidin-1-yl)nicotinonitrile; 6-(3-(1-(5-chloropyridin-2-yl)piperidin-4-yl)propoxy)-2-(methylsulfonyl)-1,2,3,4-tetrahydroisoquinoline; 2-(methylsulfonyl)-6-(3-(1-(5-(trifluoromethyl)pyridin-2-yl)piperidin-4-yl)propoxy)-1,2,3,4-tetrahydroisoquinoline; methyl 6-(4-(3-(2-(methylsulfonyl)-1,2,3,4-tetrahydroisoquinolin-6-yloxy)propyl)piperidin-1-yl)nicotinate; 6-(3-(1-(3-chloro-5-(trifluoromethyl)pyridin-2-yl)piperidin-4-yl)propoxy)-2-(methylsulfonyl)-1,2,3,4-tetrahydroisoquinoline; 6-(3-(1-(5-methoxypyridin-2-yl)piperidin-4-yl)propoxy)-2-(methylsulfonyl)-1,2,3,4-tetrahydroisoquinoline; 6-(3-(1-(5-bromopyridin-2-yl)piperidin-4-yl)propoxy)-2-(methylsulfonyl)-1,2,3,4-tetrahydroisoquinoline; 6-(3-(1-(6-chloropyridazin-3-yl)piperidin-4-yl)propoxy)-2-(methylsulfonyl)-1,2,3,4-tetrahydroisoquinoline; 6-(3-(1-(6-methylpyridazin-3-yl)piperidin-4-yl)propoxy)-2-(methylsulfonyl)-1,2,3,4-tetrahydroisoquinoline; 2-(methylsulfonyl)-6-(3-(1-(6-phenylpyridazin-3-yl)piperidin-4-yl)propoxy)-1,2,3,4-tetrahydroisoquinoline; 6-(4-(3-(2-(methylsulfonyl)-1,2,3,4-tetrahydroisoquinolin-6-yloxy)propyl)piperidin-1-yl)nicotinic acid; <<101>>6-(3-(1-(6-ethylpyridazin-3-yl)piperidin-4-yl)propoxy)-2-(methylsulfonyl)-1,2,3,4-tetrahydroisoquinoline; 2-(methylsulfonyl)-6-(3-(1-(6-propylpyridazin-3-yl)piperidin-4-yl)propoxy)-1,2,3,4-tetrahydroisoquinoline; 6-(3-(1-(6-isopropylpyridazin-3-yl)piperidin-4-yl)propoxy)-2-(methylsulfonyl)-1,2,3,4-tetrahydroisoquinoline; 6-(3-(1-(6-tert-butylpyridazin-3-yl)piperidin-4-yl)propoxy)-2-(methylsulfonyl)-1,2,3,4-tetrahydroisoquinoline; 6-(3-(1-(6-cyclopropylpyridazin-3-yl)piperidin-4-yl)propoxy)-2-(methylsulfonyl)-1,2,3,4-tetrahydroisoquinoline; 6-(3-(1-(6-methoxypyridazin-3-yl)piperidin-4-yl)propoxy)-2-(methylsulfonyl)-1,2,3,4-tetrahydroisoquinoline; 4-(2-(4-(3-(2-(methylsulfonyl)-1,2,3,4-tetrahydroisoquinolin-6-yloxy)propyl)piperidin-1-yl)pyrimidin-5-yl)morpholino; 2-(methylsulfonyl)-6-(3-(1-(pyrimidin-5-yl)piperidin-4-yl)propoxy)-1,2,3,4-tetrahydroisoquinoline; 4-(5-(4-(3-(2-(methylsulfonyl)-1,2,3,4-tetrahydroisoquinolin-6-yloxy)propyl)piperidin-1-yl)pyrimidin-2-yl)morpholino; 6-(3-(1-(2-methoxypyrimidin-5-yl)piperidin-4-yl)propoxy)-2-(methylsulfonyl)-1,2,3,4-tetrahydroisoquinoline; 2-(methylsulfonyl)-6-(3-(1-(pyridin-2-yl)piperidin-4-yl)propoxy)-1,2,3,4-tetrahydroisoquinoline; 6-(3-(1-(5-((4-methylpiperazin-1-yl)methyl)pyridin-2-yl)piperidin-4-yl)propoxy)-2-(methylsulfonyl)-1,2,3,4-tetrahydroisoquinoline; 4-((6-(4-(3-(2-(methylsulfonyl)-1,2,3,4-tetrahydroisoquinolin-6-yloxy)propyl)piperidin-1-yl)pyridin-3-yl)methyl)morpholino; 6-(3-(1-(5-methylpyridin-2-yl)piperidin-4-yl)propoxy)-2-(methylsulfonyl)-1,2,3,4-tetrahydroisoquinoline; 6-(3-(1-(5-fluoropyridin-2-yl)piperidin-4-yl)propoxy)-2-(methylsulfonyl)-1,2,3,4-tetrahydroisoquinoline; 2-(methylsulfonyl)-6-(3-(1-(pyridin-3-yl)piperidin-4-yl)propoxy)-1,2,3,4-tetrahydroisoquinoline; 6-(3-(1-(6-methylpyridin-3-yl)piperidin-4-yl)propoxy)-2-(methylsulfonyl)-1,2,3,4-tetrahydroisoquinoline; 6-(3-(1-(6-ethoxypyridin-3-yl)piperidin-4-yl)propoxy)-2-(methylsulfonyl)-1,2,3,4-tetrahydroisoquinoline; 6-(3-(1-(6-methoxypyridin-3-yl)piperidin-4-yl)propoxy)-2-(methylsulfonyl)-1,2,3,4-tetrahydroisoquinoline; 2-(methylsulfonyl)-6-(3-(1-(pyridin-4-yl)piperidin-4-yl)propoxy)-1,2,3,4-tetrahydroisoquinoline; 3-isopropyl-5-(4-(3-(2-(methylsulfonyl)-1,2,3,4-tetrahydroisoquinolin-6-yloxy)propyl)piperidin-1-yl)-1,2,4-oxadiazole; 3-isopropyl-5-(4-(2-(2-(methylsulfonyl)-1,2,3,4-tetrahydroisoquinolin-6-yloxy)ethyl)piperidin-1-yl)-1,2,4-oxadiazole; 6-(3-(1-(1H-tetrazol-5-yl)piperidin-4-yl)propoxy)-2-(methylsulfonyl)-1,2,3,4-tetrahydroisoquinoline; 6-(3-(1-(2-methyl-2H-tetrazol-5-yl)piperidin-4-yl)propoxy)-2-(methylsulfonyl)-1,2,3,4-tetrahydroisoquinoline; 6-(3-(1-(4-methyl-4H-1,2,4-triazol-3-yl)piperidin-4-yl)propoxy)-2-(methylsulfonyl)-1,2,3,4-tetrahydroisoquinoline; 6-(3-(1-(5-(1H-tetrazol-5-yl)pyridin-2-yl)piperidin-4-yl)propoxy)-2-(methylsulfonyl)-1,2,3,4-tetrahydroisoquinoline; 6-(3-(1-(5-(2-methyl-2H-tetrazol-5-yl)pyridin-2-yl)piperidin-4-yl)propoxy)-2-(methylsulfonyl)-1,2,3,4-tetrahydroisoquinoline; 6-(3-(1-(5-(1-methyl-1H-tetrazol-5-yl)pyridin-2-yl)piperidin-4-yl)propoxy)-2-(methylsulfonyl)-1,2,3,4-tetrahydroisoquinoline; Isopropyl 4-(4-hydroxy-4-(2-(methylsulfonyl)-1,2,3,4-tetrahydroisoquinolin-6-yl)butyl)piperidine-1-carboxylate; Isopropyl 4-(4-hydroxy-4-(2-(methylsulfonyl)-1,2,3,4-tetrahydroisoquinolin-6-yl)pentyl)piperidine-1-carboxylate; Isopropyl 4-(4-(dimethylamino)-4-(2-(methylsulfonyl)-1,2,3,4-tetrahydroisoquinolin-6-yl)butyl)piperidine-1-carboxylate; Isopropyl 4-(4-formamido-4-(2-(methylsulfonyl)-1,2,3,4-tetrahydroisoquinolin-6-yl)butyl)piperidine-1-carboxylate; Isopropyl 4-(4-amino-4-(2-(methylsulfonyl)-1,2,3,4-tetrahydroisoquinolin-6-yl)butyl)piperidine-1-carboxylate; Isopropyl 4-(6-methoxy-4-(2-(methylsulfonyl)-1,2,3,4-tetrahydroisoquinolin-6-yl)-6-oxohexyl)piperidine-1-carboxylate; Isopropyl 4-(6-hydroxy-4-(2-(methylsulfonyl)-1,2,3,4-tetrahydroisoquinolin-6-yl)hexyl)piperidine-1-carboxylate; 6-(1-(isopropoxycarbonyl)piperidin-4-yl)-3-(2-(methylsulfonyl)-1,2,3,4-tetrahydroisoquinolin-6-yl)hexanoic acid; Isopropyl 4-(4-methoxy-4-(2-(methylsulfonyl)-1,2,3,4-tetrahydroisoquinolin-6-yl)butyl)piperidine-1-carboxylate; Isopropyl 4-(4-fluoro-4-(2-(methylsulfonyl)-1,2,3,4-tetrahydroisoquinolin-6-yl)butyl)piperidine-1-carboxylate; Tert-Butyl 4-(4-(2-(methylsulfonyl)-1,2,3,4-tetrahydroisoquinolin-6-yl)-4-oxobutyl)piperidine-1-carboxylate; 4-(1-(5-ethylpyrimidin-2-yl)piperidin-4-yl)-1-(2-(methylsulfonyl)-1,2,3,4-tetrahydroisoquinolin-6-yl)butan-1-one; 1-methylcyclopropyl 4-(4-(2-(methylsulfonyl)-1,2,3,4-tetrahydroisoquinolin-6-yl)-4-oxobutyl)piperidine-1-carboxylate; 4-(1-(5-fluoropyridin-2-yl)piperidin-4-yl)-1-(2-(methylsulfonyl)-1,2,3,4-tetrahydroisoquinolin-6-yl)butan-1-one; 6-(4-(1-(5-ethylpyrimidin-2-yl)piperidin-4-yl)-1,1-difluorobutyl)-2-(methylsulfonyl)-1,2,3,4-tetrahydroisoquinoline; 1-methylcyclopropyl 4-(4,4-difluoro-4-(2-(methylsulfonyl)-1,2,3,4-tetrahydroisoquinolin-6-yl)butyl)piperidine-1-carboxylate; Isopropyl 4-(3-(1,2,3,4-tetrahydro-2-methanesulfonyl-5-oxo-2,6-naphthyridin-6(5H)-yl)propyl)piperidine-1-carboxylate; 6-(3-(1-(5-ethylpyrimidin-2-yl)piperidin-4-yl)propoxy)-4-methyl-2-(methylsulfonyl)-1,2,3,4-tetrahydroisoquinoline; 6-(3-(1-(5-ethylpyrimidin-2-yl)piperidin-4-yl)propoxy)-5,7-difluoro-2-(methylsulfonyl)-1,2,3,4-tetrahydroisoquinoline; 6-(3-(1-(5-ethylpyrimidin-2-yl)piperidin-4-yl)propoxy)-4,4-dimethyl-2-(methylsulfonyl)-1,2,3,4-tetrahydroisoquinoline; 1-methylcyclopropyl 4-(3-(4,4-dimethyl-2-(methylsulfonyl)-1,2,3,4-tetrahydroisoquinolin-6-yloxy)propyl)piperidine-1-carboxylate; 6-(3-(1-(5-ethylpyrimidin-2-yl)piperidin-4-yl)propoxy)-7-fluoro-2-(methylsulfonyl)-1,2,3,4-tetrahydroisoquinoline; 6-(3-(1-(5-((ethoxymethoxy)methyl)pyrimidin-2-yl)piperidin-4-yl)propoxy)-2-(methylsulfonyl)-1,2,3,4-tetrahydroisoquinoline; isopropyl 4-(2-(5,7-difluoro-2-(methylsulfonyl)-1,2,3,4-tetrahydroisoquinolin-6-yloxy)ethyl)piperidine-1-carboxylate; 6-methyl-4-(4-(3-(2-(methylsulfonyl)-1,2,3,4-tetrahydroisoquinolin-6-yloxy)propyl)piperidin-1-yl)thieno[2,3-d]pyrimidine; 6-(3-(1-(4,6-dimethoxypyrimidin-2-yl)piperidin-4-yl)propoxy)-2-(methylsulfonyl)-1,2,3,4-tetrahydroisoquinoline; isopropyl 4-(3-(1-(methylsulfonyl)-1,2,3,4-tetrahydroquinolin-5-yloxy)propyl)piperidine-1-carboxylate; isopropyl 4-(4-(1-(methylsulfonyl)-1,2,3,4-tetrahydroquinolin-5-yloxy)butyl)piperidine-1-carboxylate; 5-(4-(1-(5-ethylpyrimidin-2-yl)piperidin-4-yl)butoxy)-1-(methylsulfonyl)-1,2,3,4-tetrahydroquinoline; isopropyl 4-(4-(2-(methylsulfonyl)-1,2,3,4-tetrahydroisoquinolin-5-yloxy)butyl)piperidine-1-carboxylate; 1-methylcyclopropyl 4-(3-(5,7-difluoro-2-(methylsulfonyl)-1,2,3,4-tetrahydroisoquinolin-6-yloxy)propyl)piperidine-1-carboxylate; 6-(4-(1-(5-ethylpyrimidin-2-yl)piperidin-4-yl)butoxy)-5,7-difluoro-2-(methylsulfonyl)-1,2,3,4-tetrahydroisoquinoline; 1-methylcyclopropyl 4-(3-(2-(methylsulfonyl)-1,2,3,4-tetrahydroisoquinolin-6-yloxy)propyl)piperidine-1-carboxylate; Tert-butyl 4-(4-(hydroxyimino)-4-(2-(methylsulfonyl)-1,2,3,4-tetrahydroisoquinolin-6-yl)butyl)piperidine-1-carboxylate; Tert-butyl 4-(4-(methoxyimino)-4-(2-(methylsulfonyl)-1,2,3,4-tetrahydroisoquinolin-6-yl)butyl)piperidine-1-carboxylate; 1-methylcyclopropyl 4-(4-hydroxy-4-(2-(methylsulfonyl)-1,2,3,4-tetrahydroisoquinolin-6-yl)butyl)piperidine-1-carboxylate; and 1-methylcyclopropyl 4-(4-chloro-4-(2-(methylsulfonyl)-1,2,3,4-tetrahydroisoquinolin-6-yl)butyl)piperidine-1-carboxylate; 1-methylcyclopropyl 4-(3-(2-(methylsulfonyl)-1,2,3,4-tetrahydroisoquinolin-6-yloxy)propyl)piperazine-1-carboxylate; 6-(3-(4-(5-ethylpyrimidin-2-yl)piperazin-1-yl)propoxy)-2-(methylsulfonyl)-1,2,3,4-tetrahydroisoquinoline; tert-butyl 4-(4,5-dihydroxy-4-(2-(methylsulfonyl)-1,2,3,4-tetrahydroisoquinolin-6-yl)pentyl)piperidine-1-carboxylate; N,N-dimethyl-2-(5-(4-(3-(2-(methylsulfonyl)-1,2,3,4-tetrahydroisoquinolin-6-yloxy)propyl)piperidin-1-yl)-2H-tetrazol-2-yl)ethanamine; 2-(5-(4-(3-(2-(methylsulfonyl)-1,2,3,4-tetrahydroisoquinolin-6-yloxy)propyl)piperidin-1-yl)-2H-tetrazol-2-yl)ethanamine; methyl 2-(5-(4-(3-(2-(methylsulfonyl)-1,2,3,4-tetrahydroisoquinolin-6-yloxy)propyl)piperidin-1-yl)-2H-tetrazol-2-yl)acetate; 6-(3-(1-(2-(2-methoxyethyl)-2H-tetrazol-5-yl)piperidin-4-yl)propoxy)-2-(methylsulfonyl)-1,2,3,4-tetrahydroisoquinoline; 2-(5-(4-(3-(2-(methylsulfonyl)-1,2,3,4-tetrahydroisoquinolin-6-yloxy)propyl)piperidin-1-yl)-2H-tetrazol-2-yl)ethanol; 6-(3-(1-(5-ethylpyrimidin-2-yl)piperidin-4-yl)propoxy)-2-(2-(methylsulfonyl)ethyl)-1,2,3,4-tetrahydroisoquinoline; 1-methylcyclopropyl 4-(3-(2-(2-(methylsulfonyl)ethyl)-1,2,3,4-tetrahydroisoquinolin-6-yloxy)propyl)piperidine-1-carboxylate; 2-(methylsulfonyl)-6-(3-(1-(2-(2-(pyrrolidin-1-yl)ethyl)-2H-tetrazol-5-yl)piperidin-4-yl)propoxy)-1,2,3,4-tetrahydroisoquinoline; tert-butyl 3-(4-(4-(3-(2-(methylsulfonyl)-1,2,3,4-tetrahydroisoquinolin-6-yloxy)propyl)piperidin-1-yl)benzyloxy)propylcarbamate; 4-(2-(5-(4-(3-(2-(methylsulfonyl)-1,2,3,4-tetrahydroisoquinolin-6-yloxy)propyl)piperidin-1-yl)-2H-tetrazol-2-yl)ethyl)morpholine; 3-(4-(4-(3-(2-(methylsulfonyl)-1,2,3,4-tetrahydroisoquinolin-6-yloxy)propyl)piperidin-1-yl)benzyloxy)propan-1-amine; N,N-dimethyl-3-(5-(4-(3-(2-(methylsulfonyl)-1,2,3,4-tetrahydroisoquinolin-6-yloxy)propyl)piperidin-1-yl)-2H-tetrazol-2-yl)propan-1-amine; N,N-diethyl-2-(5-(4-(3-(2-(methylsulfonyl)-1,2,3,4-tetrahydroisoquinolin-6-yloxy)propyl)piperidin-1-yl)-2H-tetrazol-2-yl)ethanamine; 2-(methylsulfonyl)-6-(3-(1-(2-(2-(piperidin-1-yl)ethyl)-2H-tetrazol-5-yl)piperidin-4-yl)propoxy)-1,2,3,4-tetrahydroisoquinoline; 6-(3-(1-(2-(2-(4-isopropylpiperazin-1-yl)ethyl)-2H-tetrazol-5-yl)piperidin-4-yl)propoxy)-2-(methylsulfonyl)-1,2,3,4-tetrahydroisoquinoline; 1-methylcyclopropyl 4-(2-(3-(2-(methylsulfonyl)-1,2,3,4-tetrahydroisoquinolin-6-yl)-5,6-dihydro-1,4-dithiin-2-yl)ethyl)piperidine-1-carboxylate; tert-butyl 4-(3-(2-(methylsulfonyl)-1,2,3,4-tetrahydroisoquinolin-6-yloxy)propyl)piperazine-1-carboxylate; 4-(5-ethylpyrimidin-2-yl)-1-(3-(2-(methylsulfonyl)-1,2,3,4-tetrahydroisoquinolin-6-yloxy)propyl)piperazin-2-one; tert-butyl 4-(5-hydroxy-4-(2-(methylsulfonyl)-1,2,3,4-tetrahydroisoquinolin-6-yl)pentyl)piperidine-1-carboxylate; 6-(4-(1-(5-ethylpyrimidin-2-yl)piperidin-4-yloxy)pyridin-2-yl)-2-(methylsulfonyl)-1,2,3,4-tetrahydroisoquinoline; 6-(3-(1-(1H-benzo[d]imidazol-2-yl)piperidin-4-yl)propoxy)-2-(methylsulfonyl)-1,2,3,4-tetrahydroisoquinoline; 6-(3-(1-(1-methyl-1H-benzo[d]imidazol-2-yl)piperidin-4-yl)propoxy)-2-(methylsulfonyl)-1,2,3,4-tetrahydroisoquinoline; 4-(3-(2-(methylsulfonyl)-1,2,3,4-tetrahydroisoquinolin-6-yloxy)propyl)-1-(pyridin-2-yl)piperazin-2-one; 2-(1-(5-ethylpyrimidin-2-yl)piperidin-4-yloxy)-3-(2-(methylsulfonyl)-1,2,3,4-tetrahydroisoquinolin-6-yloxy)propan-1-ol; 1-methylcyclopropyl 4-(4-(2-(methylsulfonyl)-1,2,3,4-tetrahydroisoquinolin-6-yl)-3-morpholino-4-oxobutyl)piperidine-1-carboxylate.

7. The compound of claim 1 of Formula Ib: 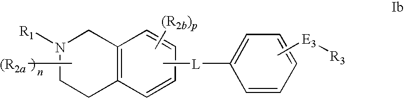
in which:
n and p are independently selected from 0, 1, 2 and 3;
E₃ is selected from a bond, O and OCH₂;
L is selected from C₁₋₁₀heteroarylene, —X₂OX₃—, —OX₂X₃—, —C(O)X₂—, —X₂X₃—, —OX₂O—, —OX₂C(O)X₃—, —OX₂C(O)OX₃—, —CR₄(NR₄)X₂—, —CR₄(NR₄C(O)R₄)X₂—, —C(═NOR₄)X₂—, —NR₄C(O)X₂—, —C(O)NR₄X₂—, —NR₄X₂—, —N(C(O)R₄)X₂— and —OC(O)NR₄X₂—; wherein X₂ and X₃ are independently selected from a bond, C₁₋₆alkylene, C₂₋₆alkenylene, C₆₋₁₀aryl, C₃₋₈cycloalkyl and C₁₋₁₀heteroarylene; R₄ is selected from hydrogen and C₁₋₆alkyl; wherein any methylene of L can have the hydrogens replaced by a radical selected from halo, hydroxy, C₁₋₄alkyl, C₁₋₄alkoxy, hydroxy-substituted-C₁₋₄alkyl and —CR₄R₄C(O)OR₄;
R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy-NR_{5c}R_{5d}, —C(O)OR_{5c} and C₆₋₁₀aryl-C₀₋₄alkyl; wherein R₅, and R_{5d} are independently selected from hydrogen and C₁₋₆alkyl;
R₂ₐ and R_{2b} are independently selected from halo, methyl, cyano and nitro; and
R₃ is selected from hydrogen, SO₂R₆ₐ, C₆₋₁₀aryl, C₁₋₁₀heteroaryl and —C(O)OR₆ₐ and —OC(O)NR₆ₐR_{6b}; wherein R₆ₐ and R_{6b} are independently selected from hydrogen, C₁₋₆alkyl and C₃₋₁₂cycloalkyl optionally substituted with C₁₋₄alkyl; wherein said heteroaryl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐR₉, C₁₋₆alkyl, C₁₋₆alkoxy and halo-substituted-C₁₋₆alkyl; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl-C₀₋₄alkyl; wherein said aryl, heteroaryl, cycloalkyl or heterocycloalkyl of R₉ is optionally substituted with 1 to 3 radicals independently selected from halo, C₁₋₄alkyl and C₁₋₄alkoxy.

8. The compound of claim 7 in which L is selected from 3,5-1,2,4-oxadiazolylene, (1,2,4-oxadiazol-5-yl)methoxy, (1,2,4-oxadiazol-5-yl)methyl, (1,2,4-oxadiazol-5-yl)ethyl, (1,2,4-oxadiazol-5-yl)propyl, phenoxy, phenoxy-methyl, —C(O)NHCH₂—, —C(O)NH(CH₂)₂—, —CH₂OCH₂—, —C(O)NH(CH₂)₃—, —CH((CH₂)₂OH)(CH₂)₃—, —CH(CH₂C(O)OCH₃)(CH₂)₃—, —C(O)(CH₂)₃—, —CH(OH)(CH₂)₃—, —CH(Cl)(CH₂)₃—, —C(CH₃)(OH)(CH₂)₃—, —CH(N(CH₃)₂)(CH₂)₃—, —CH(NH₂)(CH₂)₃—, —CH(NHC(O)H)(CH₂)₃—, —CF₂(CH₂)₃—, —O(CH₂)₂—, —(CH₂)₂—, —(CH₂)₃—, —(CH₂)₄—, —O(CH₂)₄—, —O(CH₂)₃—, —NH(CH₂)₂—, —NH(CH₂)₃—, —C(═NOCH₃)(CH₂)₃—, —C(═NOH)(CH₂)₃—, —NHC(O)(CH₂)₃—, —NH(CH₂)₄—, —NCH₃(CH₂)₄—, —N(C(O)CH₃)(CH₂)₃—, —NC₂H₅(CH₂)₃—, —NC₃H₇(CH₂)₃—, —O(CH₂)₃O—, —O(CH₂)₂O—, —CH═CH(CH₂)₂—; —CH═CH—; —OCH₂CH(CH₂OH)O—; —C(O)CH(N(CH₂)₂—O—(CH₂)₂)—(CH₂)₂—; —NCH₃(CH₂)₃—; —N(CH(CH₃)₂)(CH₂)₃—; —NHC(O)(CH₂)₂—; —CH₂—O—(CH₂)₂—; —CH₂—O—(CH₂)₃—; —CH₂O(CH₂)₄—; —CH═CHCH₂—; —CH(CH₂COOH)(CH₂)₃—; —CH(OCH₃)(CH₂)₃—; —CH(F)(CH₂)₃—; —C(OH)(CH₂OH)(CH₂)₃—; —CH(CH₂OH)(CH₂)₃—;

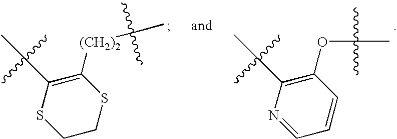

9. The compound of claim 8 in which R₁ is selected from methyl-sulfonyl, butyl-sulfonyl, phenyl-sulfonyl, isopropyl-sulfonyl, ethyl-sulfonyl, ethenyl-sulfonyl, methyl-sulfonyl-ethyl, isopropoxy-carbonyl, benzyloxy-carbonyl, ethoxy-carbonyl, methoxy-carbonyl, t-butoxy-carbonyl and trifluoromethyl-sulfonyl.

10. The compound of claim 9 in which In a further embodiment, R₃ is selected from t-butoxy-carbonyl, dimethylamino-carbonyl, methyl-sulfonyl, isopropoxy-carbonyl(ethyl)amino-methyl, isopropoxy-carbonyl-amino-methyl, benzyl(ethyl)amino-methyl, piperidinyl, quinazolinyl, isopropoxy-carbonyl, thieno[2,3-d]pyrimidin-4-yl, 4H-1,2,4-triazolyl, cyclopropoxy-carbonyl, (1,2,4-oxadiazol-5-yl), tetrazolyl, thiazolyl, triazolyl, pyrimidinyl, pyrazinyl, pyridinyl, phenyl, benzimidazolyl and pyridazinyl; wherein said cyclopropoxy, quinazolinyl, thieno[2,3-d]pyrimidinyl, thiazolyl, oxadiazolyl, tetrazolyl, pyrimidinyl, pyrazinyl, pyridinyl, phenyl, benzimidazolyl or pyridazinyl can be optionally substituted by 1 to 2 radicals independently selected from halo, cyano, methyl, methoxy-carbonyl, carboxyl, isopropyl, t-butyl, cyclopropyl, morpholino, methyl-piperazinyl-methyl, morpholino-methyl, ethoxy-methoxy-methyl, hydroxy-methyl, methoxy-ethoxy-methyl, methoxy-methoxy-methyl, ethoxy, trifluoromethyl, pentyl, phenyl, methoxy, dimethylamino, dimethylamino-methyl, dimethylamino-ethyl, aminoethyl, methoxy-carbonyl-methyl, methoxy-ethyl, hydroxyl-ethyl, pyrrolidinoethyl, t-butoxycarbonylamino-propoxy-methyl, morpholino-ethyl, aminopropoxy-methyl, dimethylamino-methyl, diethylamino-methyl, isopropyl-piperazino-ethyl, methoxy-ethoxy-ethoxy-methyl, methoxy-methyl, propyl and ethyl.

11. The compound of claim 1 selected from: 3-tert-butyl-5-(4-((2-(methylsulfonyl)-1,2,3,4-tetrahydroisoquinolin-6-yloxy)methyl)phenyl)-1,2,4-oxadiazole; 3-(2-(methylsulfonyl)-1,2,3,4-tetrahydroisoquinolin-6-yl)-5-(4-(pyrimidin-2-yl)benzyl)-1,2,4-oxadiazole; 5-(4-bromophenethyl)-3-(2-(methylsulfonyl)-1,2,3,4-tetrahydroisoquinolin-6-yl)-1,2,4-oxadiazole; 5-(4-(5-methylpyridin-2-yl)benzyl)-3-(2-(methylsulfonyl)-1,2,3,4-tetrahydroisoquinolin-6-yl)-1,2,4-oxadiazole; 5-(4-(5-methylpyridin-2-yl)phenethyl)-3-(2-(methylsulfonyl)-1,2,3,4-tetrahydroisoquinolin-6-yl)-1,2,4-oxadiazole; 5-(4-(5-bromopyrimidin-2-yl)phenethyl)-3-(2-(methylsulfonyl)-1,2,3,4-tetrahydroisoquinolin-6-yl)-1,2,4-oxadiazole; 2-(methylsulfonyl)-6-(3-(4-(pyrimidin-2-yl)phenyl)propoxy)-1,2,3,4-tetrahydroisoquinoline; 4-(5-(4-(3-(2-(methylsulfonyl)-1,2,3,4-tetrahydroisoquinolin-6-yloxy)propyl)phenyl)pyrimidin-2-yl)morpholino; 2-(methylsulfonyl)-6-(3-(4-(5-(trifluoromethyl)pyridin-2-yl)phenyl)propoxy)-1,2,3,4-tetrahydroisoquinoline; 2-(methylsulfonyl)-6-(3-(4-(pyrazin-2-yl)phenyl)propoxy)-1,2,3,4-tetrahydroisoquinoline; 6-(3-(4-(5-ethylpyrimidin-2-yl)phenyl)propoxy)-5,7-difluoro-2-(methylsulfonyl)-1,2,3,4-tetrahydroisoquinoline; 5-tert-butyl-3-(4-((2-(methylsulfonyl)-1,2,3,4-tetrahydroisoquinolin-6-yloxy)methyl)phenyl)-1,2,4-oxadiazole; 6-(4-(5-ethylpyrimidin-2-yl)phenethoxy)-2-(methylsulfonyl)-1,2,3,4-tetrahydroisoquinoline; N-benzyl-N-(4-((2-(methylsulfonyl)-1,2,3,4-tetrahydroisoquinolin-6-yloxy)methyl)benzyl)ethanamine; 6-(3-(4-(5-ethylpyrimidin-2-yl)phenyl)propoxy)-2-(methylsulfonyl)-1,2,3,4-tetrahydroisoquinoline; 6-(4-iodophenethoxy)-2-(methylsulfonyl)-1,2,3,4-tetrahydroisoquinoline; 5-tert-butyl-3-(4-(2-(2-(methylsulfonyl)-1,2,3,4-tetrahydroisoquinolin-6-yloxy)ethyl)phenyl)-1,2,4-oxadiazole; isopropyl ethyl(4-(3-(2-(methylsulfonyl)-1,2,3,4-tetrahydroisoquinolin-6-yloxy)propoxy)benzyl)carbamate; isopropyl ethyl(3-(3-(2-(methylsulfonyl)-1,2,3,4-tetrahydroisoquinolin-6-yloxy)propoxy)benzyl)carbamate; isopropyl ethyl(4-(2-(2-(methylsulfonyl)-1,2,3,4-tetrahydroisoquinolin-6-yloxy)ethoxy)benzyl)carbamate; isopropyl ethyl(3-(2-(2-(methylsulfonyl)-1,2,3,4-tetrahydroisoquinolin-6-yloxy)ethoxy)benzyl)carbamate; isopropyl 4-(2-(2-(methylsulfonyl)-1,2,3,4-tetrahydroisoquinolin-6-yloxy)ethoxy)benzylcarbamate; 6-(3-(4-(6-cyclopropylpyridazin-3-yl)phenyl)propoxy)-2-(methylsulfonyl)-1,2,3,4-tetrahydroisoquinoline; 3-(4-bromobenzyl)-5-(2-(methylsulfonyl)-1,2,3,4-tetrahydroisoquinolin-6-yl)-1,2,4-oxadiazole; 5-(2-(methylsulfonyl)-1,2,3,4-tetrahydroisoquinolin-6-yl)-3-(4-(pyrazin-2-yl)phenethyl)-1,2,4-oxadiazole; 3-(2-(4-(5-ethylpyrimidin-2-yl)cyclohexa-1,5-dienyl)ethyl)-5-(2-(methylsulfonyl)-1,2,3,4-tetrahydroisoquinolin-6-yl)-1,2,4-oxadiazole; 5-(2-(methylsulfonyl)-1,2,3,4-tetrahydroisoquinolin-6-yl)-3-(2-(4-(pyrimidin-2-yl)cyclohexa-1,5-dienyl)ethyl)-1,2,4-oxadiazole; 2-(4-(3-(2-(methylsulfonyl)-1,2,3,4-tetrahydroisoquinolin-6-yloxy)propyl)phenyl)thiazole; 6-(3-(4-(5-((methoxymethoxy)methyl)pyrimidin-2-yl)phenyl)propoxy)-2-(methylsulfonyl)-1,2,3,4-tetrahydroisoquinoline; 6-(3-(4-(5-((2-methoxyethoxy)methyl)pyrimidin-2-yl)phenyl)propoxy)-2-(methylsulfonyl)-1,2,3,4-tetrahydroisoquinoline; (2-(4-(3-(2-(methylsulfonyl)-1,2,3,4-tetrahydroisoquinolin-6-yloxy)propyl)phenyl)pyrimidin-5-yl)methanol; 4-(3-(2-(methylsulfonyl)-1,2,3,4-tetrahydroisoquinolin-6-yloxy)propyl)benzonitrile; 6-(3-(4-(1H-tetrazol-5-yl)phenyl)propoxy)-2-(methylsulfonyl)-1,2,3,4-tetrahydroisoquinoline; N,N-dimethyl-1-(2-(4-(3-(2-(methylsulfonyl)-1,2,3,4-tetrahydroisoquinolin-6-yloxy)propyl)phenyl)pyrimidin-5-yl)methanamine; 6-(3-(4-(5-ethylpyrimidin-2-yl)phenyl)propoxy)-7-fluoro-2-(methylsulfonyl)-1,2,3,4-tetrahydroisoquinoline; and 6-(3-(4-(5-ethylpyrimidin-2-yl)phenyl)propoxy)-4-methyl-2-(methylsulfonyl)-1,2,3,4-tetrahydroisoquinoline; 6-(3-(4-(5-ethylpyrimidin-2-yl)phenyl)propoxy)-1,2,3,4-tetrahydroisoquinoline; 6-(3-(4-(5-ethylpyrimidin-2-yl)phenyl)propoxy)-2-(vinylsulfonyl)-1,2,3,4-tetrahydroisoquinoline; 6-(3-(3-(5-ethylpyrimidin-2-yloxy)phenyl)propoxy)-2-(methylsulfonyl)-1,2,3,4-tetrahydroisoquinoline; 2-(methylsulfonyl)-6-(3-(4-(pyrimidin-2-yloxy)phenyl)propoxy)-1,2,3,4-tetrahydroisoquinoline; 6-(1-(4-(5-ethylpyrimidin-2-yl)phenyl)pyrrolidin-3-yloxy)-2-(methylsulfonyl)-1,2,3,4-tetrahydroisoquinoline; 6-(3-(4-(5-ethylpyrimidin-2-yl)-3-fluorophenyl)propoxy)-5,7-difluoro-2-(methylsulfonyl)-1,2,3,4-tetrahydroisoquinoline; 6-(3-(4-(5-ethylpyrimidin-2-yl)-3-fluorophenyl)propoxy)-4-methyl-2-(methylsulfonyl)-1,2,3,4-tetrahydroisoquinoline; 6-(3-(4-(5-ethylpyrimidin-2-yl)-3-fluorophenyl)propoxy)-7-fluoro-2-(methylsulfonyl)-1,2,3,4-tetrahydroisoquinoline; 6-(3-(4-(5-ethylpyrimidin-2-yl)-3-fluorophenyl)propoxy)-2-(methylsulfonyl)-1,2,3,4-tetrahydroisoquinoline; 6-(3-(4′-butylbiphenyl-4-yl)propoxy)-2-(methylsulfonyl)-1,2,3,4-tetrahydroisoquinoline; 6-(3-(4-(benzyloxy)phenyl)propoxy)-2-(methylsulfonyl)-1,2,3,4-tetrahydroisoquinoline; 4-(3-(2-(methylsulfonyl)-1,2,3,4-tetrahydroisoquinolin-6-yloxy)propyl)phenyl dimethylcarbamate; 6-(3-(4-(5-ethylpyrimidin-2-yloxy)phenyl)propoxy)-2-(methylsulfonyl)-1,2,3,4-tetrahydroisoquinoline; benzyl 6-(3-(4-(5-ethylpyrimidin-2-yl)phenyl)propoxy)-3,4-dihydroisoquinoline-2(1H)-carboxylate; 2-(methylsulfonyl)-6-(3-(4-(pyrazin-2-yloxy)phenyl)propoxy)-1,2,3,4-tetrahydroisoquinoline; 6-(3-(4-(5-ethylpyrimidin-2-yl)-3-methylphenyl)propoxy)-2-(methylsulfonyl)-1,2,3,4-tetrahydroisoquinoline; 6-(3-(4-(5-((2-(2-methoxyethoxy)ethoxy)methyl)pyrimidin-2-yl)phenyl)propoxy)-2-(methylsulfonyl)-1,2,3,4-tetrahydroisoquinoline; 6-(3-(4-(5-(methoxymethyl)pyrimidin-2-yl)phenyl)propoxy)-2-(methylsulfonyl)-1,2,3,4-tetrahydroisoquinoline; 2-(methylsulfonyl)-6-(3-(4-(pyridin-2-yloxy)phenyl)propoxy)-1,2,3,4-tetrahydroisoquinoline; 2-(methylsulfonyl)-6-(3-(4-(pyridin-3-yloxy)phenyl)propoxy)-1,2,3,4-tetrahydroisoquinoline; 2-(methylsulfonyl)-6-(3-(4-(pyridin-4-yloxy)phenyl)propoxy)-1,2,3,4-tetrahydroisoquinoline; 6-(3-(4-(4-methoxypyrimidin-2-yloxy)phenyl)propoxy)-2-(methylsulfonyl)-1,2,3,4-tetrahydroisoquinoline; 6-(3-(4-(4-methylpyrimidin-2-yloxy)phenyl)propoxy)-2-(methylsulfonyl)-1,2,3,4-tetrahydroisoquinoline; N,N-dimethyl-2-(4-(3-(2-(methylsulfonyl)-1,2,3,4-tetrahydroisoquinolin-6-yloxy)propyl)phenoxy)pyrimidin-4-amine; 3-(3-(2-(methylsulfonyl)-1,2,3,4-tetrahydroisoquinolin-6-yloxy)propyl)phenyl methanesulfonate; 3-(3-(2-(methylsulfonyl)-1,2,3,4-tetrahydroisoquinolin-6-yloxy)propyl)phenol; 2-(Methylsulfonyl)-6-(3-(3-(pyrimidin-2-yloxy)phenyl)propoxy)-1,2,3,4-tetrahydroisoquinoline; 2-(Methylsulfonyl)-6-(3-(4-(pyrimidin-2-yloxy)phenyl)propoxy)-1,2,3,4-tetrahydroisoquinoline; 6-(3-(4-(Benzyloxy)phenyl)propoxy)-2-(methylsulfonyl)-1,2,3,4-tetrahydroisoquinoline; 4-(3-(2-(Methylsulfonyl)-1,2,3,4-tetrahydroisoquinolin-6-yloxy)propyl)phenyl dimethylcarbamate; 2-(Methylsulfonyl)-6-(3-(4-(pyrazin-2-yloxy)phenyl)propoxy)-1,2,3,4-tetrahydroisoquinoline; 3-(3-(2-(Methylsulfonyl)-1,2,3,4-tetrahydroisoquinolin-6-yloxy)propyl)phenyl methanesulfonate; 4-(3-(2-(Methylsulfonyl)-1,2,3,4-tetrahydroisoquinolin-6-yloxy)propyl)phenol; 6-(3-(4-(5-ethylpyrimidin-2-yloxy)phenyl)propoxy)-2-(methylsulfonyl)-1,2,3,4-tetrahydroisoquinoline; 2-(methylsulfonyl)-6-(3-(4-(pyrimidin-5-yloxy)phenyl)propoxy)-1,2,3,4-tetrahydroisoquinoline; 2-(methylsulfonyl)-6-(3-(4-(pyridin-2-yloxy)phenyl)propoxy)-1,2,3,4-tetrahydroisoquinoline; 2-(methylsulfonyl)-6-(3-(4-(pyridin-3-yloxy)phenyl)propoxy)-1,2,3,4-tetrahydroisoquinoline; 2-(methylsulfonyl)-6-(3-(4-(pyridin-4-yloxy)phenyl)propoxy)-1,2,3,4-tetrahydroisoquinoline; 6-(3-(4-(4-methoxypyrimidin-2-yloxy)phenyl)propoxy)-2-(methylsulfonyl)-1,2,3,4-tetrahydroisoquinoline; 6-(3-(4-(4-methylpyrimidin-2-yloxy)phenyl)propoxy)-2-(methylsulfonyl)-1,2,3,4-tetrahydroisoquinoline; and N,N-dimethyl-2-(4-(3-(2-(methylsulfonyl)-1,2,3,4-tetrahydroisoquinolin-6-yloxy)propyl)phenoxy)pyrimidin-4-amine.

12. The compound of claim 1 of Formula Ic: 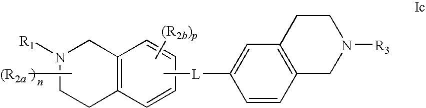
in which:
n and p are independently selected from 0, 1, 2 and 3;
L is selected from C₁₋₁₀heteroarylene, —X₂OX₃—, —OX₂X₃—, —C(O)X₂—, —X₂X₃—, —OX₂O—, —OX₂C(O)X₃—, —OX₂C(O)OX₃—, —CR₄(NR₄R₄)X₂—, —CR₄(NR₄C(O)R₄)X₂—, —C(═NOR₄)X₂—, —NR₄C(O)X₂—, —C(O)NR₄X₂—, —NR₄X₂—, —N(C(O)R₄)X₂— and —OC(O)NR₄X₂—; wherein X₂ and X₃ are independently selected from a bond, C₁₋₆alkylene, C₂₋₆alkenylene, C₆₋₁₀aryl, C₃₋₈cycloalkyl and C₁₋₁₀heteroarylene; R₄ is selected from hydrogen and C₁₋₆alkyl; wherein any methylene of L can have the hydrogens replaced by a radical selected from halo, hydroxy, C₁₋₄alkyl, C₁₋₄alkoxy, hydroxy-substituted-C₁₋₄alkyl and —CR₄R₄C(O)OR₄;
R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy-NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;
R₂ₐ and R_{2b} are independently selected from halo, methyl, cyano and nitro; and
R₃ is selected from aryl, C₁₋₁₀heteroaryl and —C(O)OR₆ₐ; wherein R₆ₐ is selected from hydrogen, C₁₋₆alkyl and C₃₋₁₂cycloalkyl optionally substituted with C₁₋₄alkyl; wherein said heteroaryl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₈ₐ, —X₅ₐR₉, C₁₋₆alkyl, C₆alkoxy and halo-substituted-C₁₋₆alkyl; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl-C₀₋₄alkyl; wherein said aryl, heteroaryl, cycloalkyl or heterocycloalkyl of R₉ is optionally substituted with 1 to 3 radicals independently selected from halo, C₁₋₄alkyl and C₁₋₄alkoxy.

13. The compound of claim 12 in which L is selected from 3,5-1,2,4-oxadiazolylene, (1,2,4-oxadiazol-5-yl)methoxy, (1,2,4-oxadiazol-5-yl)methyl, (1,2,4-oxadiazol-5-yl)ethyl, (1,2,4-oxadiazol-5-yl)propyl, phenoxy, phenoxy-methyl, —C(O)NHCH₂—, —C(O)NH(CH₂)₂—, —CH₂OCH₂—, —C(O)NH(CH₂)₃—, —CH((CH₂)₂OH)(CH₂)₃—, —CH(CH₂C(O)OCH₃)(CH₂)₃—, —C(O)(CH₂)₃—, —CH(OH)(CH₂)₃—, —CH(Cl)(CH₂)₃—, —C(CH₃)(OH)(CH₂)₃—, —CH(N(CH₃)₂)(CH₂)₃—, —CH(NH₂)(CH₂)₃—, —CH(NHC(O)H)(CH₂)₃—, —CF₂(CH₂)₃—, —O(CH₂)₂—, —(CH₂)₂—, —(CH₂)₃—, —(CH₂)₄—, —O(CH₂)₄—, —O(CH₂)₃—, —NH(CH₂)₂—, —NH(CH₂)₃—, —C(═NOCH₃)(CH₂)₃—, —C(═NOH)(CH₂)₃—, —NHC(O)(CH₂)₃—, —NH(CH₂)₄—, —NCH₃(CH₂)₄—, —N(C(O)CH₃)(CH₂)₃—, —NC₂H₅(CH₂)₃—, —NC₃H₇(CH₂)₃—, —O(CH₂)₃O—, —O(CH₂)₂O—, —CH═CH(CH₂)₂—; —CH═CH—; —OCH₂CH(CH₂OH)O—; —C(O)CH(N(CH₂)₂—O—(CH₂)₂)—(CH₂)₂—; —NCH₃(CH₂)₃—; —N(CH(CH₃)₂)(CH₂)₃—; —NHC(O)(CH₂)₂—; —CH₂—O—(CH₂)₂—; —CH₂—O—(CH₂)₃—; —CH₂—O—(CH₂)₄—; —CH═CHCH₂—; —CH(CH₂COOH)(CH₂)₃—; —CH(OCH₃)(CH₂)₃—; —CH(F)(CH₂)₃—; —C(OH)(CH₂OH)(CH₂)₃—; —CH(CH₂OH)(CH₂)₃—;

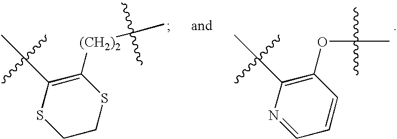

14. The compound of claim 13 in which R₁ is selected from methyl-sulfonyl, butyl-sulfonyl, phenyl-sulfonyl, isopropyl-sulfonyl, ethyl-sulfonyl, ethenyl-sulfonyl, methyl-sulfonyl-ethyl, isopropoxy-carbonyl, benzyloxy-carbonyl, ethoxy-carbonyl, methoxy-carbonyl, t-butoxy-carbonyl and trifluoromethyl-sulfonyl.

15. The compound of claim 14 in which In a further embodiment, R₃ is selected from t-butoxy-carbonyl, dimethylamino-carbonyl, methyl-sulfonyl, isopropoxy-carbonyl(ethyl)amino-methyl, isopropoxy-carbonyl-amino-methyl, benzyl(ethyl)amino-methyl, piperidinyl, quinazolinyl, isopropoxy-carbonyl, thieno[2,3-d]pyrimidin-4-yl, 4H-1,2,4-triazolyl, cyclopropoxy-carbonyl, (1,2,4-oxadiazol-5-yl), tetrazolyl, thiazolyl, triazolyl, pyrimidinyl, pyrazinyl, pyridinyl, phenyl, benzimidazolyl and pyridazinyl; wherein said cyclopropoxy, quinazolinyl, thieno[2,3-d]pyrimidinyl, thiazolyl, oxadiazolyl, tetrazolyl, pyrimidinyl, pyrazinyl, pyridinyl, phenyl, benzimidazolyl or pyridazinyl can be optionally substituted by 1 to 2 radicals independently selected from halo, cyano, methyl, methoxy-carbonyl, carboxyl, isopropyl, t-butyl, cyclopropyl, morpholino, methyl-piperazinyl-methyl, morpholino-methyl, ethoxy-methoxy-methyl, hydroxy-methyl, methoxy-ethoxy-methyl, methoxy-methoxy-methyl, ethoxy, trifluoromethyl, pentyl, phenyl, methoxy, dimethylamino, dimethylamino-methyl, dimethylamino-ethyl, aminoethyl, methoxy-carbonyl-methyl, methoxy-ethyl, hydroxyl-ethyl, pyrrolidinoethyl, t-butoxycarbonylamino-propoxy-methyl, morpholino-ethyl, aminopropoxy-methyl, dimethylamino-methyl, diethylamino-methyl, isopropyl-piperazino-ethyl, methoxy-ethoxy-ethoxy-methyl, methoxy-methyl, propyl and ethyl.

16. The compound of claim 15 selected from: 2-(5-bromopyrimidin-2-yl)-6-((2-(methylsulfonyl)-1,2,3,4-tetrahydroisoquinolin-6-yloxy)methyl)-1,2,3,4-tetrahydroisoquinoline; 2-(methylsulfonyl)-6-((2-(pyrazin-2-yl)-1,2,3,4-tetrahydroisoquinolin-6-yl)methoxy)-1,2,3,4-tetrahydroisoquinoline; 2-(6-((2-(methylsulfonyl)-1,2,3,4-tetrahydroisoquinolin-6-yloxy)methyl)-3,4-dihydroisoquinolin-2(1H)-yl)quinazoline; 2-(methylsulfonyl)-6-((2-(pyrimidin-2-yl)-1,2,3,4-tetrahydroisoquinolin-6-yl)methoxy)-1,2,3,4-tetrahydroisoquinoline; tert-butyl 6-((2-(methylsulfonyl)-1,2,3,4-tetrahydroisoquinolin-6-yloxy)methyl)-3,4-dihydroisoquinoline-2(1H)-carboxylate; isopropyl 6-((2-(methylsulfonyl)-1,2,3,4-tetrahydroisoquinolin-6-yloxy)methyl)-3,4-dihydroisoquinoline-2(1H)-carboxylate; 2-(5-ethylpyrimidin-2-yl)-6-((2-(methylsulfonyl)-1,2,3,4-tetrahydroisoquinolin-6-yloxy)methyl)-1,2,3,4-tetrahydroisoquinoline; isopropyl 6-(3-(2-(methylsulfonyl)-1,2,3,4-tetrahydroisoquinolin-6-yl)-1,2,4-oxadiazol-5-yl)-3,4-dihydroisoquinoline-2(1H)-carboxylate; and 5-(2-(methylsulfonyl)-1,2,3,4-tetrahydroisoquinolin-6-yl)-3-((2-(5-(trifluoromethyl)pyridin-2-yl)-1,2,3,4-tetrahydroisoquinolin-6-yl)methyl)-1,2,4-oxadiazole.

17. The compound of claim 1 of Formula Id: 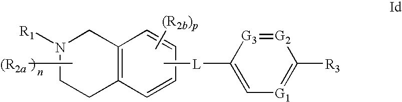
in which:
n and p are independently selected from 0, 1, 2 and 3;
L is selected from C₁₋₁₀heteroarylene, —X₂OX₃—, —OX₂X₃—, —C(O)X₂—, —X₂X₃—, —OX₂O—, —OX₂C(O)X₃—, —OX₂C(O)OX₃—, —CR₄(NR₄R₄)X₂—, —CR₄(NR₄C(O)R₄)X₂—, —C(═NOR₄)X₂—, —NR₄C(O)X₂—, —C(O)NR₄X₂—, —NR₄X₂—, —N(C(O)R₄)X₂— and —OC(O)NR₄X₂—; wherein X₂ and X₃ are independently selected from a bond, C₁₋₆alkylene, C₂₋₆alkenylene, C₆₋₁₀aryl, C₃₋₈cycloalkyl and C₁₋₁₀heteroarylene; R₄ is selected from hydrogen and C₁₋₆alkyl; wherein any methylene of L can have the hydrogens replaced by a radical selected from halo, hydroxy, C₁₋₄alkyl, C₁₋₄alkoxy, hydroxy-substituted-C₁₋₄alkyl and —CR₄R₄C(O)OR₄;
R₁ is selected from C₁₋₁₀alkyl, halo-substituted-C₁₋₁₀alkyl, C₆₋₁₀aryl, C₁₋₁₀heteroaryl, —S(O)₀₋₂R₅ₐ, —C(O)OR₅ₐ, —C(O)R₅ₐ, and —C(O)NR₅ₐR_{5b}; wherein R₅ₐ and R_{5b} are independently selected from hydrogen, C₁₋₆alkyl, C₃₋₁₂cycloalkyl, halo-substituted-C₁₋₆alkyl, C₆₋₁₀aryl-C₀₋₄alkyl and C₁₋₁₀heteroaryl; wherein said alkyl, cycloalkyl, aryl or heteroaryl of R₅ₐ or R_{5b} can be optionally substituted with 1 to 3 radicals independently selected from hydrogen, hydroxy, C₁₋₆alkyl, C₂₋₆alkenyl, halo-substituted-C₁₋₆alkyl, halo-substituted-C₁₋₆alkoxy-NR_{5c}R_{5d}, —C(O)OR_{5c} and C₆₋₁₀aryl-C₀₋₄alkyl; wherein R_{5c} and R_{5d} are independently selected from hydrogen and C₁₋₆alkyl;
R₂ₐ and R_{2b} are independently selected from halo, methyl, cyano and nitro;
G₁, G₂ and G₃ are independently selected from N and CH; with the proviso that at least one of G₁, G₂ or G₃ is N;
R₃ is selected from aryl, C₁₋₁₀heteroaryl and —C(O)OR₆ₐ; wherein R₆ₐ is selected from hydrogen, C₁₋₆alkyl and C₃₋₁₂cycloalkyl optionally substituted with C₁₋₄alkyl; wherein said heteroaryl of R₃ is optionally substituted with 1 to 3 radicals independently selected from halo, cyano, —X₅ₐNR₈ₐR_{8b}, —X₅ₐNR₈ₐR₉, —X₅ₐNR₈ₐC(O)OR_{8b}, —X₅ₐC(O)OR₈ₐ, —X₅ₐOR₈ₐ, —X₅ₐOX_{5b}OR₉ₐ, —X₅ₐR₉, C₁₋₆alkyl, C₁₋₆alkoxy and halo-substituted-C₁₋₆alkyl; wherein R₈ₐ and R_{8b} are independently selected from hydrogen and C₁₋₆alkyl; X₅ₐ and X_{5b} are independently selected from a bond and C₁₋₄alkylene; R₉ is selected from C₃₋₁₂cycloalkyl, C₃₋₈heterocycloalkyl, C₁₋₁₀heteroaryl and C₆₋₁₀aryl-C₀₋₄alkyl; wherein said aryl, heteroaryl, cycloalkyl or heterocycloalkyl of R₉ is optionally substituted with 1 to 3 radicals independently selected from halo, C₁₋₄alkyl and C₁₋₄alkoxy.

18. The compound of claim 17 in which L is selected from 3,5-1,2,4-oxadiazolylene, (1,2,4-oxadiazol-5-yl)methoxy, (1,2,4-oxadiazol-5-yl)methyl, (1,2,4-oxadiazol-5-yl)ethyl, (1,2,4-oxadiazol-5-yl)propyl, phenoxy, phenoxy-methyl, —C(O)NHCH₂—, —C(O)NH(CH₂)₂—, —CH₂OCH₂—, —C(O)NH(CH₂)₃—, —CH((CH₂)₂OH)(CH₂)₃—, —CH(CH₂C(O)OCH₃)(CH₂)₃—, —C(O)(CH₂)₃—, —CH(OH)(CH₂)₃—, —CH(CI)(CH₂)₃—, —C(CH₃)(OH)(CH₂)₃—, —CH(N(CH₃)₂)(CH₂)₃—, —CH(NH₂)(CH₂)₃—, —CH(NHC(O)H)(CH₂)₃—, —CF₂(CH₂)₃—, —O(CH₂)₂—, —(CH₂)₂—, —(CH₂)₃—, —(CH₂)₄—, —O(CH₂)₄—, —O(CH₂)₃—, —NH(CH₂)₂—, —NH(CH₂)₃—, —C(═NOCH₃)(CH₂)₃—, —C(═NOH)(CH₂)₃—, —NHC(O)(CH₂)₃—, —NH(CH₂)₄—, —NCH₃(CH₂)₄—, —N(C(O)CH₃)(CH₂)₃—, —NC₂H₅(CH₂)₃—, —NC₃H₇(CH₂)₃—, —O(CH₂)₃O—, —O(CH₂)₂O—, —CH═CH(CH₂)₂—; —CH═CH—; —OCH₂CH(CH₂OH)O—; —C(O)CH(N(CH₂)₂—O—(CH₂)₂)—(CH₂)₂—; —NCH₃(CH₂)₃—; —N(CH(CH₃)₂)(CH₂)₃—; —NHC(O)(CH₂)₂—; —CH₂—O—(CH₂)₂—; —CH₂—O—(CH₂)₃—; —CH₂—O—(CH₂)₄—; —CH═CHCH₂—; —CH(CH₂COOH)(CH₂)₃—; —CH(OCH₃)(CH₂)₃—; —CH(F)(CH₂)₃—; —C(OH)(CH₂OH)(CH₂)₃—; —CH(CH₂OH)(CH₂)₃—;

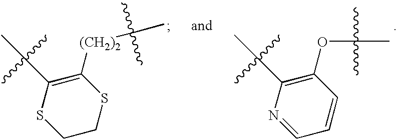

19. The compound of claim 18 in which R₁ is selected from methyl-sulfonyl, butyl-sulfonyl, phenyl-sulfonyl, isopropyl-sulfonyl, ethyl-sulfonyl, ethenyl-sulfonyl, methyl-sulfonyl-ethyl, isopropoxy-carbonyl, benzyloxy-carbonyl, ethoxy-carbonyl, methoxy-carbonyl, t-butoxy-carbonyl and trifluoromethyl-sulfonyl.

20. The compound of claim 19 in which In a further embodiment, R₃ is selected from t-butoxy-carbonyl, dimethylamino-carbonyl, methyl-sulfonyl, isopropoxy-carbonyl(ethyl)amino-methyl, isopropoxy-carbonyl-amino-methyl, benzyl(ethyl)amino-methyl, piperidinyl, quinazolinyl, isopropoxy-carbonyl, thieno[2,3-d]pyrimidin-4-yl, 4H-1,2,4-triazolyl, cyclopropoxy-carbonyl, (1,2,4-oxadiazol-5-yl), tetrazolyl, thiazolyl, triazolyl, pyrimidinyl, pyrazinyl, pyridinyl, phenyl, benzimidazolyl and pyridazinyl; wherein said cyclopropoxy, quinazolinyl, thieno[2,3-d]pyrimidinyl, thiazolyl, oxadiazolyl, tetrazolyl, pyrimidinyl, pyrazinyl, pyridinyl, phenyl, benzimidazolyl or pyridazinyl can be optionally substituted by 1 to 2 radicals independently selected from halo, cyano, methyl, methoxy-carbonyl, carboxyl, isopropyl, t-butyl, cyclopropyl, morpholino, methyl-piperazinyl-methyl, morpholino-methyl, ethoxy-methoxy-methyl, hydroxy-methyl, methoxy-ethoxy-methyl, methoxy-methoxy-methyl, ethoxy, trifluoromethyl, pentyl, phenyl, methoxy, dimethylamino, dimethylamino-methyl, dimethylamino-ethyl, aminoethyl, methoxy-carbonyl-methyl, methoxy-ethyl, hydroxyl-ethyl, pyrrolidinoethyl, t-butoxycarbonylamino-propoxy-methyl, morpholino-ethyl, aminopropoxy-methyl, dimethylamino-methyl, diethylamino-methyl, isopropyl-piperazino-ethyl, methoxy-ethoxy-ethoxy-methyl, methoxy-methyl, propyl and ethyl.

21. The compound of claim 20 selected from: 6-(3-(2-(4-ethylpiperidin-1-yl)pyrimidin-5-yl)propoxy)-2-(methylsulfonyl)-1,2,3,4-tetrahydroisoquinoline; 2-(methylsulfonyl)-6-(3-(6-phenylpyridin-3-yl)propoxy)-1,2,3,4-tetrahydroisoquinoline; 2-(methylsulfonyl)-6-(3-(5-phenylpyridin-2-yl)propoxy)-1,2,3,4-tetrahydroisoquinoline; 4-(5-(3-(2-(methylsulfonyl)-1,2,3,4-tetrahydroisoquinolin-6-yloxy)propyl)pyridin-2-yl)morpholino; 2-(Methylsulfonyl)-6-(3-(6-phenylpyridin-3-yl)propoxy)-1,2,3,4-tetrahydroisoquinoline; 2-(Methylsulfonyl)-6-(3-(5-phenylpyridin-2-yl)propoxy)-1,2,3,4-tetrahydroisoquinoline; and 4-(5-(3-(2-(Methylsulfonyl)-1,2,3,4-tetrahydroisoquinolin-6-yloxy)propyl)pyridin-2-yl)morpholine.

22. A pharmaceutical composition comprising a therapeutically effective amount of a compound of claim 1 in combination with a pharmaceutically acceptable excipient.

23. A method for modulating GPR119 activity, comprising administering to a system or a subject in need thereof, a therapeutically effective amount of the compound of claim 1 or pharmaceutically acceptable salts or pharmaceutical compositions thereof, thereby modulating said GPR119 activity.

24. The method of claim 23, wherein the compound of claim 1 directly contacts GPR119.

25. The method of claim 24, wherein the contacting occurs in vitro or in vivo.

26. A method for treating a disease or condition wherein modulation of GPR119 activity can prevent, inhibit or ameliorate the pathology and/or symptomology of the disease or condition, comprising administering to a subject a therapeutically effective amount of the compound of claim 1 or pharmaceutically acceptable salts or pharmaceutical compositions thereof.

27. The method of claim 26, wherein said disease or condition is selected from obesity, type 1 diabetes, type 2 diabetes mellitus, hyperlipidemia, idiopathic type 1 diabetes, latent autoimmune diabetes in adults, early-onset type 2 diabetes, youth-onset atypical diabetes, maturity onset diabetes of the young, malnutrition-related diabetes and gestational diabetes.

28. The method of claim 26, wherein said disease or condition is selected from coronary heart disease, ischemic stroke, restenosis after angioplasty, peripheral vascular disease, intermittent claudication, myocardial infarction, dyslipidemia, post-prandial lipemia, conditions of impaired glucose tolerance, conditions of impaired fasting plasma glucose, metabolic acidosis, ketosis, arthritis, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.
